SEQUENCE LISTING (56 sequences)

SEQ ID NO: 1 - protein, 86 aa, Homo sapiens
  source 1..86: note = HMG domain WT human Sox 1
DRVKRPMNAFMVWSRGQRRKMAQENPKMHNSEISKRLGAEWKVMSEAEKRPFIDEAKRLRALHMKEHPDYKYRPRRKTKTLLKKDK

SEQ ID NO: 2 - protein, 86 aa, Homo sapiens
  source 1..86: note = HMG domain WT human Sox 2
DRVKRPMNAFMVWSRGQRRKMAQENPKMHNSEISKRLGAEWKLLSETEKRPFIDEAKRLRALHMKEHPDYKYRPRRKTKTLMKKDK

SEQ ID NO: 3 - protein, 86 aa, Homo sapiens
  source 1..86: note = HMG domain WT human Sox 3
DRVKRPMNAFMVWSRGQRRKMALENPKMHNSEISKRLGADWKLLTDAEKRPFIDEAKRLRAVHMKEYPDYKYRPRRKTKTLLKKDK

SEQ ID NO: 4 - protein, 391 aa, Homo sapiens
  source 1..391: note = WT human Sox 1
MYSMMMETDLHSPGGAQAPTNLSGPAGAGGGGGGGGGGGGGGGAKANQDRVKRPMNAFMVWSRGQRRKMAQENPKMHNSEISKRLGAEWKVMSEAEKRPFIDEAKRLRALHMKEHPDYKYRPRRKTKTLLKKDKYSLAGGLLAAGAGGGGAAVAMGVGVGVGAAAVGQRLESPGGAAGGGYAHVNGWANGAYPGSVAAAAAAAAMMQEAQLAYGQHPGAGGAHPHAHPAHPHPHHPHAHPHNPQPMHRYDMGALQYSPISNSQGYMSASPSGYGGLPYGAAAAAAAAAGGAHQNSAVAAAAAAAAASSGALGALGSLVKSEPSGSPPAPAHSRAPCPGDLREMISMYLPAGEGGDPAAAAAAAAQSRLHSLPQHYQGAGAGVNGTVPLTHI

SEQ ID NO: 5 - protein, 317 aa, Homo sapiens
  source 1..317: note = WT human Sox 2
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 6 - protein, 446 aa, Homo sapiens
  source 1..446: note = WT human Sox 3
MRPVRENSSGARSPRVPADLARSILISLPFPPDSLAHRPPSSAPTESQGLFTVAAPAPGAPSPPATLAHLLPAPAMYSLLETELKNPVGTPTQAAGTGGPAAPGGAGKSSANAAGGANSGGGSSGGASGGGGGTDQDRVKRPMNAFMVWSRGQRRKMALENPKMHNSEISKRLGADWKLLTDAEKRPFIDEAKRLRAVHMKEYPDYKYRPRRKTKTLLKKDKYSLPSGLLPPGAAAAAAAAAAAAAAASSPVGVGQRLDTYTHVNGWANGAYSLVQEQLGYAQPPSMSSPPPPPALPPMHRYDMAGLQYSPMMPPGAQSYMNVAAAAAAASGYGGMAPSATAAAAAAYGQQPATAAAAAAAAAAMSLGPMGSVVKSEPSSPPPAIASHSQRACLGDLRDMISMYLPPGGDAADAASPLPGGRLHGVHQHYQGAGTAVNGTVPLTHI

SEQ ID NO: 7 - protein, 5 aa, Homo sapiens
  source 1..5: note = positions 43-47 of WT human SOX17 HMG
ALTLA

SEQ ID NO: 8 - protein, 22 aa, Homo sapiens
  source 1..22: note = positions 65-86 of WT SOX17
QDHPNYKYRPRRRKQVKRLKRV

SEQ ID NO: 9 - protein, 263 aa, Homo sapiens
  source 1..263: note = CTD of Sox17
EGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 10 - protein, 391 aa, synthetic construct
  source 1..391: note = Sox1A61V human
MYSMMMETDLHSPGGAQAPTNLSGPAGAGGGGGGGGGGGGGGGAKANQDRVKRPMNAFMVWSRGQRRKMAQENPKMHNSEISKRLGAEWKVMSEAEKRPFIDEAKRLRVLHMKEHPDYKYRPRRKTKTLLKKDKYSLAGGLLAAGAGGGGAAVAMGVGVGVGAAAVGQRLESPGGAAGGGYAHVNGWANGAYPGSVAAAAAAAAMMQEAQLAYGQHPGAGGAHPHAHPAHPHPHHPHAHPHNPQPMHRYDMGALQYSPISNSQGYMSASPSGYGGLPYGAAAAAAAAAGGAHQNSAVAAAAAAAAASSGALGALGSLVKSEPSGSPPAPAHSRAPCPGDLREMISMYLPAGEGGDPAAAAAAAAQSRLHSLPQHYQGAGAGVNGTVPLTHI

SEQ ID NO: 11 - protein, 317 aa, synthetic construct
  source 1..317: note = Sox2A61V human
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRVLHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 12 - protein, 446 aa, synthetic construct
  source 1..446: note = Sox3A61V human
MRPVRENSSGARSPRVPADLARSILISLPFPPDSLAHRPPSSAPTESQGLFTVAAPAPGAPSPPATLAHLLPAPAMYSLLETELKNPVGTPTQAAGTGGPAAPGGAGKSSANAAGGANSGGGSSGGASGGGGGTDQDRVKRPMNAFMVWSRGQRRKMALENPKMHNSEISKRLGADWKLLTDAEKRPFIDEAKRLRVVHMKEYPDYKYRPRRKTKTLLKKDKYSLPSGLLPPGAAAAAAAAAAAAAAASSPVGVGQRLDTYTHVNGWANGAYSLVQEQLGYAQPPSMSSPPPPPALPPMHRYDMAGLQYSPMMPPGAQSYMNVAAAAAAASGYGGMAPSATAAAAAAYGQQPATAAAAAAAAAAMSLGPMGSVVKSEPSSPPPAIASHSQRACLGDLRDMISMYLPPGGDAADAASPLPGGRLHGVHQHYQGAGTAVNGTVPLTHI

SEQ ID NO: 13 - protein, 387 aa, synthetic construct
  source 1..387: note = SOX2-17 (super-SOX, S*) human
MYNMMETELKPPGPQQTSGGGGGNSTAAAAGGNQKNSPDRVKRPMNAFMVWSRGQRRKMAQENPKMHNSEISKRLGAEWKALTLAEKRPFIDEAKRLRVLHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 14 - protein, 394 aa, synthetic construct
  source 1..394: note = Sox2-17 (super-SOX, S*) mouse
MYNMMETELKPPGPQQASGGGGGGGNATAAATGGNQKNSPDRVKRPMNAFMVWSRGQRRKMAQENPKMHNSEISKRLGAEWKALTLAEKRPFIDEAKRLRVLHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAPPPPPQQQHPAHGPGQPSPPPEALPCRDGTESNQPTELLGEVDRTEFEQYLPFVYKPEMGLPYQGHDCGVNLSDSHGAISSVVSDASSAVYYCNYPDI

SEQ ID NO: 15 - protein, 86 aa, Homo sapiens
  source 1..86: note = Human SOX-17 HMG
SRIRRPMNAFMVWAKDERKRLAQQNPDLHNAELSKMLGKSWKALTLAEKRPFVEEAERLRVQHMQDHPNYKYRPRRRKQVKRLKRV

SEQ ID NO: 16 - protein, 5 aa, Homo sapiens
  source 1..5: note = positions 24 to 28 of WT human SOX2
ENPKM

SEQ ID NO: 17 - protein, 152 aa, Homo sapiens
  source 1..152: note = POU domain human Oct4
DIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSS

SEQ ID NO: 18 - protein, 152 aa, Mus musculus
  source 1..152: note = POU domain mouse Oct4
DMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSS

SEQ ID NO: 19 - protein, 360 aa, Homo sapiens
  source 1..360: note = human WT Oct 4
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 20 - protein, 352 aa, Mus musculus
  source 1..352: note = mouse WT Oct 4
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 21 - protein, 282 aa, synthetic construct
  source 1..282: note = Oct4DeltaCTD mouse
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSS

SEQ ID NO: 22 - protein, 297 aa, synthetic construct
  source 1..297: note = OctDeltaPOUHD mouse
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRIEYSQREEYEATGTPFPGGAVSFPLPPGPHFGTPGYGSPHFTTLYSVPFPEGEAFPSVPVTALGSPMHSN

SEQ ID NO: 23 - protein, 223 aa, synthetic construct
  source 1..223: note = Oct4DeltaNTD mouse
MDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 24 - protein, 352 aa, synthetic construct
  source 1..352: note = Oct4-L80A mouse
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENAQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 25 - protein, 479 aa, Homo sapiens
  source 1..479: note = Oct2 human
MVHSSMGAPEIRMSKPLEAEKQGLDSPSEHTDTERNGPDTNHQNPQNKTSPFSVSPTGPSTKIKAEDPSGDSAPAAPLPPQPAQPHLPQAQLMLTGSQLAGDIQQLLQLQQLVLVPGHHLQPPAQFLLPQAQQSQPGLLPTPNLFQLPQQTQGALLTSQPRAGLPTQAVTRPTLPDPHLSHPQPPKCLEPPSHPEEPSDLEELEQFARTFKQRRIKLGFTQGDVGLAMGKLYGNDFSQTTISRFEALNLSFKNMCKLKPLLEKWLNDAETMSVDSSLPSPNQLSSPSLGFDGLPGRRRKKRTSIETNVRFALEKSFLANQKPTSEEILLIAEQLHMEKEVIRVWFCNRRQKEKRINPCSAAPMLPSPGKPASYSPHMVTPQGGAGTLPLSQASSSLSTTVTTLSSAVGTLHPSRTAGGGGGGGGAAPPLNSIPSVTPPPPATTNSTNPSPQGSHSAIGLSGLNPSTGPGLWWNPAPYQP

SEQ ID NO: 26 - protein, 463 aa, Mus musculus
  source 1..463: note = Oct2 mouse
MVHSSMGAPEIRMSKPLEAEKQSLDSPSEHTDTERNGPDINHQNPQNKASPFSVSPTGPSTKIKAEDPSGDSAPAAPPPPQPAQPHLPQAQLMLTGSQLAGDIQQLLQLQQLVLVPGHHLQPPAQFLLPQAQQSQPGLLPTPNLFQLPQQTQGALLTSQPRAGLPTQPPKCLEPPSHPEEPSDLEELEQFARTFKQRRIKLGFTQGDVGLAMGKLYGNDFSQTTISRFEALNLSFKNMCKLKPLLEKWLNDAETMSVDSSLPSPNQLSSPSLGFDGLPGRRRKKRTSIETNVRFALEKSFLANQKPTSEEILLIAEQLHMEKEVIRVWFCNRRQKEKRINPCSAAPMLPSPGKPTSYSPHLVTPQGGAGTLPLSQASSSLSTTVTTLSSAVGTLHPSRTAGGGGGGGGAAPPLNSIPSVTPPPPATTNSTNPSPQGSHSAIGLSGLNPSAGPGLWWNPAPYQP

SEQ ID NO: 27 - protein, 451 aa, Homo sapiens
  source 1..451: note = Oct6 human
MATTAQYLPRGPGGGAGGTGPLMHPDAAAAAAAAAAAERLHAGAAYREVQKLMHHEWLGAGAGHPVGLAHPQWLPTGGGGGGDWAGGPHLEHGKAGGGGTGRADDGGGGGGFHARLVHQGAAHAGAAWAQGSTAHHLGPAMSPSPGASGGHQPQPLGLYAQAAYPGGGGGGLAGMLAAGGGGAGPGLHHALHEDGHEAQLEPSPPPHLGAHGHAHGHAHAGGLHAAAAHLHPGAGGGGSSVGEHSDEDAPSSDDLEQFAKQFKQRRIKLGFTQADVGLALGTLYGNVFSQTTICRFEALQLSFKNMCKLKPLLNKWLEETDSSSGSPTNLDKIAAQGRKRKKRTSIEVGVKGALESHFLKCPKPSAHEITGLADSLQLEKEVVRVWFCNRRQKEKRMTPAAGAGHPPMDDVYAPGELGPGGGGASPPSAPPPPPPAALHHHHHHTLPGSVQ

SEQ ID NO: 28 - protein, 449 aa, Mus musculus
  source 1..449: note = Oct6 mouse
MATTAQYLPRGPGGGAGGTGPLMHPDAAAAAAAAAERLHAGAAYREVQKLMHHEWLGAGAGHPVGLAHPQWLPTGGGGGGDWAGGPHLEHGKAGGGGTGRADDGGGGGGFHARLVHQGAAHAGAAWAQGGTAHHLGPAMSPSPGAGGGHQPQPLGLYAQAAYPGGGGGGLAGMLAAGGGGAGPGLHHALHEDGHEAQLEPSPPPHLGAHGHAHGHAHAGGLHAAAAHLHPGAGGGGSSVGEHSDEDAPSSDDLEQFAKQFKQRRIKLGFTQADVGLALGTLYGNVFSQTTICRFEALQLSFKNMCKLKPLLNKWLEETDSSSGSPTNLDKIAAQGRKRKKRTSIEVGVKGALESHFLKCPKPSAHEITGLADSLQLEKEVVRVWFCNRRQKEKRMTPAAGAGHPPMDDVYAPGELGPGGGSASPPSAPPPPPPAALHHHHHHTLPGSVQ

SEQ ID NO: 29 - protein, 443 aa, Homo sapiens
  source 1..443: note = Brn2 human
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 30 - protein, 445 aa, Mus musculus
  source 1..445: note = Brn2 mouse
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 31 - protein, 361 aa, Homo sapiens
  source 1..361: note = Brn4 human
MATAASNPYSILSSTSLVHADSAGMQQGSPFRNPQKLLQSDYLQGVPSNGHPLGHHWVTSLSDGGPWSSTLATSPLDQQDVKPGREDLQLGAIIHHRSPHVAHHSPHTNHPNAWGASPAPNPSITSSGQPLNVYSQPGFTVSGMLEHGGLTPPPAAASAQSLHPVLREPPDHGELGSHHCQDHSDEETPTSDELEQFAKQFKQRRIKLGFTQADVGLALGTLYGNVFSQTTICRFEALQLSFKNMCKLKPLLNKWLEEADSSTGSPTSIDKIAAQGRKRKKRTSIEVSVKGVLETHFLKCPKPAAQEISSLADSLQLEKEVVRVWFCNRRQKEKRMTPPGDQQPHEVYSHTVKTDTSCHDL

SEQ ID NO: 32 - protein, 361 aa, Mus musculus
  source 1..361: note = Brn4 mouse
MATAASNPYSILSSSSLVHADSAGMQQGSPFRNPQKLLQSDYLQGVPSNGHPLGHHWVTSLSDGGPWSSTLATSPLDQQDVKPGREDLQLGAIIHHRSPHVAHHSPHTNHPNAWGASPAPNSSITSSGQPLNVYSQPGFTVSGMLEHGGLTPPPAAASTQSLHPVLREPPDHGELGSHHCQDHSDEETPTSDELEQFAKQFKQRRIKLGFTQADVGLALGTLYGNVFSQTTICRFEALQLSFKNMCKLKPLLNKWLEEADSSTGSPTSIDKIAAQGRKRKKRTSIEVSVKGVLETHFLKCPKPAAQEISSLADSLQLEKEVVRVWFCNRRQKEKRMTPPGDQQPHEVYSHTVKTDASCHDL

SEQ ID NO: 33 - protein, 470 aa, Homo sapiens
  source 1..470: note = Krueppel-like factor 4
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 34 - protein, 474 aa, Mus musculus
  source 1..474: note = Krueppel-like factor 4
MAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 35 - DNA, 10 bp, synthetic construct
  source 1..10: mol_type = other DNA; note = consensus sequence of p53-responsive element
  variation 1: replace = g
  variation 2: replace = g
  variation 3: replace = g
  variation 5: replace = t
  variation 6: replace = a
  variation 8: replace = t
  variation 9: replace = t
  variation 10: replace = t
aaagatcccc

SEQ ID NO: 36 - protein, 209 aa, Homo sapiens
  source 1..209: note = >NP_078950.1 protein lin-28 homolog A
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 37 - protein, 209 aa, Mus musculus
  source 1..209: note = >NP_665832.1 protein lin-28 homolog A
MGSVSNQQFAGGCAKAAEKAPEEAPPDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKNMQKRRSKGDRCYNCGGLDHHAKECKLPPQPKKCHFCQSINHMVASCPLKAQQGPSSQGKPAYFREEEEEIHSPALLPEAQN

SEQ ID NO: 38 - protein, 305 aa, Homo sapiens
  source 1..305: note = >NP_079141.2 homeobox protein NANOG isoform 1
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 39 - protein, 305 aa, Mus musculus
  source 1..305: note = >NP_082292.1 homeobox protein NANOG isoform 1
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI

SEQ ID NO: 40 - protein, 620 aa, Homo sapiens
  source 1..620: note = >NP_671726.2 zinc finger protein GLIS1 isoform 1
MAEARTSLSAHCRGPLATGLHPDLDLPGRSLATPAPSCYLLGSEPSSGLGLQPETHLPEGSLKRCCVLGLPPTSPASSSPCASSDVTSIIRSSQTSLVTCVNGLRSPPLTGDLGGPSKRARPGPASTDSHEGSLQLEACRKASFLKQEPADEFSELFGPHQQGLPPPYPLSQLPPGPSLGGLGLGLAGRVVAGRQACRWVDCCAAYEQQEELVRHIEKSHIDQRKGEDFTCFWAGCVRRYKPFNARYKLLIHMRVHSGEKPNKCMFEGCSKAFSRLENLKIHLRSHTGEKPYLCQHPGCQKAFSNSSDRAKHQRTHLDTKPYACQIPGCSKRYTDPSSLRKHVKAHSAKEQQVRKKLHAGPDTEADVLTECLVLQQLHTSTQLAASDGKGGCGLGQELLPGVYPGSITPHNGLASGLLPPAHDVPSRHHPLDATTSSHHHLSPLPMAESTRDGLGPGLLSPIVSPLKGLGPPPLPPSSQSHSPGGQPFPTLPSKPSYPPFQSPPPPPLPSPQGYQGSFHSIQSCFPYGDCYRMAEPAAGGDGLVGETHGFNPLRPNGYHSLSTPLPATGYEALAEASCPTALPQQPSEDVVSSGPEDCGFFPNGAFDHCLGHIPSIYTDT

SEQ ID NO: 41 - protein, 454 aa, Homo sapiens
  source 1..454: note = >NP_002458.2 myc proto-oncogene protein isoform 1
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 42 - protein, 439 aa, Mus musculus
  source 1..439: note = >NP_001170823.1 myc proto-oncogene protein isoform b
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 43 - protein, 364 aa, Homo sapiens
  source 1..364: note = >NP_001028253.1 protein L-Myc isoform 1
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 44 - protein, 816 aa, synthetic construct
  source 1..816: note = >human SOX2-P2A-KLF4 cassette for low-grade to high-grade pluripotency conversion
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMGTGATNFSLLKQAGDVEENPGPMNNSPFTMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 45 - protein, 816 aa, synthetic construct
  source 1..816: note = >human SOX2A61V-P2A-KLF4 cassette for low-grade to high-grade pluripotency conversion
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRVLHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMGTGATNFSLLKQAGDVEENPGPMNNSPFTMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 46 - protein, 886 aa, synthetic construct
  source 1..886: note = >human SOX2-17-P2A-KLF4 (S*K) cassette for low-grade to high-grade pluripotency conversion
MYNMMETELKPPGPQQTSGGGGGNSTAAAAGGNQKNSPDRVKRPMNAFMVWSRGQRRKMAQENPKMHNSEISKRLGAEWKALTLAEKRPFIDEAKRLRVLHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDVGTGATNFSLLKQAGDVEENPGPMNNSPFTMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 47 - protein, 319 aa, Mus musculus
  source 1..319: note = Mouse Sox2A61V
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRVLHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM

SEQ ID NO: 48 - DNA, 46 bp, synthetic construct
  source 1..46: mol_type = other DNA; note = DNA Oligo Fgf4
ggagaagaaaactctttgtttggatgctaatgggatactaagctcc

SEQ ID NO: 49 - DNA, 38 bp, synthetic construct
  source 1..38: mol_type = other DNA; note = DNA Oligo HoxB1
ggaggaagtgtctttgtcatgctaatgattggggctcc

SEQ ID NO: 50 - DNA, 37 bp, synthetic construct
  source 1..37: mol_type = other DNA; note = DNA Oligo Nanog
tccaccatggacattgtaatgcaaaagaagctgtaag

SEQ ID NO: 51 - DNA, 37 bp, synthetic construct
  source 1..37: mol_type = other DNA; note = DNA Oligo Oct4DE
ctatcatgcacctttgttatgcatctgccgtctgccc

SEQ ID NO: 52 - DNA, 48 bp, synthetic construct
  source 1..48: mol_type = other DNA; note = DNA Oligo Utf1
ggagaagatgagagccctcattgttatgctagtgaagtgccaagctcc

SEQ ID NO: 53 - DNA, 27 bp, synthetic construct
  source 1..27: mol_type = other DNA; note = DNA Oligo non-specific
ctgcaggtgggattaactgtgaattca

SEQ ID NO: 54 - DNA, 147 bp, synthetic construct
  source 1..147: mol_type = other DNA; note = DNA Oligo Widom+6
ctggagaatcccggtctgcaggccgctcaattggtcgtagacagctctagcaccgcttaaacgcacgtacgcgctgtcccccgcgttttaaccgccaaggggattactccctagtctccaggcctttgttatgcaaatacatcctgt

SEQ ID NO: 55 - DNA, 15 bp, synthetic construct
  source 1..15: mol_type = other DNA; note = Sox/Oct motif
ctttgttatgcaaat

SEQ ID NO: 56 - DNA, 49 bp, synthetic construct
  source 1..49: mol_type = other DNA; note = HoxBI DNA locus for MDS
agagtgattgaagtgtctttgtcatgctaatgattggggggagatggat

(19) United States
(12) Patent Application Publication
(10) Pub. No.: US 2026/0347422 A1
(43) Pub. Date: Nov. 5, 2026

(54) ENGINEERING SOX/OCT HETERODIMERIZATION TO INDUCE HIGH-GRADE DEVELOPMENTAL RESET

(71) Applicant: Max-Planck-Gesellschaft zur Förderung der Wissenschaften e.V., München (DE)

(72) Inventors: Sergiy VELYCHKO, Münster (DE); Hans R. SCHÖLER, Münster (DE); Caitlin M. MACCARTHY, Münster (DE); Vlad COJOCARU, Münster (DE); Guangming WU, Münster (DE)

(21) Appl. No.: 19/109,116

(22) PCT Filed: Sep. 6, 2023
(86) PCT No.: PCT/EP2023/074510

§ 371 Date: Mar. 5, 2025

(30) Foreign Application Priority Data
Sep. 6, 2022 (EP) 22194243.6
May 15, 2023 (EP) 23173488.0
Aug. 7, 2023 (EP) 23190084.6

(51) Int. Cl. C07K 14/475; A01K 67/00; A61K 35/545; C12N 5/073; C12N 5/074; C12N 15/63

(52) U.S. Cl. CPC C07K 14/475; A01K 67/00; A61K 35/545; C12N 5/0604; C12N 5/0696; C12N 15/63; C07K 2319/00; C12N 2501/602; C12N 2501/603; C12N 2510/00; C12N 2517/00

(57) ABSTRACT

The present invention relates to a SoxB1 factor variant comprising (a) the HMG (high-mobility group) domain of any one of the amino acid sequences of SEQ ID NOs: 1 to 3, wherein the amino acid alanine at position 61 of the HMG-domain is substituted with an amino acid selected from valine, leucine, isoleucine, phenylalanine, methionine, tryptophan or proline, preferably valine; or (b) an amino acid sequence sharing at least 82% sequence identity with the HMG domain as defined in (a), provided that the substitution as defined in (a) is retained.

The present invention relates to a SoxB1 factor variant comprising a) the HMG (high-mobility group) domain of any one of the amino acid sequences of SEQ ID NOs: 1 to 3, wherein the amino acid alanine at position 61 of the HMG-domain is substituted with an amino acid selected from valine, leucine, isoleucine, phenylalanine, methionine, tryptophan or proline, preferably valine; or b) an amino acid sequence sharing at least 82% sequence identity with the HMG domain as defined in a), provided that the substitution as defined in a) is retained.

In this specification, a number of documents are cited. The disclosure of these documents, while not considered relevant for the patentability of this invention is herewith incorporated by reference in its entirety. More specifically, all referenced documents are incorporated by reference to the same extent as if each individual document was specifically and individually indicated to be incorporated by reference.

The ability of some transcription factors, such as MyoD, to convert cell fates was known since 1987 (Davis et al., 1987). It was not until 2006 that the discovery of induced pluripotent stem cells (iPSCs) by Shinya Yamanaka brought transcription factor-based cell fate conversion into the spotlight, and rightly so (Takahashi and Yamanaka, 2006). Pluripotent cells are unique in their ability to give rise to all the tissues of an animal. The induction of pluripotency amounts to the ultimate cell rejuvenation, once thought to be impossible (Waddington, 1957). iPSC technology has already made enormous contributions to human developmental biology studies, allowed new strategies for drug discovery, and even provided a source for cell replacement therapy. However, the most radical endeavour is perhaps yet to come: recent studies by Belmonte's lab and others demonstrated the ability of reprogramming factors to reverse aging on the whole-organism level in mice (Browder et al., 2022; Chen et al., 2021; Chondronasiou et al., 2022; Lu et al., 2020; Ocampo et al., 2016; Sarkar et al., 2020), and the Reik lab showed that time-restricted reprogramming factor induction could rejuvenate cultured human cells (Gill et al., 2022).

Oct4, Sox2, Klf4, and cMyc (OSKM)—all components of the Yamanaka cocktail, control the pluripotency of the inner cell mass of the blastocyst (Nakatake et al., 2006; Wu and Schöler, 2014). This stage of mammalian development is transient: soon after emerging, the pluripotent cells of the inner cell mass commit to unidirectional differentiation. Pluripotent cells may be artificially expanded from the inner cell mass in the form of embryonic stem cells (ESCs) (Thomson et al., 1998). Oct4, Sox2, and Klf4 (OSK) are pioneer transcription factors capable of opening silent chromatin, which allows cells to maintain the plastic pluripotent state, or initiate differentiation later (King and Klose, 2017). The iPSC technology harnesses the pioneering ability of OSK to drive cell identity into the opposite direction (Soufi et al., 2012). Oct4 stands out as the master regulator of the pluripotency network. Oct4 is the only factor for which knock out in ESCs leads to an inevitable collapse of pluripotency; forced expression of Oct4 can even compensate for the loss of Sox2 (Masui et al., 2007; Nishimoto et al., 2005; Niwa et al., 2002). Oct4 is the only reprogramming factor that cannot be replaced by other members of its family (Nakagawa et al., 2008). At the same time, it was shown that overexpression of Oct4 during reprogramming leads to aberrant epigenetic changes in mouse iPSCs, worsening the developmental potential of OSKM versus SKM iPSCs (Velychko et al., 2019a). Oct4 plays divergent roles in establishing pluripotency during mouse and human development: Oct4 knockout mouse blastocysts still develop a Nanog⁺ inner cell mass, while human OCT4-null blastocysts fail to do so (Fogarty et al., 2017; Wu et al., 2013). Correspondingly, SKM induction is sufficient to induce pluripotency in mouse somatic cells (An et al., 2019; Velychko et al., 2019a), however, SKM reprogramming has not been demonstrated for human or other species.

Oct4 cooperates with Sox2 to co-regulate the majority of its targets in pluripotent cells (Chen et al., 2008). Oct4/Sox2 cooperativity is mediated by DNA allostery and by protein-protein interaction between their DNA-binding domains (Merino et al., 2015). In the beginning of reprogramming to iPSCs, when native Oct4 and Sox2 sites are inaccessible each factor often binds independently (Soufi et al., 2012), yet the sites engaged by both factors are still more likely to open (Chronis et al., 2017; Malik et al., 2019). Indeed, Oct4/Sox2 cooperativity, particularly on Hoxb1-like canonical SoxOct motifs, was shown to be essential for the induction and maintenance of pluripotency (Tapia et al., 2015). On the other hand, Sox17 cooperates with Oct4 on compressed, non-canonical SoxOct motifs and cannot induce pluripotency but rather controls primitive endoderm and germline specification (Aksoy et al., 2013; Merino et al., 2014; Ng et al., 2012, Irie et al, 2015). Jauch et al. reported that a single residue swap in Sox17, E57→K, shifts its binding preference to the canonical SoxOct motif converting Sox17 into a pluripotency inducer (Jauch et al., 2011). The Sox17 C-terminus transactivator domain (CTD) is larger and more potent than Sox2's CTD. Replacing the Sox2 CTD with that from Sox17 was shown to enhance the reprogramming ability of Sox2 (Aksoy et al., 2013).

iPSC technology remains inefficient, particularly for non-murine cells, and reprogramming often leads to epigenetic aberrations resulting in inconsistent iPSC quality. It is well-established that OSKM reprogramming commonly used for derivation iPSCs often results in loss of imprinting (LOI), which can be cancerous. As mentioned above, although a few alternative cocktails were shown to improve the fidelity of the reprogramming process in mouse, they failed to reprogram human cells, which appear to possess a stronger epigenetic barrier. Perhaps the apparent barrier is merely a testimony to the inadequacy of the wild-type factor-based reprogramming machinery we are currently employing. Thus, there is still an urgent need in the art to provide means and methods for enhancing the efficiency and quality of iPSCs reprogramming which overcome deficiencies of conventional techniques and cocktails. The present invention addresses this need and provides alternative Sox reprogramming factors and cocktails and methods for highly efficient and improved cell reprogramming.

Accordingly, the present invention relates in a first aspect to a SoxB1 factor variant comprising a) the HMG domain of any one of the amino acid sequences of SEQ ID NOs: 1 to 3, wherein the amino acid alanine at position 61 of the HMG-domain is substituted with an amino acid selected from valine, leucine, isoleucine, phenylalanine, methionine, tryptophan or proline, preferably valine; or b) an amino acid sequence sharing at least 82% sequence identity with the HMG domain as defined in a), provided that the substitution as defined in a) is retained.

Generally, a “Sox factor” refers to a member of a known family of transcription factors (TFs), which are involved in maintaining pluripotency. In general, they exert their functions by binding to regulatory elements containing a Sox DNA motif. The members of the Sox family of TFs are classified into different groups (SoxA-SoxJ) based on their level of amino acid sequence identity within their HMG-domains. All naturally occurring Sox TFs are composed of an N-terminal domain (NTD), a DNA-binding high-mobility (HMG)-domain and a C-terminal domain (CTD), containing either a transactivation or a transrepression function. Sox members belonging to the same group have a high degree of amino acid sequence identity (~70% to 95%) with respect to both, their HMG domain and the regions outside their HMG domains. In contrast, Sox proteins from different groups have only partial (≥46%) amino acid sequence identity with respect to their HMG domains (Aksoy et al., 2013). Because Sox TFs generally have similar DNA binding specificities, their ability to trigger specific biological processes is thought to be mediated by their selective interaction with specific cofactors, such as Oct4.

The term “SoxB1 factors”, as used herein, refers to the B1 subclass of Sox (TFs), including, among others, the factors Sox1, Sox2, and Sox3, which share more than 90% sequence identity in respect to their HMG domains.

As shown herein, the inventors found that replacing Sox2 with the Sox17^{EK} mutant (i.e., Sox17 E57K) in the reprogramming cocktail rescues otherwise detrimental Oct4 mutants as well as allows reprogramming with POU factors other than Oct4. Subsequently, the inventors generated a library of chimeric Sox2-Sox17 TFs to find the structural elements of Sox17 responsible for this peculiar phenotype.

The term “Sox17”, as used herein, refers to a member of the F subclass of Sox TFs (“SoxF factors”), which is a subclass of the Sox TFs, distinct from the SoxB1 factors.

It was surprisingly found by the present inventors by means of a library screen that a single residue swap in the SoxB1 factor Sox2 at the Oct/Sox interface, i.e., the replacement of alanine at position 61 of the HMG domain of Sox2 with valine, a more hydrophobic residue, significantly increases the stability of the Oct/Sox complex on canonical SoxOct motifs that regulate naïve pluripotency. The enhanced heterodimerization was observed on both naked and nucleosomal DNA using EMSA, as well as in situ in reprogrammed cells using CHIP-seq. Furthermore, Sox^{A61V} allows reprogramming with Oct4 orthologs, such as Brn2, Brn4, Oct6, Oct2 and otherwise unfunctional Oct4 mutants. Moreover, the tetraploid complementation assay—the most stringent test for pluripotency that measures the ability of iPSCs to generate the entire animal—showed that the Sox^{A61V} mutant dramatically enhances the developmental potential of OSKM iPSCs (demonstrated in the examples herein below).

In this context, it is noted that the amino acid sequences of SEQ ID NOs: 1 to 3 correspond to the HMG domains of the human wild-type SoxB1 factors Sox1, 2 and 3, respectively.

Accordingly, the SoxB1 factor variant of the first aspect of the present invention comprises a) the HMG domain of any one of Sox1, 2 or 3 (i.e. SEQ ID NO: 1, 2 or 3), wherein the amino acid alanine at position 61 is substituted with an amino acid selected from valine, leucine, isoleucine, phenylalanine, methionine, tryptophan or proline, preferably valine; or b) an amino acid sequence sharing at least 82% sequence identity with the HMG domain as defined in a), provided that the substitution at position 61 of their respective HMG domain is retained.

In a preferred embodiment of item b) of the first aspect of the present invention, the amino acid sequence sharing at least 82% sequence identity display, with increasing preference, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity and at least 99% sequence identity with the HMG domain as defined in item a) of the first aspect of the present invention.

In accordance with the present invention, the term “percent (%) sequence identity” describes the number of matches (“hits”) of identical amino acids/nucleotides of two or more aligned amino acid or nucleic acid sequences as compared to the number of amino acid residues or nucleotides making up the overall length of the template nucleic acid or amino acid sequences. In other terms, using an alignment, for two or more sequences or subsequences the percentage of amino acid residues or nucleotides that are the same (e.g. 70%, 75%, 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

The term “sequence identity”, as used herein, describes the sequence match between two (poly)peptides or nucleic acids. The (poly)peptide or nucleic acid sequences to be compared are aligned and compared for their identical aligned sequence, wherein an identical alignment means the occupation of the same position in the sequences to be compared by the same nucleobase or amino acid residue. Accordingly, the “percent identity” is a function of the number of matching positions divided by the number of positions compared and multiplied by 100%. For example, if 7 out of 10 sequence positions are identical, then the identity is 70%.

Nucleotide and amino acid sequence analysis and alignment in connection with the present invention are preferably carried out using bioinformatics tools for pair wise alignment such as EMBOSS Needle (https://www.ebi.ac.uk/Tools/psa/emboss_needle/; see also Madeira F, et al. The EMBL-EBI search and sequence analysis tools APIs in 2019. Nucleic Acids Research. 2019 July; 47(W1):W636-W641. DOI: 10.1093/nar/gkz268). The “identity” or “percent (%) identity” between two amino acid sequences can, e.g., be determined by using the Needleman-Wunsch algorithm (Needleman, S. B. and Wunsch, C D. A general method applicable to the search for similarities in the amino acid sequence of two proteins. J Mol Biol. 1970; 48(3):443-53. DOI: 10.1016/0022-2836(70)90057-4) which has been incorporated into EMBOSS Needle, using a BLOSUM62 matrix, a “gap open penalty” of 10, a “gap extend penalty” of 0.5, a false “end gap penalty”, an “end gap open penalty” of 10 and an “end gap extend penalty” of 0.5. The percent (%) identity is typically determined over the entire length of the query sequence on which the analysis is performed. Two molecules having the same primary amino acid or nucleic acid sequence are identical irrespective of any chemical and/or biological modification, e.g., glycosylation patterns. In case of nucleic acids, for example, two molecules having the same sequence but different linkage components such as thiophosphate instead of phosphate are identical by this definition. Another tool for assessing biological sequence alignments determining regions of similarity of biological sequences with the same number of residues is the NCBI BLAST algorithm (https://blast.ncbi.nlm.nih.gov/Blast.cgi; Stephen F. Altschul, Thomas L. Madden, Alejandro A. Schäffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences. The preferred method for nucleotide and amino acid sequence analysis and alignment in connection with the present invention is most preferably carried out by EMBOSS Needle, wherein the parameters are a BLOSUM62 matrix, a “gap open penalty” of 10, a “gap extend penalty” of 0.5, a false “end gap penalty”, an “end gap open penalty” of 10 and an “end gap extend penalty” of 0.5.

The amino acid sequences of wild-type SoxB1 factors, as well as the corresponding protein encoding nucleotide sequences are known for a number of species, including human and mouse Sox1, Sox2 and Sox3, and are available, e.g., from the NCBI Database: https://www.ncbi.nlm.nih.gov.

For example, the amino acid sequences of the wild-type full-length human Sox1, Sox2 and Sox3 proteins are available from the NCBI database under the following accession numbers: Sox1: NP_005977, such as NP_005977.2; Sox2: NP_003097, such as NP_003097.1; Sox3: NP_005625, such as NP_005625.2 and are defined herein by SEQ ID NOs: 4 to 6, respectively. It is understood that all sequences under the respective NCBI reference sequence numbers are included. It follows that the HMG domains of human Sox1 to Sox3 as defined by SEQ ID NOs: 1 to 3, respectively, are comprised in SEQ ID NOs: 4 to 6, respectively. e.g., the HMG domain of SEQ ID NO: 1 is comprised in SEQ ID NO: 4, etc.

In this context, it is to be noted that with the sequence identity level of at least 82% to SEQ ID NOs: 1, 2 or 3, homologs of the human Sox1, Sox2 and Sox3 HMG domain from other species are specifically also included, e.g., such as the HMG domains of Sox1, Sox2 or Sox3 from mouse, cynomolgus macaque, rat, horse, pig, bovine or other animal species.

As shown in the examples herein below, the inventors further found that the residue swap at position A61 of the HMG domain of Sox2 with an amino acid selected from valine, leucine and isoleucine, preferably valine; when combined with other beneficial elements of the Sox17 HMG domain, as defined herein with SEQ ID NO: 15, further enhances reprogramming efficiency.

Accordingly, also contemplated herein in a preferred embodiment of item a) of the first aspect of the present invention, is a SoxB1 factor variant, wherein furthermore, the a) amino acids at positions 43 to 47 in the HMG domain of any one of SEQ ID NOs: 1 to 3 are substituted with the amino acid sequence of SEQ ID NO: 7; and/or b) the amino acids at positions 65 to 86 in the HMG domain of any one of SEQ ID NOs: 1 to 3 are substituted with the amino acid sequence of SEQ ID NO: 8; and/or c) the SoxB1 factor variant comprises an amino acid sequence sharing at least 82% sequence identity with the SoxB1 factor variant of a) and/or b), provided that the substitution as defined in a) and/or b) is retained.

In accordance with item c) of the preferred embodiment described above, the amino acid sequence sharing at least 82% sequence identity displays with increasing preference at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity and at least 99% sequence identity with the SoxB1 factor variant of a) and/or b), provided that the substitution as defined in a) and/or b) is retained.

In a particularly preferred embodiment of the first aspect of the present invention, the SoxB1 factor variant is a Sox2 factor variant.

Thus, in accordance with the particularly preferred embodiment described above, the Sox2 factor variant comprises a) the HMG domain of the amino acid sequence of SEQ ID NO: 2, wherein the amino acid alanine at position 61 of the HMG domain is substituted with an amino acid selected from valine, leucine, isoleucine, phenylalanine, methionine, tryptophan or proline, preferably valine; or b) an amino acid sequence sharing at least 82% sequence identity with the HMG domain as defined in a), provided that the substitution as defined in a) is retained.

The inventors moreover found that the reprogramming efficiency can be even further enhanced when the substitutions to the HMG domain as described herein above are combined with additional beneficial elements.

Accordingly, the SoxB1 factor variant in accordance with the first aspect of the present invention may further comprise one or more additional amino acids or amino acid sequences flanking the C-terminal and/or the N-terminal end of the HMG domain. Such additional amino acids or amino acid sequences may, e.g., correspond to a naturally or non-naturally occurring N-terminal domain and/or a C-terminal domain flanking the HMG domain, respectively. The N-terminal or C-terminal domain naturally flanking the HMG domain of a Sox factor may for example be replaced by the N-terminal or C-terminal domain of another Sox factor.

Specifically, it has been found that the Sox17 C-terminal (transactivator) domain is larger and more potent than the C-terminal domain of Sox2 and that replacing the Sox2 CTD with the Sox17 CTD enhances the reprogramming ability of Sox2 (Aksoy et al., 2013).

Hence, in another preferred embodiment of the first aspect of the invention, the SoxB1 factor variant further comprises the amino acid sequence of SEQ ID NO: 9, wherein preferably the amino acid sequence of SEQ ID NO: 9 is linked to the C-terminal end of the HMG domain as defined in accordance with the first aspect of the present invention.

As used herein, the term “linked” means one (poly)peptide is attached, preferably, to another by a peptide bond between one amino acid of the (poly)peptide to one amino acid of the other (poly)peptide. The term “linked” as used herein in particular means “operably linked”, which refers to the juxtaposition of at least two (poly)peptide(s) to each other in a way that both (poly)peptides function normally and allow the possibility that at least one of the (poly)peptides can mediate a function that is affected by the other (poly)peptide.

In another preferred embodiment, the SoxB1 factor variant may also represent a full-length variant of wild-type Sox1, Sox2 or Sox3 (corresponding to SEQ ID NOs: 4 to 6) comprising a C- and N-terminal domain, wherein the amino acid alanine at position 61 within their respective HMG domains as defined by SEQ ID NO: 1, 2 or 3 is substituted with an amino acid selected from valine, leucine, isoleucine, phenylalanine, methionine, tryptophan or proline, preferably valine.

In another, or even further preferred embodiment, the amino acid alanine at position 61 within their respective HMG domains as defined by SEQ ID NOs: 1, 2 or 3 is substituted with an amino acid selected from valine, leucine, and isoleucine, most preferably valine.

In this context, it is noted that the reference to position 61 with respect to the substitution in accordance with the present invention always relates to the amino acid count starting from the respective HMG domain as defined herein.

Accordingly, in a further preferred embodiment of the first aspect of the present invention, the SoxB1 factor variant comprises or consists of any one of the amino acid sequences of SEQ ID NOs: 10 to 12; or an amino acid sequence sharing at least 82%, sequence identity with any one of SEQ ID NOs: 10 to 12, provided that the amino acid valine corresponding to position 109 of SEQ ID NO: 10, position 99 of SEQ ID NO: 11 or position 197 of SEQ ID NO: 12 is retained.

SEQ ID NOs: 10 to 12 correspond to the full-length amino acid sequences of human wild-type Sox1, Sox2 and Sox3, respectively, wherein the amino acid alanine at position 61 within their respective HMG domains as defined by SEQ ID NO: 1 to 3, respectively, is substituted with valine.

In preferred embodiments, the amino acid sequence according to the above-mentioned preferred embodiment of the first aspect of the invention sharing at least 82% sequence identity displays, with increasing preference, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, and at least 99.5% sequence identity to SEQ ID NOs: 10 to 12 and has most preferably 100% sequence identity to SEQ ID NOs: 10 to 12.

In a particularly preferred embodiment of the first aspect of the invention, the SoxB1 factor variant comprises or consists of the amino acid sequence of SEQ ID NO: 13 or SEQ ID NO: 14.

In an alternative preferred embodiment, the SoxB1 factor variant shares, with increasing preference, at least 96% sequence identity, at least 97% sequence identity, at least 98% sequence identity, at least 99% sequence identity, and at least 99.5% sequence identity, and is most preferably 100% identical to SEQ ID NOs: 13 or 14, provided that the substitution as defined in a) is retained.

Furthermore, the inventors found that other regions of Sox17, such as positions 24 to 28 of the Sox17 HMG domain significantly decreased the reprogramming efficiency of Sox2. Accordingly, it can be concluded that the reprogramming efficiency of Sox17 can be increased by the replacement of positions 24 to 28 of the HMG domain of Sox17 with positions 24 to 28 of the HMG domain of Sox2.

Thus, in a second aspect, the present invention relates to a Sox17 factor variant comprising: a) the HMG domain of the amino acid sequence of SEQ ID NO: 15, wherein the amino acids at positions 24 to 28 of SEQ ID NO: 15 are substituted with the amino acid sequence of SEQ ID NO: 16; or b) an amino acid sequence sharing at least 82%, sequence identity with the HMG domain as defined in a), provided that the substitution as defined in a) is retained.

SEQ ID NO: 15 corresponds to the amino acid sequence of the human wild-type HMG domain of Sox17. SEQ ID NO: 16 corresponds to the amino acid sequence at positions 24 to 28 of the human wild-type HMG domain of Sox2.

In preferred embodiments, the amino acid sequence according to the above-mentioned preferred embodiment of item b) of the second aspect of the invention sharing at least 82% sequence identity displays, with increasing preference, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% and at least 99% sequence identity with the HMG domain as defined in a), provided that the substitution as defined in a) is retained.

In this context, it is to be noted that with the sequence identity level of at least 82%, homologs of the human Sox17 HMG domain from other species are specifically also included, such as the HMG domains of Sox17 from mouse or cynomolgus macaque, rat, horse, pig, bovine, or other animal species.

The definitions and preferred embodiments of the first aspect of the invention, as far as being amenable for combination with the second aspect of the invention, apply mutatis mutandis to the second aspect of the invention. This applies mutatis mutandis to the further aspects of the invention as described herein below. For instance, the definitions and preferred embodiments of the first and second aspect as far as being amenable for combination with the third aspect apply mutatis mutandis to the third aspect of the invention, etc.

Importantly, the instant inventors surprisingly found that the SoxB1 factor variants of the present invention not only allow the generation of iPSCs with significantly improved developmental potential, but also allow reprogramming with otherwise detrimental Oct4 mutants and allows reprogramming with POU factors such as Brn4, Oct2, Oct6 and Brn2, which are normally incapable of iPSC generation.

Thus, in a third aspect, the present invention relates to a fusion protein comprising or consisting of a) a Sox factor and a POU factor; or b) a Sox factor and a POU domain; or c) an HMG domain and a POU factor; or d) an HMG domain and a POU domain; wherein the Sox factor is selected from: i. a SoxB1 factor or the SoxB1 factor variant of the first aspect of the present invention; or ii. Sox 17 or a Sox17 factor variant of the second aspect of the present invention; and wherein the HMG domain is selected from: iii. the HMG domain in accordance with item a) or item b) of the first aspect of the present invention; and wherein the POU factor is selected from iv. Oct4 or an Oct4 variant; or v. Oct2 or an Oct2 variant; or vi. Oct6 or an Oct6 variant; or vii. Brn2 or a Brn2 variant; or viii. Brn4 or a Brn4 variant; or (ix) other natural or synthetic POU factors; and wherein the POU domain is selected from x. the POU domain of SEQ ID NO: 17 or 18 or a variant thereof sharing at least 82% sequence identity with SEQ ID NO: 17 or 18; wherein preferably, the amino acids at positions 1 to 50, 78 and/or 82 of SEQ ID NOs: 17 or 18, respectively, are maintained.

SEQ ID NO: 17 corresponds to the amino acid sequence of the wild-type human Oct4 POU domain.

SEQ ID NO: 18 corresponds to the amino acid sequence of the wild-type mouse Oct4 POU domain.

The sequence identity of the amino acid sequence according to item x. of the third aspect of the invention sharing, with increasing preference, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, and at least 99.5% sequence identity with SEQ ID NOs: 17 or 18 and has most preferably 100% sequence identity with SEQ ID NOs: 17 or 18, respectively.

The phrase “POU domain of SEQ ID NO: 17 or 18”, as used herein, means an amino acid sequence as defined by the amino acid sequences set forth in SEQ ID NO: 17 and 18, respectively.

The term “protein” as used herein is used interchangeably with the term polypeptide and refers to polymers constructed from one or more chains of amino acid residues linked by peptide bonds. The group of “polypeptides” consists of molecules with more than 30 amino acids, which is in distinction to the group of peptides consisting of up to 30 amino acids. As used herein, the term “(poly)peptides” refers more generally to both “peptides” and “polypeptides”. The term “(poly)peptides” also refers to chemically or post-translationally modified peptides or polypeptides. Generally, the terms apply to naturally occurring amino acid polymers, as well as to amino acid polymers in which one or more amino acid residue is an artificial chemical analogue of a corresponding naturally occurring amino acid.

The term “fusion protein” as used herein refers to a synthetic, semi-synthetic or recombinant single poly(peptide) molecule that comprises all or a portion of two or more different poly(peptides). The fusion can be an N-terminal fusion, a C-terminal fusion or an internal fusion. The fusion protein furthermore can include a linker connecting two or more different poly(peptides). The skilled person is aware of linkers which may suitably be used for connecting two or more different polypeptides.

In a preferred embodiment, the fusion protein of the third aspect of the invention comprises a Sox factor and a POU factor, wherein the POU factor is located N-terminally, and the Sox factor is located C-terminally within the fusion protein.

The term “POU factors”, as used herein, refers to transcription factors (TFs) containing a bipartite DNA binding domain referred to as a POU domain, flanked by N- and C-terminal transactivator domains (NTD and CTD). The bipartite POU domain consists of a POU-specific domain (POU_{S}) and a POU-homeodomain (POU_{HD}) joined by a flexible non-conserved linker domain, which can be of variable length. The structure of the POU domain allows the binding of DNA and also participation in protein-protein interactions.

The term “other natural or synthetic POU factors” as used herein generally specifically encompasses to (poly)peptides comprising a POU domain as described herein above. The (poly)peptides may include both naturally occurring products and the products of recombinant DNA or other synthetic techniques.

Generally, POU factors have different binding profiles and different preferences for hetero-versus homodimerisation (Jerabek et al., 2017; Malik et al., 2019; Mistri et al., 2015). Examples of POU factors include POU class 2 (including Oct1 and Oct2), POU class 3 (including Oct6, Brn1, Brn2, Brn4, etc.) and POU class 5 (including Oct4) factors.

As used herein, the term “POU factors”, “POU family” or “Oct family” refers to the family of octamer (“Oct”) transcription factors which play a crucial role in, amongst others, maintaining pluripotency. POU5F1 (POU domain, class 5, transcription factor 1) also known as Oct4 is one representative of POU family. Further examples include Oct1, Oct2, Oct6, Brn2 and Brn4. Exemplary Oct4 proteins are the proteins encoded by the murine Oct4 gene (all sequences deposited under the NCBI Reference Sequence NM_013633, such as NM_013633.3) and the human Oct4 gene (all sequences deposited under the NCBI Reference Sequence NM_002701, such as NM_002701.6).

The terms “Oct4”, “OCT4”, “Oct3/4”, “Oct4 protein”, “OCT4 protein”, “POU5F1”, “Oct3”, “Oct3/4” and the like refer to any of the naturally-occurring forms of the Octamer 4 transcription factor, or variants thereof that maintain Oct4 transcription factor activity of at least, (for each value) with increasing preference, 10%, 30%, 50%, 80%, 90% or 100% activity compared to wild type Oct4 as measured by methods known in the art, such as measuring the efficiency of iPSC generation in reprogramming experiments.

In some embodiments, variants have at least 90% amino acid sequence identity across their whole sequence compared to the naturally occurring Oct4 polypeptide. In other embodiments, the Oct4 protein is the protein as identified by the Genbank reference ADW77327.1, corresponding to SEQ ID NO: 19, or the protein as identified by SEQ ID NO: 20. Oct4 variants potentially not maintaining Oct4 transcription factor activity, but comprising or consisting of the amino acid sequence of any one of the SEQ ID NOs: 21 to 24 are specifically also included.

The terms “Oct2”, “OCT2”, “Oct2 protein”, “OCT2 protein”, “POU2F2” and the like refer to any of the naturally-occurring forms of the Octamer 2 transcription factor, or variants thereof that maintain Oct2 transcription factor activity of at least, (for each value) with increasing preference, 50%, 80%, 90% or 100% activity compared to wild type Oct2 as measured by methods known in the art, such as measuring the efficiency of iPSC generation in reprogramming experiments.

In some embodiments, variants have at least 90% amino acid sequence identity across their whole sequence compared to the naturally occurring Oct2 polypeptide. In other embodiments, the Oct2 protein is the protein as identified by all sequences deposited under the NCBI Reference Sequences NP_001193954, such as NP_001193954.1 or NP_001157027, such as NP_001157027.1, corresponding to SEQ ID NO: 25 or 26, respectively.

The terms “Oct6”, “OCT6”, “Oct6 protein”, “OCT6 protein”, “POU3F1” and the like refer to any of the naturally-occurring forms of the Octamer 6 transcription factor, or variants thereof that maintain Oct6 transcription factor activity (e.g. within at least, (for each value) with increasing preference, 50%, 80%, 90% or 100% activity) compared to wild-type Oct6 as measured by methods known in the art, such as measuring the efficiency of iPSC generation in reprogramming experiments.

In some embodiments, variants have at least 90% amino acid sequence identity across their whole sequence compared to the naturally occurring Oct6 polypeptide. In other embodiments, the Oct6 protein is the protein as identified by all sequences deposited under the NCBI Reference Sequence NP_002690, such as NP_002690.3 or NP_035271, such as NP_035271.1, corresponding to SEQ ID NOs: 27 or 28, respectively.

The terms “Brn2”, “BRN2”, “Brn2 protein”, “BRN2 protein”, “N-Oct-3”, “POU3F2”, “Oct7” and the like refer to any of the naturally-occurring forms of the Brn2 transcription factor, or variants thereof that maintain Brn2 transcription factor activity of at least, (for each value) with increasing preference, 50%, 80%, 90% or 100% activity compared to wild type Brn2 as measured by methods known in the art, such as measuring the efficiency of iPSC generation in reprogramming experiments.

In some embodiments, variants have at least 90% amino acid sequence identity across their whole sequence compared to the naturally occurring Brn2 polypeptide. In other embodiments, the Brn2 protein is the protein as identified by all sequences deposited under the NCBI Reference Sequence NP_005595, such as NP_005595.2 or NP_032925, such as NP_032925.1, corresponding to SEQ ID NO: 29 or 30, respectively.

The terms “Brn4”, “BRN4”, “Brn4 protein”, “BRN4 protein”, “POU3F4”, “Oct9” and the like refer to any of the naturally-occurring forms of the Brn4 transcription factor, or variants thereof that maintain Brn4 transcription factor activity of at least, (for each value) with increasing preference, 50%, 80%, 90% or 100% activity compared to wild type Brn4 as measured by methods known in the art, such as measuring the efficiency of iPSC generation in reprogramming experiments.

In some embodiments, variants have at least 90% amino acid sequence identity across their whole sequence compared to the naturally occurring Brn4 polypeptide. In other embodiments, the Brn4 protein is the protein as identified by all sequences deposited under the NCBI Reference Sequence NP_000298, such as NP_000298.3 or, corresponding to SEQ ID NOs: 31 or 32, respectively.

The term “domain”, as used herein, means a region/part of an amino acid sequence that is capable of autonomously adopting a specific structure and/or function. Accordingly, a “domain” represents a functional domain or a structural domain of a protein, e.g., of a transcription factor. For example, the HMG domain which is involved in DNA binding, is composed of approximately 75 amino acid residues that collectively mediate the DNA-binding of chromatin-associated high-mobility group proteins.

The term “variant”, as used herein, refers to a nucleic acid or an amino acid sequence, which differs in comparison to the corresponding wild-type sequence in at least one base or amino acid residue, respectively. The difference may be artificially created, or naturally occurring. In a preferred embodiment the variant with respect to a referred (poly)peptide refers to an amino acid sequence variant of a corresponding wild-type amino acid sequence of said (poly)peptide. The variant maintains or essentially maintains the function of the wild-type form. Essentially means with increasing preference of at least 50%, at least 80% at least 90% and at least 95%.

Accordingly, in a preferred embodiment, the variant shares, with increasing preference, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, and at least 99.5% sequence identity with the corresponding wild-type amino acid sequence.

In a preferred embodiment of the third aspect of the invention, the Sox factor may be a wild-type Sox factor or Sox factor variant as defined above, preferably by the Sox factor variant as defined in SEQ ID NO: 13 or 14.

In a fourth aspect, the present invention relates to a complex, or a composition, comprising or consisting of: a) a Sox factor and a POU factor; or b) a Sox factor and a POU domain; or c) an HMG domain and a POU factor; or d) an HMG-domain and a POU domain, wherein the Sox factor, the POU factor, the HMG domain and the POU domain are selected from the Sox factor, the POU factor, the HMG domain, and the POU domain as defined in accordance with the third aspect of the invention.

In accordance with the fourth aspect of the invention, the term “complex” refers to an association of two molecules that interact with each other through bonds and/or forces (e.g., van der Waals, hydrophobic, hydrophilic forces) that are not peptide bonds. Individual members of a complex may be linked by non-covalent interactions. The “complex” according to the fourth aspect of the invention may also include a further non-covalent interaction of at least one of the molecules as defined in items a) to d) of the fourth aspect of the invention with another macromolecule, such as a nucleic acid. It will be understood that a complex can be multimeric. Each interacting molecule of a complex is herein referred to as an “individual member” or “member” of the complex.

The term “composition”, as used herein, refers to a mixture containing at least two (poly)peptides, also referred herein generally as compounds. These may or may not be complexed.

In a fifth aspect, the present invention relates to a nucleic acid molecule or a combination of nucleic acid molecules, encoding:
the SoxB1 factor variant of the first aspect of the invention, the Sox17 factor variant of the second aspect of the invention, or the fusion protein of the third aspect of the invention; and/or
the complex or composition of the fourth aspect of the invention; and/or
one or more additional reprogramming factor(s).

The term “nucleic acid molecule” in accordance with the present invention includes DNA, such as cDNA or double- or single-stranded genomic DNA and RNA, including natural and modified DNA and RNA. In this regard, “DNA” (deoxyribonucleic acid) means any chain or sequence of the chemical building blocks selected from adenine (A), guanine (G), cytosine (C) and thymine (T), called nucleotide bases, that are linked together typically by a phosphodiester bond on a deoxyribose sugar backbone. DNA can have one strand of nucleotide bases, or two complementary strands which may form a double helix structure. “RNA” (ribonucleic acid) means any chain or sequence of the chemical building blocks adenine (A), guanine (G), cytosine (C) and uracil (U), called nucleotide bases, that are linked together typically by a phosphodiester bond on a ribose sugar backbone. RNA typically has one strand of nucleotide bases, such as mRNA. Included are also single- and double-stranded hybrids molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analogue, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may additionally comprise promoters, enhancers, response elements, signal sequences, polyadenylation sequences, introns, 5′- and 3′-non-coding regions, and the like.

In a preferred embodiment of the fifth aspect of the invention, the nucleic acid molecule is an RNA, preferably a modified RNA, more preferably a modified mRNA (modRNA).

As used herein, the term “modified RNA” or “modified mRNA” refers to an RNA or mRNA, respectively which comprises at least one modified nucleoside. For example, the nucleoside uridine may be modified to pseudo uridine or N1-methyl-pseudouridine and/or the nucleoside cytosine may be modified to 5-methylcytosine, which alter the secondary structure of the RNA and can reduce recognition by the innate immune system while still allowing effective translation.

In a preferred embodiment of the fifth aspect of the invention, the reprogramming factor is selected from the group including POU factor, Klf and Myc family members.

As used herein, the term “Myc family member” refers to a member of transcription factors encoded by myc proto-oncogenes implicated in cancer. c-Myc was shown to be a transcription factor implicated in the generation of mouse iPSCs and of human iPSCs. Exemplary c-Myc proteins are the proteins encoded by the murine c-myc gene (all sequences deposited under the NCBI Reference Sequence NM_010849, such as NM_010849.4) and the human c-myc gene (all sequences deposited under the NCBI Reference Sequence NM_002467, such as NM_002467.6). n-Myc or l-Myc was also used as possible reprogramming factor replacing c-Myc. The terms “c-Myc”, “cMyc”, “C-Myc” and the like referred to herein, thus, include any of the naturally occurring forms of the c-Myc transcription factor, or variants thereof that maintain c-Myc transcription factor activity (e.g., with at least, (for each value) with increasing preference, 50%, 80%, 90% or 100% of transcription factor activity) compared to wild type c-Myc as measured by methods known in the art, such as measuring the efficiency of iPSC generation in reprogramming experiments.

In some embodiments, variants have at least 90% amino acid sequence identity across their whole sequence compared to the naturally occurring c-Myc polypeptide. In other embodiments, the c-Myc protein is the protein as identified by all sequences deposited under the NCBI Reference Sequences NP_002458, such as NP_002458.2 or NP_001170823, such as NP_001170823.1, corresponding to SEQ ID NOs: 41 or 42, respectively.

The terms “L-Myc”, “LMyc”, “L-Myc” and the like referred to herein thus include any of the natural-occurring forms of the L-Myc transcription factor, or variants thereof that maintain L-Myc transcription factor activity (e.g., with at least (for each value) 50%, 80%, 90% or 100% activity compared to wild type L-Myc) as measured by methods known in the art, such as measuring the efficiency of iPSC generation in reprogramming experiments.

In some embodiments, variants have at least 90% amino acid sequence identity across their whole sequence compared to the naturally occurring L-Myc polypeptide. In other embodiments, the L-Myc protein is the protein as identified by all sequences deposited under the NCBI reference NP_001028253, such as NP_001028253.1, corresponding to SEQ ID NO: 43.

In a sixth aspect, the present invention relates to a vector, or a combination of vectors, comprising the nucleic acid molecule or the combination of nucleic acid molecules of the fifth aspect of the invention.

The term “vector” in accordance with the invention means preferably a plasmid, cosmid, virus, bacteriophage, episomal or another vector used e.g., conventionally in genetic engineering which carries the nucleic acid molecule or the combination of nucleic acid molecules of the invention. The nucleic acid molecule or the combination of nucleic acid molecules of the invention may, for example, be inserted into several commercially available vectors.

The nucleic acid molecules inserted into the vector can e.g., be synthesized by methods known in the art, or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e.g., translation initiation codon, promoters, such as naturally associated or heterologous promoters and/or insulators; see above), optionally internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide encoding the SoxB1 factor variant, the Sox17 factor variant, the fusion protein or the components of the complex or composition of the invention is operatively linked to such expression control sequences allowing expression in prokaryotes or eukaryotic cells. The vector may further comprise nucleic acid sequences encoding further regulatory elements, for example 5′ and 3′ UTRs that can extend the longevity of mRNA Such sequences are well known to the person skilled in the art.

In a preferred embodiment of the first to fifth aspects of the present invention, the Sox family TF, the HMG domain, the POU domain, the POU factor, the fusion protein, and/or the complex or compounds of the composition, contain a nuclear localization signal (NLS), which targets the localization of said protein and complex or compounds of the composition to the nucleus. The NLS can have a monopartite or bipartite form, wherein monopartite means a single cluster of amino acids and bipartite means two clusters of amino acids that are separated by several amino acids. In a preferred embodiment of the invention, the NLS is the NLS sequence as comprised in the amino acid sequence of the wildtype Sox family TFs and/or POU domain, and/or the POU factors. For example, for wild-type human Sox2, the NLS sequence is a bipartite NLS wherein one part is found in the beginning and the other part in the end of the amino acid sequence SEQ ID NO:2. The NLS sequence is predictable from NLS databases, such as NLSdb (https://rostlab.org/services/nisdb/; Nair, R., Carer, P., Rost, B., NLSdb: database of nuclear localization signals. Nucl Acids Res, 2003, 31:397-399), prediction algorithms such as SeqNLS (http://mleg.cse.sc.edu/seqNLS/; Lin J R, Hu J. SeqNLS: nuclear localization signal prediction based on frequent pattern mining and linear motif scoring. PLoS One. 2013 Oct. 29; 8(10):e76864. doi: 10.1371/journal.pone.0076864. PMID: 24204689; PMCID: PMC3812174.), NLStradamus (Nguyen Ba, A. N., Pogoutse, A., Provart, N. et al. NLStradamus: a simple Hidden Markov Model for nuclear localization signal prediction. BMC Bioinformatics 10, 202 (2009)) or NoLS/NoD (http://www.compbio.dundee.ac.uk/www-nod/; Scott M S, Troshin P V, Barton G J. NoD: a Nucleolar localization sequence detector for eukaryotic and viral proteins. BMC Bioinformatics. 2011 Aug. 3; 12:317. doi: 10.1186/1471-2105-12-317. PMID: 21812952; PMCID: PMC3166288).

In a preferred embodiment, the vector is an episomal or other DNA vector, self-replicating RNA, unmodified or modified mRNA or a viral vector.

The term “episomal vector” or “episome”, as used herein, refers to polynucleotides introduced without integrating the vector or parts of said vector into the chromosomal DNA (integration-free) of a cell, preferably a host cell. The specific episomal vectors, as used herein, are described in the Examples herein. The skilled person is aware that the plasmid vectors used for the construction of episomal vectors are not limited to those as described herein, but may be selected by the skilled person as typically conducted in the art.

Episomes are polynucleotides which can replicate extrachromosomally in a host cell. Said polynucleotides encode exogenous and/or endogenous polypeptides in said host cell. Episomes are advantageous over integrating vector/plasmids because of their low chance of random integration and introduction of mutations into the host cell genome (see Van Craenenbroeck et al., 2000. Episomal vectors for gene expression in mammalian cells. Eur. J. Biochem. 267, 5665-5678). The episome(s) may remain intracellularly. In this case, the introduced the polynucleotide(s) encoding for a protein(s) can be permanently expressed i.e., stably/constitutively. In a more preferred embodiment of the fifth to sixth aspect of the invention, the episome(s) is/are lost after several passages of the converted/reprogrammed/reset cell, wherein the expression of the introduced polynucleotide(s) encoding for a protein(s) is non-permanent, i.e., transient.

A nucleoside-modified messenger RNA (modRNA) is synthetic messenger RNA (mRNA) that contains one or more natural or synthetic nucleoside analogues. mRNA can be introduced without integration into the chromosomal DNA of a host cell. Modified mRNA methods are non-viral RNA based methods for introduction of polynucleotides into a host cell and provide advantages over DNA-based methods for introduction of polynucleotides encoding exogenous polypeptides in said host cell. For example, the strength of protein expression is independent of promotor activity which prevents gene silencing, achieves an independent expression of each mRNA-mediated gene from one another, which results in more sophisticated stoichiometric expression of multiple genes (Oh and Kessler, 2018. Design, Assembly, Production and Transfection of Synthetic Modified mRNA, Methods. 133:29-43). A modified mRNA may be delivered into the host cell by means known to the skilled person, preferably by means protecting the modified mRNA from ribonucleotides in the host cell without impairing the effectivity of the introduction of polynucleotides as a modified mRNA, such as transfection via lipofection, electroporation, nucleofection, deliver using lipids and lipid nanoparticles (LNPs) or polymer-based nanoparticles.

Non-limiting examples of methods for protecting a modRNA from host cell ribonucleotides include packing the modRNA into a liposome for delivery and up-take into the host cell.

In a seventh aspect, the present invention relates to (a) a cell or (b) a cell derived from said cell, wherein said cell (a) comprises or said cell (b) has been modified by the presence in the cell of:
(i) the SoxB1 factor variant of the first aspect, the Sox17 factor variant of the second aspect, the fusion protein of the third aspect and/or the complex or composition of the fourth aspect of the invention, and/or
(ii) the nucleic acid molecule or the combination of nucleic acid molecules of the fifth aspect; and/or
(iii) the vector or combination of vectors of the sixth aspect of the invention,
and wherein the cell of (b) preferably retains the phenotype of the cell of (a).

It is understood that the term “cell” as used herein refers to and includes a single cell, a plurality of cells or a population of cells where context permits, unless otherwise specified.

The item “a cell (b) derived from the cell (a) wherein said cell (b) has been modified as indicated above” in accordance with the seventh aspect of the invention refers to a cell that comprises or previously comprised the SoxB1 factor variant of the first aspect, the Sox17 factor variant of the second aspect, the fusion protein of the third aspect and/or the complex or composition of the fourth aspect of the invention, the nucleic acid molecule or the combination of nucleic acid molecules of the fifth aspect of the invention or the vector or combination of vectors of the sixth aspect of the invention, wherein the cell has been modified in its characteristics according to the expression pattern of the nucleic acid molecule or the combination of nucleic acid molecules of the fifth aspect of the invention or the vector or combination of vectors of the sixth aspect of the invention. For the purposes of the present disclosure, the “cell” or “cell derived from the cell” (also referred to as cell derivative) as described herein is preferably an isolated cell. The derivative of the cell includes progeny that may or may not display the phenotype of the original cell. In particular, certain features, such as an enhanced developmental potential, a higher expression level of at least one, preferably at least two, more preferably at least three, more preferably at least four, more preferably at least five, and most preferably at least six naïve pluripotency-specific marker(s) selected from Klf17, Klf4, Sox2, Susd2, Argfx, and Dnmt3l, a higher expression level of at least one primitive endoderm-specific marker(s) selected from Gata6 and Sox17, an activated POU5f1 distal enhancer, a reactivated X chromosome in female lines, a reduced DNA methylation, an enhanced capacity to differentiate, preferably into a germline cell, and/or an enhanced capacity to contribute to development of an embryo(s) and/or animal(s), of the phenotype of the original cell carrying a SoxB1 factor variant of the first aspect, or the Sox17 factor variant of the second aspect, as described in this specification may be lost in the progeny or partially lost at least to 10%, at least to 20%, at least to 30%, at least to 40%, at least to 50%, at least to 60%, at least to 70%, at least to 80%, at least to 90% or at least to 95%.

In an eighth aspect, the present invention relates to a method for enhancing the cooperativity between a Sox factor and a POU factor, the method comprising increasing the average number and/or strength of interactions between the HMG domain of the Sox factor and the POU domain of the POU factor.

The term “cooperativity between a Sox factor and a POU factor” generally refers to the interaction between Sox factors and POU factors as described herein above on their genomic targets that leads to a higher stability of the Sox/Oct heterodimer on DNA compared to either of the monomers on the same DNA. Accordingly, POU factors cooperate with Sox factors to co-regulate their genomic targets. Oct/Sox cooperativity is generally mediated by DNA allostery and by protein-protein interactions between their respective DNA-binding domains: the POU and the HMG-domain. Importantly, as can be seen in the examples herein below, the inventors found that replacing Sox2 with either Sox2^{A61V} or Sox2-17 significantly increased Sox/Oct heterodimerization and the stability of the heterodimer complex on their target loci.

Hence, the term “enhancing the cooperativity between a Sox factor and a POU factor”, as used herein, refers to an increase in the propensity of Sox factors and Oct factors for heterodimerization and/or an increase in the stability of Sox factor/Oct factor complex on one of their genomic targets.

The interaction of a transcription factor, a complex of transcriptions factors or a composition of transcription factors, with a DNA element is measured by methods known to the skilled person, such as chromatin immunoprecipitation (ChIP), transcription factor footprinting analysis using assay for transposase-accessible chromatin with sequencing (ATAC-seq), electrophoretic mobility shift assay (EMSA), fluorescence correlation spectroscopy (FCS), molecular dynamics simulations (MDS)

Accordingly, the cooperativity of a Sox factor and a POU factor in forming a heterodimer or in the stability of a complex on one of their genomic targets can also be assessed by ChIP, ATAC-seq, EMSA, MDS, FCS. The skilled person is aware of the steps needed to perform those assays (see Zhang et al., Model-based analysis of ChIP-seq (MACS), 2008. Genome Biol 9.).

As mentioned, methods for assessing the cooperativity between Sox and Oct factors are known in the art and further include, e.g., electrophoretic mobility shift assays (EMSAs) as shown in the examples herein below and as described in Ng et al., 2012, Huang et al., 2015, assay for transposable-accessible chromatin sequencing (ATAC-Seq), isothermal titration calorimetry (ITC), surface plasmon resonance (SPR), fluorescence correlation spectroscopy (FCS), nuclear magnetic resonance (NMR) spectroscopy, cryogenic electron microscopy (cryo-EM), consecutive affinity-purification systematic evolution of ligands by exponential enrichment (CAP-SELEX), high-throughput SELEX (HT-SELEX) or Coop-seq as described in Chang et al., 2015.

Furthermore, computational molecular dynamic simulations (MDS) as described in the examples herein below may be used to assess the average number and/or strength of interactions between the Sox factor HMG-domain and the POU factor POU-domain. Alternatively, Sox/Oct factor cooperativity may also be measured indirectly by assessing their reprogramming efficiency or their ability to rescue non-cooperative variants in reprogramming experiments, wherein an increased reprogramming efficiency corresponds to increased Sox/Oct factor cooperativity.

The differentiation and age status of cells are a continuous spectrum, with the terminally differentiated/aged state at one end of this spectrum and the de-differentiated state (pluripotent state) at the other end. The term “reprogramming”, as used herein, thus refers to a process that alters or reverses the differentiation and/or age status of a cell. Reprogramming includes complete resetting, or partial resetting of the given epigenetic status of a cell. In other words, the term “reprogramming”, as used herein, encompasses any transition of the epigenetic status of a cell along the spectrum toward a less-differentiated and/or less aged state. For example, reprogramming includes reversing a multipotent cell back to a pluripotent cell or reversing a terminally differentiated cell back to either a multipotent cell or a pluripotent cell or reversing the aged status of the cell to less-aged. The term “less-differentiated state” and “less-aged”, as used herein, is thus a relative term and includes a completely de-differentiated or rejuvenated state and a partially differentiated or rejuvenated state. Methods for reprogramming, e.g., fibroblast cells to induced pluripotent stem cells (iPSCs) by expressing ectopically Oct4, Sox2 and c-Myc have been described by Takahashi and Yamanaka, 2006. Methods for reprogramming-driven age reversal (rejuvenation) was described in studies by Belmonte (for example, Ocampo et al, In Vivo Amelioration of Age-Associated Hallmarks by Partial Reprogramming. Cell. 2016; 167(7):1719-1733.12), Sinclair (Lu et al, Reprogramming to recover youthful epigenetic information and restore vision. Nature. 2020; 588(7836):124-129), Reik (Gill et al, Multi-omic rejuvenation of human cells by maturation phase transient reprogramming. 2022. Elife 11, 2021.01.15.426786) and other labs.

Accordingly, a “reprogramming factor” is a factor, most often a transcription factor, which can be used to reprogram a target cell. The term “reprogramming factor” further includes any analogous molecule that mimics the function of the factor with respect to reprogramming capacity.

The term “mimics the function”, as used herein means “retains” or “essentially retains the function”, wherein in the latter case with increasing preference at least 50%, at least 80%, at least 90% or at least 95% of the function are retained in comparison to the factor with respect to reprogramming capacity.

As used herein, the term “reprogramming efficiency” refers to the number of cells in a sample that are successfully reprogrammed to a less-differentiated state or to pluripotency relative to the total number of cells in the sample. For the newly engineered or designed factors and cocktails, the efficiency can be measured by comparing it to traditional or wild-type reprogramming factors and cocktails. Reprogramming efficiency may be measured as a function of pluripotency markers. Such pluripotency markers include, but are not limited to, the expression of transgene reporters (e.g. endogenous Oct4-GFP), pluripotency-specific marker proteins (e.g. Oct4, Sox2, Nanog, Klf17, alkaline phosphatase, SSEA1, TRA-1-60, TRA-1-81, SUSD2 etc.) and mRNA, pluripotent cell morphology and colony formation.

Interactions between the HMG domain and the POU domain in accordance with the eighth aspect of the present invention may include hydrophobic interactions, electrostatic interactions or covalent bonding.

Accordingly, the term “enhancing the cooperativity between a Sox factor and a POU factor” includes the increase of the average number and/or strength of interactions, preferably the increase of the average number of hydrophobic interactions, electrostatic interactions and/or the average strength of a covalent binding.

As shown in the examples herein below, the inventors found that the substitution of the amino acid alanine at position 61 of the HMG domain of Sox2 significantly enhanced the cooperativity between Sox2 and POU factors, including Oct4 on canonical SoxOct DNA motif.

Accordingly, in a preferred embodiment of the method of the eighth aspect of the invention, the average number and/or strength of interactions is increased by substituting the amino acid at position 61 of the HMG domain of a Sox factor with a hydrophobic amino acid selected from valine, leucine, isoleucine, phenylalanine, methionine, or tryptophan, wherein preferably, the amino acid at position 61 of the HMG domain is substituted with valine.

In a more preferred embodiment, the Sox factor is a SoxB1 factor or SoxB1 factor variant of the first aspect of the present invention.

In a more preferred embodiment of the above preferred embodiments, the amino acid at position 61 is substituted with a hydrophobic amino acid selected from valine, leucine, and isoleucine, wherein preferably the amino acid at position 61 is substituted with valine.

In a ninth aspect, the present invention relates to a method for producing induced pluripotent stem cell(s) (iPSCs) from non-pluripotent cell(s), the method comprising culturing said non-pluripotent cell(s) under conditions suitable for reprogramming said non-pluripotent cell(s) into iPSC(s), wherein said conditions comprise increasing in said non-pluripotent cells the level of: a) the SoxB1 factor variant of the first aspect of the present invention or the Sox17 factor variant of the second aspect of the present invention, and a POU factor; and/or b) the fusion protein of the third aspect of the present invention or the complex or composition of the fourth aspect of the present invention; and/or c) a SoxB1 factor variant comprising or consisting of the amino acid sequence of SEQ ID NOs: 13 or 14, and a Klf family member; and optionally:
an inhibitor of p53 function; and/or
one or more additional reprogramming factor(s).

Culturing the cell in accordance with the ninth aspect of the present invention may include contacting the cell with the factors, fusion proteins, and/or complexes or compositions as defined in items a), b) or c) of the ninth aspect of the invention so that the respective factors, fusion proteins and/or complexes or compositions are taken up by the cell, as well as transfecting or transducing the cell with nucleic acids encoding the factors, fusion proteins and/or complexes and co-expressing the factors, fusion proteins and/or complexes.

Specific culturing conditions are necessary for the culturing of (a) cell(s). Those conditions refer to the type of culture media, temperature and CO₂ Level. Mammalian cells in general are cultured at 37 °C, with 5% CO₂ level and cell type-specific culture media containing various supplements. The type of base media is typically selected from the group including low-glucose DMEM, in high-glucose DMEM, IMDM, DMEM/F12 media, Neurobasal media, etc.

Supplements may be selected from the group comprising: Fetal Bovine Serum (FBS), Knockout™ Serum Replacement (KSR), L-glutamine, penicillin, streptomycin, non-essential amino acids, sodium pyruvate, β-mercaptoethanol, growth factors (such as bFGF, EGF, Activin, or LIF), N2 supplement mix, B27 supplement mix, small molecules (such as VPA, XAV939, PD0325901, CHIR99021, Y-27632, or Go6983—HDACs, Wnt, Mek, GSK3, ROCK, and PKC inhibitors, respectively).

Pluripotent stem cells are often cultured on a layer of inactive feeder cells. Feeders are typically derived from mouse embryonic fibroblasts (MEFs) but could also be derived from other cell types.

The skilled person is aware that culturing condition are adjusted to the specific cell type, which is cultured.

For example, HEK293T cells may be cultured in low-glucose DMEM (Sigma) supplemented with 10% FBS (Capricorn Scientific), 1% Glutamax, 1% penicillin-streptomycin, 1% nonessential amino acids (all from Sigma). Mouse, human, cynomolgus and porcine fibroblasts may be cultured in high-glucose DMEM (Sigma) supplemented with 15% FBS, 1% Glutamax, 1% penicillin-streptomycin, 1% nonessential amino acids (NEAA), 1% sodium pyruvate (Sigma), 1% β-mercaptoethanol (Gibco); bovine fibroblasts may be cultured in 50:50 DMEM/F12 (Gibco) and IMDM (with HEPES, Cytiva) with 15% FBS and the same supplements. Addition of 5 ng/ml of human bFGF (Peprotech) may be used to improve cynomolgus, bovine, and porcine fibroblast cultures.

Mouse naïve pluripotent stem cells may be grown in KSR-based mouse embryonic stem cell (mESC) media: high-glucose DMEM medium supplemented with 15% KSR (Invitrogen), 1% Glutamax, 1% NEAA, 1% penicillin-streptomycin, 1% β-mercaptoethanol, and 20 ng/ml human recombinant LIF (purified in-house) on Mitomycin C-inactivated C3H MEF feeder layer. Mouse Gof18 GFP-E3 epiblast stem cells (EpiSCs) (Han et al., 2010) may be cultured in Stem Flex media (Gibco) on FBS-coated dishes.

Human pluripotent cells may be cultured in hESC media: either in DMEM/F12 supplemented with 15% KSR, 1% Glutamax, 1% NEAA, 1% penicillin-streptomycin, 1% β-mercaptoethanol and 5 ng/ml bFGF or in StemFlex media (Gibco) on Matrigel-coated dishes (Corning) or Mitomycin C-inactivated CF1 MEF feeder layer. Cynomolgus iPSCs may be cultured in StemFlex media on Mitomycin C-inactivated CF1 MEF feeder layer. Bovine and porcine iPSCs may be derived and cultured in StemFlex media supplemented with 2 μM XAV939 (Sigma) on Mitomycin C-inactivated CF1 MEF feeder layer in a hypoxic 5% O₂, 5% CO₂ incubator at 37 °C.

In a preferred embodiment, increasing the level of the SoxB1 factor variant or the Sox17 factor variant as defined in item a); the fusion protein or the complex or composition as defined in item b) and/or the SoxB1 factor variant as defined in item c); and optionally, an inhibitor of p53 function; and/or one or more additional reprogramming factor(s) in said non-pluripotent cells is achieved by (co-)expressing in said non-pluripotent cell(s) the factor variant, POU factor, fusion protein, complex, and/or Klf family member as defined in items a) to c) and optionally the inhibitor of p53 function, and/or one or more additional reprogramming factor(s).

The terms “pluripotency” and “pluripotent” as used herein refer to the ability of a cell to differentiate into cells of all three germ layers, i.e., the endoderm, mesoderm and ectoderm. Accordingly, the term “pluripotent cells” refers to undifferentiated cells that are capable under conditions that promote differentiation. Those conditions include without limitation culturing cells in differentiation media(s), treating the pluripotent cells with differentiation factors, aggregating the cells into spheroid/embryoid bodies or embryo-like structures using suspension drops or specialized culture plates, such as AggreWell, combining the cells with embryos or embryo-like structures, and/or injecting the cells in vivo of producing progeny that are derivatives of any of the three germ layers or the germline. Included in the definition of pluripotent cells are embryonic cells of various types, such as induced pluripotent stem cells (iPSCs), embryonic stem cells as well as and embryonic germ cells (EGCs). In vivo pluripotency is a transient state, which is acquired within the inner cell mass of developing pre-implantation blastocysts during the segregation of cells of the inner cell mass into the primitive endoderm and pluripotent pre-implantation native epiblasts. During the transition from pre-implantation to early post-implantation stage, also referred to as the transition from a naïve to a primed state, changes in the molecular and functional characteristics of those cells lead to a certain developmental restriction with respect to ability to self-organize, contribute to development, ability to differentiate into certain specific lineages, particularly the germline. (Weinberger L, Ayyash M, Novershtern N, Hanna J H. Dynamic stem cell states: naïve to primed pluripotency in rodents and humans. Nat Rev Mol Cell Biol. 2016 March; 17(3):155-69. doi: 10.1038/nrm.2015.28. Epub 2016 Feb 10. PMID: 26860365.).

The term “induced pluripotent stem cell (iPSC)” as used herein refers to a type of pluripotent stem cell artificially derived from a non-pluripotent cell, typically a somatic cell, such as a skin cell or blood cell. iPSCs can be generated from somatic cells by epigenetic resetting of somatic cells, e.g., by retroviral transduction of somatic cells, such as fibroblasts, hepatocytes, or gastric epithelial cells, with transcription factors, such as Oct3/4, Sox2, Klf4, and optionally c-Myc (Takahashi and Yamanaka, 2006).

Pluripotent cells can exist in multiple states of pluripotency. The best-known examples are naïve pluripotent cells and primed pluripotent cells, which resemble a pre-implantation pluripotent state and a post-implantation pluripotent state, respectively.

As used herein, the term “naïve pluripotent cells” refers to pluripotent cells typically identified by one or more or all of the following characteristics: an active Oct4 distal enhancer (Oct4DE), expresses high levels of the pluripotency factors Sox2, Oct4, Nanog, Klf2, Klf4, Klf17, Rex1, Dnmt3L, Argfx and/or Susd2; exhibit dome-shape morphology, self-renewal in response to LIF, high clonogenicity, low level of global genome methylation and a XaXa X-chromosome status, wherein Xa refers to one active X chromosome. Naïve cells contribute to development more efficiently than primed cells and are therefore more appropriate for genetic engineering (especially multiplex gene targeting), for derivation of germline and extra-embryonic lineages, modelling of development and disease, etc. However, different naïve pluripotent cell lines exhibit differences in all those properties, most importantly the developmental potential, which the inventors found to correlate with the expression of endogenous Sox2 and the level of Sox2/Oct4 heterodimerization.

In a preferred embodiment, “naïve pluripotent cells” are further characterized by two or more characteristics selected from the group comprising: an enhanced developmental potential, a higher expression level of Sox2, an activated POU5f1 distal enhancer, a reactivation of the X chromosome in female lines, a higher expression level of naïve pluripotency-specific markers, a reduced DNA methylation, an enhanced capacity to differentiate, preferably into a germline cell, and/or an enhanced capacity to develop into chimeric tissue(s), organ(s), or organism(s).

The term “high-grade naïve pluripotent cells” refers to naïve pluripotent cells of high developmental potential. The relative developmental potential can be measured by comparing the abilities of given pluripotent cells to develop to chimeric animals upon aggregation with morula or injection into a blastocyst with further transfer in utero, or development ex utero, and/or by the ability to generate all-PSC animals in a tetraploid complementation assay, and/or the ability to efficiently differentiate into all cell lineages, in particular the germline.

The “tetraploid complementation assay” generally comprises first the production of a tetraploid embryo by taking an embryo at “two-cell stage” and fuse the two cells, e.g., by applying an electrical current. The fused cell is a tetraploid and will continue to divide, wherein its daughter cells are all tetraploid. While those tetraploid embryos can develop to the blastocyst stage and may be implanted in a uterus, a living fetus cannot develop. Because the tetraploid embryos still develop functional trophectoderm that gives rise to placenta, they can serve as surrogates for cultured pluripotent stem cells that can take over the inner cell mass and generate the whole fetus. For tetraploid complementation assay, the tetraploid embryo is combined with a diploid embryonic stem cells (ESCs) or iPSCs that can develop to a fetus, wherein the fetus is exclusively derived from the ESCs or iPSCs and the extra-embryonic tissues (e.g., placenta) are derived from the tetraploid cells. The embryonic stem cell as described in the exemplary tetraploid complementation assay therefore develop into an all-ESC fetus. Instead of an embryonic stem cell, other pluripotent stem cells may be used, e.g., induced pluripotent stem cells, wherein a higher developmental potential of these pluripotent stem cells means a higher ability to generate all-iPSC animals in the above-described tetraploid complementation assay. The tetraploid complementation assay as used herein according to the present invention is described in the Examples herein below.

As used herein, the term “chimeric animal” or “animal chimera” refers to an animal composed of cells of different genetic backgrounds (or even different species in case of cross-species animal chimeras). Most commonly, in the current state of the art, chimeric animals are created for the purpose of genetic engineering of the animal by aggregating genetically edited cells with a wild-type surrogate embryo (or a surrogate embryo of a different genetic background). The partly genetically engineered chimeric animal then gives rise to fully genetically edited animals as their progeny. In the current state of the art, this is most commonly done for laboratory mice, but it could potentially be applied to all mammals. A “chimeric molecule”, “chimeric polypeptide”, refers, thus, to a molecule, or polypeptide that function as one, yet its parts are of different origin. For example, Sox2-17 (super-SOX) presented herein functions as a single transcription factor (enhanced Sox2), yet its different structural elements are derived from two transcription factors: Sox2 and Sox17. The structural elements of Sox17 were not added to Sox2, but rather some elements of Sox2 were replaced with corresponding non-conserved elements of its paralog—Sox17. Two different version of Sox2-17: mouse and human (capitalized, SOX2-17) were prepared as described in the Examples.

The term “genetically engineered animal”, as used herein, refers to any animal with purposefully edited genome for example by introducing an exogenous DNA into a cell and has become part of the genome of said animal that was derived from said cell.

The ability to contribute to the generation of chimeric animals can be determined for example by measuring the average percentage of cells within the chimeric embryos at different stages of development or after birth that were derived from the given pluripotent cells in morula aggregation/blastocyst injection assays. Differences in the ability to generate all-PSC animals can be determined by measuring the proportion of tetraploid embryos aggregated with given PSCs that give rise to full-term all-PSC animals, the proportion of tetraploid embryos aggregated with given PSCs that are born alive, the proportion of tetraploid embryos aggregated with given PSCs that initiated breathing, the proportion of tetraploid embryos aggregated with given PSCs that survive till adulthood, etc. Differenced in the ability to generate all-PSC embryos can also be evaluated using artificial embryo assembly in vitro as was recently demonstrated by Hanna and Zernicka-Goetz labs (Amadei et al. 2022). The ability to differentiate into germline can be determined by evaluating the ability of given PSCs to differentiate into germline in vitro or in vivo, for example by evaluating the percentage of cells that activate germline markers, measuring the levels of germline marker expression, or evaluating the functionality of the resulting germline. High-grade naïve pluripotent cells have higher developmental potential as measured by one or several of the above-mentioned assays, compared to low-grade naïve pluripotent cells or compared to primed pluripotent cells, which are low-grade by definition. The inventors also suggest that the pluripotency grade can be determined or estimated by measuring the levels of endogenous Sox2 capable of heterodimerizing with Oct4 in given PSCs, wherein increased numbers of Sox2/Oct4 heterodimerization indicated a higher grade of pluripotency. This can be determined or estimated by methods such as western blot, EMSA, qPCR, RNA-seq, ATAC-seq, ChIP-seq, immunostaining combined with microscopy or FACS, tracking reporter genes etc.

Accordingly, as used herein, the term “low-grade pluripotent cells” refers to low-grade naïve pluripotent cells, and primed pluripotent cells, such as induced pluripotent stem cells, embryonic stem cells, or epiblast stem cells.

The term “primed pluripotent cells” as used herein refers to cells typically identified by the following characteristics: no active Oct4 distal enhancer, an active Oct4 proximal enhancer, flattened colony morphology, high level of global genome methylation, low clonogenicity, low developmental potential, do not respond to Lif/Stat3; self-renew in response to Fgf/Erk; exhibit a XaXi X-chromosome activation status, wherein Xi is an inactive X chromosome; among others (Nichols et al., 2009). Primed pluripotent cells and are not equivalent across species, for example human “conventional” embryonic stem cells are considered primed but have various naïve features and are distinct from mouse primed epiblast stem cells. In general, primed pluripotent cells are developmentally more restricted than naïve pluripotent cells, for example mouse epiblast stem cells, which are considered primed pluripotent cells that are not differentiated, however, they downregulate certain pluripotency genes, such as Sox2, Klf2, Klf5, Nanog, Rex1, Esrrb, upregulate lineage commitment factors like Otx2 and Zic2, which “prime” a certain future differentiation (Weinberger, L., Ayyash, M., Novershtern, N. et al. Dynamic stem cell states: naïve to primed pluripotency in rodents and humans. Nat Rev Mol Cell Biol 17, 155-169 (2016). https://doi.org/10.1038/nrm.2015.28).

As used herein, the term “low-grade naïve pluripotent cells” refers to naïve pluripotent cells that exhibit lower developmental potential compared to high-grade naïve pluripotent cells. For example, the naïve pluripotent cells that cannot generate all-PSC animals are low-grade compared to high-grade naïve pluripotent cells that can generate all-PSC animals. “Low-grade” and “high-grade” are not absolute, but relative terms. Theoretically, the most high-grade PSCs would be those that can generate healthy adult all-PSC animals for 100% of tetraploid aggregated embryos. Other types of PSCs theoretically could benefit from low-to-high grade conversion methods. The inventors demonstrated that low-grade PSCs have lower expression of endogenous Sox2 or/and lower capacity to form Sox2/Oct4 heterodimer on canonical SoxOct DNA motif compared to high-grade PSCs (see Example 5, herein below).

The term “non-pluripotent cell” as used herein refers to any non-pluripotent cell that is partially differentiated or fully differentiated, including a cell in culture, an explanted cell from a subject or a cell within a subject. The non-pluripotent cell may be from any animal species, preferably from a mammal, for example, mouse, human, non-human primate, livestock, cat, dog, etc

In a preferred embodiment of the ninth aspect of the present invention, the non-pluripotent cells are selected from fibroblasts, keratinocytes, blood cells, urine-derived cells and/or any other somatic cell type.

As used herein, the term “Kruppel-like-factor (Klf) family” refers to Klf genes initially identified as a factor for the generation of mouse iPSCs and also demonstrated to be a factor for generation of human iPSCs. Members of the “Klf family” as used herein include, e.g., Klf1, Klf2, Klf4, Klf5, Klf17 or other Klf factors from human or other animal species. Exemplary Klf4 proteins are the proteins encoded by the murine Klf4 gene (all sequences deposited under the NCBI Reference Sequence NM_010637, such as NM_010637.3) and the human klf4 gene (all sequences deposited under the NCBI Reference Sequence NM_004235, such as NM_004235.6). The terms “Klf4, “KLF4”, “Klf-4” and the like as referred to herein include any of the naturally occurring forms of the Klf4 transcription factor, or variants thereof that maintain Klf4 transcription factor activity (e.g., within at least 50%, 80%>, 90% or 100% activity) compared to wild type Klf4 as measured by methods known in the art, such as measuring the efficiency of iPSC generation in reprogramming experiments, or measuring the efficiency of naïve reset.

In some embodiments, variants have at least 90% amino acid sequence identity across their whole sequence compared to the naturally occurring Klf4 polypeptide. In other embodiments, the Klf4 protein is the protein as identified by all sequences deposited under the NCBI Reference Sequence NP_001300981, such as NP_001300981.1 or NP_034767, such as NP_034767.2 or SEQ ID NOs: 33 or 34, respectively.

An “inhibitor of p53 function” as used herein may be any agent, as far as it is capable of inhibiting either (a) the function of the p53 protein or (b) the expression of the p53 gene (TP53). That is, not only agents that act directly on the p53 protein to inhibit the function thereof and agents that act directly on the p53 gene to inhibit the expression thereof, but also agents that act on a factor involved in p53 signal transduction to result in inhibition of the function of the p53 protein or the expression of the p53 gene, are also included in the scope of “an inhibitor of p53 function” as mentioned herein. Factors involved in p53 signal transduction are without limitation for example MDM2, MDMX, SIRT1, CRM1, FACT, E6 ubiquitin ligase. Agents that act on these factors are for example: Nutilin-3a (MDM2), XI-006 (MDMX), Tenovins (SIRT1), Letomycin B (CRM1), Quanacrine (FACT), and RITA (E6) (see Sanz et al., Inhibition of p53 inhibitors: progress, challenges and perspectives. J Mol Cell Biol. 2019. 11(7):586-599). Examples inhibitors of p53 function include, but are not limited to, a siRNA, a shRNA, a miRNA, an antisense nucleic acid molecule, an aptamer or a ribozyme against p53, a chemical inhibitor of p53, an antibody or antibody mimetic, an anti-p53 antagonist antibody or a nucleic acid that encodes the same and a decoy nucleic acid comprising a consensus sequence of a p53-responsive element.

Preferably, the functional inhibitor of p53 is a substance that inhibits the expression of the p53 gene, more preferably an expression vector that encodes an siRNA or shRNA against p53.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g., as an antiviral mechanism or in shaping the chromatin structure of a genome.

siRNAs found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001, Nature; 411(6836):494-8). 2′-deoxynucleotides in the 3′ overhangs are as efficient as ribonucleotides but are often cheaper to synthesize and probably more nuclease resistant.

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves the target mRNAs. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

The following type of molecules also effect RNAi: microRNAs (miRNA) and antisense nucleic acid molecules. miRNA molecules are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of p53 function after introduction into the respective cells.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

Antisense molecules, siRNAs and shRNAs of the present invention are preferably chemically synthesized using a conventional nucleic acid synthesizer. Suppliers of nucleic acid sequence synthesis reagents include Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK).

The antisense molecules, siRNAs, shRNAs may comprise modified nucleotides such as locked nucleic acids (LNAs). The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers are synthesized chemically and are commercially available. The locked ribose conformation enhances base stacking and backbone pre-organization. This significantly increases the hybridization properties (melting temperature) of oligonucleotides.

Aptamers in the art have been selected which bind nucleic acid, proteins, small organic compounds, and even entire organisms. A database of aptamers is maintained at http://aptamer.icmb.utexas.edu/. More specifically, aptamers can be classified as DNA or RNA aptamers or peptide aptamers. Whereas the former consists of (usually short) strands of oligonucleotides, the latter consist of a short variable peptide domain, attached at both ends to a protein scaffold. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamer's inherently low molecular weight. The rapid clearance of aptamers can be an advantage in applications such as in vivo diagnostic imaging. Several modifications, such as 2′-fluorine-substituted pyrimidines, polyethylene glycol (PEG) linkage, etc. are available with which the half-life of aptamers easily can be increased to the day or even week time scale.

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

Examples of chemical inhibitors of p53 include, but are not limited to, p53 inhibitors typified by pifithrin (PFT)-α and -β, which are disclosed in WO 00/44364, PFT-μ disclosed in Storm et al., 2006, analogues thereof and salts thereof (for example, acid addition salts such as hydrochlorides and hydrobromides, and the like) and the like. Of these, PFT-α and analogues thereof [2-(2-Imino-4,5,6,7-tetrahydrobenzothiazol-3-yl)-1-p-tolylethanone, HBr (product name: Pifithrin-α) and 1-(4-Nitrophenyl)-2-(4,5,6,7-tetrahydro-2-imino-3(2H)-benzothiazolyl)ethanone, HBr (product name: Pifithrin-α, p-Nitro)], PFT-β and analogues thereof [2-(4-Methylphenyl)imidazo[2,1-b]-5,6,7,8-tetrahydrobenzothiazole, HBr (product name: Pifithrin-α, Cyclic) and 2-(4-Nitrophenyl)imidazo[2,1-b]-5,6,7,8-tetrahydrobenzothiazole (product name: Pifithrin-α, p-Nitro, Cyclic)], and PFT-μ. [Phenylacetylenylsulfonamide (product name: Pifithrin-μ)] are commercially available from Merck.

The term “antibody” as used in accordance with the present invention comprises, for example, polyclonal or monoclonal antibodies. Furthermore, also derivatives or fragments thereof, which still retain the binding specificity to the target are comprised in the term “antibody”. The isotype of the antibody is not particularly limited, and is preferably IgG, IgM or IgA, particularly preferably IgG. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g., in Harlow and Lane (1988) and (1999) and Altshul et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Kohler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Surface plasmon resonance as employed in the BIAcore system can be used to increase the efficiency of phage antibodies.

As used herein, the term “antibody mimetics” refers to compounds which, like antibodies, can specifically bind antigens but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers®, trispecific binding molecules and prododies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, refers to a molecule based on the 10th extracellular domain of human fibronectin 11 (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with a differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors, and which are connected by linker peptides. Binding of target molecules occurs via the A-domain and domains with the desired binding specificity can be selected, for example, by phage display techniques. The binding specificity of the different A-domains contained in an avimer may but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule by randomising the amino acids on the binding surface (Mouratou B, Béhar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. The trispecific binding molecule is preferably a TriTac. A TriTac is a T-cell engager for solid tumors which comprised of three binding domains being designed to have an extended serum half-life and be about one-third the size of a monoclonal antibody.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects.

An anti-p53 antagonist antibody can be produced using p53 or a partial peptide thereof as an antigen, by a method of antibody or anti-serum production known per se. As examples of known anti-p53 antagonist antibodies, PAb1801 (Oncogene Science Ab-2) and DO-1 (Oncogene Science Ab-6) (Gire and Wynford-Thomas, 1998) and the like can be mentioned. A nucleic acid that encodes an anti-p53 antagonist antibody can be isolated from a hybridoma that produces an anti-p53 monoclonal antibody by a conventional method. The H-chain and L-chain genes obtained may be joined together to prepare a nucleic acid that encodes a single-chain antibody.

Still another agent that inhibits the function of the p53 protein is a decoy nucleic acid comprising a consensus sequence of p53-responsive element (e.g., Pu-Pu-Pu-G-A/T-T/A-C-Py-Py-Py (Pu: purine base, Py: pyrimidine base); SEQ ID NO:35). Such a decoy nucleic acid is commercially available (e.g., p53 transcription factor decoy (GeneDetect.com)).

In a preferred embodiment of the method of the ninth aspect of the invention, the method is performed as an in vitro or ex vivo method.

At variance with the above-described preferred embodiment of the ninth aspect of the invention, the method is performed as an in vivo method.

In a tenth aspect, the present invention relates to a reprogramming method for rejuvenating aged cell(s), tissue(s), organ(s) or organism(s), said method comprising increasing in said aged cell(s), tissue(s), organ(s) or organism(s) the level of: a) the SoxB1 factor variant of the first aspect of the invention or the Sox17 factor variant the second aspect of the invention, and a POU factor; and/or b) the fusion protein of the third aspect of the invention or the complex or composition of the fourth aspect of the invention; and/or c) the SoxB1 factor variant sharing the amino acid sequence comprising or consisting of SEQ ID NOs: 13 or 14, and a Klf family member;
and optionally:
   an inhibitor of p53 function; and/or
   one or more additional reprogramming factor(s).

Appropriate reprogramming factors are described elsewhere in the specification.

In a preferred embodiment of the tenth aspect of the invention, the method comprises increasing the level of the SoxB1 factor variant or the Sox17 factor variant and a POU factor as defined in item a); and/or the fusion protein or the complex or composition as defined in item b); and/or the SoxB1 factor variant and a Klf family member as defined in item c); and optionally:
an inhibitor of p53 function; and/or
one or more additional reprogramming factor(s)
   by (co-)expression of the factor variant, POU factor, fusion protein, complex, and/or Klf family member as defined in items a) to c) and optionally: the inhibitor of p53 function and/or one or more additional reprogramming factor(s).

In a preferred embodiment, the “rejuvenated cell(s)” are preferably rejuvenated senescent cells.

The term “rejuvenating” refers to the process of erasing epigenetic modifications that lead to cellular, tissue, organ or organism aging. Aging in cell, tissue, organ or organism can be characterized, inter alia by one or more or all of the following markers: activation of the p53/p21^{CIP1}, pRb/p16^{INK4A} or other tumor suppressor pathways in cells and tissues, cells arrested, shortening of telomere size in cells, increased expression of senescent-associated factors (e.g., β-galactosidase (SA β-Gal)), specific chromatin modification as senescence-associated heterochromatic foci (SAHF), specific secretome, reduced/altered mitochondrial activity, accumulation of epigenetic changes, changes to gene expression patterns, depletion or loss of stem cells within tissues and organs, loss or partial loss of regenerative capacity of tissues and organs, loss or partial loss of functionality, occurrence or high propensity to age-related diseases. A process of rejuvenation is observed when one or more or all of these markers of aging is/are reduced in an aged or senescent cell, aged tissue, aged organ or aged organism due to the rejuvenating process.

The term “senescent cell” refers to a cell that exhibit cell cycle arrest, elicited by replicative exhaustion due to telomere attrition or in response to stresses such as DNA damage, chemotherapeutic drugs, or aberrant expression of oncogenes. This arrest is implemented primarily through activation of p53 and the up-regulation of the cyclin-dependent kinase (CDK) inhibitors pI6^{INK4A} and p21^{CIP1} (Collado et al. 2007, Cell, 130: 223-233). A “senescent cell” may be characterized by at least one or more or all of the following characteristics: activation of the p53/p21^{CIP1} and pRb/pI6^{INK4A} tumor suppressor pathways (hereafter referred as senescence effectors), cells arrested irreversibly in GI, shortening of telomere size, expression of senescent-associated β-galactosidase activity (SA β-Gal), specific chromatin modification as senescence-associated heterochromatic foci (SAHF), specific secretome, reduced/altered overall mitochondrial activity. Irreversible cell arrest in GI may be assessed by FACS. Shortening of telomere size may be characterized by evaluating the mean terminal restriction fragment (TRF) length for example by Southern blot analysis. A method for detecting expression of senescent-associated β-galactosidase activity (SA β-Gal) is known in the art. A method for detecting expression of senescence-associated heterochromatic foci (SAHF) by indirect immunofluorescence is described in EP2694642. Overall mitochondrial activity can be evaluated by measuring the transmembrane potential generated by the proton gradient.

An aged cell is a proliferative cell that exhibits one or more of the following characteristics: upregulation of tumor suppressors, decreased ability to proliferate, changes to DNA methylation pattern, accumulation of other epigenetic changes, misregulated gene expression, decreased or lost ability to perform its function. The aging markers can be observed using techniques known in the art such as epigenetic clocks (Horvath, 2013).

In one preferred embodiment, said aged or senescent cells display at least one or more (or all) of the following characteristics of the aging phenotype: upregulation of tumor suppressors, cells arrested irreversibly in GI, expression of senescent-associated β-galactosidase activity (SA β-Gal), expression of senescence-associated heterochromatic foci (SAHF), and altered overall mitochondrial activity, changes to DNA methylation patterns, accumulation of other epigenetic changes, misregulated gene expression, decreased or lost ability to perform its function.

Aged tissue is tissue that exhibits one or multiple of the following characteristics: accumulation of the senescent or aged cells within the tissue, changes to DNA methylation patterns, accumulation of other epigenetic changes, misregulated gene expression, depletion or loss of stem cells within the tissue, decrease of loss regenerative capacity, decreased or lost ability to perform its function.

An aged organ is an organ that exhibits one or multiple of the following characteristics: accumulation of the senescent or aged cells within the organ, changes to DNA methylation patterns, accumulation of other epigenetic changes, misregulated gene expression, depletion or loss of stem cells within the organ, decrease of loss regenerative capacity, decreased or lost ability to perform its function.

An aged organism is an animal organism that exhibits one or multiple of the following characteristics: accumulation of the senescent or aged cells within the organism, changes to DNA methylation patterns in some or all of its cells and tissues, accumulation of other epigenetic changes in some or all of its cells and tissues, misregulated gene expression in some or all of its cells and tissues, depletion or loss of stem cells within one or multiple of its tissues or organs, loss of regenerative capacity of some or multiple of its tissues and organs, occurrence or high propensity of occurrence of age-related disease or diseases.

Aged or senescent cells may be from various tissues, such as from an aged human patient or non-human animal in need of autologous regenerative treatment. Methods to obtain samples from various tissues and methods to establish primary cells are well-known in the art (see e.g. Jones and Wise, Methods Mol Biol. 1997). Said aged or senescent cells include, but are not limited to, primary cells from blood, bone marrow, adipose tissue, nervous tissue, skin, skin appendages, internal organs such as heart, gut or liver, mesenchymal tissues, muscle, bone, cartilage or skeletal tissues.

Increasing the level of the factors as recited in items a) to c) in accordance with the tenth aspect of the invention may be performed by contacting the aged cells, senescent cells, aged tissues, aged organs or aged organism with the factors, fusion proteins, and/or complexes or compositions as defined in items a) to c) of the tenth aspect of the invention so that the respective factors, fusion proteins, and/or complexes or compositions are taken up by the cell, as well as transfecting or transducing the cell with nucleic acids encoding the factors, fusion proteins and/or complexes and (co-)expressing the factors, fusion proteins and/or complexes.

In a preferred embodiment of the tenth aspect of the invention, the method is performed as an in vitro or ex vivo method.

At variance with the above-described preferred embodiment of the tenth aspect of the invention, the method is an in vivo method.

In a preferred embodiment of the ninth or the tenth aspect of the present invention, the one or more additional reprogramming factors are selected from: a Klf family member, a Myc family member, Lin28, Nanog and GLIS1.

The term “Lin28” refers to a protein that is encoded by the LIN28A gene in humans. It is a marker of undifferentiated human embryonic stem cells and encodes a cytoplasmic mRNA-binding protein that binds to and enhances the translation of the IGF-2 (Insulin-like growth factor 2) mRNA. Lin28 has also been shown to bind to the let-7 pre-miRNA and block production of the mature let-7 microRNA in mouse embryonic stem cells. Yu et al. demonstrated that it is a factor in iPSCs generation, although it is not mandatory. Exemplary Lin28 is the protein encoded by murine gene (all sequences deposited under the NCBI Reference Sequence NM_145833, such as NM_145833.1) and human LIN28A gene (all sequences deposited under the NCBI Reference Sequence NM_024674, such as NM_NM_024674.6). The term “Lin28” as referred to herein includes any of the naturally occurring forms of the Lin28 transcription factor, or variants thereof that maintain Lin28 transcription factor activity (e.g., within at least 50%, 80%, 90% or 100% activity) compared to wild type Lin28 as measured by methods known in the art, such as measuring the efficiency of iPSC generation in reprogramming experiments.

In some embodiments, variants have at least 90% amino acid sequence identity across their whole sequence compared to the naturally occurring Lin28 polypeptide. In other embodiments, the Lin28 protein is the protein as identified by all sequences deposited under the NCBI references NP_078950, such as NP_078950.1 or NP_665832, such as NP_665832.1, corresponding to SEQ ID NOs: 36 and 37, respectively.

The term “at least 50%, 80%, 90% or 100% activity” or similar terms used throughout the specification is intended to mean in all instances “at least 50%, at least 80%, at least 90% or 100% activity”.

The term “Nanog” or “nanog” refers to a transcription factor critically involved with self-renewal of undifferentiated embryonic stem cells. In humans, this protein is encoded by the NANOG gene. Exemplary nanog is the protein encoded by murine gene (all sequences deposited under NCBI Reference Sequence NM_028016, such as NM_028016.3) and human Nanog gene (all sequences deposited under the NCBI Reference Sequence NM_024865, such as NM_024865.4). The term “Nanog” or “nanog” and the like as referred to herein thus includes any of the naturally-occurring forms of the Nanog transcription factor, or variants thereof that maintain Nanog transcription factor activity (e.g. within at least 50%, 80%, 90% or 100% activity) compared to wild type Nanog as measured by methods known in the art, such as measuring the efficiency of iPSC generation in reprogramming experiments.

In some embodiments, variants have at least 90% amino acid sequence identity across their whole sequence compared to the naturally occurring Nanog polypeptide. In other embodiments, the Nanog protein is the protein as identified by all sequences deposited under the NCBI references NP_079141, such as NP_079141.2 or NP_082292, such as NP_082292.1, corresponding to SEQ ID NOs: 38 and 39, respectively.

The term “GLIS1” as used herein refers to a protein that belongs to the GLIS protein family (GLI similar family) of zinc finger-type transcription factors. Forced expression of GLIS1 promotes the formation of iPSCs (Maekawa et al., 2001). Exemplary GLIS1 proteins are the proteins encoded by the GLIS1 gene in humans (all sequences deposited under the NCBI Reference Sequence NM_147193, such as NM_147193.4). The term “GLIS1” as referred to herein includes any of the naturally occurring forms of the GLIS1 transcription factor (including the isoforms), or variants thereof that maintain GLIS1 transcription factor activity (e.g., within at least 50%, 80%, 90% or 100%) activity compared to wild type GLIS1 as measured by methods known in the art, such as measuring the efficiency of iPSC generation in reprogramming experiments.

In some embodiments, variants have at least 90% amino acid sequence identity across their whole sequence compared to the naturally occurring GLIS1 polypeptide. In other embodiments, the GLIS1 protein is the protein as identified by all sequences deposited under the NCBI reference NP_671726, such as NP_671726.2, corresponding to SEQ ID NO: 40.

In a preferred embodiment of the SoxB1 factor variant as defined in item a) of the ninth or the tenth aspect of the present invention, the SoxB1 factor variant comprises or consists of the amino acid sequence of SEQ ID NOs: 13 or 14.

In a further preferred embodiment of the POU factor as defined item a) or b) of the ninth or the tenth aspect of the present invention, the POU factor is selected from: Oct4, Oct1, Oct2, Oct6, Brn1, Brn2, Brn4 or other engineered or natural POU factors that have high propensity to cooperate with Sox on canonical SoxOct motifs.

In an even more preferred embodiment of the SoxB1 factor variant, Sox17 factor variant and POU factor as defined in item a) of the ninth or the tenth aspect of the invention, the method comprises (co-)expressing in the non-pluripotent cell(s) or increasing in said aged cell(s), tissue(s), organ(s), or organism(s) the expression of: (i) the SoxB1 factor variant of the first aspect of the present invention or the Sox17 factor variant the second aspect of the invention, and Oct4; and/or (ii) the SoxB1 factor variant of the first aspect of the present invention or the Sox17 factor variant of the second aspect of the invention and Oct4 and Klf4 or GLIS1; and/or (iii) the SoxB1 factor variant of the first aspect of the present invention or the Sox17 factor variant the second aspect of the invention and Oct4, Klf4 and GLIS1; and/or (iv) the SoxB1 factor variant of the first aspect of the present invention or the Sox17 factor variant the second aspect of the invention and Oct4, Klf4 and c-Myc; and/or (v) the SoxB1 factor variant of the first aspect of the present invention or the Sox17 factor variant the second aspect of the invention and Oct4, Klf4, c-Myc and Lin28.

The skilled person is aware that the reference sequence encoding the protein/factor described herein is not limited to the NCBI Reference Sequences explicitly mentioned herein but includes all follow up NCBI Reference Sequences, respectively, encoding for the same protein/factor.

It is known to the skilled person that a protein/factor in two different mammalian species are encoded by sequences deposited by distinct NCBI Reference sequence number. The skilled person understands that the proteins/factors for the purpose of the disclosure as described herein are chosen to be from the same species, preferably human. For example, if one or more reprogramming factor(s) is/are selected from the group of human POU factor, any further reprogramming factor selected from the group of Klf and/or Myc family and/or other reprogramming factors are preferably selected from human.

The term “aged cells” as in this and the following embodiment is a synonym for senescent cells. The term “aged cell(s), tissue(s), organ(s), or organism(s)” or similar terms used throughout this specification is intended to mean in all instances “aged cell(s)”, “aged tissue(s)”, “aged organ(s)” and “aged organism(s)”.

In a preferred embodiment of the Klf family member as defined in item c) of the ninth or the tenth aspect of the present invention, the Klf family member is selected from: Klf4, Klf2, Klf17.

In a preferred embodiment of the SoxB1 factor variant and the Klf family member as defined in item c) of the ninth or the tenth aspect of the invention, the method comprises (co-)expressing in the non-pluripotent cell(s) or increasing in said aged cell(s), tissue(s), organ(s) or organism(s) the expression of: (i) the SoxB1 factor variant sharing the amino acid sequence comprising or consisting of SEQ ID NOs: 13 or 14 and Klf4; or (ii) the SoxB1 factor variant sharing the amino acid sequence comprising or consisting of SEQ ID NOs: 13 or 14, Klf4 and a Myc family member.

As used herein, the term “Myc family member” refers to factors encoded by myc proto-oncogenes implicated in cancer.

In a further preferred embodiment of the method of the ninth or the tenth aspect of the present invention, the method does not comprise (co-)expressing an inhibitor of p53 function. p53 inhibition increases the reprogramming efficiency but might decrease the quality of iPSCs because of increased cell proliferation, and reduced response to DNA damage. Highly efficient Super-SOX presented here allows reducing the number of factors required for the reprogramming. The efficiency boost allows generation of higher quality iPSCs by avoiding oncogenes, such as Myc or p53 inhibitors.

In an eleventh aspect, the present invention relates to a method for conversion of pluripotent cell(s) into high-grade naïve pluripotent cell(s), the method comprising culturing said pluripotent cell(s) under conditions suitable for conversion of said pluripotent cell(s) into high-grade naïve pluripotent cell(s), wherein said conditions comprise increasing in said pluripotent cell(s) the level of: a) a SoxB1 factor or the SoxB1 factor variant of the first aspect of the invention and a Klf family member; and/or b) the SoxB1 factor variant sharing the amino acid sequence comprising or consisting of SEQ ID NOs: 13 or 14; and optionally:
an inhibitor of p53 function; and/or
one or more additional reprogramming factor(s)

In a preferred embodiment of the eleventh aspect of the invention, the increasing of the level of a SoxB1 factor or the SoxB1 factor variant; and a Klf family member as defined in item a) and b), respectively; and optionally:
an inhibitor of p53 function; and/or
one or more additional reprogramming factor(s)
   is achieved by (co-)expression of the factor, factor variant and/or Klf family member as defined in items a) and/or b) and optionally: the inhibitor of p53 function and/or one or more additional reprogramming factor(s). Additional reprogramming factors have been defined herein above.

Culturing the cell(s) in accordance with the eleventh aspect of the present invention preferably includes culturing conditions that support/facilitate the conversion of pluripotent cells into high-grade naïve pluripotent cells and preferably allow proliferation of high-grade naïve pluripotent cells.

Culturing the cells includes specific conditions, such as naïve media, e.g. Rset (STEMCELL Technologies), PXGL (see Bredenkamp, Nicholas et al. 2019b), or other formulations (containing one or more small molecule inhibitors of the following molecules/pathways: HDACs, WNT, MEK, FGF, FGFR, GSK3, ROCK, and PKC, p38, JNK, BMP, ERK, TGFB), optionally feeder layer, and/or hypoxic conditions (5% O₂).

Culturing the cell(s) in accordance with the eleventh aspect of the present invention may include contacting the cell(s) with the SoxB1 factor variant and the Klf family member as defined in items a) and b) of the eleventh aspect of the invention so that the respective SoxB1 factor variant and Klf family member are taken up by the cell, as well as transfecting or transducing the cell with nucleic acids encoding the SoxB1 factor variant and the Klf family member and co-expressing the SoxB1 factor variant and the Klf family member.

As used according to the eleventh aspect of the invention, the term “pluripotent cell”, refers to any pluripotent cell of lower differential potential than a high-grade naïve pluripotent cell. In a preferred embodiment of the eleventh aspect of the invention, the pluripotent cell is a low-grade pluripotent cell. In another, even more preferred embodiment, the method comprises (co-)expressing in said pluripotent cell(s): a) a SoxB1 factor or the SoxB1 factor variant of the first aspect of the invention, and a Klf family member; and/or b) the SoxB1 factor variant sharing the amino acid sequence comprising or consisting of SEQ ID NOs: 13 or 14; and optionally:
an inhibitor of p53 function; and/or
one or more additional reprogramming factors.

In a preferred embodiment of the eleventh aspect of the present invention, the pluripotent cell is selected from the group consisting of a primed pluripotent stem cell, an induced pluripotent stem cell (iPSC) and an embryonic stem cell (ECS).

Furthermore, the one or more additional reprogramming factors in accordance with the eleventh aspect of the invention are preferably selected from: a POU factor, a Klf family member, a Myc family member, Lin28, Nanog and GLIS1.

In a preferred embodiment of the SoxB1 factor variant as defined in item a) of the eleventh aspect of the present invention, the SoxB1 factor is Sox2 and the SoxB1 factor variant of the first aspect of the invention is the SoxB1 factor variant sharing the amino acid sequence comprising or consisting of SEQ ID NOs: 11 or 47 or the SoxB1 factor variant comprising or consisting of the amino acid sequence of SEQ ID NOs: 13 or 14.

In another preferred embodiment of the Klf family member as defined in item a) of the eleventh aspect of the present invention, the Klf family member is selected from Klf4, Klf2 or Klf17.

In a preferred embodiment of the SoxB1 factor variant and the Klf family member as defined in item a) of the eleventh aspect of the invention, the method comprises (co-)expressing in the pluripotent cell(s): (i) the SoxB1 factor variant sharing the amino acid sequence comprising or consisting of SEQ ID NOs: 13 or 14, and Klf4; or (ii) the SoxB1 factor variant sharing the amino acid sequence comprising or consisting of SEQ ID NOs: 13 or 14, Klf4 and c-Myc; or (iii) or the SoxB1 factor variant comprising or consisting of the amino acid sequence SEQ ID NOs: 13 or 14, Klf4 and Oct4.

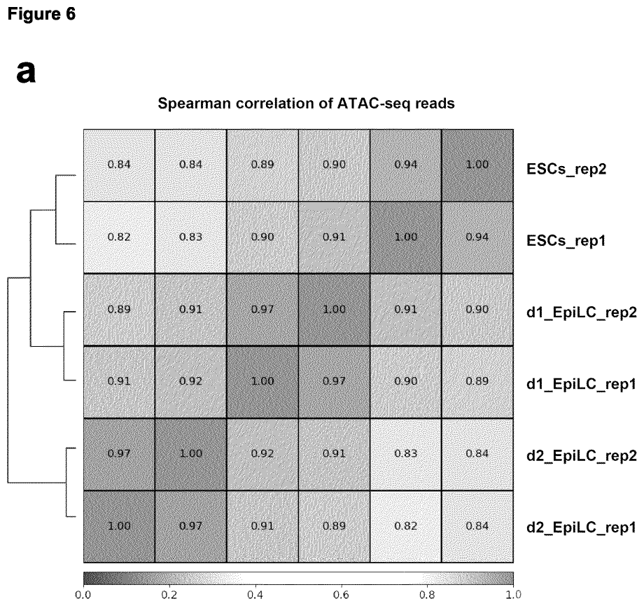

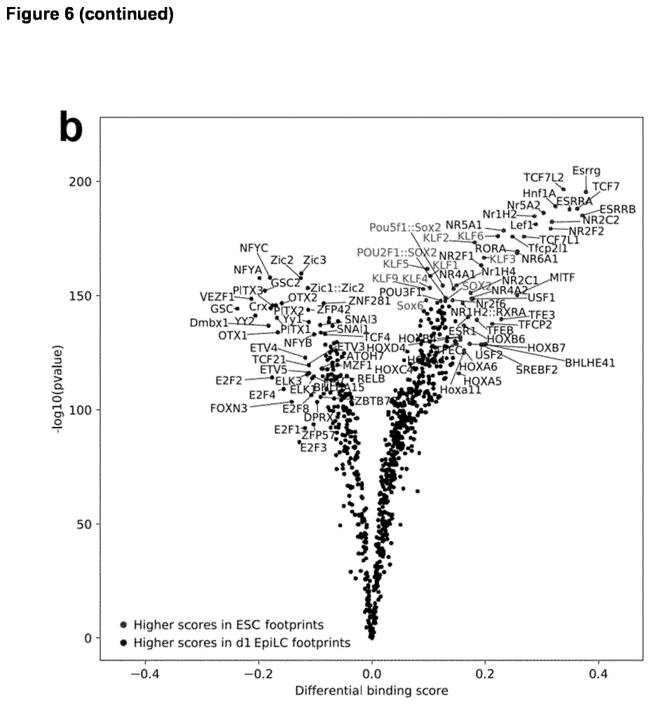

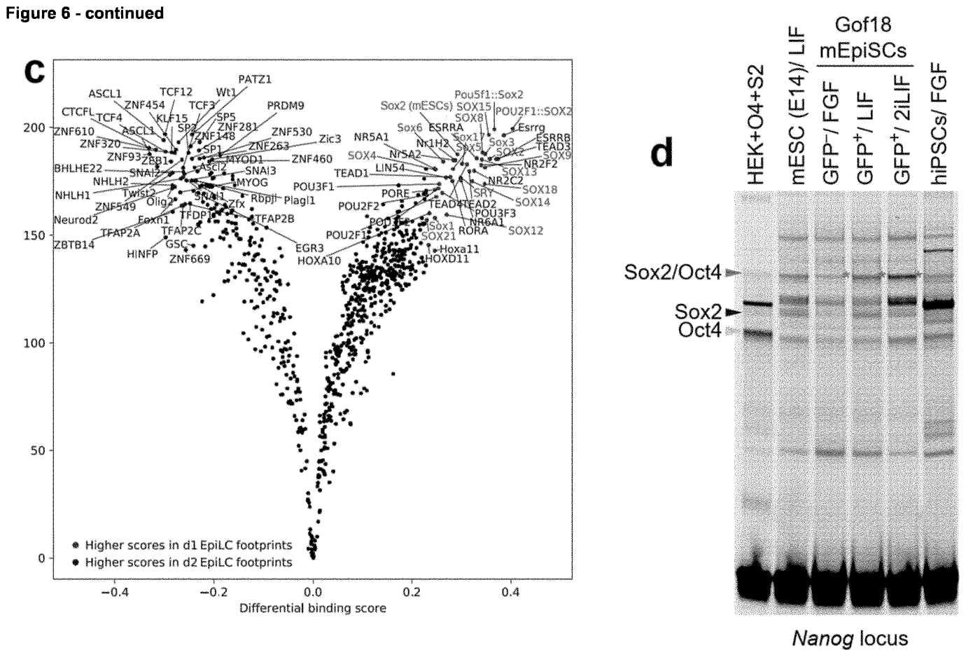

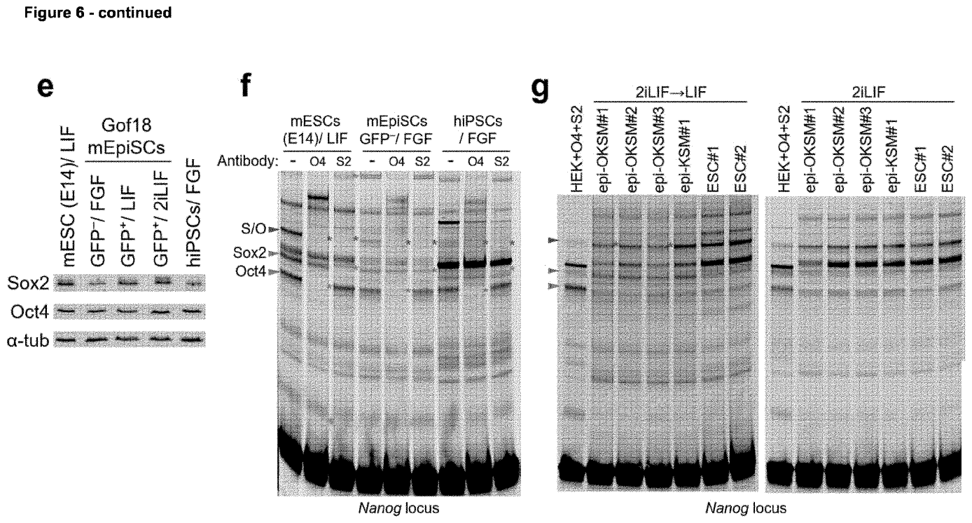

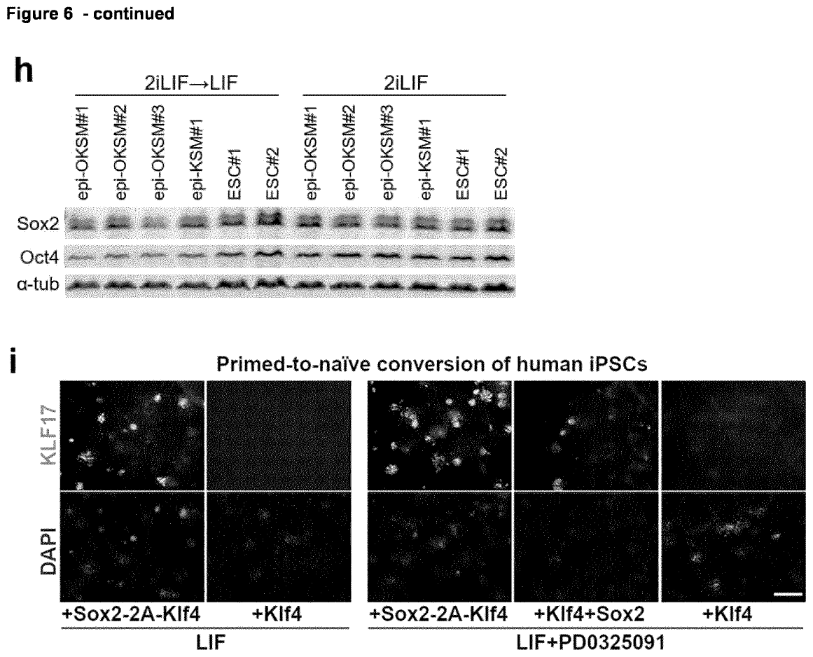

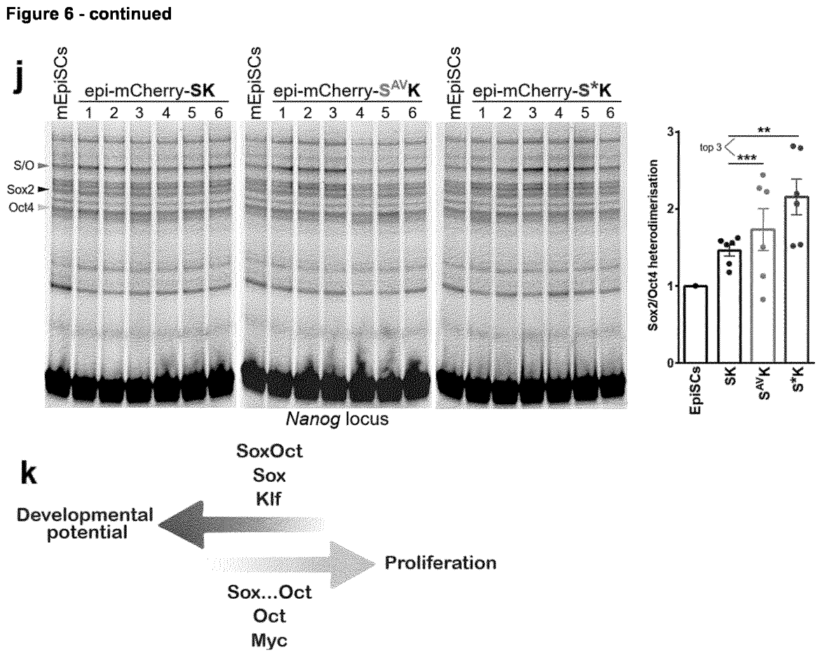

In a further preferred embodiment of the eleventh aspect of the invention, sequences encoding the SoxB1 factor or the SoxB1 factor variant of the first aspect of the present invention and the Klf family member are combined in one polycistronic cassette. One example is the SOX-P2A-Klf4 cassette as shown by the inventors in FIG. 6i-j (SEQ ID NO: 44). Further examples include the cassettes of SEQ ID NOs: 45 or 46.

In a particularly preferred embodiment of the eleventh aspect of the invention, the polycistronic cassette includes sequences encoding the SoxB1 factor or the SoxB1 factor variant, the Klf family member, and a fluorescent protein wherein the latter may be used to track the cassette expression and disappearance. One example is the mCherry-T2A-SOX-P2A-KLF4 episomal vector as shown by the inventors in the examples herein below.

The term “fluorescent protein” as used herein means a protein that exhibits low, medium or intense fluorescence upon irradiation with light of the appropriate excitation wavelength.

In a preferred embodiment of the eleventh aspect of the invention, the method is performed as an in vitro or ex vivo method.

At variance with the above-described preferred embodiment of the eleventh aspect of the invention, the method is performed as an in vivo method.

In a preferred embodiment of the ninth to eleventh aspects of the present invention, the cells are mammalian cells.

In a particularly preferred embodiment, the cells are selected from human, non-human primate, mouse, porcine, bovine, horse, dog, cat or elephant cells.

In twelfth aspect, the present invention relates to a kit of parts comprising or consisting of a) the SoxB1 factor variant of the first aspect of the invention; and/or b) the Sox17 factor variant the second aspect of the invention; and/or c) the fusion protein of the third aspect of the invention; and/or d) the complex or composition of the fourth aspect of the invention; and/or e) the nucleic acid molecule or the combination of nucleic acid molecules the fifth aspect of the invention; and/or f) the vector or the combination of vectors of the sixth aspect of the invention; and/or the cell of the seventh aspect of the invention; and optionally instructions for use of the kit.

The various components of the kit may be packaged into one or more containers such as one or more vials. The vials may, in addition to the components, comprise preservatives or buffers for storage. The kit may comprise instructions how to use the kit.

In an thirteenth aspect, the present invention relates to a method for producing rejuvenated cell(s), tissue(s), organ(s) or organism(s), the method comprising increasing in aged cell(s), tissue(s), organ(s) or organism(s) the level of: a) the SoxB1 factor variant of the first aspect of the invention or the Sox17 factor variant of the second aspect of the invention, and a POU factor; and/or b) the fusion protein of the third aspect of the invention or the complex or composition of the fourth aspect of the invention; and/or c) the SoxB1 factor variant of the first aspect of the invention, and a Klf family member; and optionally, one or more additional reprogramming factor(s); thereby producing rejuvenated cell(s), tissue(s), organ(s) or organism(s).

In a preferred embodiment of the thirteenth aspect of the invention, the increasing the level in aged cell(s), tissue(s), organ(s) or organism(s) of the factor variants and POU factor as defined in item a), and/or the fusion protein or complex or composition as defined in item b) and/or the SoxB1 factor variant and a Klf family member as defined in item c) and optionally, one or more additional reprogramming factor(s) is achieved by (co-)expressing the factor variants, the POU factor, the fusion protein, the complex and/or the Klf family member as defined in items a) to c) and optionally, one or more additional reprogramming factor(s).

Culturing the cell in accordance with the thirteenth aspect of the present invention may include contacting the aged cell(s), tissue(s), organ(s) or organism(s) with the SoxB1 factor variant or the Sox17 factor variant, and a POU factor as defined in items a) and/or the fusion protein or complex or composition as defined in item b) and/or the SoxB1 factor variant and a Klf family member of the thirteenth aspect of the invention so that the respective SoxB1 factor variant, the Sox17 factor variant, the POU factor, the fusion protein, the complex or composition and/or the Klf family member are taken up by the aged cell(s), tissue(s), organ(s) or organism(s), as well as transfecting or transducing the aged cell(s), tissue(s), organ(s) or organism(s) with nucleic acids encoding the respective SoxB1 factor variant, the Sox17 factor variant, the POU factor, the fusion protein, the complex and/or the Klf family member and (co-)expressing the respective SoxB1 factor variant, the Sox17 factor variant, the POU factor, the fusion protein, the complex and/or the Klf family member.

The culture conditions might be the same as normal for the given cell type, or may include specific factors, media supplements, nutritional supplements that facilitate the rejuvenation process.

In a preferred embodiment of the thirteenth aspect, the method further comprises optionally increasing the level of one or more additional reprogramming factor(s) and/or inhibitors, wherein the inhibitor is selected from the group of TGFbeta inhibitors, Src inhibitors and other kinase inhibitors, which may be used in combination with epigenetic modifiers. These inhibitors are known in the art and routinely used for supporting the reprogramming of aged cell(s), tissue(s), organ(s) or organism(s).

In a preferred embodiment of the SoxB1 factor variant as defined in item a) of the thirteenth aspect of the invention, the method comprises co-expressing in the pluripotent cell: the SoxB1 factor variant comprising or consisting of SEQ ID NOs: 13 or 14.

In a preferred embodiment of the method of the thirteenth aspect, the rejuvenated cells are rejuvenated senescent cells.

In a further preferred embodiment, the Klf family member is Klf4.

In a fourteenth aspect, the present invention relates to a method for producing high-grade naïve pluripotent cell(s), the method comprising increasing in low-grade pluripotent cell(s) the level of: a) a SoxB1 factor or the SoxB1 factor variant of the first aspect of the invention or the Sox17 factor variant of the second aspect of the invention, and a Klf family member; and/or b) the fusion protein of the third aspect of the invention or the complex or composition of the fourth aspect of the invention and a Klf family member; and optionally one or more additional reprogramming factor(s); thereby producing high-grade naïve pluripotent cell(s).

Increasing the level of the factors, fusion protein or complex or the composition as recited in items a) and/or b) in accordance with the fourteenth aspect of the invention may be performed by contacting the low-grade pluripotent cell(s) with the factors, fusion proteins and/or complexes or compositions as defined in items a) and/or b) of the fourteenth aspect of the invention so that the respective factor, fusion protein, and/or complex or composition is taken up by the cell(s), as well as transfecting or transducing the cell(s) with nucleic acids encoding the factor, fusion protein and/or complex and (co-)expressing the factor, fusion protein and/or complex.

In a preferred embodiment of the fourteenth aspect of the invention, the increasing the level of the referred factors or proteins is achieved by (co-)expression of the referred factors or proteins.

In a preferred embodiment of the SoxB1 factor variant and the Klf family member as defined in item a) of the fourteenth aspect of the invention, the method comprises (co-)expressing in the low-grade pluripotent cell: (i) the SoxB1 factor variant sharing the amino acid sequence comprising or consisting of SEQ ID NOs: 13 or 14, and Klf4; or (ii) the SoxB1 factor variant sharing the amino acid sequence comprising or consisting of SEQ ID NOs: 13 or 14, Klf4 and c-Myc; or (iii) or the SoxB1 factor variant comprising or consisting of the amino acid sequence SEQ ID NOs: 13 or 14, Klf4 and Oct4.

In a preferred embodiment of the method according to the fourteenth aspect of the invention, the method further comprises collecting the produced naïve pluripotent cells.

In a preferred embodiment, the Klf family member is Klf4.

In a further preferred embodiment of the method of the fourteenth aspect of the invention, the low-grade pluripotent cell(s) are iPSC(s), wherein optionally:
the iPSC(s) is/are obtained or obtainable by the method according to the ninth aspect of the invention,
the method according to the fourteenth aspect further comprises prior to step a) providing the iPSC(s) by conducting the method according to the ninth aspect of the invention.

In a preferred embodiment of the method of any one of the ninth, tenth, eleventh, thirteenth and fourteenth aspect of the invention, the (co-)expression is integration-free (co-)expression, preferably wherein the integration-free (co-) expression is from:
the nucleic acid molecule of the fifth aspect of the invention; or
the vector of the sixth aspect of the invention, wherein the vector is preferably an episomal vector.

As used herein, the term “(co-)expression” refers to the production of one or more (poly)peptide(s) which may be (i) permanent, i.e., achieved by a constitutive (co-)expression; or (ii) non-permanent, i.e., achieved by a transient (co-)expression.

The term “constitutive (co-)expression”, as used herein, refer to the permanent production of a (poly)peptide in a host cell, wherein the (poly)peptide(s) is/are encoded in polynucleotide(s) comprised in an exogenous gene and is delivered to the host cell. Delivery of genes into a host cell are for example transfection methods known to the skilled person, such as lipofection, electroporation, nucleofection, viral delivery (transduction).

Typically, constitutive (co-)expression is achieved by integration of DNA into the genome of the host cell.

In another preferred embodiment of the method of any one of the ninth, tenth, eleventh, thirteenth and fourteenth aspect of the invention, the (co-)expression is a constitutive (co-)expression.

The term “transient (co-)expression”, as used herein, refers to the non-permanent production of a (poly)peptide in a cell. The production of the (poly)peptide may be directly driven by an exogenous polynucleotide(s) encoding the (poly)peptide.

In a more preferred embodiment of the above preferred embodiment, the expression from an episomal vector, also herein referred to as episome, is a transient (co-)expression.

As used herein, the term “exogenous gene” refers to any polynucleotide sequence encoding a (poly)peptide, which:
is partly or entirely heterologous, i.e., foreign to the host cell to which it is introduced; or
is homologous to an endogenous gene of the host cell into which it is introduced but inserted in a way to influence the expression of other endogenously expressed (poly)peptide(s) in the host cell (e.g., a transcription factor protein which regulates the expression of an endogenous protein). The introduction of an exogenous gene, in other terms the delivery of polynucleotides encoding the (poly)peptide into a host cell may be conducted by transfection. The skilled person is aware of methods known in the art for transfection of a host cell. Non-limiting examples include nucleofection, lipofection, electroporation or viral delivery.

In another, or even more preferred embodiment of the above preferred embodiment, the nucleic acid molecule of the fifth aspect of the invention is an RNA, preferably a modified RNA, most preferably a modified mRNA (modRNA).

In a further preferred embodiment of the method of the fourteenth aspect of the invention, the method comprises (i) delivery of a Sox family TF, preferably a SoxB1 factor, or SoxB1 factor variant according to the first to sixth aspects of the invention and a Klf family member into a host cell, and (ii) culturing the cell or a plurality of cells as single cells under conditions suitable for producing high-grade naïve pluripotent cells.

Culturing the cells includes specific conditions, such as naïve media, e.g. Rset (STEMCELL Technologies), PXGL (Bredenkamp, et al., 2019b), or other formulations (containing one or more small molecule inhibitors of the following molecules/pathways: HDACs, WNT, MEK, FGF, FGFR, GSK3, ROCK, and PKC, p38, JNK, BMP, ERK, TGFB), optionally feeder layer, and/or hypoxic conditions (5% O₂).

In a further, or more preferred embodiment of item (i) of the above embodiment, the Sox family TF, preferably a SoxB1 factor, or SoxB1 factor variant according to the first to sixth aspects of the invention and a Klf family member are delivered as proteins, mRNA, modified RNA or plasmids into the host cell, preferably a cell according to the seventh aspect of the invention.

In another more preferred embodiment of the item (ii) of the above embodiment, the conditions suitable for producing naïve pluripotent cells, which includes high-grade and low-grade naïve pluripotent cells, include a) optionally plating the cell or a plurality of cells as single cells on inactivated feeder layer or in feeder-free conditions and b) optionally supplementing the media with small molecules, that support naïve reset, i.e., primed-to-naïve conversion (upgrade), or using cell culture media that supports naïve pluripotent cell cultures.

Small molecules according to the above embodiment are for example LIF, FGF signaling inhibitors (including MEK/ERK inhibitor such as PD0325901, or/and FGFR inhibitor such as PD173074), Wnt inhibitor such as XAV939, GSK3β inhibitor such as CHIR99021, PKC Inhibitor such as Go 6983.

The term “feeder”, as used herein, also understood as “feeder cells”, refers to cells of one type that are co-cultured with cells of another type to provide an environment in which the cells of the second type can grow. The feeder cells may optionally be from the same or from a different species than the cells they are supporting. In accordance with the above more preferred embodiment, the feeder cells may be inactivated when being co-cultured with other cells by means known to the skilled person, such as irradiation or treatment with an anti-mitotic agent such as mitomycin c, to prevent them from outgrowing the cells they are supporting.

The terms “feeder-free”, as used herein, refers to “feeder cell free” conditions, wherein in cultures or cell populations less than 5% (with decreasing preference) of the total cells in the culture are feeder cells, such as, e.g., less than 4%, less than 3%, less than 2%, less than 1%, less than 0.5%, less than 0.1% and less than 0.01%.

Cell culture media that supports naïve pluripotent cell cultures according to the above embodiment is any cell culture media typically used in the art and known by the skilled person. In a particular embodiment with the above embodiment, the cell culture media is selected from the group of self-made N2B27 media supplemented with some or all of above-mentioned factors and small molecules, or commercially available RSeT™ Medium, NaïveCult™.

In a preferred embodiment of the thirteenth and/or fourteenth aspect of the invention, the method is performed as an in vitro or ex vivo method.

At variance with the above-described preferred embodiment of the thirteenth and fourteenth aspect of the invention, the method is an in vivo method.

In a preferred embodiment of the method of any one of the ninth, tenth, eleventh, thirteenth, and fourteenth aspect of the invention, the non-pluripotent cells of the ninth aspect, the aged cells of the tenth and thirteenth aspect, the pluripotent cells of the eleventh and fourteenth aspect of the invention are mammalian cells.

In a particularly preferred embodiment of the above preferred embodiment, the non-pluripotent cells of the ninth aspect, the aged cells of the tenth and thirteenth aspect, the pluripotent cells of the eleventh and the low-grade pluripotent cells of the fourteenth aspect of the invention are selected from human, non-human primate, mouse, porcine, bovine, horse, dog, cat, elephant, or other mammalian cells.

In a fifteenth aspect, the present invention relates to an induced pluripotent cell, a high-grade naïve pluripotent cell or a rejuvenated cell, tissue, or organ produced or producible by the method according to any one of the ninth, tenth, eleventh, thirteenth and fourteenth aspect of the invention, respectively.

In a preferred embodiment of the fifteenth aspect of the invention, the induced pluripotent stem cell (iPSC) is characterized by (i) an expression of at least one, preferably at least two, more preferably at least three pluripotency-specific marker(s) selected from Oct4, Sox2, Nanog and Klf4; and/or (ii) a capacity to differentiate into each of the three primordial germ layers.

In a more preferred embodiment according to the expression as defined in item (i) of the above preferred embodiment of the fifteenth aspect of the invention, the expression is a detectable expression.

As used herein, the term “detectable expression” refers to a protein or gene expression, which is relatively or absolutely quantifiable in a sample of a cell, or directly in said cell, i.e., detectable by methods known in the art. Non-limiting examples for protein expression detection include Western blot, immunofluorescence staining, Enzyme-linked immunosorbent assay (ELISA), and mass spectrometry; and non-limiting example for gene expression detection include Northern blot, quantitative polymerase chain reaction (qPCR), DNA microarray and RNA-Seq.

In a more preferred embodiment of item (ii) of the above preferred embodiment of the fifteenth aspect of the invention, the three primordial germ layers are ectoderm, mesoderm and endoderm.

As used herein, the term “primordial germ layers” refers to embryonic germ layers.

The term “capacity to differentiate”, as interchangeably used herein with the terms “ability to differentiate”, “potential to differentiate”, and “readiness to differentiate” refers to the ability of a stem cell, or pluripotent cell to differentiate into a subset of more differentiated cells. The term “capacity to differentiate” does not encompass moving backwards along the differentiation spectrum such that a cell is produced that comprises a greater differentiation capacity than the parent cell. That is, the term “capacity to differentiate” does not encompass reprogramming methods to shift cells to a less differentiated state.

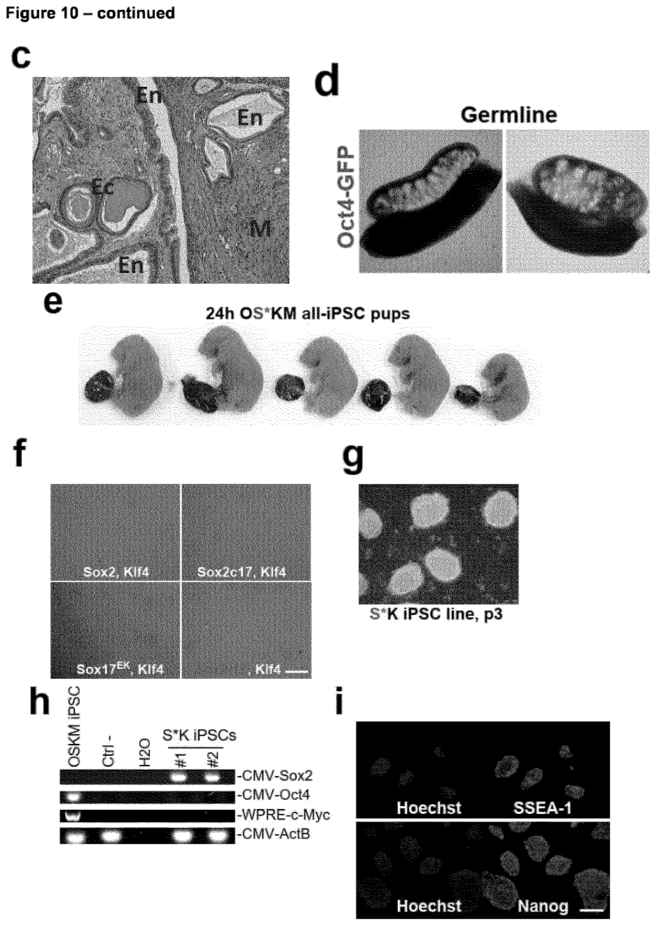

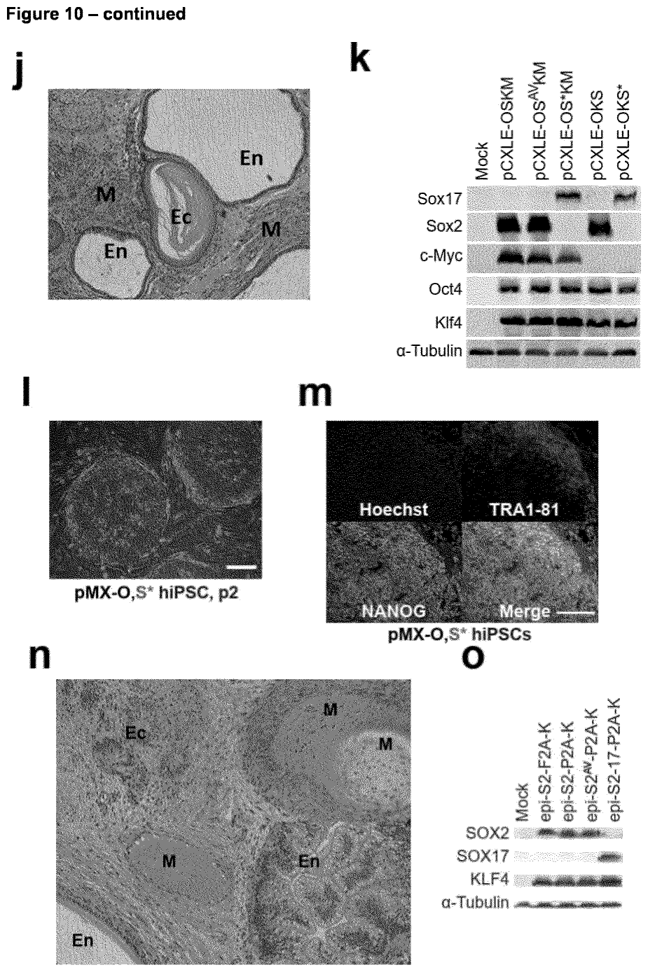

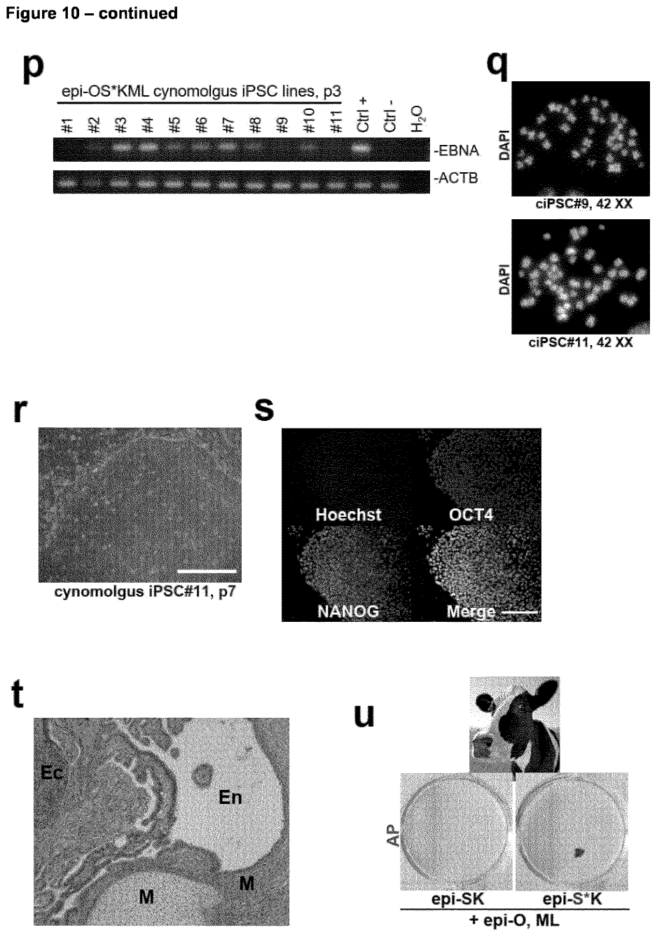

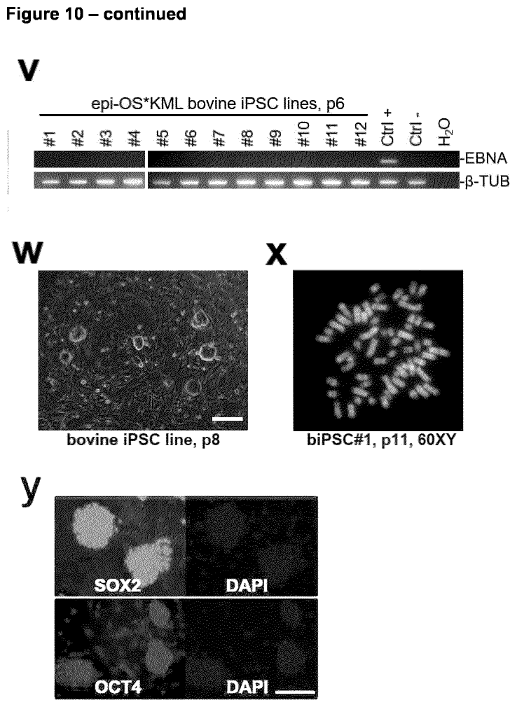

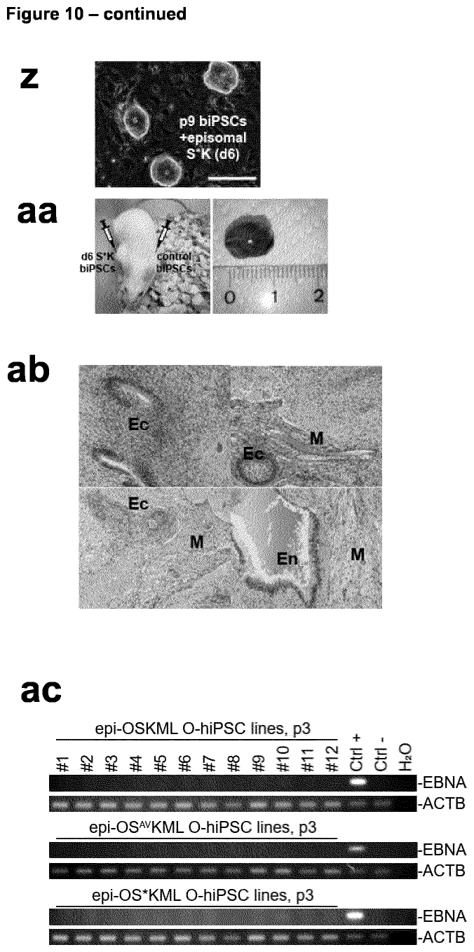

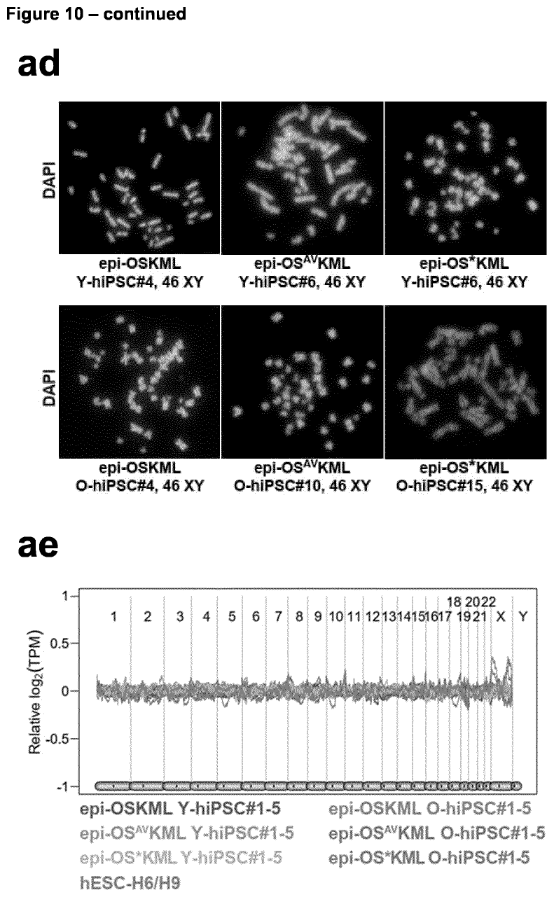

The capacity to differentiate into all three primordial germ layers can be tested by methods known to the skilled person, such as in vivo: a) a teratoma test, where the pluripotent cells are injected into immuno-compromised (SCID) mouse. After a few weeks to a few months, a tumor is formed that contains tissues derived from ectoderm, mesoderm and endoderm lineages, as shown for example in FIG. 10 c, j, n, t, aa and ab herein. b) an embryo complementation assays, where the cells are either aggregated with an early animal embryo, or injected into the blastocyst, the pluripotent cells then might integrate into the blastocyst and contribute to the development of the animal, or even form the whole animal body.

In vitro tests for determining the capacity to differentiate are, for example, a) random differentiation upon withdrawal of the growth factors that support pluripotent stem cells growth in undifferentiated condition (LIF or FGF), or exposure of the pluripotent cells to media or small molecules that induce differentiation (FCS, retinoic acid, and the like). The emergence of ectoderm-, mesoderm- and endoderm-derived lineages can be evaluated by marker gene expression (e.g., qPCR, RNA-seq, single cell sequencing, immunostainings), microscopy, functional tests, and b) directed differentiation, where the pluripotent cells are exposed to specific growth factors, cytokines, transcription factors, other media components that direct specific differentiation to a desired cell type.

In another preferred embodiment of the fifteenth aspect of the invention, the high-grade naïve pluripotent cell is characterized by at least of: (i) an enhanced developmental potential; (ii) a higher expression level of at least one, preferably at least two, more preferably at least three, more preferably at least four, more preferably at least five, most preferably six, naïve pluripotency-specific marker(s) selected from (but not limited to) Klf17, Klf4, Sox2, Susd2, Argfx, and Dnmt3l; (iii) a higher expression level of at least one primitive endoderm-specific marker(s) selected from Gata6, and Sox17; (iv) an activated POU5f1 distal enhancer; (v) a reactivated X chromosome in female lines; (vi) a reduced DNA methylation; (vii) an enhanced capacity to differentiate, preferably into germline cell; (viii) an enhanced capacity to contribute to development of an embryo(s) and/or animal(s); and/or (ix) a combination of two or more of (i) to (viii); in comparison to a corresponding non-naïve pluripotent cell or low-grade naïve pluripotent cell. It is known to the skilled person that the markers for naïve pluripotency as described above are not an exhaustive list of markers.

The term “Klf17”, as used herein above is a gene and protein name or a transcription factor from the Klf family, which regulates pre-implantation development in human and other primates. An example for human is KLF17 gene is encoded by all sequences deposited under the NCBI Reference Sequence NM_173484, such as NM_173484.4 and has been described in Boroviak et al., 2017 (Development (Cambridge, England) vol. 144, 2: 175-186. doi:10.1242/dev.145177) and in examples below.

The term “Susd2”, as used herein above is a gene and protein name, it is a cell surface marker of pre-implantation pluripotency in primates. An example for human is the SUSD2gene is encoded by all sequences deposited under the GenBank Reference: AAH33107, such as AAH33107.1 and has been described in Bredenkamp et al. (2019a) and examples below.

The term “Argfx”, as used herein above is a gene and protein name for Arginine-Fifty Homeobox transcription factor, a marker of naïve pluripotency in human and other animals. An example for human is ARGFX gene is encoded by all sequences deposited under the NCBI Reference Sequence NM_001012659, such as NM_001012659.2 and has been described in Boroviak et al., 2017 (Development (Cambridge, England) vol. 144, 2: 175-186. doi:10.1242/dev.145177) and in examples below.

The term “Dnmt3l”, as used herein above is a gene and protein name for DNA (Cytosine-5-)-Methyltransferase 3-Like protein responsible for CpG methylation—an epigenetic modification important for embryonic development. An example for human is DNMT3Lgene is encoded by all sequences deposited under the NCBI Reference Sequence NM_013369, such as NM_013369.4, and NM_175867, such as NM_175867.3 and has been described in Bi et al. 2022 and in examples below.

The term “Gata6”, as used herein above is a gene and protein name for GATA Binding Protein 6, a zinc finger transcription factor that plays important roles in development, and in particular regulates primitive endoderm fate. An example for human is GATA6 gene is encoded by all sequences deposited under the NCBI Reference Sequence NM_005257, such as NM_005257.6 and has been described in Plusa et al., 2008.

In a preferred embodiment of the above preferred embodiment, the (ix) combination of two or more of (i) to (viii) refers to at least two of, preferably at least three of, more preferably at least four of, more preferably at least five of, more preferably at least six of, more preferably at least seven of or most preferably eight of the features of (i) to (viii).

In another, even more preferred embodiment, the combination of at least two refers to the combination of all features in items (i) and (ii). In another preferred embodiment, the combination of at least two refers to the combination of the features in items (i) and (iii). In another preferred embodiment, the combination of at least two refers to the combination of the features in items (i) and (vii). In another preferred embodiment, the combination of at least two refers to the combination of the features in items (ii) and (vii). In another preferred embodiment, the combination of at least two refers to the combination of the features in items (iii) and (vii).

In another more preferred embodiment, the combination of at least three refers to the combination of the features in items (i), (ii) and (iii). In another more preferred embodiment, the combination of at least three refers to the combination of the features in items (i), (ii) and (vii). In another more preferred embodiment, the combination of at least three refers to the combination of the features in items (i), (iii) and (vii).

In another preferred embodiment, the combination of at least four refers to the combination of the features in items (i), (ii), (iii) and (iv). In another preferred embodiment, the combination of at least four refers to the combination of the features in items (i), (ii), (iii) and (vii).

In another more preferred embodiment, the combination of at least five refers to the combination of the features in items (i), (ii), (iii), (iv). and (v). In another more preferred embodiment, the combination of at least five refers to the combination of the features in items (i), (ii), (iii), (iv). and (vii).

In another more preferred embodiment, the combination of at least six refers to the combination of the features in items (i), (ii), (iii), (iv). (v) and (vi). In another more preferred embodiment, the combination of at least six refers to the combination of the features in items (i), (ii), (iii), (iv). (v) and (vii).

In another more preferred embodiment, the combination of at least seven refers to the combination of the features in items (i), (ii), (iii), (iv). (v), (vi) and (vii). In another more preferred embodiment, the combination of eight refers to the combination of the features in items (i), (ii), (iii), (iv). (v), (vi), (vii) and (viii).

The term “corresponding” in accordance with the above preferred embodiment of the fifteenth aspect of the invention means a cell from a comparable similar origin, i.e., animal, preferably a cell identical to the parent cell before reprogramming/conversion. For example, if the high-grade naïve pluripotent cell is a reprogrammed/converted human low-grade pluripotent cell, the corresponding non-naïve pluripotent cell is a human low-grade pluripotent cell selected from primed pluripotent cells, iPSCs, and embryonic stem cells.

The developmental potential as defined in item (i) of the above embodiment can be assessed by the skilled person by means described herein.

In more preferred embodiments, the expression level of the naïve pluripotency-specific marker(s) and the primitive endoderm-specific marker(s) as defined in item (ii), and (iii) of the above preferred embodiment of the fifteenth aspects of the invention, is a gene expression and/or protein expression, preferably the expression of the markers is a detectable expression of said marker proteins.

The expression levels according to the present invention can be quantified by any suitable means and methods available from the art. In general, relative and absolute quantification means and methods can be used. In absolute quantification no known standards or controls are needed. The expression level can be directly quantified. As well-known in the art, absolute quantification may rely in certain further embodiments on a predetermined standard curve. In relative quantification the expression level is quantified relative to a reference such as known control expression levels. Also, in the absence of controls, one can relatively quantify the expression level when comparing e.g., fluorescence intensities.

As used herein, the term “a higher expression level” or the term “increased expression level” refers to quantification of expression levels, e.g., levels of pluripotency-specific markers in comparison to a reference. The increase compared to the reference is at least, with increasing preference, 5 times more, at least 10 times more, at least, 20 times more, at least 30 times more, at least 40 times more, at least 50 times more, at least 60 times more, at least 70 times more, at least 80 times more, at least 900 times more, at least 100 times more, at least 110 times more, at least 120 times more, at least 130 times more, at least 140 times more, at least 150 times more, at least 160 times more, at least 170 times more, at least 180 times more, at least 190 times more, at least 200 times more, at least 210 times more, at least 220 times more, at least 230 times more, at least 240 times more, at least 250 times more, at least 260 times more, at least 270 times more, at least 280 times more, at least 290 times more, at least 300 times more.

As used herein, the term “POU5f1 distal enhancer” is also referred to as “Oct4 distal enhancer” and specifically refers to a regulatory region in the POU5F1 gene for regulating transcription of Oct4. A skilled person is able to determine the state, i.e., activation or inactivation, of regulatory regions of a gene by methods routinely employed in the art.

In accordance with the above embodiment in item (iv), the reactivated X chromosome in female lines is also described as “Xa chromosome” herein above, wherein the active status of both X chromosomes is referred to as “XaXa”. Reactivation of X chromosomes occurs on inactivated X chromosomes as an epigenetic process. During differentiation of cells, inactivation of one of the X chromosomes in female lines occur to compensate for potential dosage differences of X-linked genes between female XX and male XY cells. Inactivation of one of the X chromosomes in XX embryos happens early in mammalian development, as a result female naïve pluripotent stem cells have XaXa status, while female primed pluripotent stem cells have XaXi status. For example, mouse embryonic stem cells from the inner cell mass of the blastocyst contain two active X chromosomes, one of which will be inactivated upon exit from naïve state, during in vivo or vitro priming or differentiation. In human embryonic stem cells, the X chromosomes are already inactivated, because they represent primed pluripotent state. In human iPSCs and ESCs the inactivation state can be reversed. Reprogramming of hiPSCs or ESCs into an even more undifferentiated state, e.g., in naïve state, leads to the reversal of the inactivation, wherein at least one X chromosome is reactivated
Guo, et al., 2009; Theunissen, et al., 2016.

As used herein, the term “embryo” refers to a fertilized oocyte or alternatively to a zygote and also to cells in all stages of development from a fertilized oocyte or zygote up to the 5 or 6 days (blastocyst stage) and, in humans, up to the beginning of the third month of pregnancy. The origin of the embryo is preferably selected from the group including natural conception, in vitro fertilization (IVF), Intracytoplasmic Sperm Injection (ICSI), somatic cell nuclear transfer (SCNT) into natural or in vitro derived egg, and/or assembled from cultured cells.

In a preferred embodiment of the above preferred embodiment of item (viii), the enhanced capacity to contribute to development of an embryo(s) and/or animal(s) further comprises the enhanced capacity to contribute to development of embryo-like structures.

The term “embryo-like structures”, as used herein, refers to synthetic embryos wherein in contrast to typical embryos as defined above, the fertilization of an oocyte is not necessary. In other terms, synthetic embryos do not require an oocyte and sperm cell but rely on artificial assembly from cultured cells or a combination of embryo cells and cultured cells, mimicking the natural structures that arise in early development.

In another, even more preferred embodiment of the above preferred embodiment of item (viii), the embryo(s) and animal(s) is/are chimeric.

The term “chimeric animal” or “chimeric embryo” has been defined in this specification above.

In a sixteenth aspect, the present invention relates to an induced pluripotent cell (iPSC), a high-grade naïve pluripotent cell or a rejuvenated cell, tissue, or organ of the fifteenth aspect of the invention, for use:
as a medicament;
in regenerative medicine; and/or
in treating or preventing an age-related disease.

In a preferred embodiment of the sixteenth aspect of the invention, the treating or preventing of an age-related disease includes the slowing, inhibiting, or reversing of symptoms or the onset of progress of these symptoms.

The term “age-related disease”, as used herein, refers preferably to age-related tissue degeneration, wherein tissues include muscles, nerve and/or skin tissues.

The term “age-related disease” or the term “age-related condition”, as used herein, may also refer to any condition, disease, or disorder associated with aging such as, but not limited to, neurodegenerative diseases (e.g., Alzheimer's disease, Parkinson's disease, Huntington's disease, amyotrophic lateral sclerosis, dementia, and stroke), cardiovascular and peripheral vascular diseases (e.g., atherosclerosis, peripheral arterial disease (PAD), hematomas, calcification, thrombosis, embolisms, and aneurysms), eye diseases (e.g., age-related macular degeneration, glaucoma, cataracts, dry eye, diabetic retinopathy, vision loss), dermatologic diseases (dermal atrophy and thinning, elastolysis and skin wrinkling, sebaceous gland hyperplasia or hypoplasia, senile lentigo and other pigmentation abnormalities, graying hair, hair loss or thinning, and chronic skin ulcers), autoimmune diseases (e.g., polymyalgia rheumatica (PMR), giant cell arteritis (GCA), rheumatoid arthritis (RA), crystal arthropathies, and spondyloarthropathy (SPA)), endocrine and metabolic dysfunction (e.g., adult hypopituitarism, hypothyroidism, apathetic thyrotoxicosis, osteoporosis, diabetes mellitus, adrenal insufficiency, various forms of hypogonadism, and endocrine malignancies), musculoskeletal disorders (e.g., arthritis, osteoporosis, myeloma, gout, Paget's disease, bone fractures, bone marrow failure syndrome, ankylosis, diffuse idiopathic skeletal hyperostosis, hematogenous osteomyelitis, muscle atrophy, peripheral neuropathy, multiple sclerosis, amyotrophic lateral sclerosis (ALS), Duchenne muscular dystrophy, primary lateral sclerosis, and myasthenia gravis), diseases of the digestive system (e.g., liver cirrhosis, liver fibrosis, Barrett's esophagus), respiratory diseases (e.g., pulmonary fibrosis, chronic obstructive pulmonary disease, asthma, chronic bronchitis, pulmonary embolism (PE), lung cancer, and infections), and any other diseases and disorders associated with aging.

In a seventeenth aspect, the present invention relates to the use of the induced pluripotent cell (iPSC), the high-grade naïve pluripotent cell or the rejuvenated cell, tissue, or organ of the fifteenth aspect of the invention, for production of differentiated cell(s), tissue(s), organ(s), or organism(s).

As used herein, the term “differentiated” or the term “differentiation” in connection with cell(s), tissue(s) and/or organ(s), refers to the loss of pluripotency as defined herein in a cellular differentiation process to a specific cell type lineage. For example, a “differentiated cell” refers to a cell of a specialized cell type lineage derived from a cell of a less specialized cell type. In line with this, a differentiated cell is a non-pluripotent cell.

In a preferred embodiment of the use according to the seventeenth aspect of the invention, the differentiated cell(s), tissue(s), organ(s), or organism(s) is/are: (i) a transplant; (ii) an organoid; or (iii) cultured meat.

As used herein, the term “transplant”, also generally known as “graft”, refers to a free (unattached) cell(s), tissue(s), or organ(s) that is/are transferred from the site of origin of said cells, tissue or organ to a recipient to be integrated into a body fluid, tissue, organ or organism of or corresponding to a recipient. The term transplant further also includes the cell(s), tissue(s) or organ(s) that have been transferred.

In a more preferred embodiment of item (i) of the above preferred embodiment, the transplant is a skin graft.

In another more preferred embodiment of item (i) of the above preferred embodiment, the transplant is a replacement cell, preferably a replacement cell for a damaged and/or sick cell.

As used herein, the term “replacement cell” refers to a cell applied in “cell replacement therapy”, wherein an originally existing cell is replaced with another cell, i.e., the replacement cell.

The term “organoid”, as used herein, refers to a 3-dimensional growth of a specific type of cell(s) in vitro that retains characteristics of a tissue(s) and/or organ(s) consisting of said specific type of cells in vivo. In other terms, an organoid comprises cells of the same specific cell type(s) as found in the native tissue and/or organ, with the difference that the organoid cells were grown in vitro. Organoids are used as in research settings instead of the native tissue(s) and/or organ(s), preferably in drug screening, toxicity assays and regenerative medicine.

As used herein, the term “drug screening” refers to the identification of compounds or drugs that are useful as treatments for pathologic conditions of specific cells, tissues, or organs. The differentiated cells, tissues and/or organs may be used to be artificially induced with pathologic condition(s) that can be treated by a potential drug or compound, thereby determining the effectivity of potential new drugs or compounds.

The term “toxicity assay” means assays commonly used in the art for testing the influence of compounds by incubating the tested sample, e.g., a cell, a tissue, an organism, with the compounds and determining the survival and viability of said sample. For example, see the adherent cell differentiation and cytotoxicity assay (ACDC assay) (see Barrier et al., (2010) Evaluation of a mouse embryonic stem cell adherent cell differentiation and cytotoxicity (ACDC) assay. The Toxicologist 114: 358-9)

The term “regenerative medicine” or “regenerative therapy” refers to promoting the regenerative capacity of a cell, tissue, and/or organ. Regenerative medicine encompasses cellular and/or tissue engineering to replace, engineer, or regenerate cells, tissues, and/or organs and/or restoring or improving one or more biological function of a cell, tissue, and/or organ that is dysfunctional or impaired; as well as tissue engineering and organ regeneration.

As used herein, “regenerative capacity” refers to conversion of a cell, such as an adult somatic cell, into a dividing progenitor cell (a cell that can differentiate into a specific cell type) and differentiated tissue-specific cell. Regenerative capacity may additionally or alternatively refer to the ability of a cell, tissue, and/or organ to replicate, proliferate, regain function, and/or regenerate.

In a preferred embodiment of the use according to the cultured meat as defined in item (iii) of the above preferred embodiment of the seventeenth aspect, the cultured meat is a food product for animal or human food consumption.

In a more preferred embodiment of the use of the seventeenth aspect of the invention, the cultured meat is cultured beef, wherein the differentiated cells originate from a bovine induced pluripotent cell, a naïve pluripotent cell or a rejuvenated cell, tissue, or organ.

In another more preferred embodiment of the of the seventeenth aspect of the invention, the cultured meat is cultured pork, wherein the differentiated cells originate from a porcine induced pluripotent cell, a naïve pluripotent cell or a rejuvenated cell, tissue, or organ.

As used herein, the term “cultured meat” in connection with the present invention refers to cultivated meat not grown as a natural component of a living animal, in other terms a “cultured meat” is not obtained directly from the slaughter of a living animal.

As used herein, the term “food product” in connection with the present invention refers to a food supplement and/or a composition safe for human or animal consumption.

In a more preferred embodiment of the use according to the seventeenth aspect, the production of the differentiated cell(s), tissue(s) or organ(s) is preferably an in vitro production.

In an eighteenth aspect, the present invention relates to the use of the high-grade naïve pluripotent cell(s) obtained by the method of the fourteenth aspect of the invention for producing an embryo(s), or an animal(s), preferably a chimeric embryo(s) or a chimeric animal(s).

In a preferred embodiment of the eighteenth aspect of the invention, the embryo is an “early-stage embryo”. The term “early-stage embryo”, as used herein, means an embryo in the zygote stage, cleavage stage, morula or blastocyst stage.

In a nineteenth aspect, the present invention relates to a method for inducing high-grade naïve pluripotency within an embryo(s), thereby enhancing the viability and/or developmental potential of said embryo(s), the method comprising increasing in said embryo(s) the level(s) of: a) the SoxB1 factor variant of the first aspect of the invention or the Sox17 factor variant of the second aspect of the invention, and optionally a Klf family member and/or a POU factor; and/or b) the fusion protein of the third aspect of the invention or the complex or composition of the fourth aspect of the invention and optionally a Klf family member.

Induction of pluripotency in accordance with the nineteenth aspect of the invention comprises increasing the level of the SoxB1 factor variant, the Sox17 factor variant, optionally the Klf family member and/or the POU factor as defined in item a) and/or the fusion protein, the compounds of the complex or composition, and optionally a Klf family member and/or a POU factor as defined in item b). In accordance with the nineteenth aspect of the invention, increasing the level may be performed by contacting the embryo with the respective factors, fusion proteins and/or complexes or compositions as defined in items a) and/or b) so that the respective factors, fusion proteins and/or complexes or compositions are taken up by the embryo, as well as transfecting or transducing the embryo with nucleic acids encoding the factors, fusion proteins and/or complexes and co-expressing the factors, fusion proteins and/or complexes or compositions.

In a preferred embodiment, the Klf family member is Klf4.

In another preferred embodiment the embryo is a non-human embryo.

In a further preferred embodiment of the SoxB1 factor variant as defined in item a) of the nineteenth aspect of the invention, the method comprises (co-)expressing in the embryo: (i) the SoxB1 factor variant sharing the amino acid sequence comprising or consisting of SEQ ID NOs: 13 or 14, and Klf4; or (ii) the SoxB1 factor variant sharing the amino acid sequence comprising or consisting of SEQ ID NOs: 13 or 14, Klf4 and c-Myc; or (iii) or the SoxB1 factor variant comprising or consisting of the amino acid sequence SEQ ID NOs: 13 or 14, Klf4 and Oct4.

In a preferred embodiment of the SoxB1 factor variant and the Kif family member as defined in item a) of the eleventh aspect of the invention, the method comprises co-expressing in the pluripotent cell: (i) the SoxB1 factor variant sharing the amino acid sequence comprising or consisting of SEQ ID NOs: 13 or 14, and Klf4; or (ii) the SoxB1 factor variant sharing the amino acid sequence comprising or consisting of SEQ ID NOs: 13 or 14, Klf4 and c-Myc; or (iii) or the SoxB1 factor variant comprising or consisting of the amino acid sequence SEQ ID NOs: 13 or 14, Klf4 and Oct4.

In another preferred embodiment of the nineteenth aspect, the method refers to the induction of pluripotency in totipotent cells of a mammalian embryo(s) into high-grade naïve pluripotent cells of said embryo(s). In other terms, high-grade naïve pluripotency is induced in said embryo to aid the natural process of induction of naïve pluripotency, thereby compensating the low natural Sox2 expression of natural an embryo(s).

The viability of an embryo(s) in accordance with the nineteenth aspect can be determined by the skilled person by methods routinely employed in the art, such as non-invasive measurements of amino acid turnover, wherein the depletion/appearance of amino acids in the culture medium is assessed: the turnover of three amino acids, Asn, Gly and Leu, were significantly correlated with a clinical pregnancy and live birth (see Brison D R, et al. Identification of viable embryos in IVF by non-invasive measurement of amino acid turnover. Hum Reprod. 2004 October; 19(10):2319-24. doi: 10.1093/humrep/deh409. Epub 2004 Aug. 6. PMID: 15298971).

In a twentieth aspect, the present invention relates to the use of
a) the SoxB1 factor variant of the first aspect of the invention; and/or
b) the Sox17 factor variant the second aspect of the invention; and/or
c) the fusion protein of the third aspect of the invention; and/or
d) the complex or composition of the fourth aspect of the invention; and/or
e) the nucleic acid molecule or the combination of nucleic acid molecules the fifth aspect of the invention; and/or
f) the vector or the combination of vectors of the sixth aspect of the invention; and/or
g) the cell of the seventh aspect and/or the cell of the fifteenth aspect,
for the development of culture media for culturing or derivation of naïve pluripotent stem cells.

In a twenty-first aspect, the present invention relates to the use of
a) the SoxB1 factor variant of the first aspect of the invention; and/or
b) the Sox17 factor variant the second aspect of the invention; and/or
c) the fusion protein of the third aspect of the invention; and/or
d) the complex or composition of the fourth aspect of the invention; and/or
e) the nucleic acid molecule or the combination of nucleic acid molecules the fifth aspect of the invention; and/or
f) the vector or the combination of vectors of the sixth aspect of the invention; and/or
g) the cell of the seventh aspect and/or the cell of the fifteenth aspect,
for the development of differentiation media to derive germline and other cell types and tissues.

In a preferred embodiment of the twentieth or twenty-first aspect, one or more additional reprogramming factors may further be used for the development of a medium for culturing or derivation of naïve pluripotent stem cells or a differentiation medium. Reprogramming factors have been described above.

In a twenty-second aspect, the present invention relates to a germline differentiation medium comprising the cell of the seventh and/or the fifteenth aspect, and a molecule blocking the DNA binding or expression of a member of Oct family.

A member of the Oct family may be selected from the group of Oct2 and Oct4 in a preferred embodiment.

In a more preferred embodiment of the above preferred embodiment, a media for naïve reset further comprises an Oct family member monomer blocker, selected from the group of a siRNA, a shRNA, a miRNA, an antisense nucleic acid molecule, an aptamer or a ribozyme against Oct family member, a chemical inhibitor of Oct family member, an antibody or antibody mimetic, an anti-Oct family member antagonist antibody or a nucleic acid that encodes the same and a decoy nucleic acid comprising a consensus sequence of a Oct family member-responsive element. Antibody mimetics are selected from the group of affibodies, adnectins, anticalins, DARPins, avimers, nanofitins, affilins, Kunitz domain peptides, Fynomers®, trispecific binding molecules and prododies These polypeptides are described above.

In a twenty-third aspect, the present invention relates to a method of developing or optimizing a culture medium, comprising the step of testing whether the cell of the seventh aspect and/or the cell of the fifteenth aspect retains at least one marker of its phenotype or genotype over at least one cell cycle.

Alternatively, the testing is effected over at least one passage.

In a preferred embodiment of the twenty-third aspect, the developed or optimized medium is produced. Production may be according to conventional methods of media production.

In a twenty-fourth aspect, the present invention relates to a method of developing or optimizing a differentiation medium, preferably a germline differentiation medium, comprising the step of testing whether the cell of the seventh aspect or the cell of the fifteenth aspect displays at least one marker representative of a germline lineage or a cell type further differentiated therefrom.

In a preferred embodiment of the twenty-fourth aspect, the developed or optimized medium is produced. Production may be according to conventional methods of media production.

In a preferred embodiment of the twenty-third aspect, said at least one cell cycle or passage is at least 4, 5, 6, 7, 8, 9 or 10 cell cycles or passages.

In a preferred embodiment of the twenty-third or twenty-fourth aspect, the at least one marker representative of the induced pluripotent stem cell of the fifteenth aspect is selected from:
(i) an expression of at least one, preferably at least two, more preferably at least three, pluripotency-specific marker(s) selected from Oct4, Sox2, Nanog and Klf4; and/or
(ii) a capacity to differentiate into each of the three primordial germ layers.

In another preferred embodiment of the twenty-third or twenty-fourth aspect, the at least one marker representative of the high-grade naïve pluripotent cell of the fifteenth aspect is selected from:
(i) an enhanced developmental potential;
(ii) a higher expression level of at least one, preferably at least two, more preferably at least three, more preferably at least four, more preferably at least five, and most preferably six, naïve pluripotency-specific marker(s) selected from Klf17, Klf4, Sox2, Susd2, Argfx, and Dnmt3l;
(iii) a higher expression level of at least one primitive endoderm-specific marker(s) selected from Gata6, and Sox17;
(iv) an activated POU5f1 distal enhancer;
(v) a reactivated X chromosome in female lines;
(vi) a reduced DNA methylation;
(vii) an enhanced capacity to differentiate, preferably into a germline cell;
(viii) an enhanced capacity to contribute to development of an embryo(s) and/or animal(s); and/or
(ix) a combination of two or more of (i) to (viii);
   in comparison to a corresponding non-naïve pluripotent cell or low-grade naïve pluripotent cell.

In a preferred embodiment of the twenty-third or twenty-fourth aspect, retaining the at least one marker of the phenotype of the cell of the seventh aspect comprises retaining at least one of an enhanced developmental potential, a higher expression level of at least one, preferably at least two, more preferably at least three, more preferably at least four, more preferably at least five, and most preferably at least six naïve pluripotency-specific marker(s) selected from Klf17, Klf4, Sox2, Susd2, Argfx, and Dnmt3l, a higher expression level of at least one primitive endoderm-specific marker(s) selected from Gata6 and Sox17, an activated POU5f1 distal enhancer, a reactivated X chromosome in female lines, a reduced DNA methylation, an enhanced capacity to differentiate, preferably into a germline cell, and/or an enhanced capacity to contribute to development of an embryo(s) and/or animal(s), of the phenotype of the original cell carrying a SoxB1 factor variant of the first aspect, or the Sox17 factor variant of the second aspect.

In a twenty-fifth aspect, the present invention relates to a cell culture medium produced or producible by the method of the twenty-third aspect.

In a twenty-sixth aspect, the present invention relates to a germline differentiation medium produced or producible by the method of the twenty-fourth aspect.

The present invention also relates to a method of developing or optimizing a naïve stem cell derivation or maintenance medium, comprising the step of evaluating the levels and activity of Sox2/Oct4 heterodimer. This may be done according to conventional methods such as EMSA, Western blot, and ATAC-seq.

The present invention also relates to a method of developing or optimizing a differentiation medium (e.g., for germline differentiation), comprising the step of treating the cells to be differentiated with the SoxB1 factor variant of the invention or a cocktail comprised of SoxB1 factor or SoxB1 factor variant, Klf factor, preferably KLF4 and optionally one or more additional factor.

As regards the embodiments characterized in this specification, in particular in the claims, it is intended that the expression of the singular is plural unless the context clearly indicates otherwise. For example, a cell also means a plurality of cells and is interchangeable with the term “cell(s)”.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The Figures show:

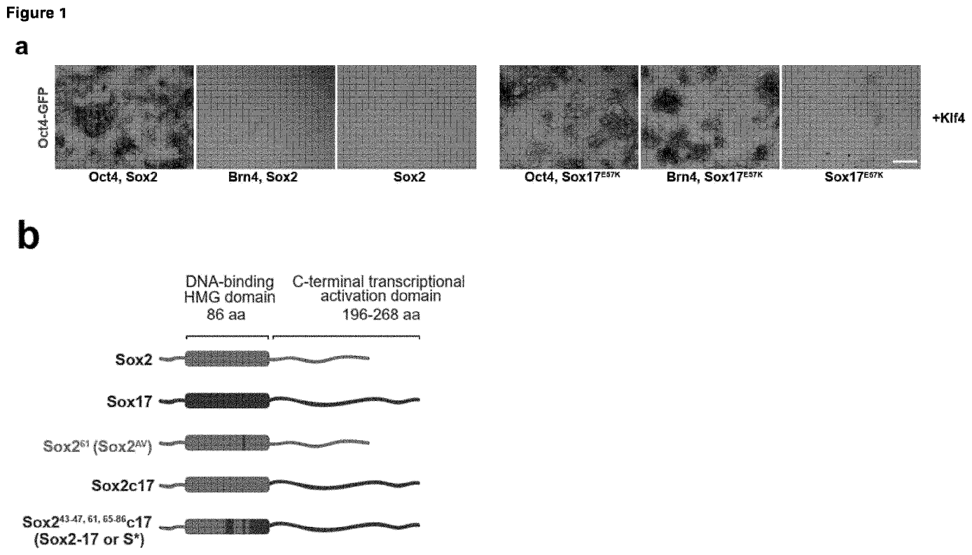

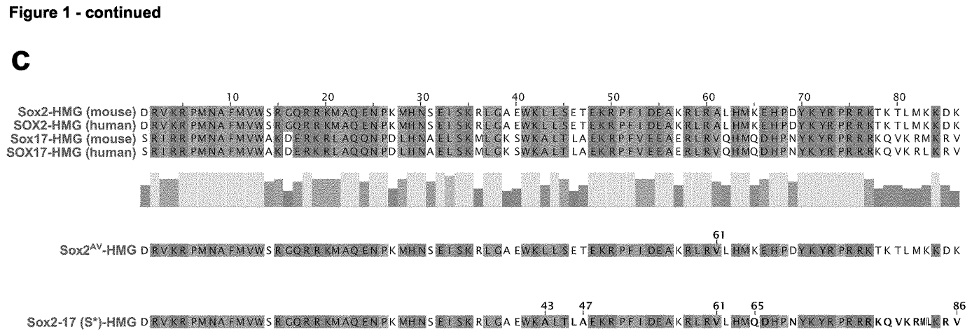

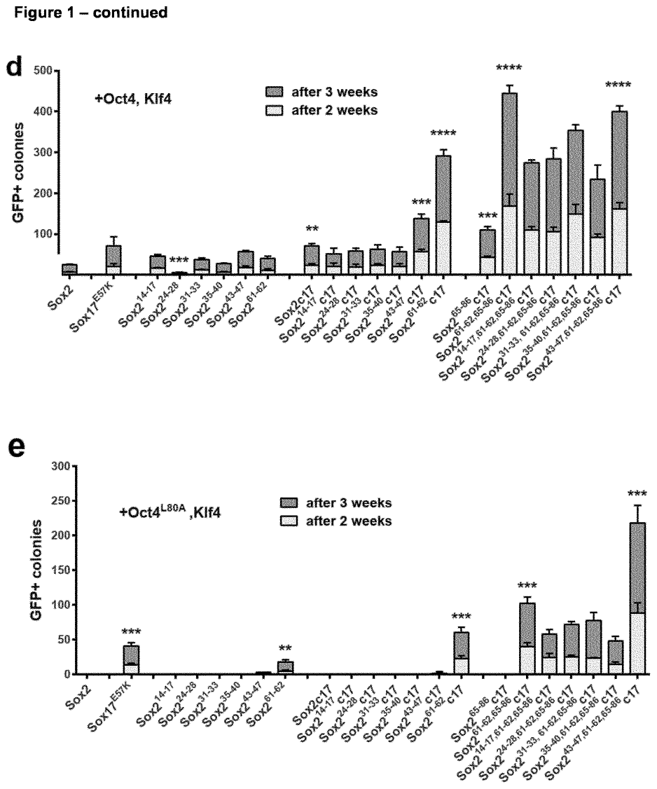

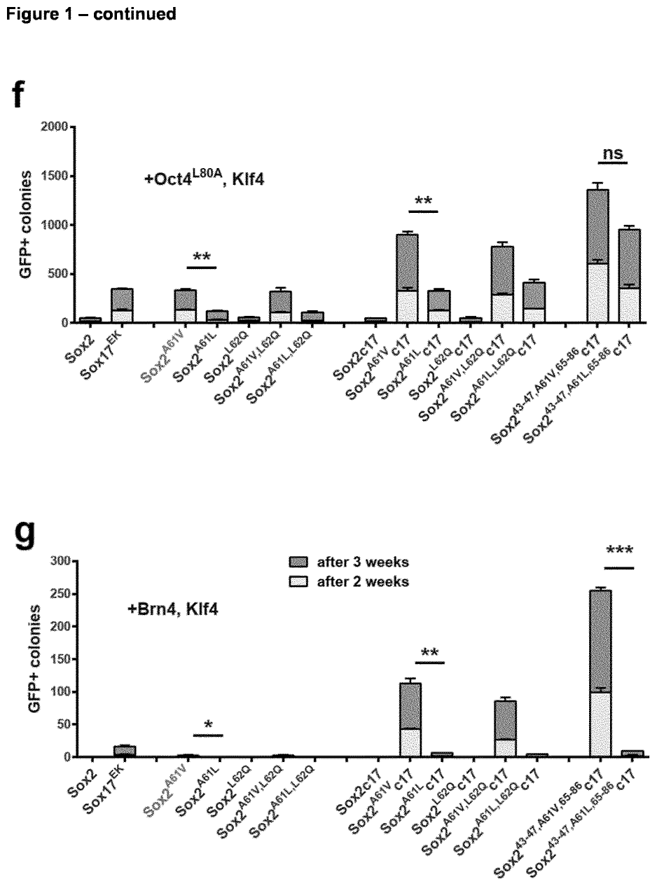

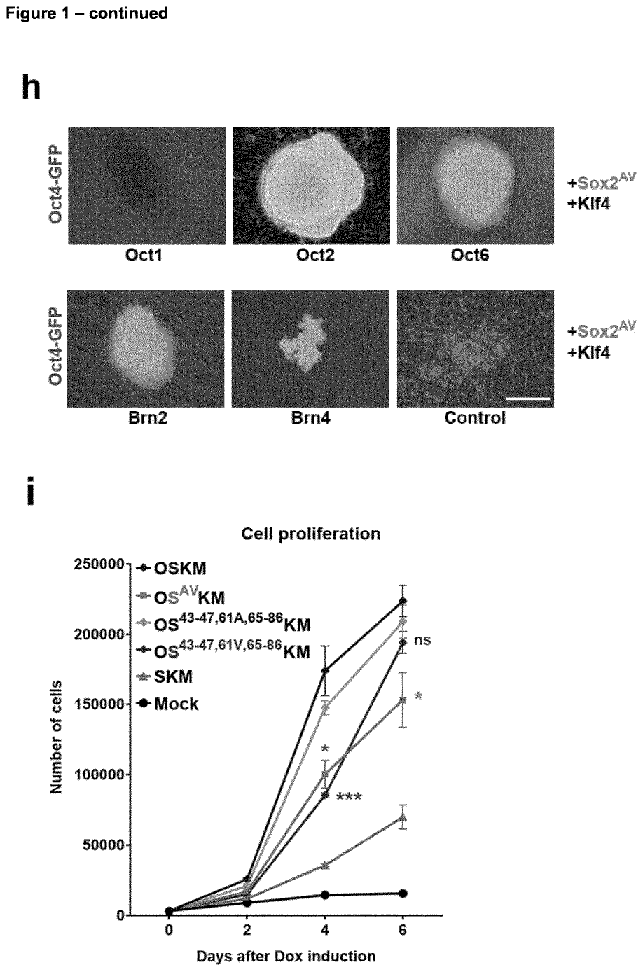

FIG. 1|Reprogramming screen of Sox2-Sox17 chimeric TF library

a, Brightfield and Oct4-GFP merged overview images showing retroviral reprogramming of MEFs carrying Oct4-GFP (OG2) reporter on 21 dpi (scale=1 mm). b, Schematic representation of Sox2 and Sox17 structures and chimeric transcription factors (TFs) generated by swapping non-conserved residues from Sox17 into Sox2. Sequence from Sox2 in blue and from Sox17 in red. c, Protein sequence alignment of DNA binding domains of mouse and human Sox2, Sox17 as well as the most crucial chimeric Sox factors of this study. d-g, Reprogramming of Oct4-GFP MEFs by retroviral vectors carrying Klf4, Sox2-Sox17 chimeric TFs, and wild-type Oct4 (d), Oct4^{L80A} linker mutant (e-f), or Brn4 (g). Error bars represent SD; n=3. Statistical significance was calculated with Student's t test. h, Representative phase-contrast and Oct4-GFP merged microscopy images of primary iPSCs colonies generated with retroviral vectors carrying different POU factors combined with Sox2^{AV} and Klf4 (Scale=200 μm). i, Cell proliferation assay, where 2×10³ MEFs were transduced with the indicated tet-inducible polycistronic constructs in 96-well plates. Error bars represent SD; n=3. The cells were counted after 2, 4, and 6 dpi. 61V was compared with 61A for each construct to calculate the statistical significance with Student's t test.

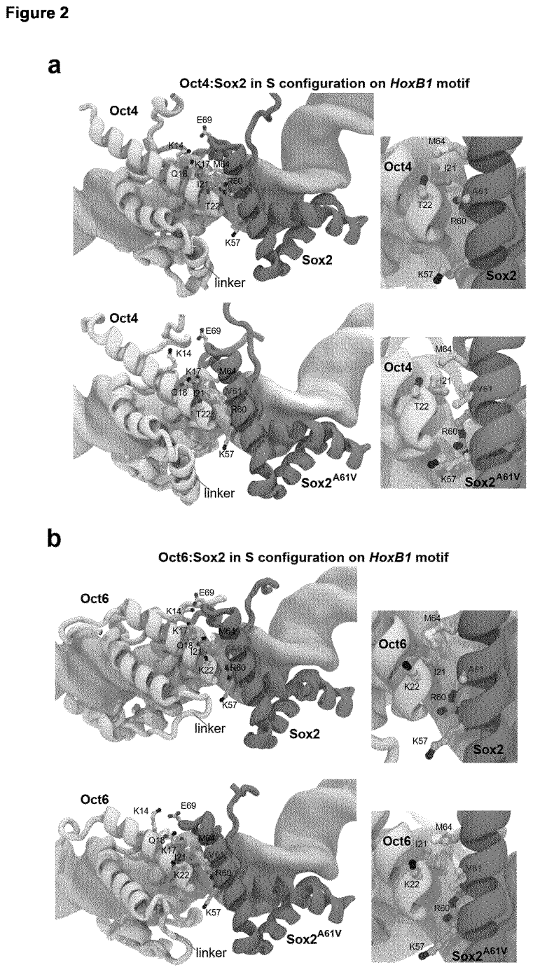

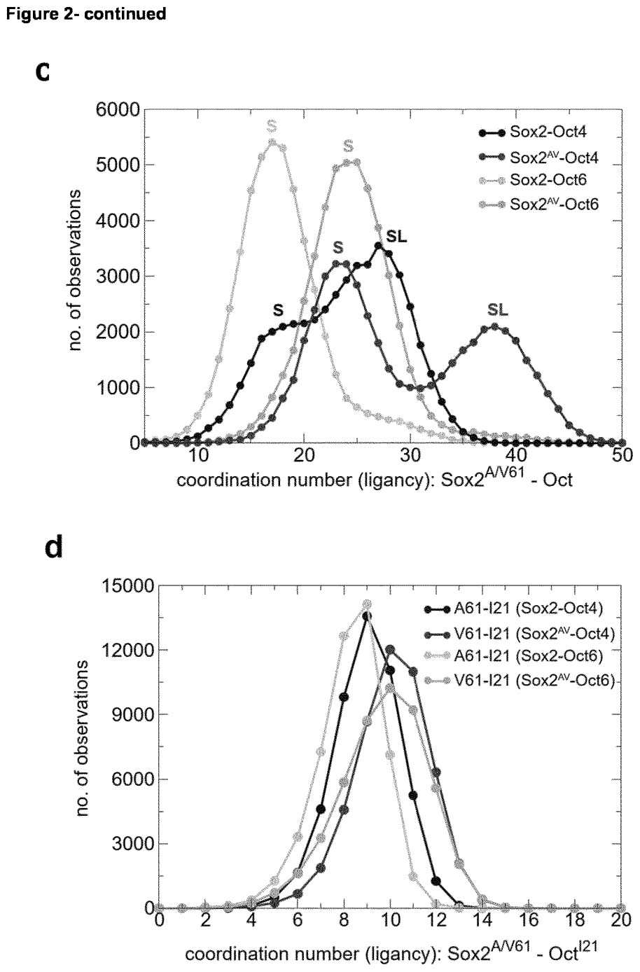

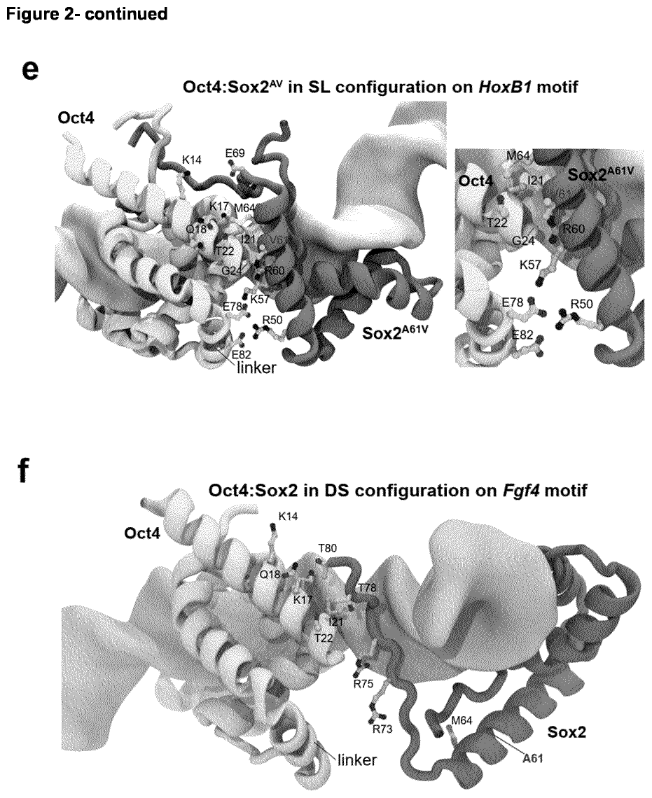

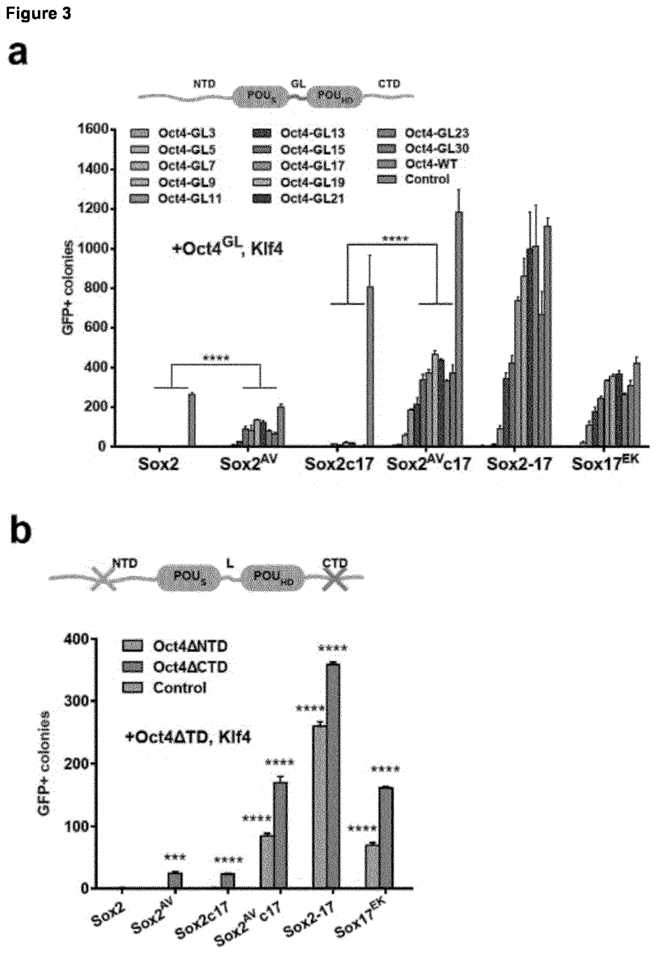

FIG. 2|Molecular dynamic simulations reveal SL configuration of Sox/Oct4

a-b, A model of Sox2/Oct4 versus Sox2^{AV}/Oct4 heterodimer (a) and Sox2/Oct6 versus Sox2^{AV}/Oct6 heterodimer (b) in POU_{S}(S) configuration on HoxB1 DNA motif. Only DNA-binding domains are shown. Oct4/Oct6 in yellow, Sox2/Sox2^{AV} in blue. c-d, Computer molecular dynamic simulations (MDS) of Sox/Oct heterodimers on HoxB1 motif. The plots show the coordination numbers (the number of contacts) between the residue 61 in Sox2 (blue) or Sox2^{A61V} (red) either with the entire DNA binding domain of Oct4 (dark) or Oct6 (light) molecule (c), or with residue 211 in Oct (d). Calculations were made for 4.8 μs of MDS of each ternary Sox/Oct/DNA complex. 4 independent 1.2 μs long simulations were performed using two different starting structural models (2 simulations per model). To ensure stochasticity, each simulation was started with a different distribution of atomic velocities. The distance threshold for a contact between two atoms was 4.5 Å. e, A model of Sox2^{AV}/Oct4 binding in POU_{S}+Linker (SL) configuration, where V61 interacts with the helix core of POU_{S} at residue G24, and two salt bridges are formed between E78 and E82 of Oct4 linker and K57 and R50 of Sox2 HMG, respectively. f, A model of Sox2/Oct4 binding in distant POU_{S} (DS) configuration on Fgf4 motif; residue 61 of Sox2 is not involved.

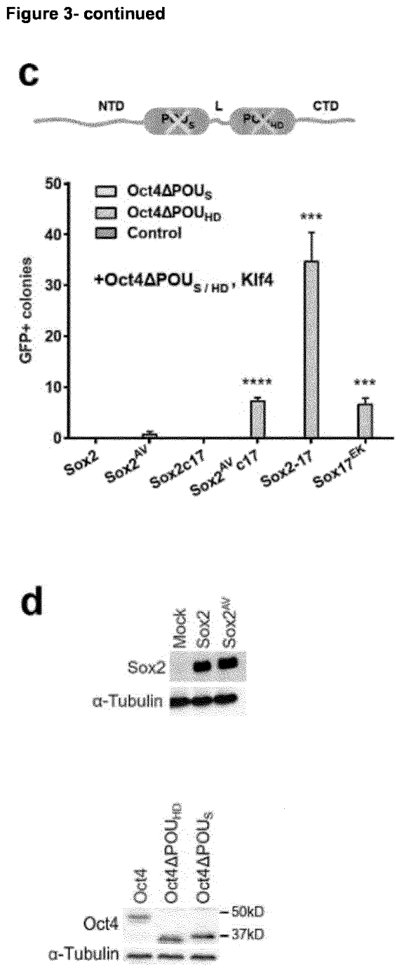

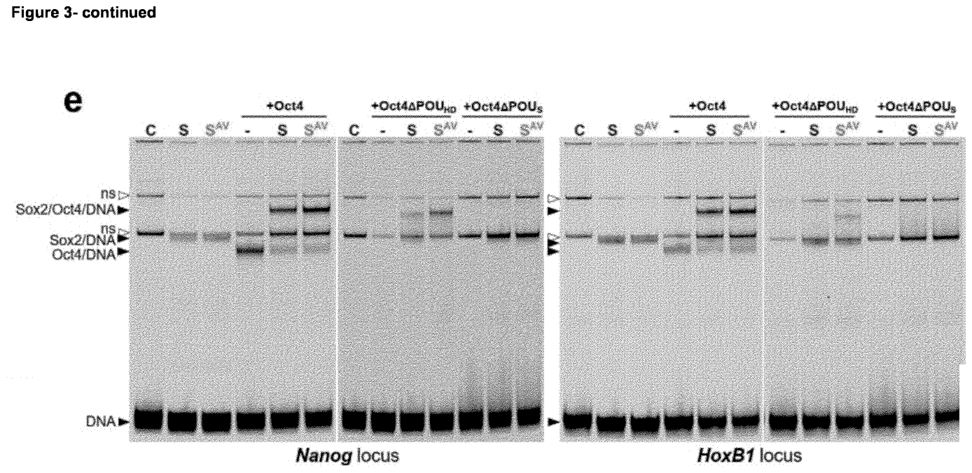

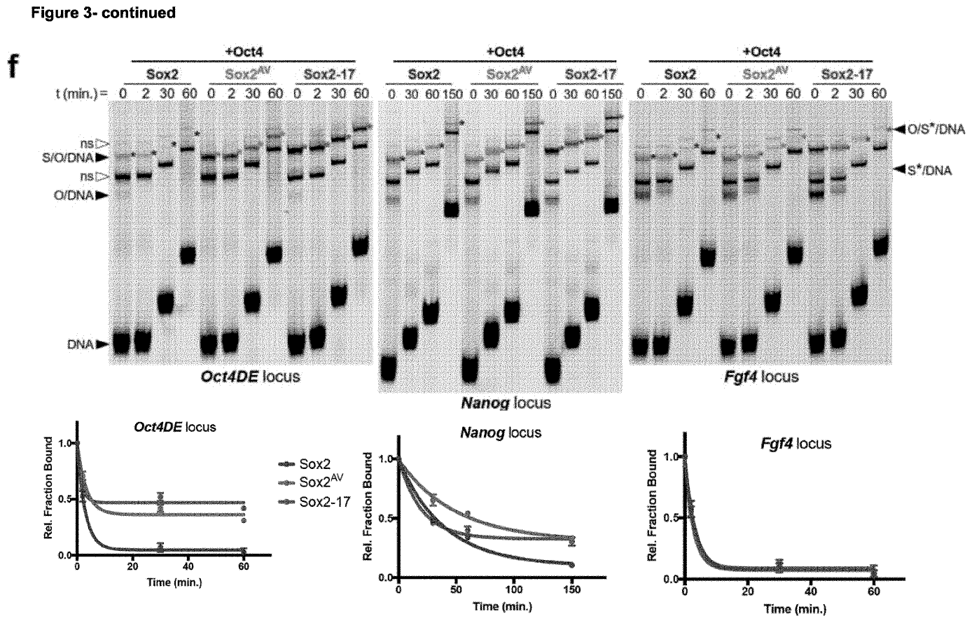

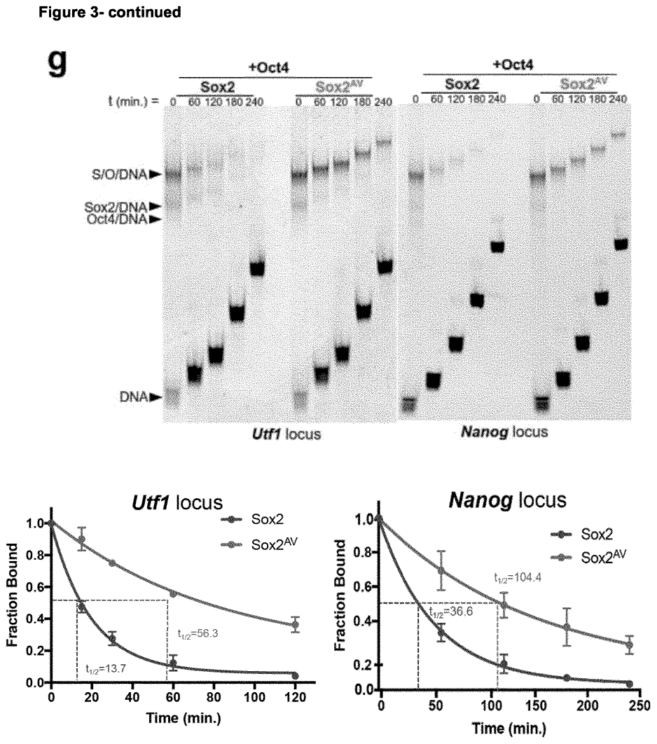

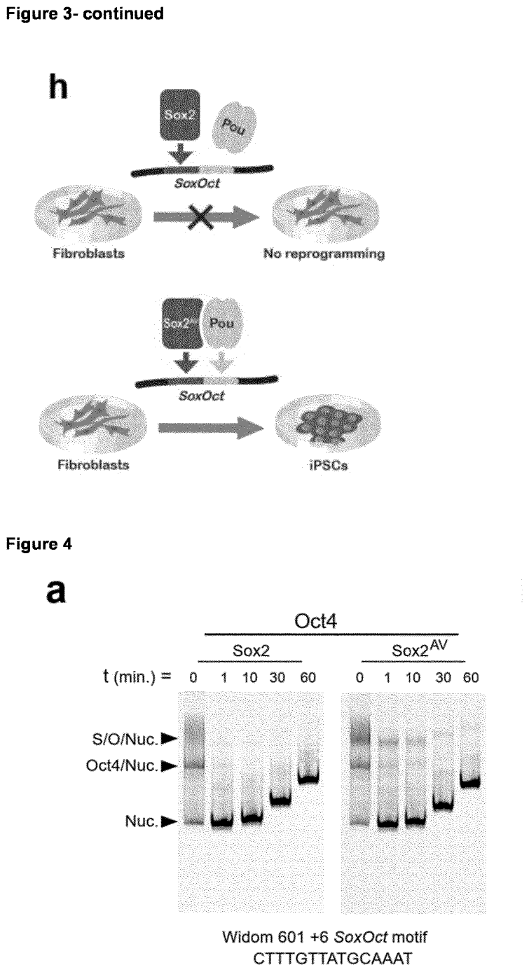

FIG. 3|Enhanced Sox/Oct cooperativity rescues non-functional POU factors in reprogramming to pluripotency

a-c, OSK reprogramming of Oct4-GFP MEFs with monocistronic retroviral vectors carrying Oct4 domain deletion mutants where the linker domain was replaced with a synthetic poly-Glycine linker (GL) of different length (3-30 residues) (a), Oct4's N- or C-terminal transactivator domain (NTD or CTD) was removed (b); POU_{S} or POU_{HD} (except for NLS) was removed (c) combined with wild-type Sox2 or Sox2-Sox17 chimeric factors. Error bars represent SD; n=3. Statistical significance was calculated with Student's t test. d, Western blot of whole-cell lysates from HEK293 used in FIG. 3e. e, Electrophoretic mobility shift assays (EMSAs) of whole-cell lysates of HEK293 cells transfected with Sox2 (S, blue), Sox2^{AV} (S^{AV}, light red) and wild-type, or mutant Oct4 in which the POU_{S} or POU_{HD} domain was removed on the Nanog promoter and HoxB1 enhancer SoxOct DNA elements labeled with Cy5. White arrow heads indicate nonspecific bands (ns) and black arrow heads indicate free DNA or DNA bound by Oct4, Sox2, or the heterodimer. f, Representative kinetic off-rate EMSAs of whole-cell lysates from HEK293 overexpressing Oct4, Sox2 (blue), Sox2^{A61V} (light red) or Sox2^{43-47,61,65-86}c17 (Sox2-17, S*, dark red) on Oct4 distal enhancer (Oct4DE), Nanog promoter, or Fgf4 enhancer DNA elements labeled with Cy5. White arrow heads indicated nonspecific bands (ns) and black arrow heads indicate free DNA or DNA bound by Oct4 (O/DNA), Sox (S/DNA), or Oct4/Sox heterodimer (O/S/DNA). Error bars on graphs represent SD; n=3. g, Representative kinetic off-rate EMSAs using purified proteins bound to the Utf1 enhancer and Nanog promoter loci labeled with Cy5. Following the binding reaction, half-life was determined by adding excess unlabeled Nanog element for the indicated time. The error bars on quantitation graphs represent SD, n=3. Ternary complex half-life t_{1/2}. h, A schematic of the enabling effect of highly cooperative Sox2^{AV} mutant on reprogramming with tissue specific POU factors or incapable Oct4 mutants.

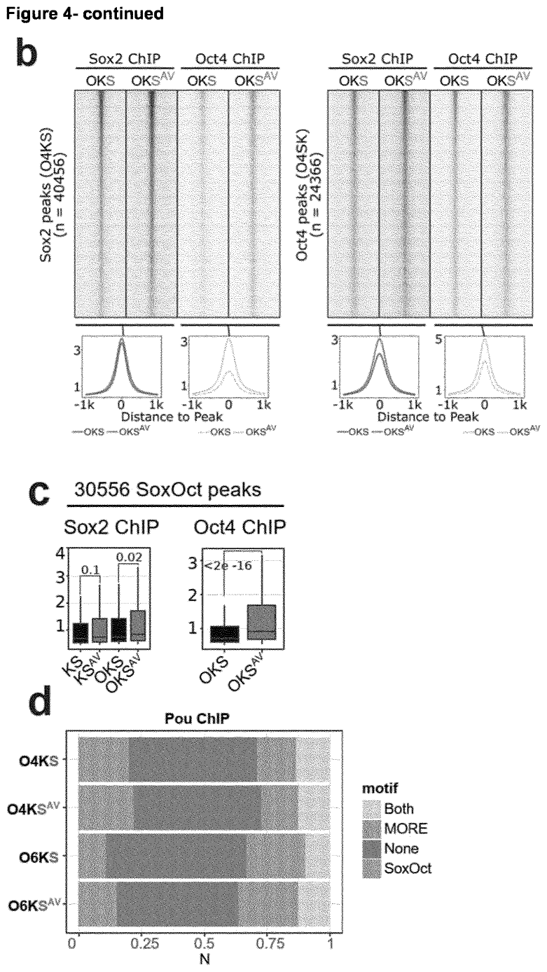

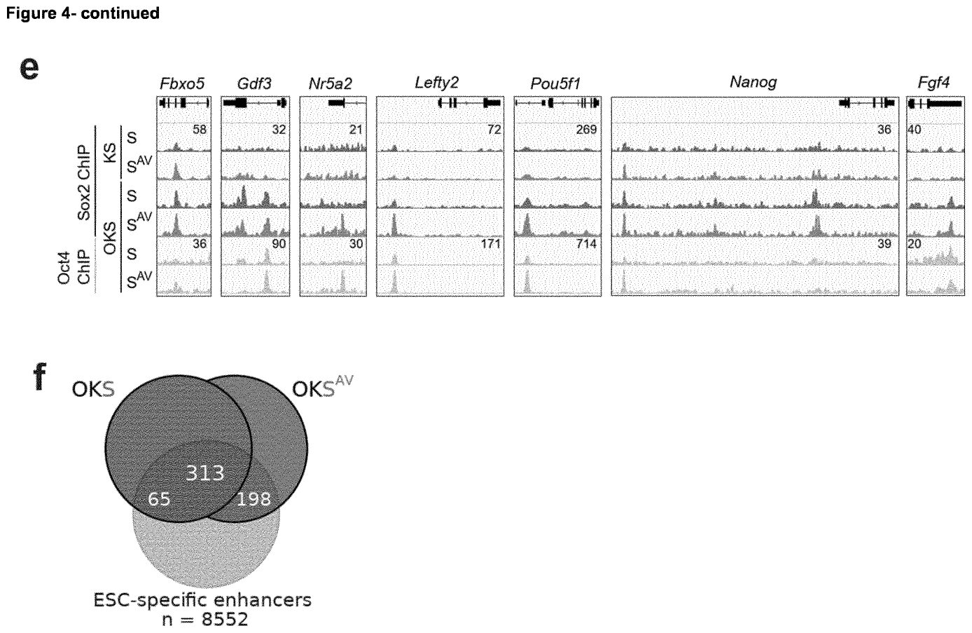

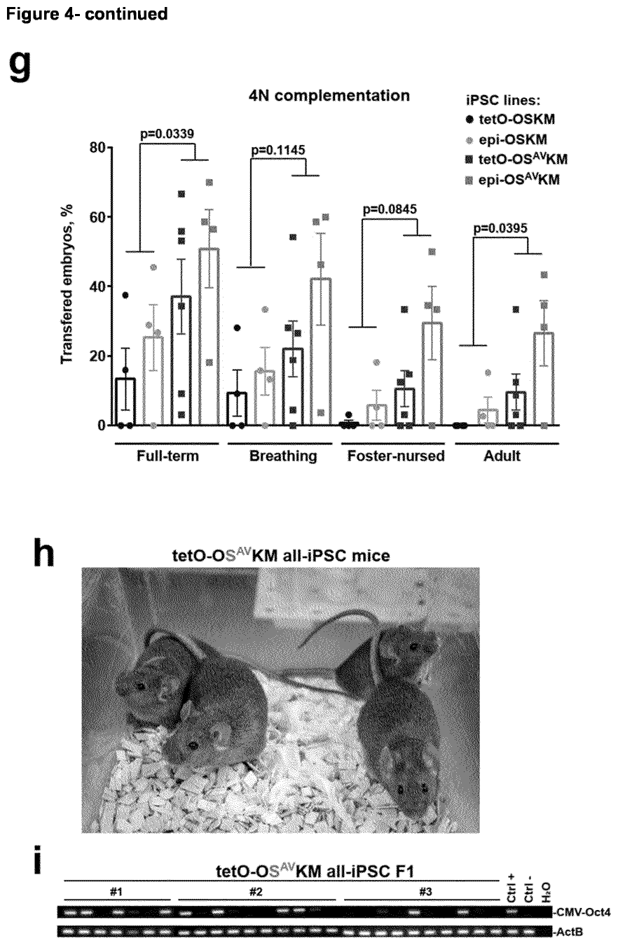

FIG. 4|Highly-cooperative Sox2^{AV} improves the developmental potential of mouse OSKM iPSCs a, Kinetic off-rate EMSAs of purified wild-type Sox2 or Sox2^{AV} in complex with Oct4 bound to the Widom 601 nucleosome sequence adapted with a SoxOct motif at superhelical location (SHL) +6. Following the binding reaction, ternary complex stability was determined by adding excess unlabeled Nanog element for the indicated time. Black arrow heads mark free nucleosome or the indicated nucleosome protein complex. b, Heat maps and read pileup plots of Sox2 and Oct4 ChIP-seq for 2 dpi tetO-OKS MEF reprogramming samples comparing Sox2^{AV} versus wild-type Sox2. c, Boxplots of quantile normalized ChIP-seq peaks for Oct4 and Sox2 for 2 dpi OKS and KS reprogramming samples. The midline indicates the median, boxes indicate the upper and lower quartiles and the whiskers indicate 1.5 times interquartile range. d, Fraction of binding sites containing SoxOct, MORE, both or none of the motifs between 2 dpi OKS reprogramming samples, where O is either Oct4 or Oct6, and S is either Sox2 or Sox2^{AV}. e, Genome browser track of Oct4 and Sox2 ChIP-seq peaks for selected pluripotency-specific loci. f, Venn diagram showing a number of ESC-specific enhancers (Shen et al., 2012) bound by Sox2 versus Sox2^{AV} on 2 dpi tetO-OKS reprogramming samples. g, Percentage of tetraploid (4N)-aggregated embryos derived from the indicated tet-inducible or integration-free episomal iPSCs that gave rise to full-term pups, pups that initiated breathing, pups that survived foster-nursing for at least 48 h, and those survived to adulthood (at least 3 months). Bars represent the mean between all tested lines for each cocktail. Error bars represent SEM. The statistical significance was determined by Mann-Whitney test. h, Adult tetO-OS^{AV}KM all-iPSC mice (9 months). i, PCR-genotyping of the progeny of tetO-OS^{AV}KM all-iPSC mice.

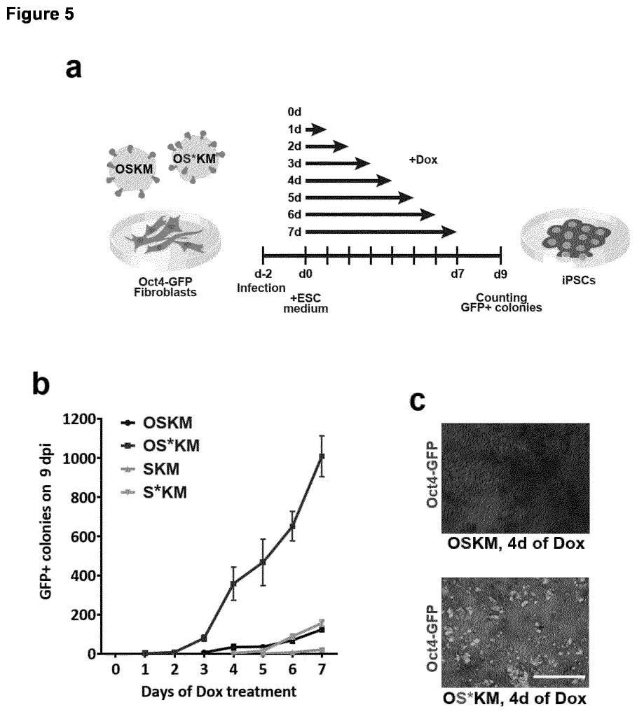

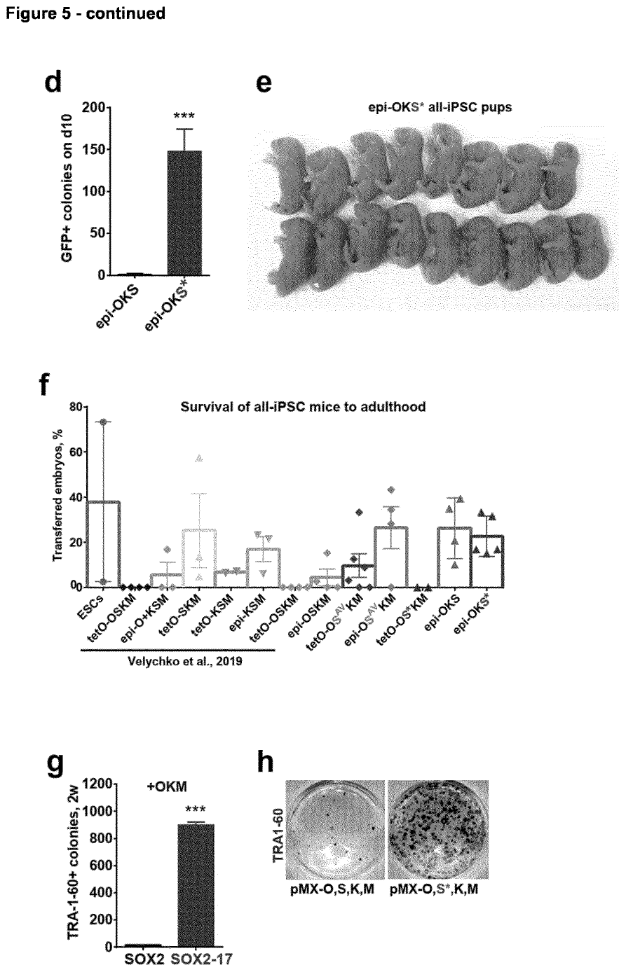

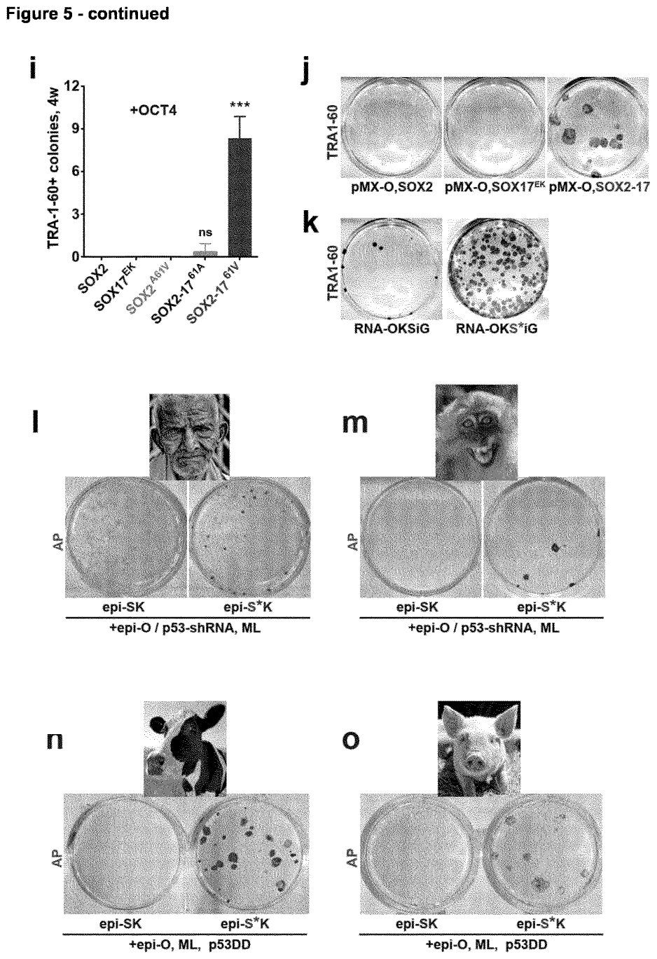

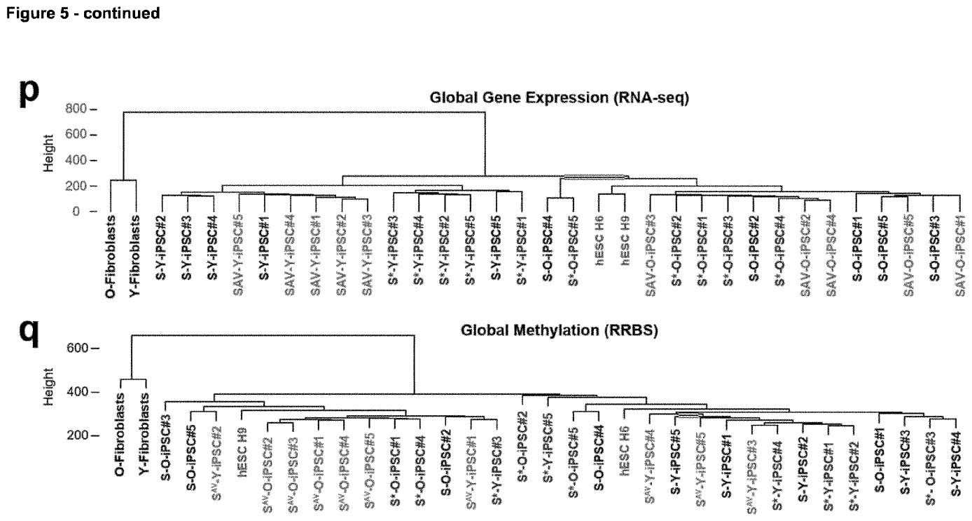

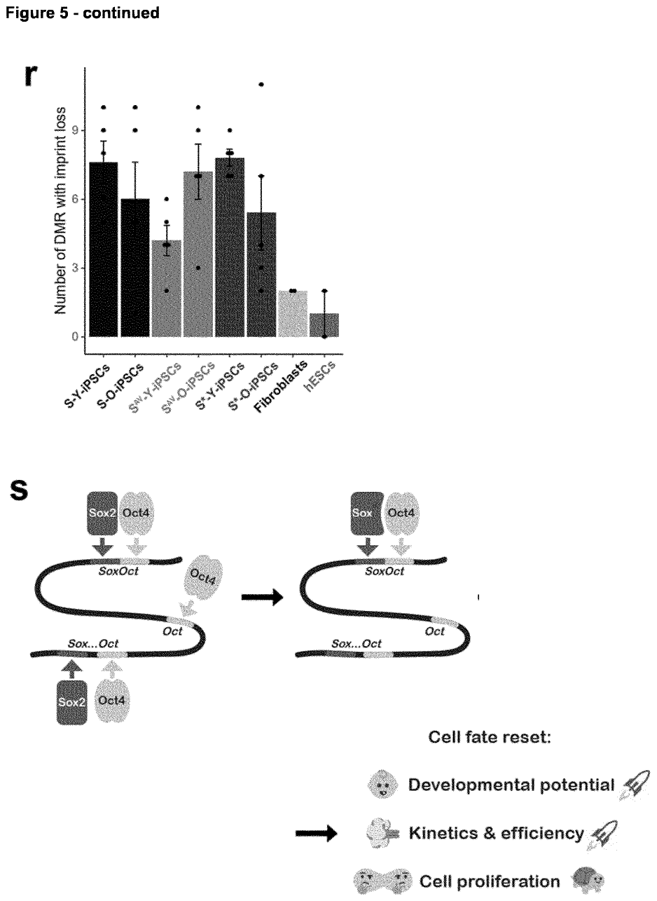

FIG. 5|Sox2-17 enhances reprogramming in five species

a, Schematic representation of time course tet-inducible lentiviral reprogramming experiment. b, Time course reprogramming of Oct4-GFP MEFs induced with OSKM or SKM carrying either Sox2 (S) or Sox2-17 (S*) for the given number of days; the iPSC colonies were counted on 9 dpi. c, Representative brightfield and Oct4-GFP merged overview images of MEFs induced for 4 days with OSKM or OS*KM, images taken on 9 dpi, scale=2 mm. d, Reprogramming of Oct4-GFP MEFs using episomal OKS (pCXLE-Oct4-P2A-Klf4-IRES-Sox) carrying either wild-type Sox2 or Sox2-17. 1.5×10⁵ cells were transfected with Fugene6. e, All-iPSC pups generated by the tetraploid (4N) complementation assay with epi-OKS* iPSC #1. 20 aggregates were transferred to two pseudopregnant CD-1 (white) females. f, Percentage of 4N-aggregated embryos that gave rise adult healthy mice (survived till at least 3 months), including previously published results (Velychko et al., 2019a). Only XY lines were plotted. Data are represented as the mean of all tested lines. Error bars represent SEM. g, Reprogramming of human fetal fibroblasts (CRL-2097) with monocistronic retroviral OSKM carrying either wild-type Sox2 or Sox2^{43-47,61,65-86}c17 (SOX2-17, S*). The volumes of viral supernatants were adjusted according to the qPCR titration. 104 transduced cells were plated on feeder layer of 6 well-plates. TRA1-60+ colonies were counted after two weeks of infection. Error bars represent SD; n=3. h, Representative whole-well scan of (g). i, Two-factor (OS) reprogramming of human fibroblasts with monocistronic retroviral vectors. TRA1-60+ colonies were counted after four weeks of infection. Error bars represent SD; n=3. j, Representative whole-well scan of (i). k, TRA1-60 staining of human fetal (CRL-2097) fibroblasts reprogrammed with self-replicating RNA (VEE) vectors carrying either OKSiG or OKS*iG reprogramming casettes. l-o, Whole-well scans of alkaline phosphatase (AP) staining for episomal reprogramming of (l) 56-year-old human male dermal fibroblasts on day 25, (m) Cynomolgus macaque (Macaca fascicularis) fibroblasts on day 25, (n) bovine fetal fibroblasts on day 21, (o) porcine fetal fibroblasts on day 21 after nucleofection. p-q, Hierarchical clustering analysis of human ESC and integration-free iPSC lines derived from human newborn foreskin (young, Y) or 56-year-old human dermal (old, O) fibroblasts using episomal OSKML (pCXLE-OCT4+shP53, L-MYC-F2A-LIN28 and SOX-P2A-KLF4 carrying SOX2, SOX^{AV}, or SOX2-17) based on: global gene expression (RNA-seq), TPM≥1 (p) or global methylome (RRBS) (q). Clustering was based on Euclidean distance. r, Comparison of the number of genes that lost imprinting in 31 DMRs according to RRBS data. s, A model of the advantageous effects of highly cooperative Sox factors on cell fate reset.

FIG. 6|Sox/Oct cooperativity in high-versus low-grade pluripotency

a, Spearman correlation of time course ATAC-seq reads for naïve-to-primed differentiation mouse ESC samples (Yang et al., 2019). b-c, TOBIAS footprinting analysis of (a) using MEME TF motif database of ESCs versus day one EpiLC samples (b), and day one versus day two EpiLC samples (c). d, EMSAs of whole-cell lysates showing endogenous levels of Sox2 and Oct4 that can bind Nanog promoter DNA element labeled with Cy5. Arrow heads indicate DNA bound by Oct4 (yellow), Sox2 (blue), or the heterodimer (green). e, Western blot of lysates used in FIG. 6d. f, A supershift assay using anti-Oct4 and anti-Sox2 antibodies to confirm the identity of protein/DNA complexes in whole-cell lysate EMSAs. g, Endogenous EMSAs of 6 PSC lines (Velychko et al., 2019a), of which epi-OKSM1 and 3 were incapable of generating all-iPSC mice. The lines were grown in 2iLIF on gelatin-coated plates, then split either in KSR-LIF media (left panel) or again in 2iLIF media (right panel). h, Western blot of lysates used in FIG. 6g. i, Immunostaining for naïve pluripotency marker KLF17 of primed-to-naïve converted human iPSCs on day 6 after transduction with constitutive lentiviral vectors (pHAGE2-EF1α). j, Endogenous EMSAs of integration-free clonal mEpiSC lines primed-to-naïve converted using episomal mCherry-T2A-SOX-KLF4 vectors carrying wild-type SOX2, SOX2^{AV}, or SOX2-17. The error bars represent SEM, statistical significance was calculated between top three out of six lines converted by each cocktail with Student's t test. k, SoxOct model of high-grade developmental reset.

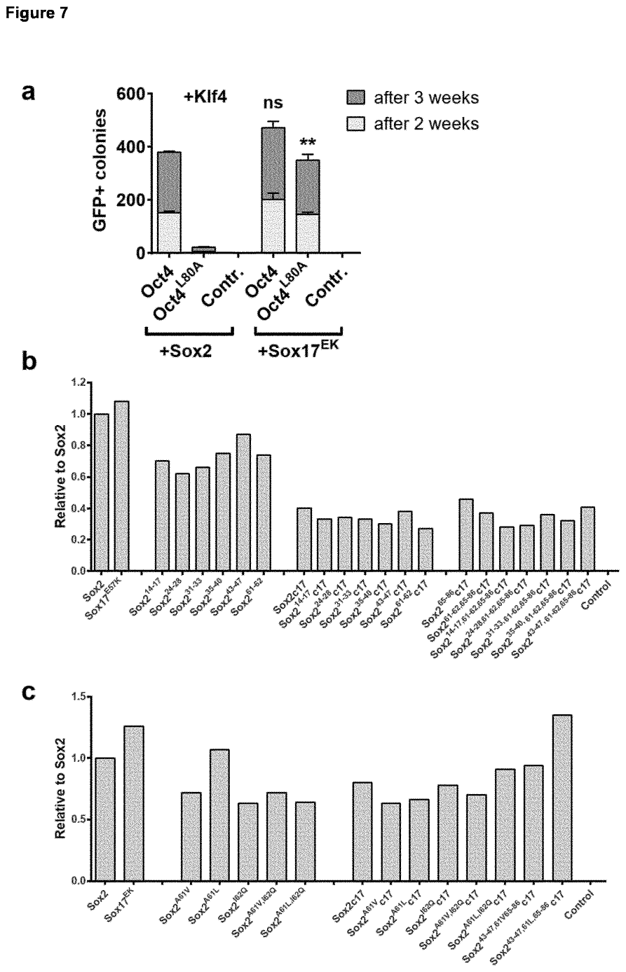

FIG. 7 (Related to FIG. 1)

a, OSK reprogramming of Oct4-GFP reporter MEFs by retroviral monocistronic Sox2 or Sox17^{EK}, combined with Klf4 and Oct4^{L80A} linker mutant. Error bars represent SD; n=3. Statistical significance was calculated with Student's t test. b-c, qPCR titration of the retroviral vectors from FIG. 1d-g.

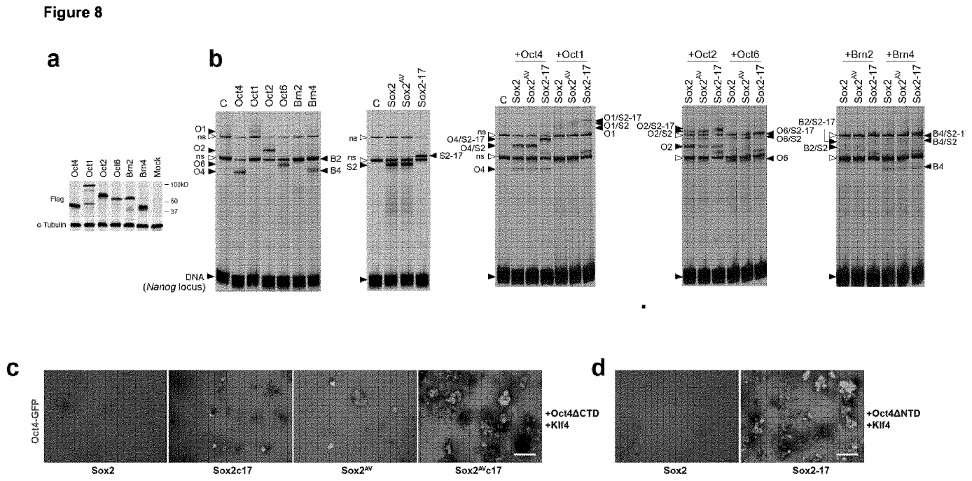

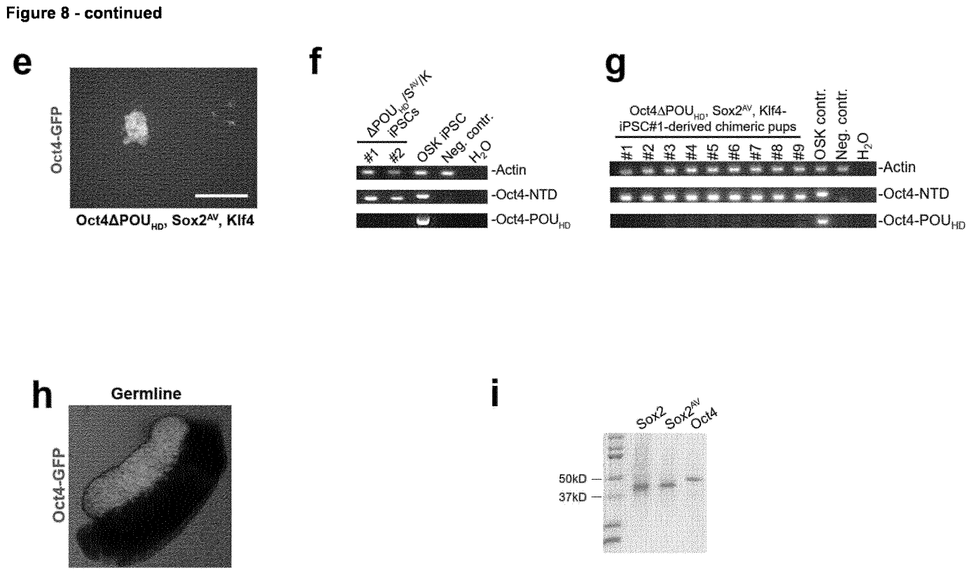

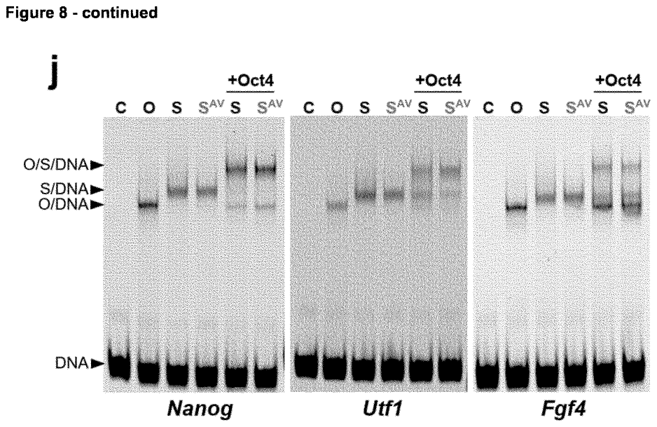

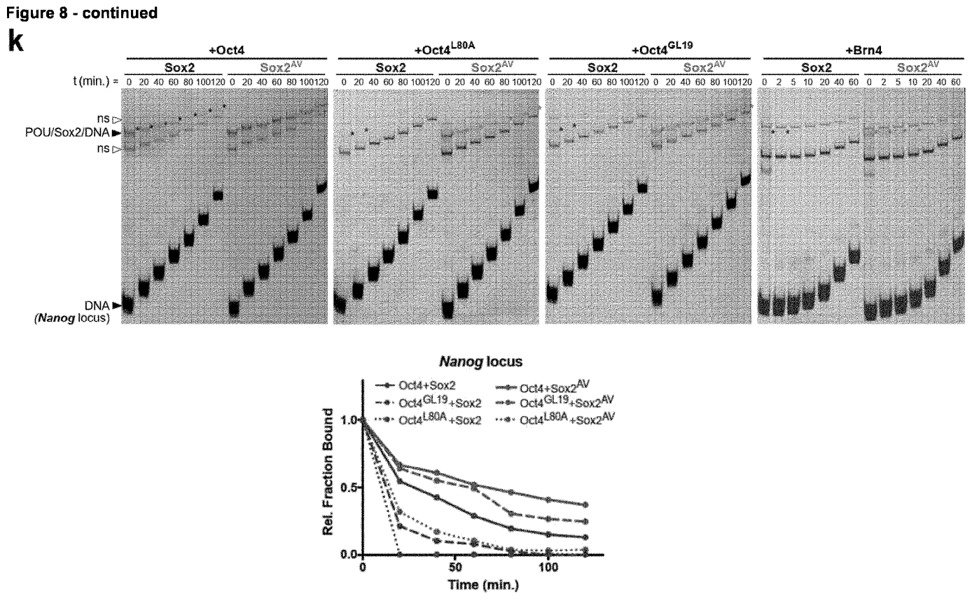

FIG. 8 (Related to FIG. 3)

a, Western blot of whole-cell lysates of HEK293 overexpressing flagged POU factors used in (b). b, EMSAs of whole-cell lysates from (a) on the Nanog promoter locus labeled with Cy5. White arrow heads indicate nonspecific bands (ns) and black arrow heads indicate free DNA or DNA bound by Oct (O/DNA), Sox (S/DNA), or both (O/S/DNA). c-d, Representative brightfield and Oct4-GFP merged overview images showing MEFs reprogrammed with Oct4 mutant without the C- (c, ACTD) or N- (d, ΔNTD) terminal transactivator domain, 21 dpi, scale=1 mm. e, A primary iPSC colony generated by the Oct4 mutant with the POU_{HD} domain removed (except the NLS), scale=100 μm. f, PCR genotyping confirming the identity of two Oct4ΔPOU_{HD}/Sox2^{AV}/Klf4 iPSC lines, g, PCR genotyping of chimeric mice generated by aggregation with Oct4ΔPOU_{HD}/Sox2^{AV}/K-generated iPSCs. h, Brightfield and Oct4-GFP merged image of embryonic day 13.5 gonad dissected from chimeric embryo from (g). i, Coomassie stained SDS-polyacrylamide gel of mouse Sox2, Sox2^{AV}, and Oct4 from insect cells used in FIG. 3g and FIG. S2j. j, EMSAs of insect cell-purified Sox2 (S, blue), Sox2^{AV} (SAV, light red), and wild-type Oct4 on the Nanog promoter, Utf1 and Fgf4 enhancer DNA elements labeled with Cy5. Arrow heads indicate free DNA or DNA bound by Oct4 (O/DNA), Sox2 (S/DNA), or the heterodimer (O/S/DNA). k, Representative kinetic off-rate EMSAs using whole-cell lysates overexpressing full-length Oct4, Oct4^{L80A}, Oct4^{GL19}, or Brn4 combined Sox2 versus Sox2^{AV} lysates on the Nanog promoter locus labeled with Cy5. Following the binding reaction, half-life was determined by adding excess unlabeled Nanog element for the indicated time. White arrow heads indicated nonspecific bands (ns) and black arrow heads indicate free DNA or DNA bound by POU/Sox heterodimer.

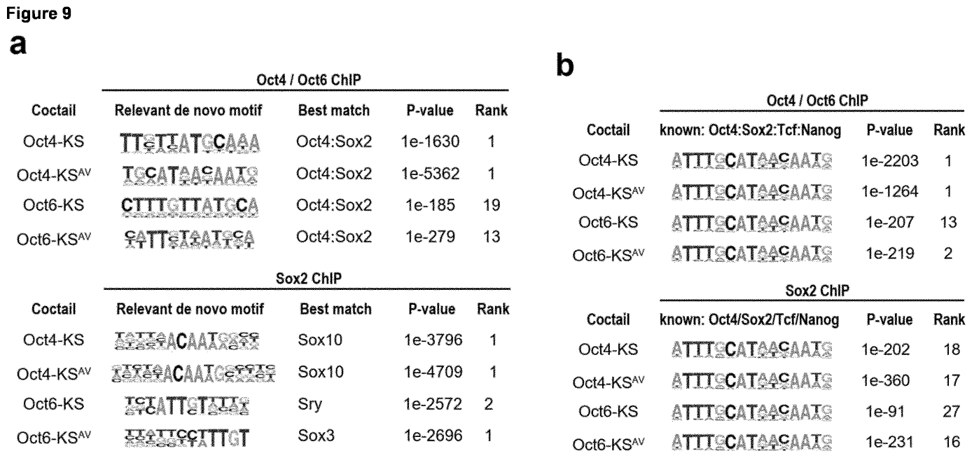

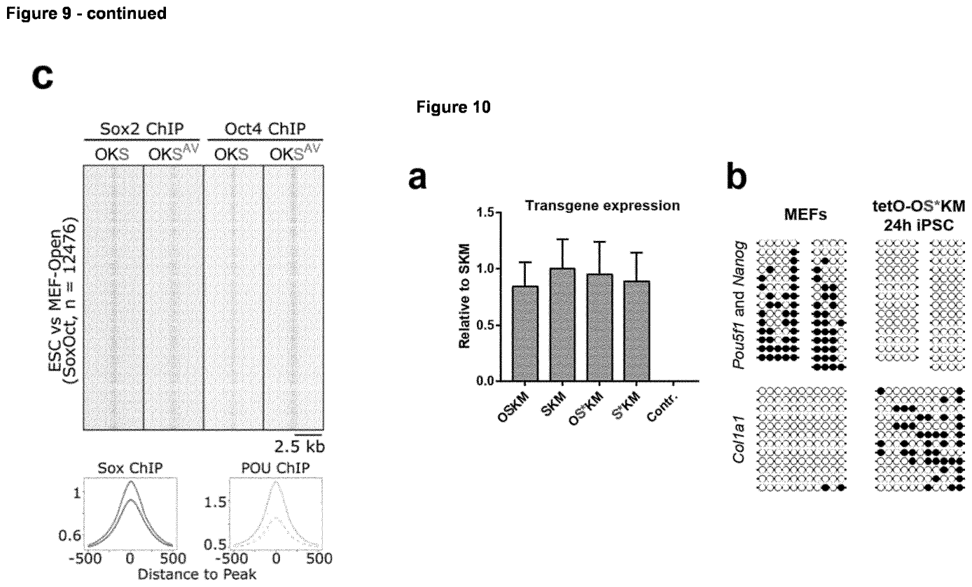

FIG. 9 (Related to FIG. 4)

a-b, HOMER (Heinz et al., 2010) de novo (a) and known SoxOct (b) motif analysis showing the enrichment β-value for Oct4, Oct6, Sox2 ChIP-seq. c, Sox2 and Oct4 ChIP-seq signal heatmaps for MEFs reprogrammed with tet-inducible OKS at 2 dpi, at the loci containing Sox2/Oct4 footprints in opened chromatin of ESCs versus MEFs determined by TOBIAS footprinting analysis of ATAC-seq data (Li et al., 2017).

FIG. 10 (Related to FIG. 5)

a, qPCR titration of tet-inducible lentiviral vectors from FIG. 5 b-c after 24 h of Dox induction. Error bars represent SD; n=3. b, Bisulfite sequencing analysis of DNA methylation in Oct4, Nanog, and Col1a1 promoters in MEFs and an iPSC line generated by inducing tetO-OS*KM for 24h. c, H&E staining of teratoma sections generated with 24 h OS*KM iPSCs with representation of the three germ layers (ectoderm—Ec: keratinizing epithelium; mesoderm—M: striated muscles; endoderm—En: cuboidal epithelium). d, Bright-field and Oct4-GFP merged images of the gonads from E13.5 24 h OS*KM iPSC chimeric embryos. e, All-iPSC pups generated by tetraploid (4N) complementation assays with 24 h OS*KM iPSC #1 line. 12 aggregates were transferred to a pseudopregnant CD-1 (white) female. f, Representative brightfield and Oct4-GFP merged overview images showing Oct4-GFP MEFs reprogrammed with tet-inducible lentiviral Sox-T2A-Klf4 vectors carrying wild-type Sox2, Sox2c17, Sox17^{EK}, or Sox2-17, 21 dpi, scale=1 mm. g, Phase-contrast and Oct4-GFP merged microscopy image of two-factor mouse S*K iPSC clonal line generated in (f) at passage three, scale=100 μm. h, PCR genotyping of two mouse S*K iPSC lines from (f-g). i, Immunostaining of a mouse S*K iPSC line for pluripotency markers Nanog and SSEA-1. Nuclei were stained with Hoechst 33342, scale=100 μm. j, H&E staining of teratoma sections generated with S*K mouse iPSC line with representation of three germ layers (ectoderm—Ec: keratinizing epithelium; mesoderm—M: striated and smooth muscles; endoderm—En: cuboidal epithelium). k, Western blot of whole-cell lysates from HEK293T transfected with pCXLE-Oct4-P2A-Sox-T2A-Klf4-E2A-cMyc, pCXLE-Oct4-P2A-Klf4-IRES-Sox episomal vectors carrying mouse Sox2, Sox2^{AV}, or Sox2-17. l, Phase-contrast microscopy image of two-factor human iPSC line generated with monocistronic retroviral OCT4 and SOX2-17 at passage two, scale=200 μm. m, Immunostaining of a human OS* iPSC line for pluripotency markers NANOG and TRA1-81. Nuclei were stained with Hoechst 33342, scale=100 μm. n, H&E staining of teratoma sections generated with OS* human iPSC line with representation of three germ layers (ectoderm—Ec: neural rosettes; mesoderm—M: cartilage, bone, endothelium; endoderm—En: gut and lung epithelium). o, Western blot of whole-cell lysates from HEK293T transfected with the original episomal pCXLE-SOX2-F2A-KLF4 construct, as well as generated in this study P2A vectors: pCXLE-SOX2-P2A-KLF4, pCXLE-SOX2^{AV}-P2A-KLF4 and pCXLE-SOX2-17-P2A-KLF4. p, PCR genotyping of episomal iPSC lines generated from cynomolgus macaque fibroblasts at passage three. q, Chromosomal spreads of two integration-free cynomolgus macaque iPSC lines. r, Phase-contrast image of integration-free cynomolgus macaque iPSC #11 from panel o at passage seven, scale=200 μm. s, Immunostaining of cynomolgus integration-free iPSC line for NANOG and OCT4. Nuclei were stained with Hoechst 33342, scale=100 μm. t, H&E staining of teratoma sections generated with cynomolgus integration-free iPSC line with representation of three germ layers (ectoderm—Ec: neural rosettes; mesoderm—M: cartilage, smooth muscles; endoderm—En: cuboidal epithelium). u, A representative whole-well scan of alkaline phosphatase (AP) staining for episomal reprogramming of bovine fetal fibroblasts on day 21 after nucleofection with episomal OSKML (omitting P53 knockdown). v, PCR genotyping of episomal OSKML bovine iPSC lines from (u) at passage six. w, Phase-contrast image of integration-free bovine iPSC line at passage eight from (v), scale=200 μm. x, A representative chromosomal spread of integration-free bovine iPSC line from (v). y, Immunostaining of bovine integration-free iPSC line for Sox2 and OCT4. Nuclei were stained with DAPI, scale=200 μm. z, Representative image of biPSC reset by episomal S*K at day 6 after nucleofection, scale=200 μm. Cells were plated on feeders in KSL-LIF+XAV939 media. aa, Teratoma generated by subcutaneous injection of S*K-reset biPSC line into SCID mouse, 5 weeks after injection. Untransfected biPSCs did not generate teratoma in 3 injection attemps. ab, H&E staining of teratoma sections generated by biPSCs (aa) with representation of three germ layers (ectoderm—Ec: neural rosettes, epidermis, squamous epithelial cells; mesoderm—M: smooth muscles, connective tissue; endoderm—En: gut epithelium). ac, PCR genotyping of episomal iPSC lines generated from dermal fibroblasts of aged male (AG04148) at passage three. ad-ae, Karyotyping of human integration-free iPSC lines generated from newborn foreskin fibroblasts (young, Y) or 56-year-old male fibroblast (old, O) using chromosomal spreads (ad) or e-karyotyping based on RNA-seq data (ae).

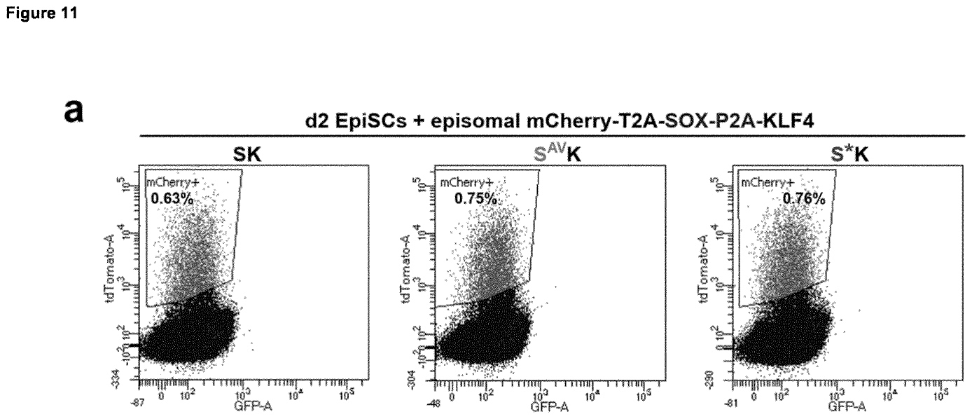

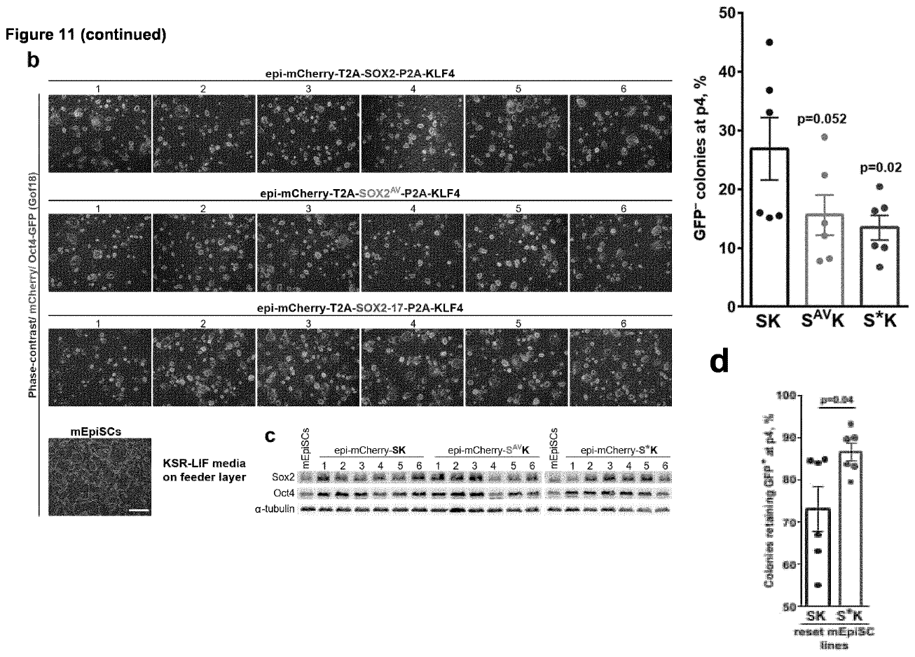

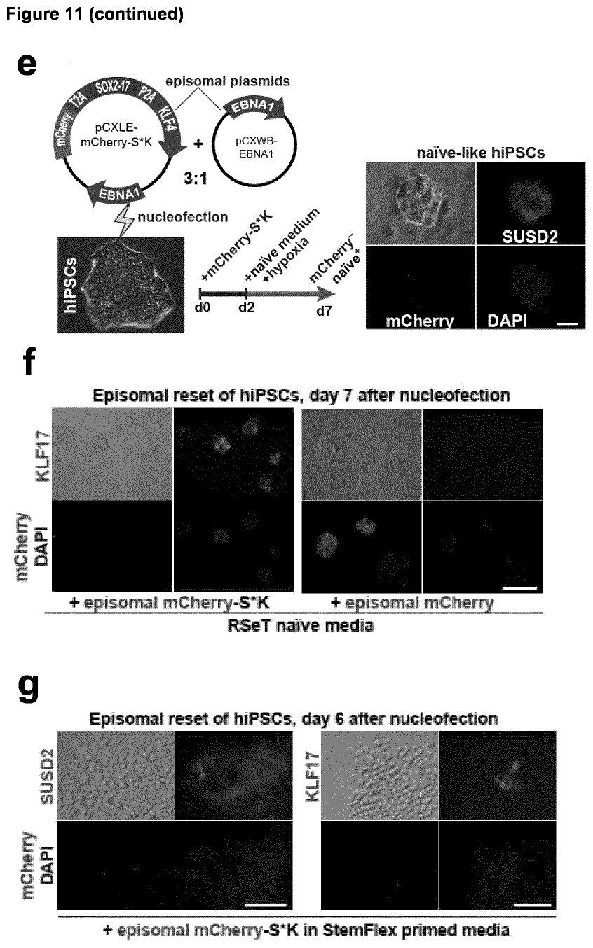

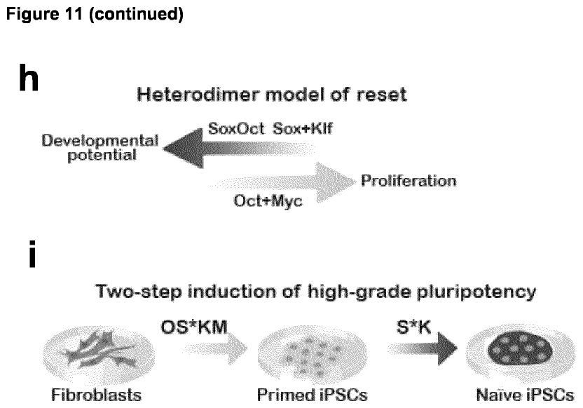

FIG. 11 (Related to FIG. 6)

a, FACS of mEpiSCs transfected with episomal mCherry-T2A-SOX-KLF4 vectors using Lipofectamin Stem reagent at day 2. b, Representative phase-contrast/epi-mCherry/Oct4-GFP merged overview images of clonal primed-to-naïve converted mEpiSCs using episomal mCherry-T2A-SOX-P2A-KLF4 vectors grown in KSR-LIF media on C3H feeder layer, at passage 4, scale=500 μm. The same number of cells were plated for each line. At least 100 colonies were quantified from three random overview images of each line, the error bars represent SEM, statistical significance calculated using Student's t test. c, Western blot of lysates used in FIG. 6j. d, Quantification of Gof18⁺ and Gof18⁻ colonies generated by mEpiSCs reset using episomal mCherry-SK or -S*K vectors at passage 4, grown in KSR-LIF media on feeders. Each point represents an average percentage of Gof18⁺ colonies for one of the six lines randomly picked for each construct. The same number of cells were plated for each line. >100 colonies were quantified from three randomized overview images of each line, the error bars represent SEM, statistical significance was calculated using Student's t-test. e, Method for human intergration-free naïve reset using episomal mCherry-S*K. Representative images of day 7 S*K-reset hiPSCs (female episomal line A18945, Gibco) stained for human naïve marker SUSD2 is shown on the right. RSeT media and 5% CO₂ used started from day 2, scale=50 μm. No SUSD2⁺ cells were detected in control nucleofection. f, Representative images of day 7 S*K-reset hiPSCs (A18945, Gibco) stained for human naïve marker KLF17, scale=50 μm. The vast majority of converted cells were mCherry-negative. No KLF17⁺ cells were detected in control nucleofections. Episomal reset of two hPSC lines (OS*KML O-hiPSC #1 and H9 hESCs) showed the same results. g, Representative images of day 6 S*K-reset hiPSCs (A18945, Gibco) stained for human naïve markers SUSD2 and KLF17, scale=50 μm. The cells were grown in primed media KLF17⁺ or SUSD2⁺ cells were detected in control nucleofections. The results were reproduced for two other hPSC lines. h, SoxOct model of high-grade developmental reset. i, A two-step protocol for induction of high-grade pluripotent cells across species.

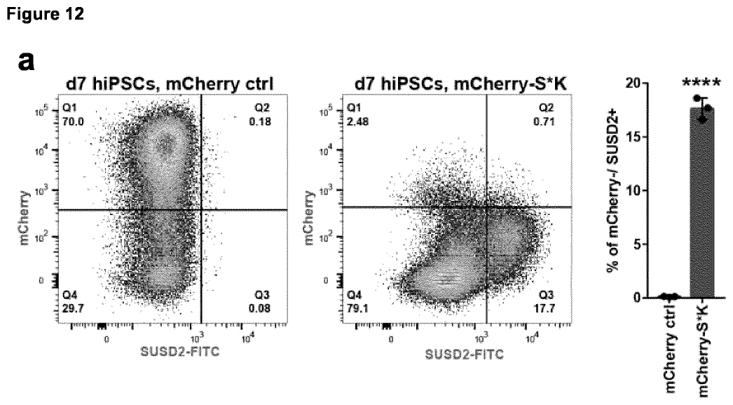

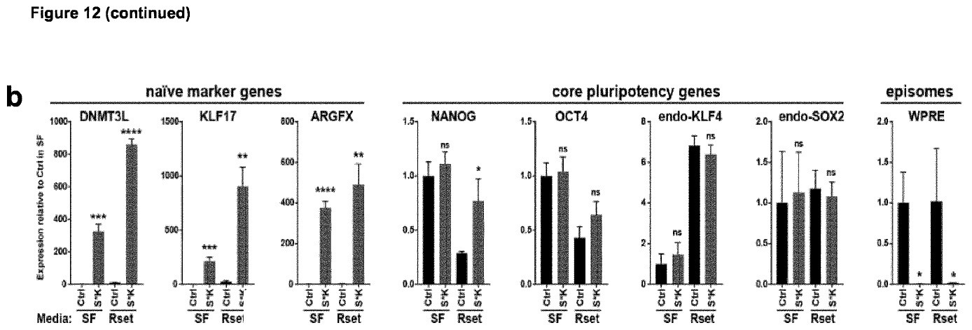

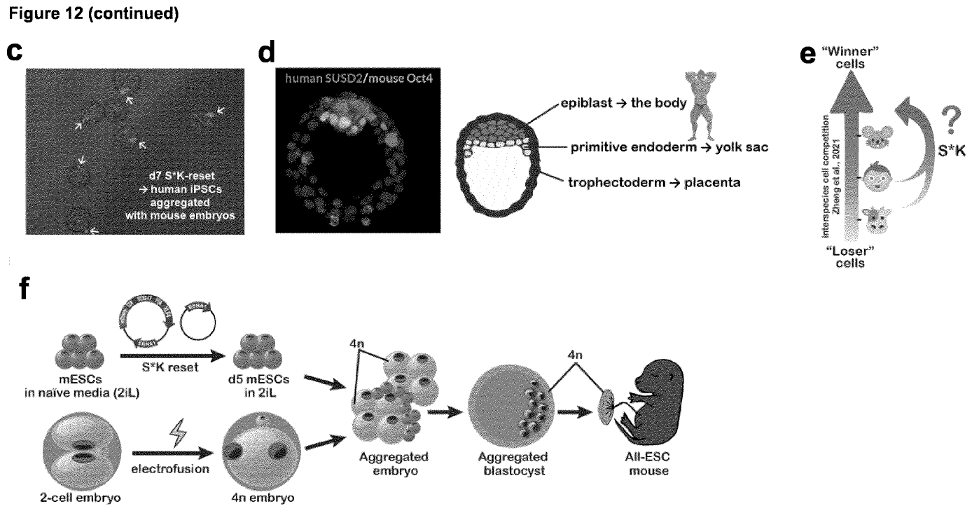

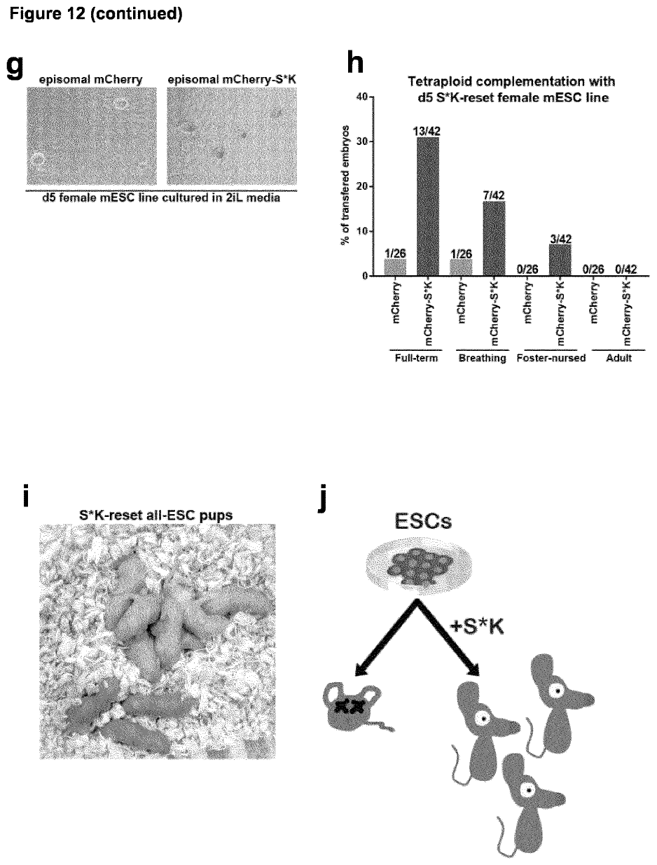

FIG. 12. (Related to FIGS. 5 and 6)

FIG. 12 provides additional details on the results shown in FIGS. 10 and 11.

a, FACS for primate-specific naïve marker SUSD2 of human iPSCs (A18945, Gibco) nucleofected with episomal pCXLE-mCherry-T2A-SOX-KLF4, and grown in Rset media, at day 7 after nucleofection and plating on feeders (FIG. 12a). Error bars represent SD; n=3; statistical significance was calculated using Student's t-test. b, RT-qPCR gene expression analysis of bulk day 7 reset samples (whole well lysates). Naïve markers were significantly upregulated even in primed media, albeit they were more upregulated in Rset media. WPRE expression confirmed the elimination of episomal vectors in S*K samples. Error bars represent SD; n=3; statistical significance was calculated using Student's t-test. c, Cross-species human/mouse morula aggregation using sorted day 7 SUSD2+ hiPSCs (A18945, Gibco) with constitutive RFP expression (CRISPR-mediated knock-in), reset with pCXLE-S*K (no mCherry). Majority of the aggregated embryos contained hiPSCs localized at ICM. Similar level of integration was achieved using hand-picked dome-shaped colonies with no sorting. d, Immunostaining of a representative cross-species chimeric E4 embryos from FIG. 11f with human-specific SUSD2 and mouse-specific Oct4 antibodies. The microscopy image showed a robust integration of human S*K-reset cells into the epiblast region of the embryo, while mouse cells appear to only contribute to primitive endoderm, e, Hypothetical model of interspecies cell competition based on Zheng et al., 2021. Rodent PSCs win the competition against primate PSCs, with bovine PSCs being the ultimate losers. We hypothesize that those results could be explained by the “primed-to-naïve” grade at which the cells of given species are stabilized in culture. It is conceivable that S*K reset could grant a “winner” status to PSC across species. f, Schematic representation of tetraploid complementation experiment to assess the possibility of enhancing the developmental potential of already naïve mouse ESCs. g, Representative phase-contrast images of day 5 S*K-reset mESCs of C57BL/6J background. The dome-shaped naïve-like morphology did not differ in pCXLE-mCherry-S*K and pCXLE-mCherry mock control samples. h, Percentage of 4N-aggregated embryos derived from ESCs from g that gave rise to full-term pups, pups that initiated breathing, pups that survived foster-nursing for at least 48 h, and those survived to adulthood (at least 3 months). Bars are representing the mean survival of all transferred embryos; numbers are shown on top. 8 times more pups were born from pCXLE-mCherry-S*K-nucleofected cells compared to pCXLE-mCherry control. i, All-iPSC pups generated by tetraploid complementation assay e with S*K-reset female mESCs. j, Schematic simplification results in e-j.

The examples illustrate the invention.

### EXAMPLE 1—MATERIAL AND METHODS

### Mice

All mice used were bred and housed at the mouse facility of the Max Planck Institute in Münster. Animal handling was in accordance with MPI animal protection guidelines.

### Vector Construction

The pMX-Sox2/Sox17 chimeric transcription factor vectors were based on Addgene ID 13367 (Takahashi and Yamanaka, 2006) and the tet-inducible pHAGE2-tetO-Oct4-P2A-Sox2-17-T2A-Klf4-E2A-cMyc (OS*KM) and pHAGE-tetO-Sox2-17-T2A-Klf4-E2A-cMyc (S*KM) vectors were based on Addgene ID 136551 and 136541, respectively (Velychko et al., 2019a).

The self-replicating RNA vector T7-VEE-OKS*iG was based on Addgene ID 58974 (Yoshioka et al., 2013). The mouse episomal vectors pCXLE-Oct4-P2A-Klf4-IRES-Sox2 (OKS) and pCXLE-Oct4-P2A-Klf4-IRES-Sox2-17 (OKS*), and human episomal vectors pCXLE-SOX2-P2A-KLF4 (SK), pCXLE-SOX2^{AV}-P2A-KLF4 (S^{AV}K), pCXLE-SOX2-17-P2A-KLF4 (S*K), pCXLE-mCherry-E2A-SOX2-P2A-KLF4 were based on Addgene ID 27078 (Okita et al., 2013), except the inefficient self-cleaving peptide F2A was replaced with P2A to avoid protein fusion. pCXLE plasmids showed much better yields when grown in Stbl2 competent E. coli (Invitrogen).

The protein sequences of mouse and human Sox2^{A61V} and Sox2-17, where the HMG-box domain is indicated in uppercase and Sox17 parts are indicated in bold, as follows:

>Mouse Sox2^{A61V}
(SEQ ID NO: 47)
mynmmetelkppgpqqasgggggggnataaatggnqknspDRVKR
PMNAFMVWSRGQRRKMAQENPKMHNSEISKRLGAEWKLLSETEKR
PFIDEAKRLRVLHMKEHPDYKYRPRRKTKTLMKKDKytlpgglla
pggnsmasgvgvgaglgagvnqrmdsyahmngwsngsysmmqeql
gypqhpglnahgaaqmqpmhrydvsalqynsmtssqtymngspty
smsysqqgtpgmalgsmgsvvkseasssppvvtssshsrapcqag
dlrdmismylpgaevpepaapsrlhmaqhyqsgpvpgtaingtlp
lshm
>Human Sox2^{A61V}
(SEQ ID NO: 11)
mynmmetelkppgpqqtsgggggnstaaaaggnqknspDRVKRPM
NAFMVWSRGQRRKMAQENPKMHNSEISKRLGAEWKLLSETEKRPF
IDEAKRLRVLHMKEHPDYKYRPRRKTKTLMKKDKytlpggllapg
gnsmasgvgvgaglgagvnqrmdsyahmngwsngsysmmqdqlgy
pqhpglnahgaaqmqpmhrydvsalqynsmtssqtymngsptysm
sysqqgtpgmalgsmgsvvkseasssppvvtssshsrapcqagdl
rdmismylpgaevpepaapsrlhmsqhyqsgpvpgtaingtlpls
hm
>Mouse Sox2-17
(SEQ ID NO: 14)
mynmmetelkppgpqqasgggggggnataaatggnqknspDRVKR
PMNAFMVWSRGQRRKMAQENPKMHNSEISKRLGAEWKALTLAEKR
PFIDEAKRLRVLHMQDHPNYKYRPRRRKQVKRMKRVeggflhalv
epqagalgpeggrvamdglglpfpepgypagpplmsphmgphyrd
cqglgapaldgyplptpdtspldgveqdpaffaaplpgdcpaagt
ytyapvsdyavsveppagpmrvgpdpsgpampgilappsalhlyy
gamgspaasagrgfhaqpqqplqpqapppppqqqhpahgpgqpsp
ppealpcrdgtesnqptellgevdrtefeqylpfvykpemglpyq
ghdcgvnlsdshgaissvvsdassavyycnypdi
>Human Sox2-17
(SEQ ID NO: 13)
mynmmetelkppgpqqtsgggggnstaaaaggnqknspDRVKRPM
NAFMVWSRGQRRKMAQENPKMHNSEISKRLGAEWKALTLAEKRPF
IDEAKRLRVLHMQDHPNYKYRPRRRKQVKRLKRVeggflhglaep
qaaalgpeggrvamdglglqfpeqgfpagppllpphmgghyrdcq
slgappldgyplptpdtspldgvdpdpaffaapmpgdcpaagtys
yaqvsdyagppeppagpmhprigpepagpsipgllappsalhvyy
gamgspgagggrgfqmqpqhqhqhqhqhhppgpgqpspppealpc
rdgtdpsqpaellgevdrtefeqylhfvckpemglpyqghdsgvn
lpdshgaissvvsdassavyycnypdv

All the relevant constructs will be available on Addgene.

### Cell Culture

HEK293T cells were cultured in low-glucose DMEM (Sigma) supplemented with 10% FBS (Capricorn Scientific), 1% Glutamax, 1% penicillin-streptomycin, 1% nonessential amino acids (all from Sigma). Mouse, human, cynomolgus and porcine fibroblasts were cultured in high-glucose DMEM (Sigma) supplemented with 15% FBS, 1% Glutamax, 1% penicillin-streptomycin, 1% nonessential amino acids (NEAA), 1% sodium pyruvate (Sigma), 1% β-mercaptoethanol (Gibco); bovine fibroblasts were cultured in 50:50 DMEM/F12 (Gibco) and IMDM (with HEPES, Cytiva) with 15% FBS and the same supplements; 5 ng/ml of human bFGF (Peprotech) was used to improve cynomolgus, bovine, and porcine fibroblast cultures.

Mouse naïve pluripotent stem cells were grown in KSR-based mouse embryonic stem cell (mESC) media: high-glucose DMEM medium supplemented with 15% KSR (Invitrogen), 1% Glutamax, 1% NEAA, 1% penicillin-streptomycin, 1% β-mercaptoethanol, and 20 ng/ml human recombinant LIF (purified in-house) on Mitomycin C-inactivated C3H MEF feeder layer. For 4N-complementation experiments the KSR-LIF media was supplemented with 2i (1 mM PD0325901 and 3 mM CHIR99021) for one passage. Mouse Gof18 GFP-E3 epiblast stem cells (EpiSCs) (Han et al., 2010) were cultured in Stem Flex media (Gibco) on FBS-coated dishes.

Human pluripotent cells were cultured in hESC media: either in DMEM/F12 supplemented with 15% KSR, 1% Glutamax, 1% NEAA, 1% penicillin-streptomycin, 1% β-mercaptoethanol and 5 ng/ml bFGF or in StemFlex media (Gibco) on Matrigel-coated dishes (Corning) or Mitomycin C-inactivated CF1 MEF feeder layer. Cynomolgus iPSCs were cultured in StemFlex media on Mitomycin C-inactivated CF1 MEF feeder layer. Bovine and porcine iPSCs were derived and cultured in StemFlex media supplemented with 2 μM XAV939 (Sigma) on Mitomycin C-inactivated CF1 MEF feeder layer in a hypoxic 5% O₂, 5% CO₂ incubator at 37 °C; other cells were cultured in normoxic conditions. They were split on FBS-coated dishes with no feeders for karyotyping.

Pluripotent stem cells of all five species were passed using Accutase (Sigma). 10 μM Rho-associated kinase inhibitor (Y-27632, Abcam) was added for the first 24 h after passaging of primed pluripotent cells of all five species (extended to 48 h for mouse EpiSCs). The cells were routinely tested for Mycoplasma contamination and tested negative.

iPSCs Generation

Mouse reprogramming experiments were done as described before (Velychko et al., 2019a, 2019b). Briefly, for retrovirus production monocistronic pMX-Oct4, Sox, and Klf4 vectors were co-transfected with pCL-Eco (Addgene ID 12371) (Naviaux et al., 1996) in HEK293T cells with FuGENE6 (Promega) using low volume transfection protocol (Steffen et al., 2017). For lentivirus production pHAGE2-tetO vectors were co-transfected with PAX2 and VSV. The viral supernatants were harvested after two and three days, filtered (Millex-HV 0.45 μm; Millipore) aliquoted and stored at −80 °C. For reprogramming, Oct4-GFP MEFs (OG2 or Rosa26TA-Gof18) were plated on gelatin-coated 12-well plates at 3×10⁴ cells per well in fibroblast media. A few hours later the cells were infected with titer-adjusted volumes of each viral supernatant supplemented with 6 μg/ml (final concentration) of protamine sulfate (Sigma). After two days, the media was replaced with mouse ESC media.

For human retroviral reprogramming, 48 h after infection, the transduced cells were split on a CF1 feeder layer at 104 per 6-well plate. After one week, fibroblast media was changed to hESC media.

For mouse episomal reprogramming 10⁵ of Oct4-GFP (Rosa26TA-Gof18) MEFs were plated on gelatin-covered 6-well plates overnight, and transfected with 1.5 μg of pCXLE-OKS or OKS* combined with 0.5 μg of pCXWB-EBNA1 (Addgene ID 37624) with FuGENE6.

Human self-replicating RNA-based reprogramming was performed as previously described (Yoshioka et al., 2013). Briefly, the T7-VEE constructs were digested with MluI and then in vitro transcribed using RiboMAX Large Scale RNA Production System Kit (Promega). The transcripts were 2′-O-methylated, capped, and poly(A)-tailed using respective CELLSCRIPT kits following the manufacturer's protocol. For reprogramming, 1 μg of RNA replicons were transfected into 105 fibroblasts on 6-well plates using RiboJuice (Sigma) in the presence of 100 ng/ml B18R (Promega). The media was supplemented with 0.5 mM VPA, 5 μM EPZ to enhance the very inefficient RNA-based reprogramming. The reprogramming worked more efficiently when no puromycin selection was used. After two weeks, the cells were sorted for TRA-1-60 and plated on CF1 feeder layer in human ESC media without B18R.

Human and cynomolgus episomal reprogramming was done as previously described (Kime et al., 2015). Briefly, 5×10⁵ human newborn foreskin fibroblasts (Young, Y) (Shahbazi et al., 2016), 56-year-old male dermal fibroblasts (Old, 0, AG04148), or cynomolgus (MHH Hannover) were nucleofected with 3 μg of plasmid DNA mix: pCXLE-SOX2-P2A-KLF4 or pCXLE-SOX2-17-P2A (made for this study), pCXLE-L-MYC-F2A-LIN28 (ML, Addgene ID 27080), pCXLE-hOCT4-shTP53 (Addgene ID 27077), pCXWB-EBNA1 using Lonza NHDF Nucleofector kit (U-23 program), and plated in ROCKi-containing fibroblast media on CF1 feeder layer at different densities.

For cattle (bovine) reprogramming, 10⁶ bovine fetal fibroblasts (GOF 451-1) (Wuensch et al., 2007) or porcine fetal fibroblasts (Nowak-Imialek et al., 2011) were nucleofected with 6 μg of plasmid DNA mix: pCXLE-SOX2-P2A-KLF4 or pCXLE-SOX2-17-P2A, pCXLE-L-MYC-F2A-LIN28, pCXLE-hOCT4 (Addgene ID 27076), pCXLE-p53DD (Addgene ID 41859), and pCXWB-EBNA1 using the human protocol. For bovine reprogramming, pCXLE-p53DD could be omitted.

The virus supernatant volumes were adjusted according to qPCR titration using common WPRE or 3′UTR primers normalized to Rpl37a (Velychko et al., 2019a). All the tetO lines were screened for promoter leaking, only those with minimal leaking were selected for characterization. The newly generated iPSC lines (mouse, human, cynomolgus, and cow) were karyotyped using DAPI staining of metaphase spreads, only the lines with correct chromosomal numbers were selected for characterization. As we reported before (Velychko et al., 2019a), no difference in aneuploidy occurrence was observed between different cocktails. Similar to other studies, we only tested the quality of male iPSCs for this work.

iPSCs were characterized as previously described, except for bovine iPSCs (biPSCs), which did not generate teratomas in SCID mice in our two injection attempts. For the third attempt, we injected control biPSCs, or S*K biPSCs (nucleofected with episomal pCXLE-SOX2-17-P2A-KLF4 one week before) into the left and right sides of the same mouse, respectively. A teratoma arose only on the right side one month later (FIG. 10z-ab).

### Primed-to-Naïve Conversion

For primed-to-naïve conversion (pluripotency upgrade), human iPSCs were transduced with monocistronic or polycistronic pHAGE2-EF1α lentiviral vectors carrying reprogramming factors. After two days, the cells were passed at low density (10³ cells per 24-well plate) on inactivated C3H feeder layer in mESC media supplemented with ROCKi with or without 2i. 24 h later the media was changed to mESC media without ROCKi with or without 2i. Six days after passing, the cells were fixed and stained for KLF17 (HPA024629, ATLAS, 1:500). PD0325901, but not CHIR99021 or PD0325901+CHIR99021 (2i) increased the number of KLF17+ colonies.

For integration-free pluripotency upgrade, 3×10⁵ of GFP-negative Gof18 E3 mouse epiblast stem cells (mEpiSC) (Han et al., 2010) cells were seeded in StemFlex+ROCKi media on FBS-coated 12-well plates and simultaneously transfected with 2 μg of episomal pCXLE-mCherry, pCXLE-mCherry-T2A-SOX2-P2A-KLF4, pCXLE-mCherry-T2A-SOX2^{AV}-P2A-KLF4, or pCXLE-mCherry-T2A-SOX2-17-P2A-KLF4 using 4 μL of Lipofectamine Stem Reagent (Invitrogen) according to manufacturer's instructions; after 48 h the cells were sorted for mCherry and plated in mouse ESC media +ROCKi on inactivated C3H feeder layer at 104 per 12-well plate. ~30% of sorted cells survived; of those ~50% of SK/S^{AV}K/S*K-transfected colonies grew dome-shaped and were GFP+ already on day four after passing. The GFP+ colonies were picked and clonally expanded for further characterization.

### Integration-Free Primed to Naïve Reset

For integration-free reset, 3×10⁵ of GFP-negative Gof18 E3 mouse epiblast stem cells (mEpiSC) 10⁶ cells were seeded in StemFlex+ROCKi media on FBS-coated 12-well plates and simultaneously transfected with 2 μg of episomal pCXLE-mCherry, pCXLE-mCherry-T2A-SOX2-P2A-KLF4 or pCXLE-mCherry-T2A-SOX2-17-P2A-KLF4 (Addgene ID 193293, 193296, and 193294, respectively) using 4 μL of Lipofectamine Stem Reagent (Invitrogen) according to manufacturer's instructions; after 48 h the cells were sorted for mCherry and plated in mESC media +ROCKi on an inactivated C3H feeder layer at 104 per 12-well plate. ~30% of sorted cells survived; of those ~50% of SK/SAVK/S*K-transfected colonies grew dome-shaped and were GFP+/mCherry− already on day 4 after passing. The GFP+ colonies were picked and clonally expanded for further characterization. Lipofections with RNA synthesized from T7-VEE-CFP-e2a-SOX2-p2a-KLF4 and T7-VEE-CFP-e2a-SOX2-17-p2a-KLF4 (Addgene ID 193358 and 193360, respectively) delivered the same results (data not shown).

For human iPSC (hiPSCs, episomal A18945 from Gibco) or ESCs (hESCs, H9) naïve reset, 0.5×10⁶ primed cells were nucleofected with 6 μg of pCXLE-mCherry-T2A-SOX2-17-P2A-KLF4 or pCXLE-mCherry control mixed with pCXWB-EBNA1 (Addgene ID 37624) in 3:1 ratio; with Nucleofector 2b (program B-016), and Lonza Human Stem Cell Nucleofector™ Kit 1 (Catalog #: VPH-5012) according to manufacturer's protocol. For EMSA experiments, pCXLE-SOX2-17-P2A-KLF4 (Addgene ID 193290) versus control empty pCXLE plasmid were used. The nucleofected cells were plated on feeders or on FBS-coated dish in StemFlex+ROCKi media and cultured in hypoxic 5% O2, 5% CO₂ incubator at 37 °C. On the second day, the media was changed to StemFlex. Optionally, human naïve media (RSeT™, STEMCELL Technologies) were added on day 3. RSeT worked well on feeders, while StemFlex yielded more KLF17*and SUSD2* colonies in feeder-free conditions.

The following plasmids were used to constitutively label the A18945 hiPSC line for mouse/human chimera experiments: AAVS1-Pur-CAG-mCherry (Addgene #80946), gRNA_AAVS1-T2 (Addgene #41818), pX330-U6-Chimeric_BB-CBh-hSpCas9 (Addgene #42230). For assessing embryo development contribution, we aggregated the S*K-reset hiPSCs with mouse morulae at E2.5 as previously described (Eakin et al., 2006). After 2 days of culture (E4.5), the chimeric embryos were stained for human-specific SUSD2 (Biolegend 327401) and mouse-specific Oct4 (D6C8T, Cell Signaling). All the experiments were performed in accordance with ISSCR guidelines.

### Tetraploid (4N) Complementation Assay

### 1. Preparation of Tetraploid Embryos

Super ovulated B6C3 F1 females were mated with CD1 males. E1.5 embryos at the two-cell stage are flushed from the oviducts and collected in M2 medium.

After the equilibration in fusion solution (0.3 M D-mannitol, 50 μM CaCl2, 0.3% BSA (Sigma)) 50-75 embryos are placed between the electrodes of a 250 μm gap electrode chamber (BLS Ltd.) containing 0.3 M mannitol with 0.3% BSA and fused with a Cellfusion CF-150/B apparatus (BLS Ltd.) with 0.5 mm Micro fusion slide (BTX-450). An initial electrical field of 2V is applied to the embryos followed by one peak pulses of 60V for 50 μs. Embryos are transferred back into KSOM-aa medium and immediately into a 37 °C incubator with 5% CO₂. Embryos are observed for fusion after 15 to 60 minutes. The fused tetraploid embryos are cultured to the 4-cell stage for 24 h under the same conditions.

2. Aggregation of iPSCs with Zona-Free Embryos
(1) Preparation of aggregation plates for mouse embryos chimera production 1 h before aggregation:
   Using a KSOM medium filled 100 μl-pipette, make 4 rows of microdrops (roughly 3 mm in diameter) in a 35 mm dish (Falcon, Cat. No. 35-3001), two drops in the first and fourth, five drops in the second and third rows.
   Cover the whole plate with paraffin oil.
   Sterilize the aggregation needle (BLS Ltd.) with 70% ethanol.
   Press the aggregation needle into the plastic through the paraffin oil and culture medium, while making a circle movement to create a tiny scoop of about 300 μm in diameter with a clear smooth wall. Six to ten holes can be made within each droplet.
(2) iPSCs are aggregated and cultured with denuded 4-cell stage mouse tetraploid embryos as reported with a slight modification (Nagy et al., 1993):
   Clumps of loosely connected iPSCs (15-20 cells in each) from short trypsin-treated day two iPSC cultures were chosen and transferred into microdrops of KSOM medium under mineral oil; each clump is placed in a depression in the microdrop. Meanwhile, batches of 30-50 embryos were briefly incubated in acidified Tyrode's solution (Hogan et al., 1986) until dissolution of their zona pellucida. Two embryos were place on the iPSC clump. All aggregates are assembled in this manner, and cultured overnight at 37 °C, 5% CO₂.
   After 24 h of culture, the majority of aggregates have formed blastocysts. Ten to fourteen embryos were transferred into one uterine horn of a 2.5 dpc pseudopregnant recipient. Mature CD-1 females were used as pseudopregnant foster mothers with a weight of 30+ g.

### Mammalian Cell Overexpression and Whole Cell Lysate (WCL) Generation

HEK293T cells cultured on 10 cm dishes were transfected with 10 μg of pLVTHM or pHAGE2 vectors under the control of an EF1α promoter and containing the wild-type or mutant versions of Oct4 or Sox2 with Fugene6 (Promega) using a low volume protocol (Steffen et al., 2017). Three days after transfection, the cells were dissociated from the plate using Accutase (Sigma), collected, counted, and washed with PBS. WCLs were generated by five cycles of freeze-thawing pellets resuspended in 12.5 μL per million cells in lysis buffer (20 mM HEPES-KOH pH 7.8, 150 mM NaCl, 0.2 mM EDTA pH 8, 25% glycerol, 1 mM DTT, and cOmplete™ protease inhibitor cocktail (Merck). After disruption, lysates were spun at 14k RCF at 4 °C for 10 min. After centrifugation, pellets were discarded and the supernatants transferred to a new tube for further analysis. Protein concentrations were estimated by diluting samples in 0.1% SDS solution, measuring A₂₃₀ and A₂₆₀, and applying the equation:

Conc. (μg/μL)=(0.183*A₂₃₀−0.075*A₂₆₀)*dilution factor

All samples were diluted to 1 μg/μL, aliquoted, snap frozen, and stored at −80 °C. Western blots were run to compare expression levels between mutants. Expression was evaluated by Quantity One® (v4.6.7, Bio-Rad) densitometry to adjust for equal amounts of expression using WCL of untransfected cells to maintain total protein content, when necessary.

### Western Blot Analysis

5-10 μg of total protein was combined with Laemmli sample buffer, heated, and loaded onto 12% mini SDS-polyacrylamide gel (SDS-PAG) using the Towbin buffer system (Towbin et al., 1979). Gels were run initially at 15V for 15 minutes to load samples into the stacking gel and then 50 V for 30-60 minutes to resolve the proteins of interest. Samples were transferred to Immobilin®-FL PVDF membranes (Merck Millipore Ltd.) at 4 °C under 300V for 2h. Membranes were blocked for one hour at room temperature in 5% skim milk (Sigma) dissolved in PBS with 0.1% Tween-20 (PBS-T) and incubated overnight at 4 °C with rotation in the primary antibody diluted in blocking solution. The following day the membrane was washed three times in PBS-T and then incubated in secondary antibody diluted in blocking solution for one hour at 25 °C. The following antibodies were used: polyclonal goat anti-Oct4 N-19 (sc-8628, Santa Cruz Biotechnology) or monoclonal mouse anti-Oct4 (611203, BD Biosciences), polyclonal goat anti-Sox2 (sc-17320, Santa Cruz Biotechnology), monoclonal mouse anti-alpha tubulin (T6199, Sigma), 647-conjugated anti-goat (Alexafluor), and 647-conjugated anti-mouse (Alexafluor). Western blots signal was detected using Fujifilm FLA-9000 fluorescence scanner (Fujifilm).

### Insect Cell Expression and Protein Purification

The coding sequence of full-length Mus musculus Sox2 or Sox2^{AV} was cloned into pCoofy27 plasmid with a N-terminal 6×His-tag using SLIC as previously described: forward primer 3C, reverse primer ccdB (Scholz et al., 2013). Plasmids were then transformed into DH10EMBacY (a gift from Dr. Imre Berger) for baculovirus plasmid DNA amplification (Trowitzsch et al., 2010). Bacmids were purified using Macherey-Nagel Xtra BAC100 (Düren) and then transfected into a suspension of Sf9 cells at 0.8×10⁶ cells/mL grown in serum-free EX-CELL® 420 medium containing L-glutamine (Sigma) and incubated at 26 °C with shaking for virus production. Cells were monitored daily for increased cell size and GFP fluorescence. Once ~90% of cells were GFP+, viral suspensions were spun down and then filtered through 0.22 mm. Viral supernatants were expanded once before used for infection, filtered aliquots were stored at −80 °C.

Optimal protein expression conditions were determined empirically. Mid-log phase High Five™ insect cells were split to 10⁶ cells/mL in 2 L and then infected with 10-12 mL of P1 baculovirus from previous steps per liter of cells. Following incubation at 28 °C for 96 h with shaking, cell pellets were collected by centrifugation. Pellets were resuspended in lysis buffer (20 mM HEPES pH 7.5, 300 mM NaCl, 30 mM Imidazole, 5% glycerol, 0.1% Triton X-100, cOmplete™ protease inhibitor cocktail (Merck), and 1 mM DTT), frozen and thawed once, then sonicated at 4 °C using a probe sonicator (Bandelin Sonopuls, Bandelin Eletronics). Pellets were resuspended in inclusion body wash buffer (20 mM HEPES pH 7.5, 200 mM NaCl, 1 mM EDTA, 1% Triton X-100, cOmplete™ protease inhibitor cocktail (Merck), and 1 mM DTT) and subject to four cycles of dounce homogenization followed by centrifugation for 20 min. at 18k RCF and 4 °C, twice with inclusion body wash buffer and twice in buffer without Triton X-100. The final pellet was cut twice in DMSO and then incubated for 30 min at 25 °C. Unfolding buffer (7 M guanidine hydrochloride, 20 mM Tris-HCl pH 7.5, 5 mM DTT) was added to the pellet and incubated while rotating for 1 h at 25 °C. Nickel sepharose slurry (GE Healthcare) was washed and equilibrated in binding buffer, then supernatant was added and incubated at 4 °C overnight with rotation. Proteins were eluted using the unfolding buffer with additional 500 mM imidazole. Eluate fractions were checked with SDS-PAGE and relevant fractions pooled. Using 7 kDa molecular weight cut off (MWCO) dialysis tubing, pooled fractions were dialyzed for three buffer changes of at least six h for each volume of refolding buffer at 4 °C (7 M urea, 20 mM Na Acetate pH 5.2, 200 mM NaCl, 1 mM EDTA, and 5 mM DTT). Following centrifugation to remove any insoluble material, the supernatant was dialyzed (7 kDa MWCO) in refolding buffer with decreasing amounts of urea: 1 hour 6 M urea, 2 h 4 M, 2 h 2 M, and 1 hour in size exclusion chromatography (SEC) buffer (50 mM Tris-HCl pH 7.4, 150 mM NaCl, 1 mM EDTA, 5% glycerol). Eluate was centrifuged to remove any precipitate before loading onto HiLoad 16/60 Superdex 200 SEC column (GE Healthcare).

The coding sequence for full-length Oct4 from M. musculus was cloned into the pOPIN expression vector using the SLIC method and Phusion Flash High-Fidelity PCR Master Mix (Finnzymes/New England Biolabs). SLIC reactions were then transformed into One Shot™ OmniMAC™ 2 T1® Chemically Competent E. coli (ThermoFisher Scientific). After sequencing, the pOPIN-cHis-Oct4 construct was co-transfected with flashBACULTRA™ bacmid DNA (Oxford Expression Technologies) into Sf9 cells (ThermoFisher Scientific) using Cellfectin II® (ThermoFisher Scientific) to generate recombinant baculovirus. Mid-log phase Sf9 cells were used to amplify the virus. Suspension High Five™ cells were infected with P3 virus for two days at 27 °C and 120 rpm shaking. After expression, crude lysates were purified on a HiTrap TALON column (GE Healthcare), cleaved on the column with 3C protease followed by size exclusion chromatography (HiLoad Superdex 200, GE Healthcare). The final product was collected in 25 mM HEPES pH 7.8, 150 mM NaCl, 1 mM TCEP, and 5% glycerol with ~95% purity confirmed by SDS-PAGE. Fractions were checked with SDS-PAGE, pooled, and finally quantified using the NanoDrop spectrophotometer (ND-1000, ThermoFisher Scientific) and the Protein A₂₈₀ program using specific molecular weight and extinction coefficients for either Sox2 or Oct4. Unless otherwise indicated all chemicals were from Sigma-Aldrich.

### Electrophoretic Mobility Shift Assays (EMSAs)

DNA probes were generated by annealing complementary 5′ labeled Cy5 oligos (Metabion International AG) followed by purification from 10% polyacrylamide gels. For binding reactions, WCL (2-4 ug of total protein) or purified proteins were incubated in binding buffer (25 mM HEPES-KOH pH 8, 50 mM NaCl, 0.5 mM EDTA, 0.07% Triton X-100, 4 mg/mL BSA, 7 mM DTT, and 10% glycerol) and 70 nM Cy5-dsDNA at 37 °C for 1h. Samples were then loaded onto 6% native polyacrylamide gels (37.5/1 acrylamide/bis-acrylamide) containing 0.3× Tris-borate EDTA and 5% glycerol and run at 10 mA/gel in running buffer of the same composition. Gels were imaged using Fujifilm FLA-9000 fluorescence scanner using (Fujifilm). Fraction bound was determined by densitometry of raw data using Quantity One® (v4.6.7, Bio-Rad) and the following equation for specific bands and then normalized: F_{B}=DNA_{bound}/(DNA_{bound}+DNA_{unbound}). Half-life was calculated using fraction bound as a function of protein concentration from at least two independent experiments, error bars represent SD.

Equations for decay were determined using nonlinear regression in Prism 7 for Mac (version 7.0a) and only used when goodness of fit, evaluated by R² values, was 0.95 or greater.

For competition experiments, pre-formed protein/DNA or protein/nucleosome complexes (see binding conditions above) were loaded onto native gels (t=0) and then incubated with unlabeled double stranded DNA containing the Nanog locus. Protein dissociation was monitored by removing aliquots of the reaction at the given time points and loading them onto a running gel. Protein complex stability was highly variable thus conditions for competition assays were determined empirically and can be found in the tables below.

| Name | Sequence | Ref. |
|---|---|---|
| Fgf4 | GGAGAAGAAAACTCTTTGTTTGGATGCTAATGGGATACTAAG | (Ambrosetti et al. |
| | CTCC (SEQ ID NO: 48) | 1997) |
| HoxB1 | GGAGGAAGTGTCTTTGTCATGCTAATGATTGGGGCTCC | (Di Rocco et al., |
| | (SEQ ID NO: 49) | 2001) |
| Nanog | TCCACCATGGACATTGTAATGCAAAAGAAGCTGTAAG(SEQ | (Soufi et al., 2015) |
| | ID NO: 50) | |
| Oct4DE | CTATCATGCACCTTTGTTATGCATCTGCCGTCTGCCC (SEQ | (Okumura- |
| | ID NO: 51) | Nakanishi et al., |
| | | 2005) |
| Utf1 | GGAGAAGATGAGAGCCCTCATTGTTATGCTAGTGAAGTGCC | (Nishimoto et al., |
| | AAGCTCC (SEQ ID NO: 52) | 1999) |
| Non- | CTGCAGGTGGGATTAACTGTGAATTCA (SEQ ID NO: 53) | (Soufi et al., 2015) |
| specific | | |
| Widom+6 | CTGGAGAATCCCGGTCTGCAGGCCGCTCAATTGGTCGTAGA | (Michael et al., |
| | CAGCTCTAGCACCGCTTAAACGCACGTACGCGCTGTCCCCC | 2020) |
| | GCGTTTTAACCGCCAAGGGGATTACTCCCTAGTCTCCAGGC | |
| | CTTTGTTATGCAAATACATCCTGT (SEQ ID NO: 54) | |

| | Competition: WCL | | |
|---|---|---|---|
| | Oct4DE | Nanog | Fgf4 |
| Target Conc. | 35 nM | 35 nM | 35 nM |
| Competitor Conc. | 350 nM | 14,000 nM | 350 nM |
| Temp. competitor incubation | 25 °C | 32 °C | 25 °C |

| | Competition: Purified Protein | | |
|---|---|---|---|
| | Nanog | Utf1 | Widom + 6 Nuc. |
| Target Conc. | 20 nM | 20 nM | 20 nM |
| Oct4 Conc. | 20 nM | 20 nM | 104 nM |
| Sox2 Conc. | 20 nM | 20 nM | 52 nM |
| Competitor Conc. | 16,000 nM | 4,000 nM | 1,000 nM |
| Temp. competitor incubation | 37 °C | 37 °C | 16 °C |

### Nucleosome Assembly

The nucleosome DNA sequence Widom +6 consists of 147 bp of the established Widom 601 sequence (Lowary and Widom, 1998) with a Sox/Oct motif (CTTTGTTATGCAAAT (SEQ ID NO: 55)) at super helical location +6, with the nucleosome dyad being zero (Michael et al 2021). DNA was purchased double stranded from IDT (Coralville) and labeled using Cy-5 conjugated primers via PCR, as previously described (Michael et al., 2020). Nucleosomes were assembled from DNA:octamer ratios ranging from 1:1.2-1:1.6 with purified full-length D. melanogaster histone octamer (Klinker et al., 2014) using the salt-gradient dialysis method previously described (Luger et al., 1999), final buffer composition: 10 mM HEPES pH 7.6, 50 mM NaCl, 1 mM EDTA, and 0.5 mM DTT. Following dialysis, nucleosomes were heat shifted at 37 °C for 2h. Nucleosome quality and concentration were evaluated using native PAGE run with a histone-free DNA standard curve made from the parent DNA. Histone stoichiometry was checked by 22% SDS-PAGE followed by coomassie staining (R-250; SERVA). Nucleosome were stored at 4 °C in the dark and used for no longer than three weeks from the date of assembly.

### Molecular Dynamics Simulation (MDS)

The model of the Oct4-Sox2 heterodimer bound to a regulatory DNA element from the Hoxb1 enhancer that was previously built (Esch et al., 2013) was used as template for building new models for the Sox2/Oct4, Sox2^{A61V}/Oct4, Sox2/Oct6, Sox2^{A61V}/Oct6. Using MODELLER (https://salilab.org/modeller/), the sequences were adapted, each model of the Oct factor was extended with 4 and 8 residues at the N- and C-termini respectively and similarly, each model of the Sox factor was extended with 4 and 5 residues. 100 models were built for each ternary complex using a “slow” optimization procedure that included a “slow” MD refinement as defined in MODELLER. The models were ranked using an energetic score (“DOPE”) and selected 2 models for each complex for MD simulations. In each of these models, the DNA was extended by 16 and 18 base pairs at the 5′ and 3′ ends using the mouse Hoxb1 sequence (Aksoy et al., 2013b). The final sequence in the model was:

(SEQ ID NO: 56)
5′-AGAGTGATTGAAGTGTCTTTGTCATGCTAAT
GATTGGGGGGAGATGGAT-3′.

Then, the systems were solvated in a truncated octahedron periodic box of SPCE water with the distance between any protein-DNA atom to the box edges larger than 12 Å. 73 neutralizing Na+ ions and 150 mM KCl (225 K+ and 225 Cl− ions) were added. For the ions the parameters developed by Li and Merz (Li et al., 2015) were used. The Amber-ff14SB (Maier et al., 2015) and the Amber-parmbsc1 (Ivani et al., 2015) force fields were used for proteins and DNA respectively. Each system was energy minimized and equilibrated with a protocol described previously (Jerabek et al., 2017). With each model 2 independent, 1.2 μs long MD simulations were performed by assigning different velocity distributions before the equilibration (in total 4×1.2 μs=4.8 μs per system). Periodic boundary conditions were applied in the isothermic-isobaric (NPT) ensemble with a timestep of 2 fs. The temperature was maintained at 300 K with Langevin Dynamics (damping coefficient of 0.1 ps-1). The pressure was maintained at 1 atm with the Nose Hoover Langevin Piston method with the period and decay of 1.2 and 1.0 ps respectively. The direct calculation of the non-bonded interactions was truncated at 10 Å and the chemical bonds of hydrogens were kept rigid with the SHAKE algorithm. Long range electrostatics were calculated using the particle mesh Ewald algorithm. All simulations were performed in NAMD (Phillips et al., 2005). Snapshots were selected for analysis every 10 ps.

The coordination number between two atom selections describes the number contacts between the selections using a continuous switching function with a distance threshold for contact formation as implemented in the COLVAR module of NAMD. The mathematical formula is:

$$c = {\sum }_{i} {\sum }_{j} \frac{1 - {( {d}_{ij} / {d}_{0} )}^{n}}{1 - {( {d}_{ij} / {d}_{0} )}^{m}}$$

where i,j=a pair of atoms, one from each selection; dᵢⱼ=the distance between i and j; d₀=the distance threshold (4.5 Å); n,m=exponents describing the switching from contact to no contact (n=6,m=12)

### NGS and Bioinformatic Analysis

For ChIP-seq experiments, Rosa26TA-Gof18 MEFs were infected with titrated volumes of pHAGE2-tetO-Klf4-IRES-Sox2/Sox2^{AV} with or without pHAGE2-tetO-Oct4/Oct6. After 48 h, the media was replaced with fibroblast media supplemented with doxycycline (dox). The samples were collected 48 h after dox-induction. RNA-seq and RRBS were performed for human iPSC at passage 10-12, human ESCs at passage 35-36 grown in StemFlex media on matrigel, human fibroblasts at passage 11-15 were grown on gelatin-coated dishes in fibroblast media. The sample processing and data analysis for RNA-seq, ChIP-seq, RRBS were performed as described before (Keshet and Benvenisty, 2021; Malik et al., 2019; Velychko et al., 2019a).

For footprinting analysis, briefly, the publicly available data (ArrayExpress: E-MTAB-7207) were aligned to mm10 genome using bowtie2 (Langmead and Salzberg, 2012) using “—very-sensitive-X 2000—no-mixed” options; the mitochondrial and duplicate reads were removed, and the reads were sorted and indexed using samtools (Danecek et al., 2021); spearman correlation was plotted using deeptools (Ramírez et al., 2016); the peaks were called using macs2 using “-g mm-f BAMPE—call-summits—cutoff-analysis—keep-dup all-B” options (Zhang et al., 2008); the output of macs2 was used for TOBIAS footprinting analysis (Bentsen et al., 2020) using ENCODE blacklist (Amemiya et al., 2019), JASPAR MEME motif database (Castro-Mondragon et al., 2022) with some additional custom motifs (Malik et al., 2019).

### EXAMPLE 2—DEFINING THE STRUCTURAL ELEMENTS OF SOX17 THAT BENEFIT INDUCTION OF PLURIPOTENCY

Oct4 (Pou5f1) is the only TF of the POU (Pit1, Oct1/Oct2, UNC-86) family that can induce pluripotency in both mouse and human cells (Takahashi and Yamanaka, 2006; Takahashi et al., 2007; Yu et al., 2007), while other members of the family, such as the ubiquitously expressed Oct1 (Pou2f1), neural Oct6 (Pou3f1), and Brn4 (Pou3f3 or Oct9) cannot (Jerabek et al., 2017; Nakagawa et al., 2008; Velychko et al., 2019b). POU factors have different binding profiles and different preferences for hetero-versus homodimerisation (Jerabek et al., 2017; Malik et al., 2019; Mistri et al., 2015). In the instant inventor's search to find what makes Oct4 unique among POU factors, they discovered that the Sox17^{E57K} mutant (Jauch et al., 2011), but not wild type Sox2, can efficiently generate iPSCs in combination with Brn4 (FIG. 1 a). POU factors consist of a DNA-binding domain (POU domain), flanked by N- and C-terminal transactivator domain (NTD and CTD). The POU domain is bipartite, consisting of a POU-specific domain (POU_{S}) and a POU-homeodomain (POU_{HD}) joined by a flexible non-conserved linker. Oct4's but not Oct1's linker contains an alpha-helix near its N-terminus (Esch et al., 2013; Klemm et al., 1994). Replacement of the Oct4 linker with those from other POU factors, or even the point mutant L80→A in the linker helix is detrimental for induction and maintenance of pluripotency or supporting normal development (Chen et al., 2020a; Esch et al., 2013; Han et al., 2022a). Surprisingly, Sox17^{EK} could also rescue the reprogramming ability of Oct4^{L80A} (FIG. 7a).

A library of chimeric TFs was constructed, where the non-conserved residues from Sox17 were swapped into Sox2 to find the structural elements responsible for the rescuing phenomenon (FIG. 1b-c). Monocistronic retroviral supernatants were used for reprogramming of mouse embryonic fibroblasts (MEFs) carrying endogenous Oct4-GFP reporter (OG2); the volumes of viral supernatants were adjusted based on qPCR titration (FIG. 7b-c). Initial screening results pointed to amino acids 61-62 of Sox17 as the most crucial (FIG. 1d). The Sox17-CTD enhanced reprogramming with wild-type Oct4, but could not rescue Oct4^{L80A} (Sox2c17 chimera, FIG. 1 d-e) unless combined with the 61-62 region (Sox2⁶¹⁻⁶²c17 chimera, FIG. 1d-e). Two other Sox17 regions, 43-47 and 65-86, also boosted the reprogramming efficiency, particularly when combined with 61-62 (FIG. 1 d-e). Other Sox17 elements (e.g., 24-28) significantly decreased the reprogramming efficiency of Sox2 (FIG. 1d). A second screen determined that a single A61V swap in Sox2 rescues both Oct4^{L80A} and Brn4, while L62Q was inconsequential (FIG. 1f-g). Sox2^{A61V} also allowed reprogramming with Oct2 (Pou2f2), Oct6, and Brn2 (Pou3f2 or Oct7) (FIG. 1h). Oct1 (Pou2f1) was the only tested POU factor that did not yield iPSC colonies when co-expressed with Sox2^{A61V} and Klf4 in MEFs (FIG. 1h). An A61L mutation was also tested, as leucine has even higher hydrophobicity than valine. While Sox2^{A61L} performed better than wild type Sox2, it was significantly worse than Sox2^{A61V} (from now on Sox2^{AV}), especially in rescuing Brn4 (FIG. 1f-g). Notably, a complex chimeric Sox factor, Sox2^{43-47,61,65-86}c17 (Sox2-17 or S* from now on), that combines 16 beneficial Sox17 residues in Sox2 HMG-box domain and a complete CTD swap (FIG. 1b-c) exceeded the reprogramming ability of Sox2, Sox2c17, or Sox17^{EK} (FIG. 1f-g). Cell proliferation is essential for the induction of pluripotency; cMyc, GATA factors, and the SV40 large T antigen increase reprogramming efficiency by boosting cell proliferation (Rand et al., 2018; Velychko et al., 2019a). However, the A61V mutation has the opposite effect: four-factor induction with Sox2^{AV} or Sox2-17 resulted in significantly lower cell proliferation compared to their respective 61A variants (FIG. 1i). The repressive effect on cell proliferation explains why A61V, while being able to rescue nonfunctional Oct4 mutants, does not by itself increase reprogramming efficiency with wild-type Oct4 (FIG. 1d). The efficiency boost comes only from the synergy between A61V and the stronger Sox17 transactivator (FIG. 1d-g).

### EXAMPLE 3: SOX2^{A61V} ENHANCES BOTH SOX/OCT COOPERATIVITY AND THE DEVELOPMENTAL POTENTIAL OF IPSCS

A61 is located in the third helix of the high-mobility group (HMG) domain of Sox2 that faces towards POU_{S} domain when the heterodimer is bound to a canonical SoxOct motif (FIG. 2a-b). Compared to alanine, valine has two additional methyl groups, making it more hydrophobic. Molecular dynamic simulations (MDS) of the Sox/Oct heterodimer on HoxB1 SoxOct element showed that swapping A to V increases the average number of hydrophobic interactions between Sox2-HMG and Oct4- or Oct6-POU_{S} (FIG. 2c). The I21 residue of both Oct factors engaged with 61V the most (FIG. 2d), which could potentially increase the cooperativity of Sox2^{A}V with POU factors. The simulations also revealed a novel structural rearrangement of the Sox2/Oct4 heterodimer that lead to higher Oct-Sox ligancy and, therefore, potentially higher cooperativity. This rearrangement was not observed when Oct4 was replaced with Oct6 (FIG. 2c). In this configuration, stabilized by A61V, the residue 61 interacts with the core of the helix near G24 of Oct4-POU_{S} and residues R50 and K57 of Sox2-HMG form salt bridges with E82 and E78 of Oct4 linker (FIG. 2e). The instant inventors term this structure of Sox2/Oct4 heterodimer SL configuration, as it involves both the POU_{S} and Linker of Oct4, as opposed to S configuration, which involves only the POU_{S}. Yet another configuration described for Sox/Oct heterodimer on the rarer Fgf4 motif was also modeled, where Sox and Oct sites are spaced by a three base-pair gap. Sox2 and Oct4 cooperate more distantly on the Fgf4 motif, forming a distant S (DS) configuration that involves Sox2's T78 and T80, but not A61 (FIG. 2f).

FLAG-tagged Oct1, Oct2, Oct4, Oct6, Brn2, and Brn4 were overexpressed in HEK293 cells and after confirming their comparable expression (FIG. 8a) the lysates were used for electromobility shift assays (EMSA) on the Nanog promoter locus. POU monomer binding was comparable for all tested factors with the exception of reduced binding by Oct1, but Oct4 showed the strongest heterodimerization capacity with Sox2 (FIG. 8b). There were no differences in monomer binding between wild-type Sox2, Sox2^{AV}, and Sox2-17, but both Sox2^{AV} and Sox2-17 enhanced heterodimerization with all the tested POU factors (FIG. 8b). To find which domains of Oct4 remain vital for generating iPSCs when combined with Sox2^{AV} and other chimeric Sox factors all 17 residues of Oct4 linker domain were replaced with synthetic poly-glycine linkers of different length (GL3-30) (FIG. 3a). Such flexible linkers were detrimental for reprogramming with wild-type Sox2, but Oct4^{GL15-30} mutants were rescued by Sox2^{AV}, and the efficiency was further increased by Sox2-17 (FIG. 3a). Second, either NTD and CTD of Oct4, both of which were previously shown to be crucial for reprogramming (Kim et al., 2020), were truncated. Indeed, neither Oct4ΔNTD nor Oct4ΔCTD could generate iPSCs when combined with wild-type Sox2 (FIG. 3b, S2c-d). However, Sox2^{AV} or Sox2c17 could rescue Oct4ΔCTD, and Sox2^{AV}c17, Sox17^{EK}, or Sox2-17 rescued either of the transactivator deletion mutants (FIG. 3b, 8c-d). These results show that increased Sox/Oct cooperativity, especially when combined with a stronger Sox transactivator, can compensate for the loss of transactivation power by Oct4 in reprogramming. Third, none of the chimeric Sox factors could rescue the deletion of Oct4-POU_{S} domain, which is directly involved in Sox/Oct interaction (FIG. 3c). Remarkably, Sox2^{AV} could even give rise to a few GFP+ colonies with Oct4ΔPOU_{HD}, where the POU_{HD} DNA-binding domain was truncated leaving the RKRKR peptide at its N-terminus that serves as nuclear localization signal (FIG. 3c, FIG. 8e). PCR-genotyping confirmed the identity of two clonal Oct4ΔPOU_{HD}/Sox2^{AV}/Klf4 iPSC lines (FIG. 8f) and they were able to contribute to chimeric mice (FIG. S2g) including the germline (FIG. 8h).

Oct4 and Sox2 mutants were overexpressed in HEK293 cells, equal expression was confirmed (FIG. 3d), and EMSA experiments were performed. Again, monomer binding showed no difference between Sox2^{A}V and wild-type Sox2 on either HoxB1 or Nanog DNA elements (FIG. 3e), however, 61V significantly increased the heterodimerization with wild-type Oct4 and partially rescued the DNA binding of the Oct4ΔPOU_{HD} mutant (FIG. 3e), in concordance with the reprogramming results (FIG. 3c, FIG. 8e-h). The Oct4ΔPOU_{HD} rescue data are of particular interest because of previous debated reports on the role of POU subdomains in Oct4's pioneering function. While a nucleosome does not pose a problem for Sox2 (Dodonova et al., 2020), it does hinder canonical Oct4 binding because POU_{S} and POU_{HD} engage opposite sides of DNA inevitably clashing with the histone core (Huertas et al., 2020; Michael et al., 2020; Soufi et al., 2015). This has prompted speculations that Oct4 engages closed chromatin with just one of its domain (Soufi et al., 2015) or even that POU_{S} alone is involved in chromatin opening (Michael et al., 2020). On the other hand, it was shown that while Oct4 uses either the POU_{S} and POU_{HD} to recognize specific sequences on nucleosomes, the other domain scans unspecifically creating barriers to nucleosome closing (MacCarthy et al., 2021). The data presented here shows that removing POU_{HD} abolishes both Oct4 DNA binding on SoxOct motifs (FIG. 3e) and the reprogramming efficiency (FIG. 3c compared to 3a-b), highlighting the importance of POU_{HD} even in the context of highly-cooperative Sox factors. It is very unlikely that stable and, therefore, consequential Oct4 binding could be achieved with just one of its subdomains in the context of wild-type Sox2 (FIG. 3e). To compare the stability of Sox/Oct4 heterodimers on different natural regulatory SoxOct DNA elements, off-rate EMSA experiments were performed, where an excess of unlabeled DNA is added to pre-formed Sox/Oct/DNA complex and samples are loaded onto a gel over a time course. Both Sox2^{AV} and Sox2-17 dramatically enhanced the heterodimer stability on both Oct4 distal enhancer (Oct4DE) and Nanog promoter elements, but they showed similar to wild-type Sox2 stability on Fgf4 locus (FIG. 3f). On both Oct4DE and Nanog probes, a proportion of heterodimers disappear almost immediately, while the remaining heterodimers were extraordinary stable (FIG. 3f), again suggesting there could be two distinct Sox/Oct configurations as with our MDS-based results (FIG. 2). Oct4, Sox2, and Sox2^{AV} purified from insect cells (FIG. 8i) were used for EMSAs on three natural SoxOct elements: Nanog promoter, Utf1 and Fgf4 enhancers (FIG. 3g, FIG. 8j). Sox2 and Sox2^{A}V monomer binding as well as heterodimerization on Fgf4 were similar, while the heterodimerization on Nanog and Utf1 were enhanced by A61V mutation (FIG. 8j). The off-rate EMSAs on Nanog and Utf1 elements are reminiscent of our whole-cell lysate experiments: A61V increased the stability of Sox2/Oct4/DNA complexes by 3 to 4 times (FIG. 3g). In agreement with the MD simulations (FIG. 2), Sox2^{AV} also strongly increased the otherwise diminished heterodimer stability with Oct4 linker mutants and Brn4 (FIG. S2k), explaining the rescuing ability in reprogramming experiments (FIG. 1f-h, FIG. 3a). It can be concluded that POU factors and Oct4 mutants that are normally incapable of reprogramming to pluripotency can be rescued by forced cooperation with Sox2 (FIG. 3h).

Both Oct4 and Sox2 are pioneer factors capable of binding and activating gene expression within inaccessible chromatin (Soufi et al., 2012; Teif et al., 2012). A modified Widom 601 nucleosome sequence adapted with a SoxOct motif at superhelical location (SHL) +6 was assembled, which was used to resolve the Oct4/Sox2/nucleosome complex by cryo-electron microscopy (Michael et al., 2020). Sox2^{AV} dramatically enhanced the stability of Oct4/Sox2/nucleosome complex (FIG. 4a). Chromatin immunoprecipitation with sequencing (ChIP-seq) was then performed to identify the binding sites at very early stage of reprogramming—two days after doxycycline (Dox)-induction of KS or OKS in MEFs. HOMER motif enrichment analysis (Heinz et al., 2010) (FIG. 9a-b) showed that A61V samples were more enriched in SoxOct motif in both Oct4/Sox2^{AV}/K and Oct6/Sox2^{AV}/K Oct ChIP but there was no big difference for Sox2 ChIP. Overall, Sox2 peaks did not significantly differ between Sox2 and Sox2^{AV} in either KS or OKS samples (FIG. 4b-c), suggesting that 61V does not change the binding profile of Sox2 itself. On the other hand, Oct ChIP for either Oct4 or Oct6 showed an increased intensity and number of SoxOct peaks (FIG. 4b-c), as well as significantly increased proportion of SoxOct to Oct and OctOct (MORE) peaks for OKS cocktails carrying A61V mutant (FIG. 4d). TOBIAS footprinting analysis of published ATAC-seq datasets was used for ESC versus MEF samples (Li et al., 2017) to determine the relevant binding sites of Oct4 and Sox2 that lead to chromatin opening (FIG. 9c). 61V did not change binding of Sox2 but significantly increased recruitment of Oct4 to the SoxOct sites that open over the course of reprogramming (FIG. 9c). The stronger binding by Oct4/Sox2^{AV} compared to Oct4/Sox2 is illustrated by increased signal for both Oct4 and Sox2 ChIP at the prominent pluripotency targets such as Nanog, Pou5f1, Nr5a2, Gdf3, and Lefty2 (FIG. 4e). Consistent with our computer modeling (FIG. 2f) and EMSA results (FIG. 3f), binding to Fgf4 locus was not affected (FIG. 4e).

In ESCs, Oct4 and Sox2 regulate genes cooperatively; the majority of pluripotency genes contain SoxOct motifs in their regulatory elements (Chen et al., 2014), while Oct4 alone binding enhances cell division rate (Lee et al., 2010). Overexpression of Oct4 in somatic cells induces hyperproliferation (Hochedlinger et al., 2005). Accordingly, OSKM induction in MEFs caused a much stronger boost of cell proliferation compared to SKM induction (FIG. 1i) (Velychko et al., 2019a). In the beginning of reprogramming, when Oct4 and Sox2 are overexpressed in somatic cells they tend to bind mostly independently engaging thousands of non-native genomic loci (Chen et al., 2016; Chronis et al., 2017; Li et al., 2019; Soufi et al., 2012). The instant inventors hypothesized that enhancing Sox/Oct cooperativity could improve the reprogramming process, as cooperativity between TFs increases their specificity (Von Hippel et al., 1996). Indeed, already on day two of OKS induction, Sox2^{AV} engaged 511 of ESC-specific super-enhancers, compared to 378 for wild-type Sox2 (FIG. 4f). To assess the effect of enhanced Sox2/Oct4 cooperativity on the developmental potential of iPSCs, we performed tetraploid (4N)-complementation assays for iPSC lines generated using lentiviral tet-inducible or episomal delivery methods, both carrying polycistronic OSKM with either wild-type Sox2 or Sox2^{AV}. In both cases, iPSC lines generated with Sox2^{AV} gave rise to significantly more fully-developed all-iPSC pups (more than two-fold difference) (FIG. 4g, Table 1). The benefit of the highly-cooperative Sox2^{AV} was particularly evident for the survival of the all-iPSC mice. As we and others previously reported (Buganim et al., 2014; Chen et al., 2015a; Velychko et al., 2019a), OSKM all-iPSC mice rarely survive to adulthood: none of the tetO-OSKM all-iPSC mice reached maturity, while three out of five tested tetO-OS^{AV}KM iPSC lines gave rise to adult mice (FIG. 4g-h, Table 1). The tetO-OS^{AV}KM all-iPSC mice were fertile; PCR genotyping confirmed the inheritance of the transgene in their progeny (FIG. 4i). Episomal (epi-) vectors deliver milder overexpression, giving rise to better quality integration-free iPSCs, even in the presence of exogenous Oct4 (Velychko et al., 2019a). Yet only 4.2% of transferred epi-OSKM all-iPSC embryos survived until adulthood, compared to 22.2% of OS^{AV}KM all-iPSC embryos. Thus, enhancing Sox2/Oct4 cooperativity significantly improves the developmental potential of mouse OSKM iPSCs.

### EXAMPLE 4: ENGINEERED SUPER-SOX ENHANCES REPROGRAMMING ACROSS SPECIES

The attention was then turned to Sox2-17—the most efficient chimeric Sox factor of this study that includes 61V along with three other structural elements of Sox17 (FIG. 1b-c). Sox2-17 was cloned into tet-inducible polycistronic OSKM or SKM reprogramming cassettes and comparable levels of expression were confirmed using qPCR (FIG. 10a). Time-course experiments with restricted Dox-induction (FIG. 5a) showed that Sox2-17 dramatically enhances in the kinetics and efficiency of reprogramming (FIG. 5b). While at least three days of OSKM induction is needed to yield the first iPSC colonies, OS*KM carrying Sox2-17 gives rise to iPSC colonies after just 24 h of induction—the fastest reprogramming reported to date. Clonally expanded 24 h iPSC lines lost methylation of Nanog and Pou5f1 promoters, and acquired methylation of fibroblast-specific Col1a1 promoter (FIG. 10b). 24 h iPSCs could differentiate into all three germ layers in teratoma assays (FIG. S4c), contribute to chimeric mice (including the germline) (FIG. 10d), and even successfully generate live-born all-iPSC pups in 4N complementation assays (FIG. 10e, Table 1). When induced for just 3-4 days, OS*KM gave rise to 10 to 200 times as many colonies as OSKM, depending on the quality of fibroblasts and the levels of overexpression (FIG. 5b-c). Sox2-17 could even generate mouse two-factor iPSCs with Klf4 alone (S*K cocktail) albeit with low efficiency, while Sox2, Sox2c17, or Sox17EK could not (FIG. 10f). Clonal S*K iPSC lines displayed normal morphology (FIG. S4g) and PCR genotyping confirmed the identity of the two lines we tested (FIG. S4h). S*K iPSCs stained positive for pluripotency markers Nanog and SSEA-1 (FIG. 10i), and gave rise to three germ layers in a teratoma assay (FIG. 10j). These data suggested that Sox2-17 requires shorter time, lower dosage, and a reduced number of co-factors to successfully induce pluripotency, which could be particularly beneficial for much less efficient integration-free reprogramming methods. Episomal Myc-free polycistronic Oct4-P2A-Klf4-IRES-Sox vectors were generated, carrying either wild-type Sox2 or Sox2-17 (epi-OKS and epi-OKS*, respectively) and the correct expression was confirmed with western blotting (FIG. 10k). Sox2-17 enhanced epi-OKS reprogramming of MEFs by a striking 150 times (FIG. 5d), giving rise to high-quality iPSCs that could generate all-iPSC mice in 4N complementation assays with up to 77% efficiency (FIG. 5e, Table 1). Remarkably, all 10 tested integration-free epi-OKS and OKS* iPSC lines gave rise to healthy adult all-iPSC mice with the survival rate similarly high for both Sox2 and Sox2-17 (FIG. 5f, Table 1).

A human version of SOX2-17 (FIG. 1c) was tested for reprogramming human fetal fibroblasts by delivery of retroviral monocistronic OSKM (FIG. 5g-h). The transduced cells were plated on inactivated feeders, and, after two weeks, stained for the human pluripotency marker TRA1-60. SOX2-17 gave rise to 56 times more iPSC colonies compared to SOX2: 8.9% and 0.16% overall reprogramming efficiency, respectively (FIG. 5g-h). It was found that SOX2-17 can reprogram human cells even when combined with OCT4 alone (OS* cocktail), albeit with low efficiency (FIG. 5i-j). It was shown that 61V within SOX2-17 is crucial for enabling two-factor reprogramming (FIG. 5i), nevertheless, SOX17^{EK} did not give rise to a single human OS iPSC colony (FIG. 4i-j). Human clonal OS* iPSCs showed normal morphology (FIG. 10l), expressed pluripotency markers NANOG and TRA1-81 (FIG. 10m), and could differentiate into tissues of three germ layers in teratoma assays (FIG. 10n). Human self-replicating RNA (Yoshioka et al., 2013) OKS*iG vector encoding for OCT4, KLF4, SOX2-17, and GLIS1 also gave ~50 times more TRA1-60+ colonies comparing to wild-type SOX2 (FIG. 5k). Moreover, using the RNA-OKS*iG vector, integration-free iPSCs from a Parkinson's patient's dermal fibroblasts were successfully generated and characterized, which could not yield iPSCs using original vector with wild-type SOX2 (Rosety et al., Sci Adv 2022, in press). SOX2-17 was also cloned into the episomal SOX2-F2A-KLF4 vector (Okita et al., 2011), replacing the original F2A self-cleaving peptide with P2A to reduce formation of a fusion poly-protein (Velychko et al., 2019b). Western blotting confirmed equal expression and the correct cleaving of SOX2/SOX2^{AV}/SOX2-17 and KLF4 (FIG. 10o). SOX2-17-P2A-KLF4 (S*K) combined with OCT4/shTP53, and L-MYC/LIN28 (OS*KML) vectors demonstrated much improved performance, particularly in reprogramming aged human dermal fibroblasts, where some reprogramming attempts using SOX2-carrying vector did not yield a single alkaline phosphatase-positive (AP*) colony (FIG. 5l). To test if the super-SOX could facilitate reprogramming for other species, the episomal vectors were tested in generating iPSCs from cynomolgus macaque (Macaca fascicularis), an important non-human primate model (Han et al., 2022b; Wunderlich et al., 2012, 2014), for which the ESCs have been derived (Chen et al., 2015b; Fu et al., 2020) but no transgene-independent iPSCs have been reported. The wild-type episomal OSKML vectors failed to give rise to any cynomolgus iPSC (ciPSCs) colonies during multiple attempts, while the same cocktail containing SOX2-17 gave rise to a few putative AP⁺ iPSC-like colonies (FIG. 5m). While most of hiPSC lines lose the episomal vectors before the third passage, only three out of 11 generated ciPSC lines completely lost the transgenes after three passages (FIG. 10p). Two of the three lines that had the correct chromosomal number were characterized (FIG. 10q). Integration-free ciPSCs displayed morphology similar to that of hiPSCs, except they were more prone to differentiation in the middle of colonies and could only be maintained on a feeder layer (FIG. 10r). They expressed pluripotency markers NANOG and OCT4 (FIG. 10s) and could differentiate into three germ layers in teratoma assays (FIG. 10t). It was also attempted to reprogram bovine and porcine fibroblasts using XAV939-containing bFGF-based media recently developed by the Smith lab to derive and maintain cattle ESCs (Kinoshita et al., 2021). Episomal reprogramming using wild-type SOX2 failed in both species but the same cocktail containing SOX2-17 efficiently generated AP⁺ iPSC-like colonies for both cow (FIG. 5n) and pig (FIG. 5o). Bovine iPSCs (biPSCs) could even be generated without inhibiting p53, albeit with low efficiency (FIG. 10u). We established 12 of such epi-OS*KML biPSC lines (without P53DD), which all lost the episomal vectors by passage 6 (FIG. 10v). They could be passed at least 11 times, maintaining ESC-like morphology (FIG. 10w) and correct number of chromosomes (FIG. 10x), and stained positive for SOX2 and OCT4 (FIG. 10y). biPSCs stained positive for SOX2 and OCT4 (FIG. 10y), and could differentiate into three germ layers in the teratoma assay (FIG. 10z-ab). Therefore, SOX2-17 allowed the first ever generation of integration-free virus-free bovine iPSCs with potential applications in the cultivated beef industry, livestock genome editing and beyond.

To investigate the effects of SOX2^{AV} and SOX2-17 on the faithfulness of human reprogramming, 30 hiPSC lines were generated and characterized using episomal reprogramming of new-born foreskin (young, Y) and 56-year-old male dermal (old, O) fibroblasts. All the selected clonal iPSC lines were tested integration-free (FIG. 10z) with normal karyotypes (FIG. S4aa-ab). Hierarchical clustering of global gene expression based on RNA-seq showed that all the iPSCs clustered far from the fibroblasts and close to the hESCs (FIG. 5p). The gene expression differences between the lines arose from the cell source more than the SOX factors used. Reduced representation bisulfite sequencing (RRBS) (Meissner, 2005) was performed to analyze the methylome of hiPSCs. All the lines clustered far from fibroblasts and close to hESCs (FIG. 5q).

Loss of imprinting (LOI) is a common potentially cancerous (Holm et al., 2005; Jelinic and Shaw, 2007) irreversible (Hiura et al., 2013) epigenetic aberration afflicting iPSC technology for both mouse and human (Bar et al., 2017; Carey et al., 2011; Keshet and Benvenisty, 2021; Takikawa et al., 2013). Multiple studies reported a correlation between LOI and poor developmental outcome of all-iPSC embryos in 4N complementation experiments (Buganim et al., 2014; Carey et al., 2011; Chen et al., 2015a; Stadtfeld et al., 2012; Takikawa et al., 2013; Velychko et al., 2019a). 31 differentially methylated regions (DMRs) were analyzed and it was found that all the lines and even the original fibroblasts had different levels of LOI, with SOX2^{AV} hiPSCs derived from young fibroblasts and SOX2-17 hiPSCs derived from old fibroblasts exhibiting on average the lowest levels of LOI among the iPSC lines (FIG. 5r). It is concluded that highly-cooperative Sox factors can allow high-efficiency high-quality reprogramming across species (FIG. 5s).

### EXAMPLE 5: SOX/OCT HETERODIMERIZATION IS AT THE CORE OF NAÏVE PLURIPOTENCY

The ESC derivative of mouse pre-implantation inner cell mass and their iPSC counterparts grown in LIF containing media are called “naïve” (Nichols and Smith, 2009). Mouse naïve lines exhibit the highest developmental potential of all cultured cells—they can contribute to chimeric animals, some, but not all naïve lines are even capable of generating all-PSC mice in 4N complementation assays. This, however, is not true for “primed” PSCs of other species including human, which are more similar to mouse epiblast stem cells (mEpiSCs) (Tesar et al., 2007). Interestingly, Oct4DE is active in naïve but not primed pluripotent cells (Choi et al., 2016; Gafni et al., 2013; Yeom et al., 1996), and the highly-cooperative Sox factors dramatically increase stability of Sox2/Oct4 heterodimer on Oct4DE element (FIG. 3f). Inspired by these results, it was hypothesized that Sox/Oct cooperativity could be at the core of naïve pluripotency. Previously published time-course ATAC-seq dataset of naïve-to-primed transition samples generated for mouse ESCs upon exposure to bFGF media (Yang et al., 2019) were analyzed. Spearman correlation of sequencing reads confirmed the reproducibility between replicates and showed that the most significant transition happens between day one (d1) and day two (d2) of differentiation into epiblast-like cells (EpiLCs) (FIG. 6a). Footprinting analysis using TOBIAS (Bentsen et al., 2020) showed the most dominant depleted footprints on d1 were those of estrogen-related receptor (Esrr) and Klf factors (FIG. 6b). This is not surprising, as members of both Kif (Klf4) and Esrr (Essrb) families are capable of converting mouse EpiSCs to naïve ESCs (Adachi et al., 2018; Guo et al., 2009). The d1 samples also had a significant depletion of Sox/Oct footprints (FIG. 6b), which became the most depleted of all TF footprints between d1 and d2 of transition (FIG. 6c). Whole-cell lysate EMSAs was performed on the SoxOct element of Nanog promoter to measure the levels of heterodimerization of naturally expressed Sox2 and Oct4 in E14 mESC line grown in KSR-LIF media, GFP-E3 mEpiSC line harboring Oct4-GFP reporter (Gof18) grown in bFGF-containing hESC media, primed-to-naïve converted sorted Oct4-GFP⁺ mEpiSCs grown in KSR-LIF or the same media containing 2i (Mek and GSK-3 inhibitors, PD0325901 and CHIR99021, respectively (Ying et al., 2008)), as well as human iPSCs grown in conventional bFGF media (FIG. 6d). The endogenous EMSA results echoed the footprinting analysis: mEpiSCs had much lower levels of Sox2 and Oct4 capable of heterodimerizing compared to mESCs but the heterodimerization in EpiSCs was restored to ESC level upon prime-to-naïve conversion (FIG. 6d). The heterodimerization (but also Oct4 alone binding) was further enhanced in the presence of 2i (FIG. 6d). The hiPSCs had similar to mEpiSCs low Sox2/Oct4 heterodimerization capacity but also higher level of Oct4 alone binding. Western blot analysis showed that the lack of Sox2/Oct4 heterodimerization was due to lower Sox2 expression in both mouse and human primed cells; Oct4 expression levels were equal in mEpiSCs and mESC-LIF samples but higher in 2iLIF samples and in hiPSCs (FIG. 6e). The identity of Sox2, Oct4, and Sox2/Oct4 bands was confirmed in all tested lines using antibody super shift assays (FIG. 6f). To find if Sox2/Oct4 heterodimerization could also explain the vast difference in developmental potential among iPSC lines, six integration-free mouse PSC lines were tested, which were characterized in details in a previous study (Velychko et al., 2019a). Two out of six iPSC lines (epi-OKSM #1 and epi-OKSM #3) were incapable of generating full-term all-iPSC pups (4N-off, “poor quality”), while the other four (epi-OKSM #2, epi-KSM #1, ESC #1 and ESC #2) could generate adult all-PSC mice (Velychko et al., 2019a). The cells were first adopted to feeder-free conditions with several passages in 2iLIF media, then passed either with or without 2i and harvested for endogenous EMSA experiments. Of the cells grown in LIF-alone media, two developmentally incapable iPSC lines had the lowest capacity for Sox2/Oct4 heterodimerization (FIG. 6g) and the lowest Sox2 expression (FIG. 6h) among the six lines, demonstrating a similar pattern to EpiSCs (FIG. 6d-e). However, the same lines passaged in 2iLIF showed no difference in either Sox2/Oct4 heterodimerization or Sox2 expression (FIG. 6g-h). This suggests that pluripotent cell lines could be stabilized at different levels of Sox2 expression and Sox2/Oct4 heterodimerization: from Sox-high “very naïve” to different shades of “moderately naïve” and “primed” cultures with progressively lower developmental potency (FIG. 6a-h). We and others previously described capturing different grades of pluripotency (Bernemann et al., 2011; Kinoshita et al., 2020), but the role differential Sox2 expression and Sox2/Oct4 heterodimerization has not received much attention. The widely used 2i media seems to equalize the heterodimer content between different grades of naïve cells but fails to stably reprogram the cell fate: the mediocre Sox-low lines revert to being mediocre after 2i withdrawal (FIG. 6g-h). So, how can the low-grade pluripotent cells such as mouse EpiSCs or conventional pluripotent cultures be stably “upgraded” in most other species including human?

Mouse EpiSCs could be converted to naïve ESCs by overexpression of Klf4 (Guo et al., 2009). A similar conversion was attempted for human cells but it was found that Klf4 alone is not sufficient (FIG. 6i). A screen of different combinations of Yamanaka factors showed that overexpression of Sox2 and Klf4 (SK) is sufficient to convert conventional hiPSCs into dome-shaped colonies positive for human naïve pluripotency marker KLF17 (Guo et al., 2016; Kilens et al., 2018; Lea et al., 2021; Shahbazi et al., 2017) even in the absence of small molecule inhibitors (FIG. 6i). Similar to SKM reprogramming in mouse (An et al., 2019; Velychko et al., 2019a), combining Sox2 and Klf4 in one bicistronic vector increased the number of KLF17⁺ colonies (FIG. 6i). Supplementing KSR-LIF media with Mek inhibitor additionally enhanced the efficiency of the conversion (FIG. 6i). The SK cocktail was used for further experiments employing Gof18 mEpiSCs as a model. The goal was to understand integration-free pluripotency upgrade and to understand the role of Sox/Oct cooperativity in SK-based primed-to-naïve conversion. mCherry was cloned into the human episomal reprogramming vectors to generate pCXLE-mCherry-T2A-SOX-P2A-KLF4 carrying SOX2, SOX2^{AV}, or SOX2-17. The episomal Cherry-SK vectors were delivered into mEpiSCs using lipofection, the cells were sorted for mCherry⁺/Oct4-GFP⁻ on day 2 (FIG. 11a) and plated in KSR-LIF media on inactivated feeders. The majority of surviving cells formed dome-shaped colonies that were Oct4-GFP⁺ and mCherry⁻ already on day 4 after plating (day 6 after transfection); six individual Oct4-GFP⁺/mCherry⁻ colonies were picked for each of the three cocktails and clonally expanded for further characterization (FIG. 11b). Regardless of the Sox version, the SK-converted lines exhibited big differences in Sox2/Oct4 heterodimerization determined by endogenous EMSAs (FIG. 6j), which correlated with differences in Sox2 expression determined by western blotting (FIG. 11c), likely representing different grades of pluripotency. A transient overexpression of wild-type SK reset the Sox2/Oct4 heterodimerization by maximum of 62% compared to the Oct4-GFP⁻ mEpiSC control grown in the same conditions (FIG. 6j). Both highly-cooperative Sox factors appeared to be much more potent in resetting the Sox2/Oct4 heterodimerization: 3 out of 6 S^{AV}K lines had 120-140% increase in heterodimerization, and two out of six S*K lines had a striking 180% boost (FIG. 6j). S*K-converted naïve lines also had significantly lower propensity to spontaneously lose Oct4-GFP⁺ status during passaging (FIG. 11 b, d), suggesting that super-SOX delivered a more stable reset.

The episomal mCherry-S*K or mCherry alone plasmids (FIG. 11e) were nucleofected into hiPSCs grown in primed media (StemFlex), and, optionally, changed the media to human naïve media (RSeT) on day 2 (FIG. 11e). The cells were stained for human naïve pluripotency markers KLF17 and SUSD2 on day 7. Under naïve media conditions, most of the colonies nucleofected with episomal mCherry-S*K generated dome-shaped colonies that were SUSD2⁺, KLF17⁺, and mCherry−, while none of the control-transfected cells were positive for these markers (FIG. 11e-f). Similar to the mEpiSC reset, mCherry-S*K plasmid was eliminated from the cells within a few days (FIG. 11e-g), while the mCherry alone plasmid persisted (FIG. 11f). Surprisingly, transient S*K expression could give rise to partially SUSD2⁺ and KLF17⁺ hiPSC colonies even in primed media without feeders (FIG. 11h).These data suggest that decreasing Sox2/Oct4 driven chromatin opening could be responsible for diminished developmental potential upon priming pluripotent cells in early development (FIG. 11e), SK reprogramming can reverse the process and highly-cooperative Sox factors markedly promote this reset (FIG. 11 i).

### EXAMPLE 6: SOX2-17+KLF4 COCKTAIL ENHANCES THE DEVELOPMENTAL POTENTIAL OF IPSCS AND ESCS ACROSS SPECIES

The episomal S*K reset protocol for human iPSCs (female episomal hiPSC line A18945, Gibco, FIGS. 11e and 12a) was further optimized. As described above in Example 5 (FIG. 11e) hiPSCs were co-nucleofected with pCXLE-mCherry-S*K and pCXWB-EBNA1 (to increase the longevity of the episomal expression) and plated on a dense feeder layer in primed media (StemFlex, Thermofisher). On the second day, the media was changed to human naïve media (Rset, STEMCELL Technologies) and the cells were transferred to a hypoxic incubator (5% O₂). By day 7, within just one passage, S*K-treated cells generated dome-shaped colonies that tested positive for human naïve pluripotency markers SUSD2 (FIG. 11e, 12a) and KLF17 (FIG. 11f) (Bredenkamp et al., 2019a; Wojdyla, et al., 2020; Bi, et al., 2022.). On average, 18% of day 7 S*K hiPSCs were SUSD2⁺ and mCherry⁻ confirming the unprecedented efficiency of the reset and the transgene-independent status of the generated naïve cells. While most of the S*K-reset colonies were KLF17⁺, none of the mCherry control-transfected cells were positive for either SUSD2 or KLF17 (FIG. 11f, 12a). Unexpectedly, transient overexpression of S*K gave rise to SUSD2⁺ and KLF17⁺ hiPSC colonies even in conventional feeder-free culture conditions in primed media (FIG. 11g). Naïve reset in primed media has never been demonstrated for other TF cocktails (Takashima et al., 2014; Liu et al., 2017; Theunissen et al., 2014; Hanna et al., 2010; Yamauchi et al., 2020, Qin et al., 2016).

RT-qPCR was performed to assess expression of key naïve pluripotency genes. S*K reset led to a significant upregulation of DNMT3L, KLF17, and ARGFX in both primed and naïve media, with stronger upregulation in naïve media. On the other hand, the Rset naïve media alone did not increase naïve gene expression with the exception of 6-fold upregulation of KLF4 (FIG. 12b). Both FACS and qPCR data for WPRE confirmed that mCherry-S*K plasmid was eliminated from the cells by day 7, while the mCherry alone plasmid persisted (FIG. 12a, b), suggesting that S*K-reset might trigger transgene silencing mechanisms, which were described for mouse naïve cells (Yang et al., 2015). The episomal S*K naïve reset results were reproduced for two other human lines: H9 ESCs and OS*KML O-hiPSC #1 (generated for this study).

To test the developmental potential of our putative naïve hiPSCs we used sorted SUSD2⁺ S*K-reset cells at day 7 for aggregations with mouse embryos at morula stage (E2.5) as described before (Eakin et al., 2006). hiPSC line was marked with constitutive RFP expression to assess the level of cross-species chimera contribution. Astonishingly, human cells were detected in ICMs of the majority of aggregated embryos (FIG. 12c). The chimerism was confirmed with co-staining of the aggregated embryos at E4.5 with human-specific SUSD2 and mouse-specific Oct4 antibodies. Human SUSD2+ cells were integrated in ICMs of 6 out of 11 tested aggregated embryos; intriguingly, in one case, the immunostaining indicated a dramatic takeover of the entire epiblast region by S*K-reset hiPSCs (FIG. 12d). The results for mouse/human chimera experiments were reproduced with another hiPSC line (OS*KML O-hiPSC #1, data not shown).

While super-SOX allowed generation of episomal bovine iPSCs (biPSCs), they failed to generate teratomas in in severe combined immunodeficient (SCID) mice in a few injection attempts. Similarly, cultured bovine ESCs do not readily give rise to teratomas (communication with Jun Wu). As mentioned above in Example 4, to test if S*K-reset could enhance the developmental potential of biPSCs, we injected control biPSCs, or S*K-reset biPSCs into the left and right sides of the same mouse, respectively. 5 weeks later, a teratoma arose only on the right side and it contained tissues representing all three embryonic germ layers (FIG. 10 aa).

Finally, it was explored whether S*K reset could improve the developmental potential of “poor quality” naïve female mouse ESC line (mESCs, C57BL/6J background) grown in 2iL media, which was incapable of efficient generation of all-ESC mice in our previous tetraploid complementation experiments (FIG. 12f). Nucleofection of mESCs with either episomal mCherry-S*K or mCherry vectors gave rise to dome-shaped naïve-like colonies (FIG. 12g). The manually picked colonies at day 5 post nucleofection were used for tetraploid complementation experiments (FIG. 12f). Amazingly, the S*K-reset cells gave rise to 8 times more full-term all-ESC pups compared to control (FIG. 12 h-j). Three S*K-reset all-ESC pups survived foster-nursing, while the only control all-ESC pup died soon after birth (FIG. 12i). The in vivo evidence for enhanced development potential for three unrelated species presented in this section, most importantly the birth of S*K-reset all-ESC animals, provide convincing proof for our proposed “Heterodimer model” of pluripotency (FIG. 11h).

### DISCUSSION

iPSC technology remains inefficient particularly for non-murine cells and reprogramming often leads to epigenetic aberrations resulting in inconsistent iPSC quality (Carey et al., 2011; Hiura et al., 2013; Keshet and Benvenisty, 2021; Takikawa et al., 2013). A few alternative cocktails were shown to improve the fidelity of reprogramming process in mouse (Buganim et al., 2014; Chen et al., 2015a; Velychko et al., 2019a) but failed to reprogram human cells, which appear to possess a stronger epigenetic barrier (Kim et al., 2021). Perhaps the apparent barrier is merely testimony to the inadequacy of the wild-type factor-based reprogramming machinery we are currently employing.

Many studies addressed Oct4's uniqueness among POU factors by swapping domains between Oct4 and other POU factors as well as introducing mutations (Jerabek et al., 2017; Kim et al., 2020; Van Leeuwen et al., 1997; Nishimoto et al., 2003; Roberts et al., 2021; Tapia et al., 2012; Velychko et al., 2019b). The instant inventors and others determined the importance and uniqueness of Oct4-POU_{S}, POU_{HD}, linker, and CTD domains. Here, it is reported that the residue swap at the Sox/Oct interface of Sox2, A61V, increases the stability of the Sox/Oct complex on canonical SoxOct motifs that control pluripotency genes. 61V, especially when combined with the stronger Sox17 transactivator, can rescue the otherwise disabling deletion of the Oct4 linker, POU_{HD}, NTD, or CTD domains. In the context of the highly-cooperative Sox factor, Oct4-POU_{S} domain that contains the Sox/Oct interface (FIG. 2) proved to be the most crucial for reprogramming (FIG. 3a-c). The 17 residue-long linker peptide connects the two DNA-binding subdomains of Oct4-POU domain; all the resolved POU structures so far suggest that the linker is not directly involved in the DNA-binding (FIG. 2a-b), yet it appears to be important for both reprogramming to pluripotency and for development (Chen et al., 2020b; Esch et al., 2013; Han et al., 2022a). Until now, the effects of the linker mutations on Sox/Oct stability have not been tested. It was found here that Oct4 linker mutants such as L80A or a complete replacement with flexible poly-glycine peptides, reduced stability of the Sox/Oct heterodimer and that the highly cooperative Sox2^{AV} partially rescued both the stability of the heterodimer and their reprogramming ability (FIG. 1f, 8k, 3a), similarly Sox2^{AV} enables Sox/POU heterodimerization and reprogramming with tissue-specific POU factors (FIG. 1g-h, FIG. 8b,k). The MDS revealed a previously undescribed SL configuration of the Sox2/Oct4 heterodimer on a canonical SoxOct motif, where Oct4 residues 78E and 82E form salt bonds with Sox2's 57K and 50R, respectively (FIG. 2c, e). Sox2^{K57E} mutant was reported to be detrimental for iPSC generation and disrupts Sox2/Oct4 heterodimerization on canonical SoxOct motifs but not on Fgf4 (Jauch et al., 2011; Reményi et al., 2003a). The experiments showed that Sox2^{AV} and Sox2-17 enhanced cooperativity with Oct4 on the Nanog promoter, Pou5f1 distal enhancer (Oct4DE), HoxB1 and Utf1 enhancer loci, all containing canonical SoxOct motifs (FIG. 3, FIG. 8); Sox2^{AV} enhanced heterodimerization on a nucleosomal SoxOct element (FIG. 4a) and in early reprogramming samples in situ (FIG. 4b-f). However, consistent with the model prediction (FIG. 2f), neither Sox2^{AV} nor Sox2-17 changed cooperativity on the Fgf4 enhancer motif (FIG. 3f, FIG. 8j, FIG. 4e), which has three additional base pairs spaced between Sox and Oct elements (Reményi et al., 2003b). Fgf4 regulates cell proliferation and differentiation in the epiblast (Kunath et al., 2007; Niswander and Martin, 1992), and inhibiting the Fgf4 pathway with PD0325901 facilitates maintenance of naïve pluripotent cells (Kunath et al., 2007). Therefore, the specific enhancement of S and SL, but not DS configuration of Sox2/Oct4 heterodimer by A61V could be the key for its much-improved performance in reprogramming. Additional enhancement of the SL configuration and/or targeted disruption of DS binding on Fgf4 could further improve the super-SOX-based developmental reset technology.

The animal evolutionary tree suggests that of the Sox2/Oct4 couple, in the beginning there was Sox2. Both 50R and 57K are already present in sponges, where SoxB factors control early embryogenesis (Fortunato et al., 2012); V at position 61 could also be found in Sox factors of these most primitive animals. 57K and 61A of Sox2 are conserved in hydrozoans, where SoxB genes are expressed in stem cells that give rise to neuroectoderm (default) and the germline (Bosch and David, 1987; Jager et al., 2011; Siebert et al., 2019). POU5 emerged much later in the evolutionary tree—it is an innovation of vertebrates, where it cooperates with Sox2 to control early development (Leichsenring et al., 2013; White et al., 2016a). Interestingly, while the linker is the least conserved part of POU factors, the negative charges at positions 78 and 82 of POU5 linker are already present in jawless hagfish (Sukparangsi et al., 2022). The computer simulations and functional data on the A61V mutant proves that the most significant feature that distinguish Oct4 from other POU factors is its ability to form a stable heterodimer with Sox2, which had already been in control of early embryogenesis in lower animals. Sox17, a master regulator of the germline, also cooperates with Oct4 but prefers compressed rather than canonical SoxOct motifs (Irie et al., 2015; Jauch et al., 2011; Merino et al., 2014). The germline and pluripotent fates share many features, highlighted by the possibility to derive mouse pluripotent cells from germline stem cells (Kanatsu-Shinohara et al., 2004, 2008; Ko et al., 2009). The structural elements of Sox17 that facilitate induction of pluripotency evolved independently from Sox2, plausibly to specify our most precious lineage.

The instant inventors and others have reported engineering enhanced reprogramming factors that increased the efficiency of mouse reprogramming (Aksoy et al., 2013; Hirai et al., 2012; Tan et al., 2021a; Veerapandian et al., 2018) but none of the studies showed a substantial gain of efficiency or faithfulness of iPSC generation beyond the mouse model. All alternative cocktails that improved the quality of mouse iPSCs decreased the efficiency of reprogramming, making them impractical even if they did work for human (Buganim et al., 2014; Chen et al., 2015a; Velychko et al., 2019a). The instant inventors combined the structural elements of Sox2 and Sox17 to build a Sox2-17 chimeric TF that enhances reprogramming in five tested species: mouse, human, cynomolgus macaque, cow, and pig (FIG. 5, FIG. 10). Remarkably, just 24 h induction with our modified Yamanaka cocktail could generate mouse iPSCs that support full-term development, demonstrating the speediest TF-driven cell fate reset reported to date (FIG. 5a-b, FIG. 10a-e). It was shown that Sox2-17 enhances integration-free three-factor reprogramming efficiency in mouse by 150 times without worsening the developmental potential of the resulting iPSCs (FIG. 5d-f, Table 1). A point mutation in Sox2, A61V, significantly increased the developmental potential of mouse OSKM iPSCs, marked by both a higher rate of full-term development of all-iPSC embryos and the survival of the all-iPSC mice to maturity (FIG. 4g-i, Table 1). OSKM and OSK cocktails not only can induce a complete cell fate reset in the dish but also reverse aging of various animal tissues (Browder et al., 2022; Chen et al., 2021; Chondronasiou et al., 2022; Lu et al., 2020; Ocampo et al., 2016; Sarkar et al., 2020). However, a very much expected lifespan extension for wild-type mice has not yet been demonstrated (Ocampo et al., 2016). It is reasonable to assume, that cocktails that induce iPSCs of higher quality (FIG. 4g-i, 5f) and can support normal development of tissues to adulthood even with likely background leaking of tet-inducible promoter (Meyer-Ficca et al., 2004), could also outperform wild-type factors in reversing animal aging.

Notably, all the reprogramming cocktails that improved the developmental potential of mouse iPSCs, including OSKM versus OKSM (Carey et al., 2011), OSK (Buganim et al., 2014), and SKM versus OSKM (Velychko et al., 2019a), as well as the highly-cooperative Sox2 introduced in this study, also reduce the rate of cell division (FIG. 1i). Some cell-culture interventions improving the quality of iPSCs also inhibit cell division (Liu et al., 2014). It is therefore tempting to theorize that the secret of a perfect reset lies in limiting cell proliferation (FIG. 6k). This can be achieved by 1) enhancing Oct/Sox cooperativity, as in OS^{AV}KM reprogramming; 2) increasing the Sox2 to Oct4 ratio, as in SKM reprogramming, but also in Oct4 heterozygous knockout ESCs (Karwacki-Neisius et al., 2013); 3) depleting or omitting Myc (or other proliferation-inducing factors), as in OKS (FIG. 5f, FIG. 10k) (Buganim et al., 2014), but also in OSKM versus OKSM cassettes (Carey et al., 2011) and in Myc depleted ESCs (Scognamiglio et al., 2016); 4) reducing the expression levels of reprogramming factors, as in episomal versus viral vectors (FIG. 5f) (Velychko et al., 2019a). Following the episomal reprogramming protocols, the construct carrying shRNA against TP53 (Okita et al., 2011) was used to generate human iPSC lines for this study, which knocks down the major tumor suppressor and boosts cell proliferation. This likely caused loss of genetic imprints in the human iPSC lines (FIG. 5r). Considering the mouse developmental potential results presented here (FIG. 5f), knocking down our genome guardian, is likely detrimental to the quality of iPSCs and should be avoided.

Mouse naïve pluripotent cells are the most developmentally potent cultures currently available. Mice likely evolved (or preserved) the unusual stability of their naïve pluripotency fate to enable a blastocyst-stage embryonic arrest, known as diapause (Boroviak et al., 2015). Extraordinary developmental potential of mouse naïve pluripotent cells, as well as their increased capacity for homologous recombination repair, has allowed for unprecedented genetic engineering of this species (Thomas and Capecchi, 1987). Mouse remains the only species for which generation of all-iPSCs animals has been reported (Boland et al., 2009; Kang et al., 2009; Zhao et al., 2009); and even germline competence has been reported only for mouse and rat (Hamanaka et al., 2011), highlighting the limitations of today's technologies. Naïve pluripotent cells have decreased level of Myc compared to primed (Bernemann et al., 2011; Ying et al., 2008), which could be a direct effect of high levels of Sox2 (Metz et al., 2022) in naïve cells (FIG. 6e). Here, we demonstrated that Sox2/Oct4 heterodimerization is diminished during naïve-to-primed differentiation (FIG. 6b-c) and is restored during primed-to-naïve conversion (FIG. 6d). For the first time, a common mechanism for diminished developmental potential in primed versus naïve and 4N-off versus 4N-on pluripotent cells (FIG. 6d-h) was found. The Sox/Oct model of developmental reset presented here (FIG. 6k) is in line with other studies placing Sox2 at the top of pluripotency network hierarchy (Buganim et al., 2012; Chronis et al., 2017; Liu et al., 2015; Luo et al., 2021; Malik et al., 2019; Tremble et al., 2021; White et al., 2016b), however, the key role of Sox2 and Sox2/Oct4 heterodimerization in highly developmentally potent (naïve) pluripotency has never been elucidated before. We found that even transient episomal expression of S*K is sufficient to generate naïve-like hiPSCs in both naïve and in primed human media (FIG. 11e-h, FIG. 12). Episomal S*K reset also allowed us to generate teratoma-capable biPSCs (FIG. 10z-ab), providing the first in vivo evidence of enhanced developmental potential for non-murine cells. Finally, the S*K reset could increase the developmental potential of already naïve mouse ESCs, boosting their ability to generate all-ESC mice (FIG. 10z-ab, 12d). Such unprecedented results across species have never been achieved with any other naïve reset technology. The in vivo evidence for naïve reset in three species is the ultimate proof for the “Heterodimer model” of naïve-to-primed pluripotency continuum. The model explains the workings of each Yamanaka factor in pluripotency: high levels of Sox2 and Klf4 expression, as well as Sox2/Oct4 dimerization promote the pre-implantation naïve state featuring the highest developmental potential, while decreased Sox2 level leads to reduced Sox2/Oct4 dimerization, with Oct4 alone and Myc binding promoting cell proliferation and priming.

Long-term culture in naïve media leads to epigenetic abnormalities and loss of germline competency for both human and mouse (Keshet and Benvenisty, 2021; Alves-Lopez et al., 2023; Choi et al., 2017). Our work offers an alternative: We hereby propose a protocol for induction of high-grade pluripotency across species: 1) generation of primed iPSCs using OS*KM; 2) naïve reset of the primed cells using S*K (FIG. 11i). Moreover, the existing primed cultures of different species could be efficiently reset using a brief exposure of episomes or mRNA encoding for S*K (FIG. 12). We postulate that engineered factors designed to induce higher-quality iPSCs with improved efficiency, may surpass the performance of wild-type factors in terms of age reversal. The A61V mutant discovered here could allow us to elucidate the role of Sox/Oct dimerization in rejuvenation via partial reprograming, enhancing our understanding of the process. It will certainly be interesting to explore how super-SOX-containing cocktails affect animal tissues in vivo. While a lot of research recently is focused on reversing aging, a similarly intriguing direction is delaying the whole animal development. Indeed, all intelligent mammals and birds are born premature and experience long childhoods. Compared to other primates, our species has the most protracted development, which has undoubtedly played a role in the evolution of our intelligence and self-domestication, allowing us to build complex societies (Smith, 1995; Ramirez and de Castro, 2004). If we could further postpone the development, particularly during childhood, we might see the extension of the health span (Müller et al., 2002; Yuan et al., 2012), an increased window of deep learning opportunities (Liquin et al., 2022), leading to further improvement of our cognitive and social development that could benefit civilization as a whole. Delaying the development of young animals could be both more beneficial and a more feasible approach compared to age reversal. However, the practicality and ethical implications of these potential applications require further exploration and discussion.

In early animal development, unidirectionality is likely achieved by a negative feedback loop limiting the return to high-Sox2 state (Ormsbee Golden et al., 2013). Reprogramming with SK cocktail can reset the high level of Sox2 expression (FIG. 11c), which restores Sox2/Oct4 heterodimerization (FIG. 6j) and activates naïve pluripotency markers (FIG. 6i). The specific enhancement of S and SL, but not DS configuration by A61V (FIG. 2, 3f) could be the key for the enhanced developmental potential of OSAVKM iPSCs (FIG. 4g-i, Table 1). Additional targeted disruption of Oct4 monomer binding, as well as Sox/Oct binding in DS configuration on the Fgf4-like motifs could allow further improvement of the reprogramming technology. Interestingly, it is well-known that mouse female pluripotent stem cells have lower developmental potential compared to their male counterparts (Arez et al., 2020; Di et al., 2015). The model described herein suggests that the reason for heighten developmental potential in male versus female pluripotent lines could be expression of Sex determining region of Y (Sry) in XY lines (Koopman et al., 1991). Sry has a binding motif similar to Sox2 and similar to other Sox TFs, Sry footprints were depleted in primed versus naïve cells (FIG. 6b-c). The high-SOX hypothesis could also explain the increased survival of male versus female embryos in mammals (Carvalho et al., 1996; Orzack et al., 2015) and higher occurrence of glioblastoma and other types of Sox-driven cancers in men (Carrano et al., 2021). The abundance of Sox footprints in the open chromatin of naïve versus primed cells suggest a more general developmental trend, where various ratios of Sox factors and their partners predispose stem cells towards specific lineages. Therefore, it is plausible that Sox17+Klf4 cocktail may redistribute the Oct4 binding sites to compressed SoxOct motifs inducing the formation of primitive endoderm (Merino et al., 2014; Aksoy et al., 2013), similarly to how Sox2+Klf4 cocktail induces the pre-implantation epiblast fate (FIG. 6i). The prospect that the mechanisms uncovered here could allow to derive developmentally-capable pluripotent cells across species is intriguing, yet, the ramifications of this new understanding of developmental reset will reach far beyond the pluripotency field.

A small number of cells of the inner cell mass can make a disproportionally high contribution to animal development (Bolton et al., 2016). Therefore, it is likely that the developmental potential of a given pluripotent line is determined by a Sox-high subpopulation and not the average Sox2 expression determined in our bulk endogenous EMSA and western blot experiments. More work is needed to address the heterogeneity within pluripotent lines, the mechanisms regulating Sox2 expression and functionality, and the possibilities to enrich and stabilize the Sox-high pluripotent cell cultures with presumably upgraded developmental potential. Highly cooperative or excessive Sox factors, besides enhancing Sox/Oct heterodimerization, may also participate in the developmental reset in other ways, e.g. cellular remodeling by recruiting the antagonist of aging Parp1 (Liu and Kraus, 2017) or silencing the retroviral elements (Velychko et al., 2019a). The naïve reset protocol requires further optimization to increase the longevity of S*K expression. For instance, using modRNA for delivery might help circumvent the imminent silencing of episomes in naïve cells (see FIG. 12b). A culture medium that could support the long-term maintenance of mouse ESC-like naïve cells exhibiting high levels of Sox2/Oct4 dimerization remains to be formulated (Keshet and Benvenisty, 2021; Alves-Lopez et al., 2023). The S*K-reset naïve PSCs of different species require further molecular and functional characterization. Key molecular features like DNA methylation and X chromosome status of the S*K-reset cells remain unexplored. Most importantly, we need to conduct embryology experiments in livestock and primate species to address the possibility of germline transmission and high-grade chimerism beyond mouse and rat. So far, no naïve reset technology has enabled reproducible human/mouse chimerism beyond implantation (Gafni et al., 2013).

The herein engineered super-SOX harvests the beneficial naturally evolved structural elements of two major regulators of development, Sox2 and Sox17. It is likely that even more efficient reprogramming factors could be built by means of intelligent design and directed evolution (Tan et al., 2021b; Veerapandian et al., 2018). These data suggest that enhancing cooperativity between key co-factors should be one of the goals of future designers.

TABLE 1
Tetraploid complementation results

| | | | | | | Survived |
|---|---|---|---|---|---|---|
| | | Aggregates | Full-Term | | Survived | after 3 |
| PSC line | Sex | transferred | pups | Breathing | after 48 h | months |
| tetO-OSKM #1 | Male | 30 | 0 | 0 | 0 | 0 |
| tetO-OSKM #2 | Male | 32 | 12 | 9 | 1 | 0 |
| tetO-OSKM #3 | Male | 44 | 7 | 4 | 0 | 0 |
| tetO-OSKM #4 | Male | 44 | 0 | 0 | 0 | 0 |
| tetO-OS^{AV}KM #1 | Male | 24 | 16 | 13 | 8 | 8 |
| tetO-OS^{AV}KM #2 | Male | 32 | 11 | 9 | 1 | 1 |
| tetO-OS^{AV}KM #3 | Male | 34 | 19 | 9 | 5 | 3 |
| tetO-OS^{AV}KM #4 | Male | 32 | 1 | 0 | 0 | 0 |
| tetO-OS^{AV}KM #5 | Male | 32 | 17 | 6 | 4 | 4 |
| tetO-OS^{AV}KM #6 | Male | 44 | 4 | 2 | 0 | 0 |
| episomal OSKM #1 | Male | 33 | 15 | 11 | 6 | 5 |
| episomal OSKM #2 | Male | 30 | 8 | 4 | 0 | 0 |
| episomal OSKM #3 | Male | 42 | 0 | 0 | 0 | 0 |
| episomal OSKM #4 | Male | 38 | 11 | 6 | 2 | 1 |
| episomal OS^{AV}KM #1 | Male | 55 | 10 | 2 | 0 | 0 |
| episomal OS^{AV}KM #2 | Male | 39 | 22 | 18 | 13 | 11 |
| episomal OS^{AV}KM #3 | Male | 29 | 17 | 17 | 10 | 10 |
| episomal OS^{AV}KM #4 | Male | 30 | 21 | 18 | 15 | 13 |
| 24 h tetO-OS*KM #1 | Female | 12 | 5 | 3 | 2 | 0 |
| 24 h tetO-OS*KM #2 | Male | 24 | 0 | 0 | 0 | 0 |
| tetO-OS*KM #1 | Male | 24 | 12 | 9 | 8 | 0 |
| tetO-OS*KM #2 | Male | 18 | 3 | 3 | 0 | 0 |
| episomal OKS #1 | Male | 29 | 19 | 18 | 9 | 6 |
| episomal OKS #2 | Male | 28 | 12 | 12 | 12 | 11 |
| episomal OKS #3 | Male | 20 | 13 | 10 | 2 | 2 |
| episomal OKS #4 | Male | 20 | 13 | 11 | 8 | 7 |
| episomal OKS* #1 | Male | 30 | 23 | 17 | 7 | 5 |
| episomal OKS* #2 | Male | 20 | 11 | 6 | 3 | 3 |
| episomal OKS* #3 | Male | 30 | 12 | 10 | 6 | 5 |
| episomal OKS* #4 | Male | 18 | 10 | 10 | 8 | 6 |
| episomal OKS* #5 | Male | 19 | 8 | 8 | 6 | 6 |
| D5 Ctrl ESCs | Female | 26 | 1 | 1 | 0 | 0 |
| D5 S*K-reset ESCs | Female | 42 | 13 | 7 | 3 | 0 |

### REFERENCES

Adachi, K., Kopp, W., Wu, G., Heising, S., Greber, B., Stehling, M., Araúzo-Bravo, M. J., Boerno, S. T., Timmermann, B., Vingron, M., et al. (2018). Esrrb Unlocks Silenced Enhancers for Reprogramming to Naive Pluripotency. Cell Stem Cell 23, 266-275.e6.
Aksoy, I., Jauch, R., Eras, V., Chng, W. B. A., Chen, J., Divakar, U., Ng, C. K. L., Kolatkar, P. R., and Stanton, L. W. (2013). Sox transcription factors require selective interactions with oct4 and specific transactivation functions to mediate reprogramming. Stem Cells 31, 2632-2646.
Alves-Lopes, J. P., Wong, F. C. K., Tang, W. W. C., Gruhn, W. H., Ramakrishna, N. B., Jowett, G. M., Jahnukainen, K., and Surani, M. A. (2023). Specification of human germ cell fate with enhanced progression capability supported by hindgut organoids. Cell Rep. 42, 111907. 10.1016/j.celrep.2022.111907.
Amadei, G., Handford, C. E., Qiu, C., De Jonghe, J., Greenfeld, H., Tran, M., Martin, B. K., Chen, D.-Y., Aguilera-Castrejon, A., Hanna, J. H., et al. (2022). Synthetic embryos complete gastrulation to neurulation and organogenesis. Nature 0-1.
Ambrosetti, D. C., Basilico, C., and Dailey, L. (1997). Synergistic activation of the fibroblast growth factor 4 enhancer by Sox2 and Oct-3 depends on protein-protein interactions facilitated by a specific spatial arrangement of factor binding sites. Mol. Cell. Biol. 17, 6321-6329.
Amemiya, H. M., Kundaje, A., and Boyle, A. P. (2019). The ENCODE Blacklist: Identification of Problematic Regions of the Genome. Sci. Reports 2019 91 9, 1-5.
An, Z., Liu, P., Zheng, J., Si, C., Li, T., Chen, Y., Ma, T., Zhang, M. Q., Zhou, Q., and Ding, S. (2019). Sox2 and Klf4 as the Functional Core in Pluripotency Induction without Exogenous Oct4. Cell Rep. 29, 1986-2000.e8.
Arez, M., Eckersley-Maslin, M., Klobuc̆ar, T., Gilsa Lopes, J. von, Krueger, F., Raposo, A. C., Gendrel, A. V., de Jesus, B. B., and da Rocha, S. T. (2020). Sex of donor cell and reprogramming conditions predict the extent and nature of imprinting defects in mouse iPSCs. BioRxiv.
Barakat T S et al. Stable X chromosome reactivation in female human induced pluripotent stem cells. Stem Cell Reports. 2015 Feb. 10; 4(2):199-208. doi: 10.1016/j.stemcr.2014.12.012. Epub 2015 Jan. 29. PMID: 25640760; PMCID: PMC4325229
Bar, S., Schachter, M., Eldar-Geva, T., and Benvenisty, N. (2017). Large-Scale Analysis of Loss of Imprinting in Human Pluripotent Stem Cells. Cell Rep. 19, 957-968.
Barrier M, Jeffay S, Nichols H, Hunter S (2010) Evaluation of a mouse embryonic stem cell adherent cell differentiation and cytotoxicity (ACDC) assay. The Toxicologist 114: 358-9.
Bentsen, M., Goymann, P., Schultheis, H., Klee, K., Petrova, A., Wiegandt, R., Fust, A., Preussner, J., Kuenne, C., Braun, T., et al. (2020). ATAC-seq footprinting unravels kinetics of transcription factor binding during zygotic genome activation. Nat. Commun. 11.
Bernemann, C., Greber, B., Ko, K., Sterneckert, J., Han, D. W., Araúzo-Bravo, M. J., and Schöler, H. R. (2011). Distinct developmental ground states of epiblast stem cell lines determine different pluripotency features. Stem Cells 29, 1496-1503.
Bi, Y., Tu, Z., Zhou, J., Zhu, X., Wang, H., Gao, S., and Wang, Y. (2022). Cell fate roadmap of human primed-to-naïve transition reveals preimplantation cell lineage signatures. Nat. Commun. 13, 1-15. 10.1038/s41467-022-30924-1.
Boroviak T, Nichols J. Primate embryogenesis predicts the hallmarks of human naïve pluripotency. Development. 2017 Jan. 15; 144(2):175-186. doi: 10.1242/dev.145177. PMID: 28096211; PMCID: PMC5430762.
Bredenkamp, N., Stirparo, G. G., Nichols, J., Smith, A., and Guo, G. (2019a). The Cell-Surface Marker Sushi Containing Domain 2 Facilitates Establishment of Human Naive Pluripotent Stem Cells. Stem Cell Reports 12, 1212-1222. 10.1016/j.stemcr.2019.03.014.
Bredenkamp N, Yang J, Clarke J, Stirparo G G, von Meyenn F, Dietmann S, Baker D, Drummond R, Ren Y, Li D, Wu C, Rostovskaya M, Eminli-Meissner S, Smith A, Guo G. (2019b) Wnt Inhibition Facilitates RNA-Mediated Reprogramming of Human Somatic Cells to Naive Pluripotency. Stem Cell Reports. 13(6):1083-1098. doi: 10.1016/j.stemcr.2019.10.009. Epub 2019 Nov. 7. PMID: 31708477.
Brison D R, Houghton F D, Falconer D, Roberts S A, Hawkhead J, Humpherson P G, Lieberman B A, Leese HJ. Identification of viable embryos in IVF by non-invasive measurement of amino acid turnover. Hum Reprod. 2004 October; 19(10):2319-24. doi: 10.1093/humrep/deh409. Epub 2004 Aug. 6. PMID: 15298971.
Browder, K. C., Reddy, P., Yamamoto, M., Haghani, A., Guillen, I. G., Sahu, S., Wang, C., Luque, Y., Prieto, J., Shi, L., et al. (2022). In vivo partial reprogramming alters age-associated molecular changes during physiological aging in mice. Nat. Aging 2, 243-253.
Buganim, Y., Markoulaki, S., van Wietmarschen, N., Hoke, H., Wu, T., Ganz, K., Akhtar-Zaidi, B., He, Y., Abraham, B. J., Porubsky, D., et al. (2014). The Developmental Potential of iPSCs Is Greatly Influenced by Reprogramming Factor Selection. Cell Stem Cell 15, 295-309.
Carey, B. W., Markoulaki, S., Hanna, J. H., Faddah, D. a., Buganim, Y., Kim, J., Ganz, K., Steine, E. J., Cassady, J. P., Creyghton, M. P., et al. (2011). Reprogramming factor stoichiometry influences the epigenetic state and biological properties of induced pluripotent stem cells. Cell Stem Cell 9, 588-598.
Castro-Mondragon, J. A., Riudavets-Puig, R., Rauluseviciute, I., Berhanu Lemma, R., Turchi, L., Blanc-Mathieu, R., Lucas, J., Boddie, P., Khan, A., Perez, N. M., et al. (2022). JASPAR 2022: The 9th release of the open-access database of transcription factor binding profiles. Nucleic Acids Res. 50, D165-D173.
Chen, J., Zhang, Z., Li, L., Chen, B.-C., Revyakin, A., Hajj, B., Legant, W., Dahan, M., Lionnet, T., Betzig, E., et al. (2014). Single-Molecule Dynamics of Enhanceosome Assembly in Embryonic Stem Cells. Cell 156, 1274-1285.
Chen, J., Gao, S., Gao, Y., Huang, H., Xu, K., Chen, X., Jiang, Y., Li, H., Gao, S., Tao, Y., et al. (2015a). The combination of Tet1 with Oct4 generates high-quality mouse-induced pluripotent stem cells. Stem Cells 33, 686-698.
Chen, J., Chen, X., Li, M., Liu, X., Gao, Y., Kou, X., Zhao, Y., Zheng, W., Zhang, X., Huo, Y., et al. (2016). Hierarchical Oct4 Binding in Concert with Primed Epigenetic Rearrangements during Somatic Cell Reprogramming. Cell Rep. 1-15.
Chen, K., Long, Q., Xing, G., Wang, T., Wu, Y., Li, L., Qi, J., Zhou, Y., Ma, B., Schöler, H. R., et al. (2020a). Heterochromatin loosening by the Oct4 linker region facilitates Klf4 binding and iPSC reprogramming. EMBO J. 39, 1-16.
Chen, K., Long, Q., Xing, G., Wang, T., Wu, Y., Li, L., Qi, J., Zhou, Y., Ma, B., Schöler, H. R., et al. (2020b). Heterochromatin loosening by the Oct4 linker region facilitates Klf4 binding and iPSC reprogramming. EMBO J. 39, 1-16.
Chen, X., Xu, H., Yuan, P., Fang, F., Huss, M., Vega, V. B., Wong, E., Orlov, Y. L., Zhang, W., Jiang, J., et al. (2008). Integration of External Signaling Pathways with the Core Transcriptional Network in Embryonic Stem Cells. Cell 133, 1106-1117.
Chen, Y., Niu, Y., Li, Y., Ai, Z., Kang, Y., Shi, H., Xiang, Z., Yang, Z., Tan, T., Si, W., et al. (2015b). Generation of Cynomolgus Monkey Chimeric Fetuses using Embryonic Stem Cells. Cell Stem Cell 17, 116-124.
Chen, Y., Lüttmann, F. F., Schoger, E., Schöler, H. R., Zelarayen, L. C., Kim, K.-P., Haigh, J. J., Kim, J., and Braun, T. (2021). Reversible reprogramming of cardiomyocytes to a fetal state drives heart regeneration in mice. Science (80-.). 373, 1537-1540.
Choi, J., Huebner, A. J., Clement, K., Walsh, R. M., Savol, A., Lin, K., Gu, H., Di Stefano, B., Brumbaugh, J., Kim, S., et al. (2017). Prolonged Mek1/2 suppression impairs the developmental potential of embryonic stem cells. Nature 1, 1-7. 10.1038/nature23274.
Chondronasiou, D., Gill, D., Mosteiro, L., Urdinguio, R. G., Berenguer-Llergo, A., Aguilera, M., Durand, S., Aprahamian, F., Nirmalathasan, N., Abad, M., et al. (2022). Multi-omic rejuvenation of naturally aged tissues by a single cycle of transient reprogramming. Aging Cell 21, 1-19.
Chronis, C., Fiziev, P., Papp, B., Butz, S., Bonora, G., Sabri, S., Ernst, J., and Plath, K. (2017). Cooperative Binding of Transcription Factors Orchestrates Reprogramming. Cell 168, 442-459.e20.
Danecek, P., Bonfield, J. K., Liddle, J., Marshall, J., Ohan, V., Pollard, M. O., Whitwham, A., Keane, T., McCarthy, S. A., Davies, R. M., et al. (2021). Twelve years of SAMtools and BCFtools. Gigascience 10, 1-4.
Davis, R. L., Weintraub, H., and Lassar, A. B. (1987). Expression of a single transfected cDNA converts fibroblasts to myoblasts. Cell 51, 987-1000.
Di, K.-Q., Gao, S., Cui, L.-F., Chang, G., Wu, F.-J., Ren, L.-K., An, L., Miao, K., Tan, K., Tao, L., et al. (2015). Generation of Fully Pluripotent Female Murine-Induced Pluripotent Stem Cells1. Biol. Reprod. 92, 1-8.
Dodonova, S. O., Zhu, F., Dienemann, C., Taipale, J., and Cramer, P. (2020). Nucleosome-bound SOX2 and SOX11 structures elucidate pioneer factor function. Nature 580, 669-672.
Drozd, A. M., Walczak, M. P., Piaskowski, S. et al. (2015) Generation of human iPSCs from cells of fibroblastic and epithelial origin by means of the oriP/EBNA-1 episomal reprogramming system. Stem Cell Res Ther 6, 122.
Eakin, G. S., and Hadjantonakis, A. (2006). Production of chimeras by aggregation of embryonic stem cells with diploid or tetraploid mouse embryos. 1. 10.1038/nprot.2006.173.
Eggan, K., Rode, A., Jentsch, I., Samuel, C., Hennek, T., Tintrup, H., Zevnik, B., Erwin, J., Loring, J., Jackson-Grusby, L., et al. (2002). Male and female mice derived from the same embryonic stem cell clone by tetraploid embryo complementation. Nat. Biotechnol. 20, 455-459.
Esch, D., Vahokoski, J., Groves, M. R., Pogenberg, V., Cojocaru, V., vom Bruch, H., Han, D., Drexler, H. C. a., Araúzo-Bravo, M. J., Ng, C. K. L., et al. (2013). A unique Oct4 interface is crucial for reprogramming to pluripotency. Nat. Cell Biol. 15, 1-9.
Fogarty, N. M. E., McCarthy, A., Snijders, K. E., Powell, B. E., Kubikova, N., Blakeley, P., Lea, R., Elder, K., Wamaitha, S. E., Kim, D., et al. (2017). Genome editing reveals a role for OCT4 in human embryogenesis. Nature 550, 67-73.
Fu, R., Yu, D., Ren, J., Li, C., Wang, J., Feng, G., Wang, X., Wan, H., Li, T., Wang, L., et al. (2020). Domesticated cynomolgus monkey embryonic stem cells allow the generation of neonatal interspecies chimeric pigs. Protein Cell 11, 97-107.
Fujii, H., and Hamada, H. (1993). A CNS Specific POU Transcription Is Required for Establishing Mammalian Neural Cell lineages. Neuron 11, 1197-1206.
Gafni, O., Weinberger, L., Mansour, A. A., Manor, Y. S., Chomsky, E., Ben-Yosef, D., Kalma, Y., Viukov, S., Maza, I., Zviran, A., et al. (2013). Derivation of novel human ground state naïve pluripotent stem cells. Nature 504, 282-286. 10.1038/nature12745.
Gill, D., Parry, A., Santos, F., Okkenhaug, H., Todd, C. D., Hernando-Herraez, I., Stubbs, T. M., Milagre, I., and Reik, W. (2022). Multi-omic rejuvenation of human cells by maturation phase transient reprogramming. Elife 11, 2021.01.15.426786.
Guo, G., Yang, J., Nichols, J., Hall, J. S., Eyres, I., Mansfield, W., and Smith, A. (2009). Klf4 reverts developmentally programmed restriction of ground state pluripotency. Development 136, 1063-1069.
Han, D., Wu, G., Chen, R., Drexler, H. C. A., MacCarthy, C. M., Kim, K.-P., Adachi, K., Gerovska, D., Mavrommatis, L., Bedzhov, I., et al. (2022a). A balanced Oct4 interactome is crucial for maintaining pluripotency. Sci. Adv. 8, eabe4375.
Han, D. W., Tapia, N., Joo, J. Y., Greber, B., Araúzo-Bravo, M. J., Bernemann, C., Ko, K., Wu, G., Stehling, M., Do, J. T., et al. (2010). Epiblast stem cell subpopulations represent mouse embryos of distinct pregastrulation stages. Cell 143, 617-627.
Han, L., Wei, X., Liu, C., Volpe, G., Zhuang, Z., Zou, X., Wang, Z., Pan, T., Yuan, Y., Zhang, X., et al. (2022b). Cell transcriptomic atlas of the non-human primate Macaca fascicularis. Nature.
Hanna, J., Cheng, A., and Saha, K. (2010). Human embryonic stem cells with biological and epigenetic characteristics similar to those of mouse ESCs. PNAS, 1-6. 10.1073/pnas.1004584107/-/DCSupplemental.www.pnas.org/cgi/doi/10.1073/pnas.1004584107.
Heinz, S., Benner, C., Spann, N., Bertolino, E., Lin, Y. C., Laslo, P., Cheng, J. X., Murre, C., Singh, H., and Glass, C. K. (2010). Simple Combinations of Lineage-Determining Transcription Factors Prime cis-Regulatory Elements Required for Macrophage and B Cell Identities. Mol. Cell 38, 576-589.
Hirai, H., Katoku-Kikyo, N., Karian, P., Firpo, M., and Kikyo, N. (2012). Efficient iPS cell production with the MyoD transactivation domain in serum-free culture. PLoS One 7, e34149.
Hiura, H., Toyoda, M., Okae, H., Sakurai, M., Miyauchi, N., Sato, A., Kiyokawa, N., Okita, H., Miyagawa, Y., Akutsu, H., et al. (2013). Stability of genomic imprinting in human induced pluripotent stem cells. BMC Genet. 14, 32.
Hochedlinger, K., Yamada, Y., Beard, C., and Jaenisch, R. (2005). Ectopic expression of Oct-4 blocks progenitor-cell differentiation and causes dysplasia in epithelial tissues. Cell 121, 465-477.
Hod Y. A simplified ribonuclease protection assay. Biotechniques. 1992 December; 13(6):852-4. PMID: 1282345.
Hogan, B., Costantini, F., and Lacy, E. (1986). Manipulating the mouse embryo: a laboratory manual (Cold Spring Harbor Laboratory Press).
Holm, T. M., Jackson-Grusby, L., Brambrink, T., Yamada, Y., Rideout, W. M., and Jaenisch, R. (2005). Global loss of imprinting leads to widespread tumorigenesis in adult mice. Cancer Cell 8, 275-285.
Horvath, S. DNA methylation age of human tissues and cell types. Genome Biol 14, 3156 (2013). https://doi.org/10.1186/gb-2013-14-10-r115
Huertas, J., MacCarthy, C. M., Schöler, H. R., and Cojocaru, V. (2020). Nucleosomal DNA Dynamics Mediate Oct4 Pioneer Factor Binding. Biophys. J. 1-17.
Irie, N., Weinberger, L., Tang, W. W .C., Kobayashi, T., Viukov, S., Manor, Y. S., Dietmann, S., Hanna, J. H., and Surani, M. A. (2015). SOX17 Is a Critical Specifier of Human Primordial Germ Cell Fate. Cell 160, 253-268. Jauch, R., Aksoy, I., Hutchins, A. P., Ng, C. K. L., Tian, X. F., Chen, J., Palasingam, P., Robson, P., Stanton, L. W., and Kolatkar, P. R. (2011). Conversion of Sox17 into a pluripotency reprogramming factor by reengineering its association with Oct4 on DNA. Stem Cells 29, 940-951.
Jelinic, P., and Shaw, P. (2007). Loss of imprinting and cancer. J. Pathol. 211, 261-268.
Jerabek, S., Ng, C. K., Wu, G., Arauzo-Bravo, M. J., Kim, K., Esch, D., Malik, V., Chen, Y., Velychko, S., MacCarthy, C. M., et al. (2017). Changing dimerization preferences converts Oct6 into a pluripotency inducer. EMBO Rep. 18, 319-333.
Kanatsu-Shinohara, M., Inoue, K., Lee, J., Yoshimoto, M., Ogonuki, N., Miki, H., Baba, S., Kato, T., Kazuki, Y., Toyokuni, S., et al. (2004). Generation of pluripotent stem cells from neonatal mouse testis. Cell 119, 1001-1012.
Kanatsu-Shinohara, M., Lee, J., Inoue, K., Ogonuki, N., Miki, H., Toyokuni, S., Ikawa, M., Nakamura, T., Ogura, A., and Shinohara, T. (2008). Pluripotency of a single spermatogonial stem cell in mice. Biol. Reprod. 78, 681-687.
Karwacki-Neisius, V., Göke, J., Osorno, R., Halbritter, F., Ng, J. H., Weiße, A. Y., Wong, F. C. K., Gagliardi, A., Mullin, N. P., Festuccia, N., et al. (2013). Reduced Oct4 Expression Directs a Robust Pluripotent State with Distinct Signaling Activity and Increased Enhancer Occupancy by Oct4 and Nanog. Cell Stem Cell 12, 531-545.
Keshet, G., and Benvenisty, N. (2021). Large-scale analysis of imprinting in naïve human pluripotent stem cells reveals recurrent aberrations and a potential link to FGF signaling. Stem Cell Reports 16, 2520-2533.
Kim, K.-P., Wu, Y., Yoon, J., Adachi, K., Wu, G., Velychko, S., MacCarthy, C. M., Shin, B., Röpke, A., Arauzo-Bravo, M. J., et al. (2020). Reprogramming competence of OCT factors is determined by transactivation domains. Sci. Adv. 6, eaaz7364.
Kim, K.-P., Choi, J., Yoon, J., Bruder, J. M., Shin, B., Kim, J., Arauzo-Bravo, M. J., Han, D., Wu, G., Han, D. W., et al. (2021). Permissive epigenomes endow reprogramming competence to transcriptional regulators. Nat. Chem. Biol. 17, 47-56.
Kime, C., Rand, T. A., Ivey, K. N., Srivastava, D., Yamanaka, S., and Tomoda, K. (2015). Practical Integration-Free Episomal Methods for Generating Human Induced Pluripotent Stem Cells. Curr. Protoc. Hum. Genet. 87, 21.2.1-21.2.21.
King, H. W., and Klose, R. J. (2017). The pioneer factor OCT4 requires the chromatin remodeller BRG1 to support gene regulatory element function in mouse embryonic stem cells. Elife 6, 1-24.
Klemm, J. D., Rould, M. A., Aurora, R., Herr, W., and Pabo, C. O. (1994). Crystal structure of the Oct-1 POU domain bound to an octamer site: DNA recognition with tethered DNA-binding modules. Cell 77, 21-32.
Klinker, H., Haas, C., Harrer, N., Becker, P. B., and Mueller-Planitz, F. (2014). Rapid purification of recombinant histones. PLoS One 9.
Ko, K., Tapia, N., Wu, G., Kim, J. B., Bravo, M. J. A., Sasse, P., Glaser, T., Ruau, D., Han, D. W., Greber, B., et al. (2009). Induction of Pluripotency in Adult Unipotent Germline Stem Cells. Cell Stem Cell 5, 87-96.
Kunath, T., Saba-El-Leil, M. K., Almousailleakh, M., Wray, J., Meloche, S., and Smith, A. (2007). FGF stimulation of the Erk1/2 signalling cascade triggers transition of pluripotent embryonic stem cells from self-renewal to lineage commitment. Development 134, 2895-2902.
Langmead, B., and Salzberg, S. L. (2012). Fast gapped-read alignment with Bowtie 2. Nat. Methods 9, 357-359.
Lea R A, McCarthy A, Boeing S, Fallesen T, Elder K, Snell P, Christie L, Adkins S, Shaikly V, Taranissi M, Niakan KK. KLF17 promotes human naïve pluripotency but is not required for its establishment. Development. 2021 Nov. 15; 148(22):dev199378. doi: 10.1242/dev.199378. Epub 2021 Nov. 15. PMID: 34661235; PMCID: PMC8645209.
Lee, J., Go, Y., Kang, I., Han, Y.-M., and Kim, J. (2010). Oct-4 controls cell-cycle progression of embryonic stem cells. Biochem. J. 426, 171-181.
Van Leeuwen, H. C., Strating, M. J., Rensen, M., De Laat, W., and Van Der Vliet, P. C. (1997). Linker length and composition influence the flexibility of Oct-1 DNA binding. EMBO J. 16, 2043-2053.
Li, H., Ta, N., Long, C., Zhang, Q., Li, S., Liu, S., Yang, L., and Zuo, Y. (2019). The spatial binding model of the pioneer factor Oct4 with its target genes during cell reprogramming. Comput. Struct. Biotechnol. J. 17, 1226-1233.
Lin J R, Hu J. SeqNLS: nuclear localization signal prediction based on frequent pattern mining and linear motif scoring. PLoS One. 2013 Oct. 29; 8(10):e76864. doi: 10.1371/journal.pone.0076864. PMID: 24204689; PMCID: PMC3812174.
Liquin, E. G., and Gopnik, A. (2022). Children are more exploratory and learn more than adults in an approach-avoid task. Cognition 218, 104940. 10.1016/j.cognition.2021.104940.
Liu, K., Wang, F., Ye, X., Wang, L., Yang, J., Zhang, J., and Liu, L. (2014). KSR-based medium improves the generation of high-quality mouse iPS cells. PLoS One 9.
Liu, S., Bou, G., Sun, R., Guo, S., Xue, B., Wei, R., Cooney, A. J., and Liu, Z. (2015). Sox2 is the faithful marker for pluripotency in pig: Evidence from embryonic studies. Dev. Dyn. 244, 619-627.
Liu, X., Nefzger, C. M., Rossello, F. J., Chen, J., Knaupp, A. S., Firas, J., Ford, E., Pflueger, J., Paynter, J. M., Chy, H. S., et al. (2017). Comprehensive characterization of distinct states of human naive pluripotency generated by reprogramming. Nat. Methods 14, 1055-1062. 10.1038/nmeth.4436.
Liu, Z., and Kraus, W. L. (2017). Catalytic-Independent Functions of PARP-1 Determine Sox2 Pioneer Activity at Intractable Genomic Loci. Mol. Cell 65, 589-603.e9.
Lowary, P. T., and Widom, J. (1998). New DNA sequence rules for high affinity binding to histone octamer and sequence-directed nucleosome positioning. J. Mol. Biol. 276, 19-42.
Lu, Y., Brommer, B., Tian, X., Krishnan, A., Meer, M., Wang, C., Vera, D. L., Zeng, Q., Yu, D., Bonkowski, M. S., et al. (2020). Reprogramming to recover youthful epigenetic information and restore vision. Nature 588, 124-129.
Luger, K., Rechsteiner, T. J., and Richmond, T. J. (1999). Expression and purification of recombinant histones and nucleosome reconstitution. Methods Mol. Biol. 119, 1-16.
Luo, L., Shi, Y., Wang, H., Wang, Z., Dang, Y., Li, S., Wang, S., and Zhang, K. (2021). Base editing in bovine embryos reveals a species-specific role of SOX2 in regulation of pluripotency. BioRxiv 2021.11.10.468023.
Madeira F, et al. The EMBL-EBI search and sequence analysis tools APIs in 2019. Nucleic Acids Research. 2019 July; 47(W1):W636-W641. DOI: 10.1093/nar/gkz268
Malik, V., Glaser, L. V, Zimmer, D., Velychko, S., Weng, M., Holzner, M., Arend, M., Chen, Y., Srivastava, Y., Veerapandian, V., et al. (2019). Pluripotency reprogramming by competent and incompetent POU factors uncovers temporal dependency for Oct4 and Sox2. Nat. Commun. 10, 3477.
Masui, S., Nakatake, Y., Toyooka, Y., Shimosato, D., Yagi, R., Takahashi, K., Okochi, H., Okuda, A., Matoba, R., Sharov, A. a, et al. (2007). Pluripotency governed by Sox2 via regulation of Oct3/4 expression in mouse embryonic stem cells. Nat. Cell Biol. 9, 625-635.
Merino, F., Ng, C. K. L., Veerapandian, V., Schöler, H. R., Jauch, R., and Cojocaru, V. (2014). Structural Basis for the SOX-Dependent Genomic Redistribution of OCT4 in Stem Cell Differentiation. Structure 22, 1274-1286.
Merino, F., Bouvier, B., and Cojocaru, V. (2015). Cooperative DNA Recognition Modulated by an Interplay between Protein-Protein Interactions and DNA-Mediated Allostery. PLOS Comput. Biol. 11, e1004287.
Michael, A. K., Grand, R. S., Isbel, L., Cavadini, S., Kozicka, Z., Kempf, G., Bunker, R. D., Schenk, A. D., Graff-Meyer, A., Pathare, G. R., et al. (2020). Mechanisms of OCT4-SOX2 motif readout on nucleosomes. Science (80-.). 0074, eabb0074.
Mistri, T. K., Devasia, A. G., Chu, L. T., Ng, W. P., Halbritter, F., Colby, D., Martynoga, B., Tomlinson, S. R., Chambers, I., Robson, P., et al. (2015). Selective influence of Sox2 on POU transcription factor binding in embryonic and neural stem cells. EMBO Rep. 16, 1177-1191.
Müller, H. G., Chiou, J. M., Carey, J. R., and Wang, J. L. (2002). Fertility and life span: Late children enhance female longevity. Journals Gerontol.—Ser. A Biol. Sci. Med. Sci. 57, 202-206. 10.1093/gerona/57.5.B202.
Nagy, A., Rossant, J., Nagy, R., Abramow-Newerly, W., and Roder, J. C. (1993). Derivation of completely cell culture-derived mice from early-passage embryonic stem cells. Proc. Natl. Acad. Sci. U.S.A 90, 8424-8428.
Nair, R., Carer, P., Rost, B., NLSdb: database of nuclear localization signals. Nucl Acids Res, 2003, 31:397-399.
Nakagawa, M., Koyanagi, M., Tanabe, K., Takahashi, K., Ichisaka, T., Aoi, T., Okita, K., Mochiduki, Y., Takizawa, N., and Yamanaka, S. (2008). Generation of induced pluripotent stem cells without Myc from mouse and human fibroblasts. Nat. Biotechnol. 26, 101-106.
Nakatake, Y., Fukui, N., Iwamatsu, Y., Masui, S., Takahashi, K., Yagi, R., Yagi, K., Miyazaki, J., Matoba, R., Ko, M. S. H., et al. (2006). Klf4 Cooperates with Oct3/4 and Sox2 To Activate the Lefty1 Core Promoter in Embryonic Stem Cells. Mol. Cell. Biol. 26, 7772-7782.
Naviaux, R. K., Costanzi, E., Haas, M., and Verma, I. M. (1996). The pCL vector system: rapid production of helper-free, high-titer, recombinant retroviruses. J. Virol. 70, 5701-5705.
Needleman, S. B. and Wunsch, CD. A general method applicable to the search for similarities in the amino acid sequence of two proteins. J Mol Biol. 1970; 48(3):443-53. DOI: 10.1016/0022-2836(70)90057-4
Ng, C. K. L., Li, N. X., Chee, S., Prabhakar, S., Kolatkar, P. R., and Jauch, R. (2012). Deciphering the Sox-Oct partner code by quantitative cooperativity measurements. Nucleic Acids Res. 40, 4933-4941.
Nguyen Ba, A. N., Pogoutse, A., Provart, N. et al. NLStradamus: a simple Hidden Markov Model for nuclear localization signal prediction. BMC Bioinformatics 10, 202 (2009). https://doi.org/10.1186/1471-2105-10-202
Nishimoto, M., Fukushima, A., Okuda, A., and Muramatsu, M. (1999). The gene for the embryonic stem cell coactivator UTF1 carries a regulatory element which selectively interacts with a complex composed of Oct-3/4 and Sox-2. Mol. Cell. Biol. 19, 5453-5465.
Nishimoto, M., Miyagi, S., Katayanagi, T., Tomioka, M., Muramatsu, M., and Okuda, A. (2003). The embryonic Octamer factor 3/4 displays distinct DNA binding specificity from those of other Octamer factors. Biochem. Biophys. Res. Commun. 302, 581-586.
Nishimoto, M., Miyagi, S., Yamagishi, T., Sakaguchi, T., Niwa, H., Muramatsu, M., and Okuda, A. (2005). Oct-3/4 Maintains the Proliferative Embryonic Stem Cell State via Specific Binding to a Variant Octamer Sequence in the Regulatory Region of the UTF1 Locus. Mol. Cell. Biol. 25, 5084-5094.
Niswander, L., and Martin, G. R. (1992). Fgf-4 expression during gastrulation, myogenesis, limb and tooth development in the mouse. Development 114, 755-768.
Niwa, H., Masui, S., Chambers, I., Smith, A. G., and Miyazaki, J. (2002). Phenotypic Complementation Establishes Requirements for Specific POU Domain and Generic Transactivation Function of Oct-3/4 in Embryonic Stem Cells. Mol. Cell. Biol. 22, 1526-1536.
Nowak-Imialek, M., Kues, W. A., Petersen, B., Lucas-Hahn, A., Herrmann, D., Haridoss, S., Oropeza, M., Lemme, E., Scholer, H. R., Carnwath, J. W., et al. (2011). Oct4-enhanced green fluorescent protein transgenic pigs: a new large animal model for reprogramming studies. Stem Cells Dev 20, 1563-1575.
Ocampo, A., Reddy, P., Martinez-Redondo, P., Platero-Luengo, A., Hatanaka, F., Hishida, T., Li, M., Lam, D., Kurita, M., Beyret, E., et al. (2016). In Vivo Amelioration of Age-Associated Hallmarks by Partial Reprogramming. Cell 167, 1719-1733.e12.
Oh and Kessler, 2018. Design, Assembly, Production and Transfection of Synthetic Modified mRNA, Methods. 133:29-43
Okita, K., Matsumura, Y., Sato, Y., Okada, A., Morizane, A., Okamoto, S., Hong, H., Nakagawa, M., Tanabe, K., Tezuka, K., et al. (2011). A more efficient method to generate integration-free human iPS cells. Nat. Methods 8, 409-412.
Okita, K., Yamakawa, T., Matsumura, Y., Sato, Y., Amano, N., Watanabe, A., Goshima, N., and Yamanaka, S. (2013). An efficient nonviral method to generate integration-free human-induced pluripotent stem cells from cord blood and peripheral blood cells. Stem Cells 31, 458-466.
Okumura-Nakanishi, S., Saito, M., Niwa, H., and Ishikawa, F. (2005). Oct-3/4 and Sox2 regulate Oct-3/4 gene in embryonic stem cells. J. Biol. Chem. 280, 5307-5317.
Parker and Barnes, Meth. Mol. Biol., 106:247-283, 1999
Plusa B, Piliszek A, Frankenberg S, Artus J, Hadjantonakis AK. Distinct sequential cell behaviours direct primitive endoderm formation in the mouse blastocyst. Development. 2008 September; 135(18):3081-91. doi: 10.1242/dev.021519. PMID: 18725515; PMCID: PMC2768606.
Ptak, G. E., Tacconi, E., Czernik, M., Toschi, P., Modlinski, J. A., and Loi, P. (2012). Embryonic diapause is conserved across mammals. PLoS One 7.
Qin, H., Hejna, M., Liu, Y., Percharde, M., Wossidlo, M., Blouin, L., Durruthy-Durruthy, J., Wong, P., Qi, Z., Yu, J., et al. (2016). YAP Induces Human Naive Pluripotency. Cell Rep. 14, 2301-2312. 10.1016/j.celrep.2016.02.036.
Ramírez, F., Ryan, D. P., Grüning, B., Bhardwaj, V., Kilpert, F., Richter, A. S., Heyne, S., Dündar, F., and Manke, T. (2016). deepTools2: a next generation web server for deep-sequencing data analysis. Nucleic Acids Res. 44, W160-W165.
Ramirez Rozzi, F. V., and Bermudez de Castro, J. M. (2004). Surprisingly rapid growth in Neanderthals. Nature 428, 936-939. 10.1038/nature02428.
Rand, T. A., Sutou, K., Tanabe, K., Jeong, D., Nomura, M., Kitaoka, F., Tomoda, E., Narita, M., Nakamura, M., Nakamura, M., et al. (2018). MYC Releases Early Reprogrammed Human Cells from Proliferation Pause via Retinoblastoma Protein Inhibition. Cell Rep. 23, 361-375.
Reményi, A., Lins, K., Nissen, L. J., Reinbold, R., Schöler, H. R., and Wilmanns, M. (2003). Crystal structure of a POU_HMG_DNA ternary complex suggests differential assembly of Oct4 and Sox2 on two enhancers.pdf. 2048-2059.
Roberts, G. A., Ozkan, B., Gachulincová, I., O'Dwyer, M. R., Hall-Ponsele, E., Saxena, M., Robinson, P. J., and Soufi, A. (2021). Dissecting OCT4 defines the role of nucleosome binding in pluripotency. Nat. Cell Biol.
Di Rocco, G., Gavalas, A., Pöpper, H., Krumlauf, R., Mavilio, F., and Zappavigna, V. (2001). The Recruitment of SOX/OCT Complexes and the Differential Activity of HOXA1 and HOXB1 Modulate the Hoxb1 Auto-regulatory Enhancer Function. J. Biol. Chem. 276, 20506-20515.
Sanz G, Singh M, Peuget S, Selivanova G. Inhibition of p53 inhibitors: progress, challenges and perspectives. J Mol Cell Biol. 2019 Jul. 19; 11(7):586-599. doi: 10.1093/jmcb/mjz075. PMID: 31310659; PMCID: PMC6735775.Sarkar, T. J., Quarta, M., Mukherjee, S., Colville, A., Paine, P., Doan, L., Tran, C. M., Chu, C. R., Horvath, S., Qi, L. S., et al. (2020). Transient non-integrative expression of nuclear reprogramming factors promotes multifaceted amelioration of aging in human cells. Nat. Commun. 11, 1-12.
Scholz, J., Besir, H., Strasser, C., and Suppmann, S. (2013). A new method to customize protein expression vectors for fast, efficient and background free parallel cloning. BMC Biotechnol. 13, 1-11.
Scognamiglio, R., Cabezas-Wallscheid, N., Thier, M. C., Altamura, S., Reyes, A., Prendergast, A. M., Baumgartner, D., Carnevalli, L. S., Atzberger, A., Haas, S., et al. (2016). Myc Depletion Induces a Pluripotent Dormant State Mimicking Diapause. Cell 164, 668-680.
Scott M S, Troshin P V, Barton G J. NoD: a Nucleolar localization sequence detector for eukaryotic and viral proteins. BMC Bioinformatics. 2011 Aug. 3; 12:317. doi: 10.1186/1471-2105-12-317. PMID: 21812952; PMCID: PMC3166288.
Shahbazi, E., Moradi, S., Nemati, S., Satarian, L., Basiri, M., Gourabi, H., Zare Mehrjardi, N., Günther, P., Lampert, A., Handler, K., et al. (2016). Conversion of Human Fibroblasts to Stably Self-Renewing Neural Stem Cells with a Single Zinc-Finger Transcription Factor.
Smith, B. H. (1995). Toward a Life History of the Hominidae. Annu. Rev. Anthropol. 24, 257-279. 10.1146/annurev.anthro.24.1.257.
Soufi, A., Donahue, G., and Zaret, K. S. (2012). Facilitators and impediments of the pluripotency reprogramming factors' initial engagement with the genome. Cell 151, 994-1004.
Soufi, A., Garcia, M. F., Jaroszewicz, A., Osman, N., Pellegrini, M., Zaret, K. S., Soufi, A., Garcia, M. F., Jaroszewicz, A., Osman, N., et al. (2015). Pioneer Transcription Factors Target Partial DNA Motifs on Nucleosomes to Initiate Reprogramming. Cell 161, 1-14.
Stadtfeld, M., Apostolou, E., Ferrari, F., Choi, J., Walsh, R. M., Chen, T., Ooi, S. S. K., Kim, S. Y., Bestor, T. H., Shioda, T., et al. (2012). Ascorbic acid prevents loss of Dlk1-Dio3 imprinting and facilitates generation of all-iPS cell mice from terminally differentiated B cells. Nat. Genet. 44, 398-405, S1-2.
Stark, R., Grzelak, M. & Hadfield, J. RNA sequencing: the teenage years. Nat Rev Genet 20, 631-656 (2019). https://doi.org/10.1038/s41576-019-0150-2
Takahashi, K., and Yamanaka, S. (2006). Induction of pluripotent stem cells from mouse embryonic and adult fibroblast cultures by defined factors. Cell 126, 663-676.
Takashima, Y., Guo, G., Loos, R., Nichols, J., Ficz, G., Krueger, F., Oxley, D., Santos, F., Clarke, J., Mansfield, W., et al. (2014). Resetting Transcription Factor Control Circuitry toward Ground-State Pluripotency in Human. Cell 158, 1254-1269. 10.1016/j.cell.2014.08.029.
Takikawa, S., Ray, C., Wang, X., Shamis, Y., Wu, T. Y., and Li, X. (2013). Genomic imprinting is variably lost during reprogramming of mouse iPS cells. Stem Cell Res. 11, 861-873.
Tan, D. S., Chen, Y., Gao, Y., Bednarz, A., Wei, Y., Malik, V., Ho, D. H.-H., Weng, M., Ho, S. Y., Srivastava, Y., et al. (2021a). Directed Evolution of an Enhanced POU Reprogramming Factor for Cell Fate Engineering. Mol. Biol. Evol. 1-29.
Tan, D. S., Chen, Y., Gao, Y., Bednarz, A., Wei, Y., Malik, V., Ho, D. H. H., Weng, M., Ho, S. Y., Srivastava, Y., et al. (2021b). Directed Evolution of an Enhanced POU Reprogramming Factor for Cell Fate Engineering. Mol. Biol. Evol. 38, 2854-2868.
Tanaka, S., and Kamachi, Y. (2004). Interplay of SOX and POU factors in regulation of the Nestin gene in neural primordial cells. . . . Cell. Biol. 24, 8834-8846.
Tapia, N., Reinhardt, P., Duemmler, A., Wu, G., Araúzo-Bravo, M. J., Esch, D., Greber, B., Cojocaru, V., Rascon, C. A., Tazaki, A., et al. (2012). Reprogramming to pluripotency is an ancient trait of vertebrate Oct4 and Pou2 proteins. Nat. Commun. 3, 1279.
Tapia, N., MacCarthy, C., Esch, D., Gabriele Marthaler, A., Tiemann, U., Araúzo-Bravo, M. J., Jauch, R., Cojocaru, V., and Schöler, H. R. (2015). Dissecting the role of distinct OCT4-SOX2 heterodimer configurations in pluripotency. Sci. Rep. 5, 13533.
Tarazi, S., Aguilera-Castrejon, A., Joubran, C., Ghanem, N., Ashouokhi, S., Roncato, F., Wildschutz, E., Haddad, M., Oldak, B., Gomez-Cesar, E., et al. (2022). Post-gastrulation synthetic embryos generated ex utero from mouse naïve ESCs. Cell 1-17.
Teif, V. B., Vainshtein, Y., Caudron-Herger, M., Mallm, J.-P., Marth, C., Hofer, T., and Rippe, K. (2012). Genome-wide nucleosome positioning during embryonic stem cell development. Nat. Struct. Mol. Biol. 19, 1185-1192.
Theunissen, T. W., Powell, B. E., Wang, H., Mitalipova, M., Faddah, D. A., Reddy, J., Fan, Z. P., Maetzel, D., Ganz, K., Shi, L., et al. (2014). Systematic identification of culture conditions for induction and maintenance of naïve human pluripotency. Cell Stem Cell 15, 471-487. 10.1016/j.stem.2014.07.002.
Thomson, J. A., Itskovitz-Eldor, J., Shapiro, S. S., Waknitz, M. A., Swiergiel, J. J., Marshall, V. S., and Jones, J. M. (1998). Embryonic Stem Cell Lines Derived from Human Blastocysts. Science (80-.). 282, 1145-1147.
Towbin, H., Staehelin, T., and Gordon, J. (1979). Electrophoretic transfer of proteins from polyacrylamide gels to nitrocellulose sheets: procedure and some applications. Proc. Natl. Acad. Sci. 76, 4350-4354.
Tremble, K. C., Stirparo, G. G., Bates, L. E., Maskalenka, K., Stuart, H. T., Jones, K., Andersson-Rolf, A., Radzisheuskaya, A., Koo, B.-K., Bertone, P., et al. (2021). Sox2 modulation increases naïve pluripotency plasticity. IScience 24, 102153.
Trowitzsch, S., Bieniossek, C., Nie, Y., Garzoni, F., and Berger, I. (2010). New baculovirus expression tools for recombinant protein complex production. J. Struct. Biol. 172, 45-54.
Van Craenenbroeck et al., 2000. Episomal vectors for gene expression in mammalian cells. Eur. J. Biochem. 267, 5665-5678
Veerapandian, V., Ackermann, J. O., Srivastava, Y., Malik, V., Weng, M., Yang, X., and Jauch, R. (2018). Directed Evolution of Reprogramming Factors by Cell Selection and Sequencing. Stem Cell Reports 11, 593-606.
Velychko, S., Adachi, K., Kim, K.-P. P., Hou, Y., MacCarthy, C. M., Wu, G., and Schöler, H. R. (2019a). Excluding Oct4 from Yamanaka Cocktail Unleashes the Developmental Potential of iPSCs. Cell Stem Cell 25, 737-753.e4.
Velychko, S., Kang, K., Kim, S. M., Kwak, T. H., Kim, K. P., Park, C., Hong, K., Chung, C. H., Hyun, J. K., MacCarthy, C. M., et al. (2019b). Fusion of Reprogramming Factors Alters the Trajectory of Somatic Lineage Conversion. Cell Rep. 27, 30-39.e4.
Waddington, C. H. (1957). The strategy of the genes: a discussion of some aspects of theoretical biology. Allen & Unwin.
Weinberger, L., Ayyash, M., Novershtern, N. et al. Dynamic stem cell states: naive to primed pluripotency in rodents and humans. Nat Rev Mol Cell Biol 17, 155-169 (2016).
Weis et al., Trends in Genetics, 8:263-264, 1992
Weissbein, U., Schachter, M., Egli, D., and Benvenisty, N. (2016). Analysis of chromosomal aberrations and recombination by allelic bias in RNA-Seq. Nat. Commun. 7, 1-8.
White, M. D., Angiolini, J. F., Alvarez, Y. D., Kaur, G., Zhao, Z. W., Mocskos, E., Bruno, L., Bissiere, S., Levi, V., and Plachta, N. (2016). Long-Lived Binding of Sox2 to DNA Predicts Cell Fate in the Four-Cell Mouse Embryo. Cell 165, 75-87.
Wojdyla, K., Collier, A. J., Fabian, C., Nisi, P. S., Biggins, L., Oxley, D., and Rugg-Gunn, P. J. (2020). Cell-Surface Proteomics Identifies Differences in Signaling and Adhesion Protein Expression between Naive and Primed Human Pluripotent Stem Cells. Stem Cell Reports 14, 972-988. 10.1016/j.stemcr.2020.03.017.
Wu, G., and Schöler, H. R. (2014). Role of Oct4 in the early embryo development. Cell Regen. 3, 7.
Wu, G., Han, D., Gong, Y., Sebastiano, V., Gentile, L., Singhal, N., Adachi, K., Fischedick, G., Ortmeier, C., Sinn, M., et al. (2013). Establishment of totipotency does not depend on Oct4A. Nat. Cell Biol. 15, 1089-1097.
Wu, X., Goodyear, S. M., Tobias, J. W., Avarbock, M. R., and Brinster, R. L. (2011). Spermatogonial Stem Cell Self-Renewal Requires ETV5-Mediated Downstream Activation of Brachyury in Mice. Biol. Reprod. 85, 1114-1123.
Wuensch, A., Habermann, F. a, Kurosaka, S., Klose, R., Zakhartchenko, V., Reichenbach, H.-D., Sinowatz, F., McLaughlin, K. J., and Wolf, E. (2007). Quantitative monitoring of pluripotency gene activation after somatic cloning in cattle. Biol. Reprod. 76, 983-991.
Wunderlich, S., Haase, A., Merkert, S., Beier, J., Schwanke, K., Schambach, A., Glage, S., Göhring, G., Curnow, E. C., and Martin, U. (2012). Induction of pluripotent stem cells from a cynomolgus monkey using a polycistronic simian immunodeficiency virus-based vector, differentiation toward functional cardiomyocytes, and generation of stably expressing reporter lines. Cell. Reprogram. 14, 471-484.
Wunderlich, S., Kircher, M., Vieth, B., Haase, A., Merkert, S., Beier, J., Göhring, G., Glage, S., Schambach, A., Curnow, E. C., et al. (2014). Primate iPS cells as tools for evolutionary analyses. Stem Cell Res. 12, 622-629.
Yamauchi, K., Ikeda, T., Hosokawa, M., Nakatsuji, N., Kawase, E., Chuma, S., Hasegawa, K., and Suemori, H. (2020). Overexpression of Nuclear Receptor 5A1 Induces and Maintains an Intermediate State of Conversion between Primed and Naive Pluripotency. Stem Cell Reports 14, 506-519. 10.1016/j.stemcr.2020.01.012.
Yang, B. X. X., El Farran, C. A., Guo, H. C. C., Yu, T., Fang, H. T. T., Wang, H. F. F., Schlesinger, S., Seah, Y. F. S. F. S., Goh, G. Y. L. Y. L., Neo, S. P. P., et al. (2015). Systematic Identification of Factors for Provirus Silencing in Embryonic Stem Cells. Cell 163, 230-245. 10.1016/j.cell.2015.08.037.
Yang, S. H., Andrabi, M., Biss, R., Murtuza Baker, S., Iqbal, M., and Sharrocks, A. D. (2019). ZIC3 Controls the Transition from Naive to Primed Pluripotency. Cell Rep. 27, 3215-3227.e6.
Yang, X.-X., Malik, V., and Jauch, R. (2014). Reprogramming cells with synthetic proteins. Asian J. Androl. 0, 0.
Ying, Q.-L., Wray, J., Nichols, J., Batlle-Morera, L., Doble, B., Woodgett, J., Cohen, P., and Smith, A. (2008). The ground state of embryonic stem cell self-renewal. Nature 453, 519-523.
Yoshioka, N., Gros, E., Li, H. R., Kumar, S., Deacon, D.C., Maron, C., Muotri, A. R., Chi, N. C., Fu, X. D., Yu, B. D., et al. (2013). Efficient generation of human iPSCs by a synthetic self-replicative RNA. Cell Stem Cell 13, 246-254.
Yuan, R., Meng, Q., Nautiyal, J., Flurkey, K., Tsaih, S. W., Krier, R., Parker, M. G., Harrison, D. E., and Paigen, B. (2012). Genetic coregulation of age of female sexual maturation and lifespan through circulating IGF1 among inbred mouse strains. Proc. Natl. Acad. Sci. U.S.A 109, 8224-8229. 10.1073/pnas.1121113109.
Zhang, Y., Liu, T., Meyer, C. A., Eeckhoute, J., Johnson, D. S., Bernstein, B. E., Nussbaum, C., Myers, R. M., Brown, M., Li, W., et al. (2008). Model-based analysis of ChIP-Seq (MACS). Genome Biol. 9.
Zhao, S., Nichols, J., Smith, A. G., and Li, M. (2004). SoxB transcription factors specify neuroectodermal lineage choice in ES cells. Mol. Cell. Neurosci. 27, 332-342.
Zheng, C., Hu, Y., Sakurai, M., Pinzon-Arteaga, C. A., Li, J., Wei, Y., Okamura, D., Ravaux, B., Barlow, H. R., Yu, L., et al. (2021). Cell competition constitutes a barrier for interspecies chimerism. Nature 592, 272-276. 10.1038/s41586-021-03273-0.
Zhu, Q., Song, L., Peng, G., Sun, N., Chen, J., Zhang, T., Sheng, N., Tang, W., Qian, C., Qiao, Y., et al. (2014). The transcription factor Pou3f1 promotes neural fate commitment via activation of neural lineage genes and inhibition of external signaling pathways. Elife 3, 1-21.

1. A SoxB1 factor variant comprising:
a) the HMG (high mobility group) domain of any one of the amino acid sequences of SEQ ID NOs: 1 to 3, wherein the amino acid alanine at position 61 is substituted with an amino acid selected from valine, leucine, isoleucine, phenylalanine, methionine, tryptophan or proline, preferably valine; or
b) an amino acid sequence sharing at least 82% sequence identity with the HMG domain as defined in (a), provided that the substitution as defined in (a) is retained.

2. The SoxB1 factor variant of claim 1a), wherein furthermore,
a) the amino acids at positions 43 to 47 in the HMG domain of any one of SEQ ID NOs: 1 to 3 are substituted with the amino acid sequence of SEQ ID NO: 7; and/or
b) the amino acids at positions 65 to 86 in the HMG domain of any one of SEQ ID NOs: 1 to 3 are substituted with the amino acid sequence of SEQ ID NO: 8; and/or
c) the SoxB1 factor variant comprises an amino acid sequence sharing at least 82% sequence identity with the SoxB1 factor variant of a) and/or b), provided that the substitution as defined in a) and/or b) is retained.

3. The SoxB1 factor variant of claim 1 or 2, wherein the SoxB1 factor variant is a Sox2 factor variant.

4. The SoxB1 factor variant of any one of claims 1 to 3, wherein the SoxB1 factor variant further comprises the amino acid sequence of SEQ ID NO: 9, wherein preferably the amino acid sequence of SEQ ID NO: 9 is linked to the C-terminal end of the HMG domain as defined in claim 1 or 2.

5. The SoxB1 factor variant of claim 4, wherein the SoxB1 factor variant comprises or consists of the amino acid sequence of SEQ ID NO: 13 or 14, or a SoxB1 factor variant sharing at least 96% sequence identity with the amino acid sequence of SEQ ID NO: 13 or 14.

6. A Sox17 factor variant comprising:
(a) the HMG domain of the amino acid sequence of SEQ ID NO: 15, wherein the amino acids at positions 24 to 28 of SEQ ID NO: 15 are substituted with the amino acid sequence of SEQ ID NO: 16; or
(b) an amino acid sequence sharing at least 82% sequence identity with the HMG domain as defined in (a), provided that the substitution as defined in (a) is retained.

7. A fusion protein comprising or consisting of:
a) a Sox factor and a POU factor; or
b) a Sox factor and a POU domain; or
c) an HMG domain and a POU factor; or
d) an HMG domain and a POU domain;
wherein
the Sox factor is selected from:
i. a SoxB1 factor or the SoxB1 factor variant of any one of claims 1 to 5; or
ii. Sox17 or the Sox17 factor variant of claim 6;
the HMG domain is selected from:
iii. the HMG domain of claim 1 a) or b);
the POU factor is selected from:
iv. Oct4 or an Oct4 variant; or
v. Oct2 or an Oct2 variant; or
vi. Oct6 or an Oct6 variant; or
vii. Brn2 or a Brn2 variant; or
viii. Brn4 or a Brn4 variant; or
ix. other natural or synthetic POU factors;
the POU domain is selected from:
x. the POU domain of SEQ ID NO: 17 or 18 or a variant thereof sharing at least 82% sequence identity with SEQ ID NO: 17 or 18;
wherein preferably, the amino acids at positions 1 to 50, 78 and/or 82 of SEQ ID NO: 17 or 18, respectively, are maintained.

8. A complex, or a composition, comprising or consisting of:
a) a Sox factor and a POU factor; or
b) a Sox factor and a POU domain; or
c) an HMG domain and a POU factor; or
d) an HMG domain and a POU domain, wherein
the Sox factor, the HMG domain, the POU factor, and the POU domain are selected from the Sox factor, the HMG domain, the POU factor and the POU domain as defined in claim 7.

9. A nucleic acid molecule, or a combination of nucleic acid molecules, encoding:
the SoxB1 factor variant of any one of claims 1 to 5, the Sox17 factor variant of claim 6, or the fusion protein of claim 7; and/or
the complex of claim 8; and/or
one or more additional reprogramming factor(s).

10. A vector or a combination of vectors comprising the nucleic acid molecule or the combination of nucleic acid molecules of claim 9.

11. A cell which is (a) a cell or (b) a cell derived from said cell, wherein said cell (a) comprises or said cell (b) has been modified by the presence in the cell of:
(i) the SoxB1 factor variant of any one of claims 1 to 5, the Sox17 factor variant of claim 6, or the fusion protein of claim 7, and/or the complex or composition of claim 8; and/or
(ii) the nucleic acid molecule or the combination of nucleic acid molecules of claim 9; and/or
(iii) the vector or combination of vectors of claim 10
and wherein the cell of (b) preferably retains the phenotype of the cell of (a).

12. A method for enhancing the cooperativity between a Sox factor and a POU factor, the method comprising increasing the average number and/or strength of interactions between the HMG domain of the Sox factor and the POU domain of the POU factor.

13. The method of claim 12, wherein the average number and/or strength of interactions is increased by substituting the amino acid at position 61 of the HMG domain with a hydrophobic amino acid selected from valine, leucine, isoleucine, phenylalanine, methionine, tryptophan or proline, wherein preferably, the amino acid at position 61 of the HMG domain is substituted with valine.

14. A method for producing induced pluripotent stem cell(s) (iPSC(s)) from non-pluripotent cell(s), the method comprising culturing said non-pluripotent cell(s) under conditions suitable for reprogramming said non-pluripotent cell(s) into iPSC(s), wherein said conditions comprise increasing in said non-pluripotent cells the level of, preferably co-expressing in said non-pluripotent cell(s):
a) the SoxB1 factor variant of any one of claims 1 to 5 or the Sox17 factor variant of claim 6, and a POU factor; and/or
b) the fusion protein of claim 7 or the complex or composition of claim 8; and/or
c) the SoxB1 factor variant of claim 5 and a Klf family member;
and optionally:
an inhibitor of p53 function; and/or
one or more additional reprogramming factor(s).

15. A reprogramming method for rejuvenating aged cell(s), tissue(s), organ(s) or organism(s), the method comprising increasing in said aged cell(s), tissue(s), organ(s) or organism(s) the level of, preferably by (co-)expressing in said aged cell(s), tissue(s), organ(s) or organism(s):
a) the SoxB1 factor variant of any one of claims 1 to 5 or the Sox17 factor variant of claim 6, and a POU factor; and/or
b) the fusion protein of claim 7 or the complex or composition of claim 8; and/or
c) the SoxB1 factor variant of claim 5, and a Klf family member;
and optionally:
an inhibitor of p53 function; and/or
one or more additional reprogramming factor(s).

16. A method for conversion of pluripotent cell(s) into high-grade naïve pluripotent cell(s), the method comprising culturing said pluripotent cell(s) under conditions suitable for conversion of said pluripotent cells into high-grade naïve pluripotent cell(s), wherein said conditions comprise increasing in said pluripotent cell(s) the level of, preferably by (co-) expressing in said pluripotent cell(s):
a) a SoxB1 factor or the SoxB1 factor variant of any one of claims 1 to 5 and a Klf family member; and/or
b) the SoxB1 factor variant of claim 5;
and optionally:
an inhibitor of p53 function; and/or
one or more additional reprogramming factor(s).

17. A kit of parts comprising or consisting of
a) the SoxB1 factor variant of any one of claims 1 to 5; and/or
b) the Sox17 factor variant of claim 6; and/or
c) the fusion protein of claim 7; and/or
d) the complex or composition of claim 8; and/or
e) the nucleic acid molecule or the combination of nucleic acid molecules of claim 9; and/or
f) the vector or the combination of vectors of claim 10; and/or
g) the cell of claim 11;
and optionally instructions for use of the kit.

18. A method for producing rejuvenated cell(s), tissue(s), organ(s) or organism(s), the method comprising increasing in aged cell(s), tissue(s), organ(s) or organism(s) the level of, preferably by (co-)expressing in said aged cell(s), tissue(s), organ(s) or organism(s):
a) the SoxB1 factor variant of any one of claims 1 to 5 or the Sox17 factor variant of claim 6, and a POU factor; and/or
b) the fusion protein of claim 7 or the complex or composition of claim 8; and/or
c) the SoxB1 factor variant of claim 5, and a Klf family member;
and optionally, one or more additional reprogramming factor(s);
thereby producing rejuvenated cell(s), tissue(s), organ(s) or organism(s).

19. A method for producing high-grade naïve pluripotent cell(s), the method comprising increasing in low-grade pluripotent cell(s) the level of, preferably by (co-)expressing in said low-grade pluripotent cell(s):
a) a SoxB1 factor or the SoxB1 factor variant of any one of claims 1 to 5 or the Sox17 factor variant of claim 6, and a Klf family member; and/or
b) the fusion protein of claim 7 or the complex or composition of claim 8 and a Klf family member;
and optionally one or more additional reprogramming factor(s);
thereby producing naïve pluripotent cell(s);

20. The method of claim 19, wherein the low-grade pluripotent cell(s) are iPSC(s);
wherein optionally:
the iPSC(s) is/are iPSC(s) obtained or obtainable by the method of claim 14; or
the method of claim 19 further comprises prior to step a) providing the iPSCs by conducting the method of claim 14.

21. The method of any one of claims 14 to 16 and 18 to 20, wherein the (co-)expression is integration-free (co-)expression, preferably wherein the integration-free (co-) expression is from:
the nucleic acid molecule of claim 9; or
the vector of claim 10, wherein the vector is preferably an episomal vector.

22. An induced pluripotent stem cell (iPSC), a high-grade naïve pluripotent cell, or a rejuvenated cell, tissue or organ, produced or producible by the method of any one of claims 14 to 16 and 18 to 21, respectively.

23. The induced pluripotent stem cell (iPSC) of claim 22, wherein the cell is characterized by:
(i) an expression of at least one, preferably at least two, more preferably at least three, pluripotency-specific marker(s) selected from Oct4, Sox2, Nanog and Klf4; and/or
(ii) a capacity to differentiate into each of the three primordial germ layers.

24. The high-grade naïve pluripotent cell of claim 22, wherein the cell is characterized by:
(i) an enhanced developmental potential;
(ii) a higher expression level of at least one, preferably at least two, more preferably at least three, more preferably at least four, more preferably at least five, and most preferably six, naïve pluripotency-specific marker(s) selected from Klf17, Klf4, Sox2, Susd2, Argfx, and Dnmt3l;
(iii) a higher expression level of at least one primitive endoderm-specific marker(s) selected from Gata6, and Sox17;
(iv) an activated POU5f1 distal enhancer;
(v) a reactivated X chromosome in female lines;
(vi) a reduced DNA methylation;
(vii) an enhanced capacity to differentiate, preferably into a germline cell;
(viii) an enhanced capacity to contribute to development of an embryo(s) and/or animal(s); and/or
(ix) a combination of two or more of (i) to (viii);
in comparison to a corresponding non-naïve pluripotent cell or low-grade naïve pluripotent cell.

25. The induced pluripotent stem cell (iPSC), high-grade naïve pluripotent cell or rejuvenated cell, tissue or organ of any one of claims 22 to 24 for use:
as a medicament;
in regenerative medicine; and/or
in treating or preventing an age-related disease.

26. Use of the induced pluripotent stem cell (iPSC), the high-grade naïve pluripotent cell or the rejuvenated cell, tissue, or organ of any one of claims 22 to 24, for producing of differentiated cell(s), tissue(s), organ(s), or organism(s).

27. The use of claim 26, wherein the differentiated cell(s), tissue(s), or organ(s) is/are:
(i) a transplant;
(ii) an organoid; or
(iii) cultured meat.

28. Use of the high-grade naïve pluripotent cell(s) obtained by the method of any one of claims 19 to 21 for producing an embryo(s) or an animal(s), preferably a chimeric embryo(s) or chimeric animal(s).

29. A method for inducing high-grade naïve pluripotency within an embryo(s), thereby enhancing the viability and/or developmental potential of said embryo(s), the method comprising increasing in said embryo(s) the level(s) of:
a) the SoxB1 factor variant of any one of claims 1 to 5 or the Sox17 factor variant of claim 6, and optionally a Klf family member and/or a POU factor;
and/or b) the fusion protein of claim 7 or the complex of claim 8, and optionally a Klf family member.

30. Use of
a) the SoxB1 factor variant of any one of claims 1 to 5; and/or
b) the Sox17 factor variant of claim 6; and/or
c) the fusion protein of claim 7; and/or
d) the complex or composition of claim 8; and/or
e) the nucleic acid molecule or the combination of nucleic acid molecules of claim 9; and/or
f) the vector or the combination of vectors of claim 10, and/or
g) the cell of claims 11 and/or 22,
for the development of culture media for culturing or derivation of naïve pluripotent stem cells.

31. Use of
a) the SoxB1 factor variant of any one of claims 1 to 5; and/or
b) the Sox17 factor variant of claim 6; and/or
c) the fusion protein of claim 7; and/or
d) the complex or composition of claim 8; and/or
e) the nucleic acid molecule or the combination of nucleic acid molecules of claim 9; and/or
f) the vector or the combination of vectors of claim 10, and/or
g) the cell of claim 11 and/or 22,
for the development of differentiation media to derive germline and other cell types and tissues.

32. A germline differentiation medium comprising the cell of claim 11, and a molecule blocking the DNA binding or expression of a member of Oct family.

33. A method of developing or optimizing a culture medium, comprising the step of testing whether the cell of claim 11 and/or 22 retains at least one marker of its phenotype or genotype over at least one cell cycle or passage.

34. A method of developing or optimizing a differentiation medium, preferably a germline differentiation medium, comprising the step of testing whether the cell of claim 11 or 22 displays at least one marker representative of a germline lineage or a cell type further differentiated therefrom.

35. The method of claim 33, wherein said at least one cell cycle or passage is at least 2, 3, 4, 5, 6, 7, 8, 9 or 10 cell cycles or passages.

36. The method of claim 33, 34 or 35, wherein the at least one marker representative of the induced pluripotent stem cell of claim 22 is selected from:
(i) an expression of at least one, preferably at least two, more preferably at least three, pluripotency-specific marker(s) selected from Oct4, Sox2, Nanog and Klf4; and/or
(ii) a capacity to differentiate into each of the three primordial germ layers.

37. The method of claim 33, 34 or 35, wherein the at least one marker representative of the high-grade naïve pluripotent cell of claim 22 is selected from:
(i) an enhanced developmental potential;
(ii) a higher expression level of at least one, preferably at least two, more preferably at least three, more preferably at least four, more preferably at least five, and most preferably six, naïve pluripotency-specific marker(s) selected from Klf17, Klf4, Sox2, Susd2, Argfx, and Dnmt3l;
(iii) a higher expression level of at least one primitive endoderm-specific marker(s) selected from Gata6, and Sox17;
(iv) an activated POU5f1 distal enhancer;
(v) a reactivated X chromosome in female lines;
(vi) a reduced DNA methylation;
(vii) an enhanced capacity to differentiate, preferably into a germline cell;
(viii) an enhanced capacity to contribute to development of an embryo(s) and/or animal(s); and/or
(ix) a combination of two or more of (i) to (viii);
in comparison to a corresponding non-naïve pluripotent cell or low-grade naïve pluripotent cell.

38. The method of any one of claims 33 to 35, wherein retaining the at least one marker of the phenotype of the cell of claim 11 comprises retaining at least one of an enhanced developmental potential, a higher expression level of at least one, preferably at least two, more preferably at least three, more preferably at least four, more preferably at least five, and most preferably at least six naïve pluripotency-specific marker(s) selected from Klf17, Klf4, Sox2, Susd2, Argfx, and Dnmt3l, a higher expression level of at least one primitive endoderm-specific marker(s) selected from Gata6 and Sox17, an activated POU5f1 distal enhancer, a reactivated X chromosome in female lines, a reduced DNA methylation, an enhanced capacity to differentiate, preferably into a germline cell, and/or an enhanced capacity to contribute to development of an embryo(s) and/or animal(s), of the phenotype of the original cell carrying a SoxB1 factor variant of the any one of claims 1 to 5, or the Sox17 factor variant of claim 6.

39. The method of any one of claims 33 to 38 further comprising producing the developed or optimized medium.

40. A cell culture medium produced or producible by the method of any one of claims 33 or 35 to 39.

41. A germline differentiation medium produced or producible by the method of any one of claims 34 to 39.
